SEQUENCE LISTING (44 sequences)

SEQ ID NO: 1 - DNA, 393 bp, synthetic construct
  source 1..393: mol_type = other DNA
ggtgaagctgcaggagtcaggacctagcctggtgaagccttctcagtctctgtccctcacctgcactgtcactggcttctcaatcacccgtgattatgcctggaactggatccggcagtttccaggaaacaaagtggagtggatgggtaacataaattacagtggtaccactaactacaacccatctctcgaaagtcgaatctctatcactcgagacacatccaagaaccagttcttcctgcagttgaattctgtgactactgaggacacagccacatattactgtgtaggatgggcggtaaattatggtttggactactggggtccaggaacctcagtcaccgtctcctcagccaaaacgacacccaagcttgtctatccactggcccctgg

SEQ ID NO: 2 - DNA, 395 bp, synthetic construct
  source 1..395: mol_type = other DNA
ccaggggccagtggatagacaagcttgggtgtcgttttggctgaggagacggtgactgaggttcctggaccccagtagtccaaaccataatttaccgcccatcctacacagtaatatgtggctgtgtcctcagtagtcacagaattcaactgcaggaagaactggttcttggatgtgtctcgagtgatagagattcgactttcgagagatgggttgtagttagtggtaccactgtaatttatgttacccatccactccactttgtttcctggaaactgccggatccagttccaggcataatcacgggtgattgagaagccagtgacagtgcaggtgagggacagagactgagaaggcttcaccaggctaggtcctgactcctgcagcttcacctc

SEQ ID NO: 3 - DNA, 395 bp, synthetic construct
  source 1..395: mol_type = other DNA
gaggtgaagctgcaggagtcaggacctagcctggtgaagccttctcagtctctgtccctcacctgcactgtcactggcttctcaatcacccgtgattatgcctggaactggatccggcagtttccaggaaacaaagtggagtggatgggtaacataaattacagtggtaccactaactacaacccatctctcgaaagtcgaatctctatcactcgagacacatccaagaaccagttcttcctgcagttgaattctgtgactactgaggacacagccacatattactgtgtaggatgggcggtaaattatggtttggactactggggtccaggaacctcagtcaccgtctcctcagccaaaacgacacccaagcttgtctatccactggcccctgg

SEQ ID NO: 4 - DNA, 394 bp, synthetic construct
  source 1..394: mol_type = other DNA
ccaggggccagtggatagacaagcttgggtgtcgttttggctgaggagacggtgactgaggttcctggaccccagtagtccaaaccataatttaccgcccatcctacacagtaatatgtggctgtgtcctcagtagtcacagaattcaactgcaggaagaactggttcttggatgtgtctcgagtgatagagattcgactttcgagagatgggttgtagttagtggtaccactgtaatttatgttacccatccactccactttgtttcctggaaactgccggatccagttccaggcataatcacgggtgattgagaagccagtgacagtgcaggtgagggacagagactgagaaggcttcaccaggccaggtccagactcctgcagctgcacct

SEQ ID NO: 5 - DNA, 388 bp, synthetic construct
  source 1..388: mol_type = other DNA
agctgcaggagtctggacctggcctggtgaagccttctcagtctctgtccctcacctgcactgtcactggcttctcaatcacccgtgattatgcctggaactggatccggcagtttccaggaaacaaagtggagtggatgggtaacataaattacagtggtaccactaactacaacccatctctcgaaagtcgaatctctatcactcgagacacatccaagaaccagttcttcctgcagttgaattctgtgactactgaggacacagccacatattactgtgtaggatgggcggtaaattatggtttggactactggggtccaggaacctcagtcaccgtctcctcagccaaaacgacacccaagcttgtctatccactggcccctgg

SEQ ID NO: 6 - DNA, 394 bp, synthetic construct
  source 1..394: mol_type = other DNA
ccaggggccagtggatagacaagcttgggtgtcgttttggctgaggagacggtgactgaggttcctggaccccagtagtccaaaccataatttaccgcccatcctacacagtaatatgtggctgtgtcctcagtagtcacagaattcaactgcaggaagaactggttcttggatgtgtctcgagtgatagagattcgactttcgagagatgggttgtagttagtggtaccactgtaatttatgttacccatccactccactttgtttcctggaaactgccggatccagttccaggcataatcacgggtgattgagaagccagtgacagtgcaggtgagggacagagactgagaaggcttcaccaggccaggtccagactcctgcagcttgacct

SEQ ID NO: 7 - DNA, 367 bp, synthetic construct
  source 1..367: mol_type = other DNA
cagatgcagcttcaggagtcgggacctggcctggtgaagccttctcagtctctgtccctcacctgcactgtcactggcttctcaatcacccgtgattatgcctggaactggatccggcagtttccaggaaacaaagtggagtggatgggtaacataaattacagtggtaccactaactacaacccatctctcgaaagtcgaatctctatcactcgagacacatccaagaaccagttcttcctgcagttgaattctgtgactactgaggacacagccacatattactgtgtaggatgggcggtaaattatggtttggactactggggtccaggaacctcagtcaccgtctcctcaaacttaattaacg

SEQ ID NO: 8 - DNA, 367 bp, synthetic construct
  source 1..367: mol_type = other DNA
cgttaattaagtttgaggagacggtgactgaggttcctggaccccagtagtccaaaccataatttaccgcccatcctacacagtaatatgtggctgtgtcctcagtagtcacagaattcaactgcaggaagaactggttcttggatgtgtctcgagtgatagagattcgactttcgagagatgggttgtagttagtggtaccactgtaatttatgttacccatccactccactttgtttcctggaaactgccggatccagttccaggcataatcacgggtgattgagaagccagtgacagtgcaggtgagggacagagactgagaaggcttcaccaggccaggtcccgactcctgaagctgcacatc

SEQ ID NO: 9 - DNA, 367 bp, synthetic construct
  source 1..367: mol_type = other DNA
gaggtgcagctgaaggagtcgggacctggcctggtgaagccttctcagtctctgtccctcacctgcactgtcactggcttctcaatcacccgtgattatgcctggaactggatccggcagtttccaggaaacaaagtggagtggatgggtaacataaattacagtggtaccactaactacaacccatctctcgaaagtcgaatctctatcactcgagacacatccaagaaccagttcttcctgcagttgaattctgtgactactgaggacacagccacatattactgtgtaggatgggcggtaaattatggtttggactactggggtccaggaacctcagtcaccgtctcctcaaacttaattaacg

SEQ ID NO: 10 - DNA, 367 bp, synthetic construct; identical to SEQ ID NO: 9
  source 1..367: mol_type = other DNA

SEQ ID NO: 11 - DNA, 370 bp, synthetic construct
  source 1..370: mol_type = other DNA
agatggatccagttggtgcagcatcagcccgttttatttccagcttggtcccccctccgaacgtgtgaggaaaatgtgtaccttgcacgcagtaataaactcccaaatcctcagcctccactctgctgattttcagtgtaaaatctgttcctgatccactgccaatgaacctgtcagggactccagagtccagtttagacaccagatagattaggcgctttggagactggcctggcctctgtaataaccaattcaaataggtttttccattactatataagaggctctgacttgacctgcaagagatagaggctggttgtccaaaggtaaccgacaaagtgagttcatcctgtgttatcacgatatcacc

SEQ ID NO: 12 - DNA, 374 bp, synthetic construct
  source 1..374: mol_type = other DNA
gggaagatggatccagttggtgcagcatcagcccgttttatttccagcttggtcccccctccgaacgtgtgaggaaaatgtgtaccttgcacgcagtaataaactcccaaatcctcagcctccactctgctgattttcagtgtaaaatctgttcctgatccactgccaatgaacctgtcagggactccagagtccagtttagacaccagatagattaggcgctttggagactggcctggcctctgtaataaccaattcaaataggtttttccattactatataagaggctctgacttgacctgcaagagatagaggctggttgtccaaaggtaaccgacaaagtgagttcatcttgtgtcatcacgatatcacc

SEQ ID NO: 13 - DNA, 374 bp, synthetic construct
  source 1..374: mol_type = other DNA
gggaagatggatccagttggtgcagcatcagcccgttttatttccagcttggtcccccctccgaacgtgtgaggaaaatgtgtaccttgcacgcagtaataaactcccaaatcctcagcctccactctgctgattttcagtgtaaaatctgttcctgatccactgccaatgaacctgtcagggactccagagtccagtttagacaccagatagattaggcgctttggagactggcctggcctctgtaataaccaattcaaataggtttttccattactatataagaggctctgacttgacctgcaagagatagaggctggttgtccaaaggtaaccgacaaagtgagttcatcctgtgttatcacgatatcacc

SEQ ID NO: 14 - DNA, 349 bp, synthetic construct
  source 1..349: mol_type = other DNA
gatgttgtgatgacccagaatccactcactttgtcggttacctttggacaaccagcctctatctcttgcaggtcaagtcagagcctcttatatagtaatggaaaaacctatttgaattggttattacagaggccaggccagtctccaaagcgcctaatctatctggtgtctaaactggactctggagtccctgacaggttcattggcagtggatcaggaacagattttacactgaaaatcagcagagtggaggctgaggatttgggagtttattactgcgtgcaaggtacacattttcctcacacgttcggaggggggaccaagctggaaatcaaacgttcggccgtcg

SEQ ID NO: 15 - DNA, 349 bp, synthetic construct
  source 1..349: mol_type = other DNA
cgacggccgaacgttttatttccagcttggtcccccctccgaacgtgtgaggaaaatgtgtaccttgcacgcagtaataaactcccaaatcctcagcctccactctgctgattttcagtgtaaaatctgttcctgatccactgccaatgaacctgtcagggactccagagtccagtttagacaccagatagattaggcgctttggagactggcctggcctctgtaataaccaattcaaataggtttttccattactatataagaggctctgacttgacctgcaagagatagaggctggttgtccaaaggtaaccgacaaagtgagtggattctgggtcatcacaacatc

SEQ ID NO: 16 - DNA, 342 bp, synthetic construct
  source 1..342: mol_type = other DNA
cgacggccgaacgtttgatttccagcttggtcccccctccgaacgtgtaagctccctaatgtgctgacagtaataggttgcagcatcctcctcctccacaggatggatgttgagggtgaagtctgtcccagacccactgccactgaacctggcagggaccccagattctaggttggatacaagatagatgaggagtctgggtggctgtcctggtttctgttggttccagtgcatataactatagccagatgtactgacacttttgctggccctgtatgagatggtggccctctgccccagagatacagctaaggaagcaggagactgtgacatcacaatgtc

SEQ ID NO: 17 - DNA, 343 bp, synthetic construct
  source 1..343: mol_type = other DNA
cgacggccgaacgtttgatttccagcttggtcccccctccgaacgtgtaagctccctaatgtgctgacagtaataggttgcagcatcctcctcctccacaggatggatgttgagggtgaagtctgtcccagacccactgccactgaacctggcagggaccccagattctaggttggatacaagatagatgaggagtctgggtggctgtcctggtttctgttggttccagtgcatataactatagccagatgtactgacacttttgctggccctgtatgagatggtggccctctgccccagagatacagctaaggaagcaggagactgtgtcagcacaatgtcd

SEQ ID NO: 18 - protein, 118 aa, synthetic construct
EVQLQESGPGLVKPSQSLSLTCTVTGFSITRDYAWNWIRQFPGNKVEWMGNINYSGTTNYNPSLESRISITRDTSKNQFFLQLNSVTTEDTATYYCVGWAVNYGLDYWGPGTSVTVSS

SEQ ID NO: 19 - protein, 124 aa, synthetic construct
GDIVMTQDELTLSVTFGQPASISCRSSQSLLYSNGKTYLNWLLQRPGQSPKRLIYLVSKLDSGVPDRFIGSGSGTDFTLKISRVEAEDLGVYYCVQGTHFPHTFGGGTKLEIKRADAAPTGSIF

SEQ ID NO: 20 - protein, 114 aa, synthetic construct
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWKSNVRPS

SEQ ID NO: 21 - protein, 128 aa, synthetic construct
EVQLQESGPXGLVKPSQSLSLTCTVTGFSITXXXRDYAWNWIRQFPGNKVEWMGNINYSXXXGTTNYNPSLEXSRISITRDTSKNQFFLQLNSVTTEDTZTYYCVGWAVNXXYGLDYWGPGTSVTVSS

SEQ ID NO: 22 - protein, 128 aa, synthetic construct
DIVMTQDELTLSVTFGQPASISCRSSQSLLYSXNGKTYLNWLLQRPGQSPKRLIYLVXXXXXXXSKLDSGVPXDRFIGSGXXSGTDFTLKISRVEAEDLGVYYCVQGTHXXXXFPHTFGGGTKLEIKX

SEQ ID NO: 23 - protein, 117 aa, synthetic construct
DIVLTQSPASLAVSLGQRATISYRASKSVSTXXSGYSYMHWNQQKPGQPPRLLIYLVXXXXXXXSNLESGVPXARFSGSGXXSGTDFTLNIHPVEEEDAATYYCQHIRELXTRSEGG

SEQ ID NO: 24 - protein, 130 aa, synthetic construct
VKLQESGPSLVKPSQSLSLTCTVTGFSITRDYAWNWIRQFPGNKVEWMGNINYSGTTNYNPSLESRISITRDTSKNQFFLQLNSVTTEDTATYYCVGWAVNYGLDYWGPGTSVTVSSAKTTPKLVYPLAP

SEQ ID NO: 25 - protein, 131 aa, synthetic construct
EVKLQESGPSLVKPSQSLSLTCTVTGFSITRDYAWNWIRQFPGNKVEWMGNINYSGTTNYNPSLESRISITRDTSKNQFFLQLNSVTTEDTATYYCVGWAVNYGLDYWGPGTSVTVSSAKTTPKLVYPLAP

SEQ ID NO: 26 - protein, 131 aa, synthetic construct; identical to SEQ ID NO: 25

SEQ ID NO: 27 - protein, 130 aa, synthetic construct
VQLQESGPGLVKPSQSLSLTCTVTGFSITRDYAWNWIRQFPGNKVEWMGNINYSGTTNYNPSLESRISITRDTSKNQFFLQLNSVTTEDTATYYCVGWAVNYGLDYWGPGTSVTVSSAKTTPKLVYPLAP

SEQ ID NO: 28 - protein, 128 aa, synthetic construct
LQESGPGLVKPSQSLSLTCTVTGFSITRDYAWNWIRQFPGNKVEWMGNINYSGTTNYNPSLESRISITRDTSKNQFFLQLNSVTTEDTATYYCVGWAVNYGLDYWGPGTSVTVSSAKTTPKLVYPLAP

SEQ ID NO: 29 - protein, 130 aa, synthetic construct
VKLQESGPGLVKPSQSLSLTCTVTGFSITRDYAWNWIRQFPGNKVEWMGNINYSGTTNYNPSLESRISITRDTSKNQFFLQLNSVTTEDTATYYCVGWAVNYGLDYWGPGTSVTVSSAKTTPKLVYPLAP

SEQ ID NO: 30 - protein, 122 aa, synthetic construct
QMQLQESGPGLVKPSQSLSLTCTVTGFSITRDYAWNWIRQFPGNKVEWMGNINYSGTTNYNPSLESRISITRDTSKNQFFLQLNSVTTEDTATYYCVGWAVNYGLDYWGPGTSVTVSSNLIN

SEQ ID NO: 31 - protein, 122 aa, synthetic construct
DVQLQESGPGLVKPSQSLSLTCTVTGFSITRDYAWNWIRQFPGNKVEWMGNINYSGTTNYDVQLQESGPGLVKPSQSLSLTCTVTGFSITRDYAWNWIRQFPGNKVEWMGNINYSGTTNYIN

SEQ ID NO: 32 - protein, 122 aa, synthetic construct
EVQLKESGPGLVKPSQSLSLTCTVTGFSITRDYAWNWIRQFPGNKVEWMGNINYSGTTNYNPSLESRISITRDTSKNQFFLQLNSVTTEDTATYYCVGWAVNYGLDYWGPGTSVTVSSNLIN

SEQ ID NO: 33 - protein, 122 aa, synthetic construct; identical to SEQ ID NO: 32

SEQ ID NO: 34 - protein, 118 aa, synthetic construct; identical to SEQ ID NO: 18

SEQ ID NO: 35 - protein, 123 aa, synthetic construct
GDIVITQDELTLSVTFGQPASISCRSSQSLLYSNGKTYLNWLLQRPGQSPKRLIYLVSKLDSGVPDRFIGSGSGTDFTLKISRVEAEDLGVYYCVQGTHFPHTFGGGTKLEIKRADAAPTGSI

SEQ ID NO: 36 - protein, 124 aa, synthetic construct; identical to SEQ ID NO: 19

SEQ ID NO: 37 - protein, 124 aa, synthetic construct
GDIVITQDELTLSVTFGQPASISCRSSQSLLYSNGKTYLNWLLQRPGQSPKRLIYLVSKLDSGVPDRFIGSGSGTDFTLKISRVEAEDLGVYYCVQGTHFPHTFGGGTKLEIKRADAAPTGSIF

SEQ ID NO: 38 - protein, 116 aa, synthetic construct
DVVMTQNPLTLSVTFGQPASISCRSSQSLLYSNGKTYLNWLLQRPGQSPKRLIYLVSKLDSGVPDRFIGSGSGTDFTLKISRVEAEDLGVYYCVQGTHFPHTFGGGTKLEIKRSAV

SEQ ID NO: 39 - protein, 116 aa, synthetic construct; identical to SEQ ID NO: 38

SEQ ID NO: 40 - protein, 124 aa, synthetic construct; identical to SEQ ID NO: 19

SEQ ID NO: 41 - protein, 114 aa, synthetic construct
DIVMSQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWKSNVRPS

SEQ ID NO: 42 - protein, 114 aa, synthetic construct; identical to SEQ ID NO: 20

SEQ ID NO: 43 - protein, 114 aa, synthetic construct; identical to SEQ ID NO: 20

SEQ ID NO: 44 - RNA, 3983 nt, Homo sapiens
  source 1..3983: mol_type = mRNA
gctcaaaagttcagaagttttcccaatgcttccttaagcggctggcgcgcgagagaccgagaaaaggtgacgcggggcccgggcaggcggccggcgcgcggccccccccccccccgccctggttatttggccgccttcgccggcagctcagggcagagtctcctggaaggcgcaggcagtgtggcgagaagggcgcctgcttgttctttctttttgtctgctttcccccgtttgcgcctggaagctgcgccgcgagttcctgcaaggcggtctgccgcggccgggcccggccttctcccctcgcagcgaccccgcctcgcggccgcgcgggccccgaggtagcccgaggcgccggaggagccagccccagcgagcgccgggagaggcggcagcgcagccggacgcacagcgcagcgggccggcaccagctcggccgggcccggactcggactcggcggccggcgcggcgcggcccggcccgagcgagggtggggggcggcgggcggcgcggggcggcggcgagcgggggccatgcaggcgcgctactccgtgtccagccccaactccctgggagtggtgccctacctcggcggcgagcagagctactaccgcgcggcggccgcggcggccgggggcggctacaccgccatgccggcccccatgagcgtgtactcgcaccctgcgcacgccgagcagtacccgggcggcatggcccgcgcctacgggccctacacgccgcagccgcagcccaaggacatggtgaagccgccctatagctacatcgcgctcatcaccatggccatccagaacgccccggacaagaagatcaccctgaacggcatctaccagttcatcatggaccgcttccccttctaccgggacaacaagcagggctggcagaacagcatccgccacaacctctcgctcaacgagtgcttcgtcaaggtgccgcgcgacgacaagaagccgggcaagggcagctactggacgctggacccggactcctacaacatgttcgagaacggcagcttcctgcggcggcggcggcgcttcaagaagaaggacgcggtgaaggacaaggaggagaaggacaggctgcacctcaaggagccgcccccgcccggccgccagcccccgcccgcgccgccggagcaggccgacggcaacgcgcccggtccgcagccgccgcccgtgcgcatccaggacatcaagaccgagaacggtacgtgcccctcgccgccccagcccctgtccccggccgccgccctgggcagcggcagcgccgccgcggtgcccaagatcgagagccccgacagcagcagcagcagcctgtccagcgggagcagccccccgggcagcctgccgtcggcgcggccgctcagcctggacggtgcggattccgcgccgccgccgcccgcgccctccgccccgccgccgcaccatagccagggcttcagcgtggacaacatcatgacgtcgctgcgggggtcgccgcagagcgcggccgcggagctcagctccggccttctggcctcggcggccgcgtcctcgcgcgcggggatcgcacccccgctggcgctcggcgcctactcgcccggccagagctccctctacagctccccctgcagccagacctccagcgcgggcagctcgggcggcggcggcggcggcgcgggggccgcggggggcgcgggcggcgccgggacctaccactgcaacctgcaagccatgagcctgtacgcggccggcgagcgcgggggccacttgcagggcgcgcccgggggcgcgggcggctcggccgtggacgaccccctgcccgactactctctgcctccggtcaccagcagcagctcgtcgtccctgagtcacggcggcggcggcggcggcggcgggggaggccaggaggccggccaccaccctgcggcccaccaaggccgcctcacctcgtggtacctgaaccaggcgggcggagacctgggccacttggcgagcgcggcggcggcggcggcggccgcaggctacccgggccagcagcagaacttccactcggtgcgggagatgttcgagtcacagaggatcggcttgaacaactctccagtgaacgggaatagtagctgtcaaatggccttcccttccagccagtctctgtaccgcacgtccggagctttcgtctacgactgtagcaagttttgacacaccctcaaagccgaactaaatcgaaccccaaagcaggaaaagctaaaggaacccatcaaggcaaaatcgaaactaaaaaaaaaaaatccaattaaaaaaaacccctgagaatattcaccacaccagcgaacagaatatccctccaaaaattcagctcaccagcaccagcacgaagaaaactctattttcttaaccgattaattcagagccacctccactttgccttgtctaaataaacaaacccgtaaactgttttatacagagacagcaaaatcttggtttattaaaggacagtgttactccagataacacgtaagtttcttcttgcttttcagagacctgctttcccctcctcccgtctcccctctcttgccttcttccttgcctctcacctgtaagatattattttatcctatgttgaagggagggggaaagtccccgtttatgaaagtcgctttctttttattcatggacttgttttaaaatgtaaattgcaacatagtaatttatttttaatttgtagttggatgtcgtggaccaaacgccagaaagtgttcccaaaacctgacgttaaattgcctgaaactttaaattgtgctttttttctcattataaaaagggaaactgtattaatcttattctatcctcttttctttctttttgttgaacatattcattgtttgtttattaataaattaccattcagtttgaatgagacctatatgtctggatactttaatagagctttaattattacgaaaaaagatttcagagataaaacactagaagttacctattctccacctaaatctctgaaaaatggagaaaccctctgactagtccatgtcaaattttactaaaagtctttttgtttagatttattttcctgcagcatcttctgcaaaatgtactatatagtcagcttgctttgaggctagtaaaaagatatttttctaaacagattggagttggcatataaacaaatacgttttctcactaatgacagtccatgattcggaaattttaagcccatgaatcagccgcggtcttaccacggtgatgcctgtgtgccgagagatgggactgtgcggccagatatgcacagataaatatttggcttgtgtattccatataaaattgcagtgcatattatacatccctgtgagccagatgctgaatagatattttcctattatttcagtcctttataaaaggaaaaataaaccagtttttaaatgtatgtatataattctcccccatttacaatccttcatgtattacatagaaggattgcttttttaaaaatatactgcgggttggaaagggatatttaatctttgagaaactattttagaaaatatgtttgtagaacaattatttttgaaaaagatttaaagcaataacaagaaggaaggcgagaggagcagaacattttggtctagggtggtttctttttaaaccattttttcttgttaatttacagttaaacctaggggacaatccggattggccctcccccttttgtaaataacccaggaaatgtaataaattcattatcttagggtgatctgccctgccaatcagactttggggagatggcgatttgattacagacgttcgggggggtggggggcttgcagtttgttttggagataatacagtttcctgctatctgccgctcctatctagaggcaacacttaagcagtaattgctgttgcttgttgtcaaaatttgatcattgttaaaggattgctgcaaataaatacactttaatttcagtcaaaaa

(19) United States
(12) Patent Application Publication
(10) Pub. No.: US 2025/0283178 A1
(43) Pub. Date: Sep. 11, 2025

(54) FOXC1 EXPRESSION AS A COMPLEMENTARY DIAGNOSTIC ASSAY FOR PREDICTING EFFICACY OF ONCOLOGY DRUGS AND DRUG COMBINATION REGIMENS AND METHODS FOR USE

(71) Applicant: 3N DIAGNOSTICS LTD., Belfast (GB)

(72) Inventors: Partha S. Ray, Evanston, IL (US); Roberto Fagnani, San Diego, CA (US)

(73) Assignee: 3N DIAGNOSTICS LTD., Belfast (GB)

(21) Appl. No.: 18/862,858

(22) PCT Filed: May 4, 2023
(86) PCT No.: PCT/IB23/54671

§ 371 Date: Nov. 4, 2024

Related U.S. Application Data
(60) Provisional application No. 63/364,187, filed on May 4, 2022.

(51) Int. Cl. C12Q 1/6886; A61K 45/06; C07K 16/18; C12Q 1/686

(52) U.S. Cl. CPC C12Q 1/6886; A61K 45/06; C07K 16/18; C12Q 1/686; C07K 2317/31; C07K 2317/565; C07K 2317/622; C12Q 2600/106

(57) ABSTRACT

Antibodies for determining the level of FOXC1 protein in a subject's tumor and methods of using FOXC1 antibodies to predict clinical efficacy of a cancer therapeutic, predict prognosis of a subject receiving the cancer therapeutic, and treat the subject based on the predictions of clinical efficacy and prognosis.

### CROSS-REFERENCE TO RELATED APPLICATION

This application claims the benefit of U.S. Provisional Patent Application No. 63/364,187, filed May 4, 2022, which is incorporated herein by reference in its entirety.

### SEQUENCE LISTING

This application contains an ST.26 compliant Sequence Listing, which is submitted concurrently in xml format via EFS-Web or Patent Center and is hereby incorporated by reference in its entirety. The .xml copy, created on May 2, 2023, is named 3ND Sequence Listing 144654.8001.W000.xml and is 52 KB in size.

### BACKGROUND

Cancer screening strategies have proven to be successful in decreasing cancer-specific mortality in a variety of cancers (Tabar et al., 1985; Labrie et al., 1999; Mapp et al., 1999; Andrae et al., 2012). However, the clinical cure rates of patients who are diagnosed with cancer despite such screening measures still largely remain far from ideal (Sung et al., 2021). The ability to potentially monitor cancer recurrence and metastasis events in real-time has now become possible with the development of various liquid biopsy modalities (circulating tumor cells, extracellular vesicles, cell-free DNA, etc.) (Keup et al., 2021). However, if current rates of cancer disease progression and exert meaningful decreases in cancer morbidity and mortality are to be favorably impacted, there is a need to improve upon the ability to predict which newly-diagnosed patients are at the highest risk of suffering recurrence and metastasis events in the future. Biopsy-tissue derived molecular markers that predict heightened future metastasis risk still present the most pragmatic solution to this problem. This is because detection of such biomarkers utilizing standard immunohistochemistry (IHC) has a much higher likelihood of widespread global adoption owing to superior cost effectiveness and greater ease of integration into existing workflow of diagnostic pathology laboratories (Swanson, 2015; Sheffield, 2016).

Traditional clinical factors alone (such as TNM staging criteria) are no longer prognostically adequate for this purpose and there is a need for new prognostic biomarkers that are superior in their ability to predict cancer recurrence and metastasis risk (Burke, 2004). Absent such markers, the ability to pinpoint and identify which patients stand to derive the greatest clinical benefit of new therapeutic approaches being tested in clinical trials will be lost. As a result of not being able to “enrich” clinical trial populations with those patients who are most likely to derive survival benefit, it will ostensibly dilute the measured therapeutic efficacy in such trials, and may erroneously label a tested approach as being ineffective (Wang et al., 2009). Such biomarkers, once identified, may or may not play a functional or mechanistic role in driving the underlying aggressive biology contributing to the observed adverse outcomes. For this reason, recurrence/metastasis risk prediction needs to be performed in conjunction with the identification of the pivotal underlying molecular drivers responsible for increasing the probability of suffering a recurrence/metastasis event. This would allow formulation of a rational therapeutic strategy, based on targeting the identified underlying molecular driver mechanism, to ultimately derive clinical benefit.

### BRIEF DESCRIPTION OF THE DRAWINGS

FIG. 1. CLINICAL HALLMARKS OF FOXC1-OVEREXPRESSING BREAST CANCER—METASTATIC RECURRENCE DESPITE ADEQUATE SURGICAL RESECTION, LYMPH NODE ASSESSMENT AND REMOVAL, RADIATION THERAPY AND CHEMOTHERAPY. For a biomarker to be associated with such aggressive behavior, resistant to common therapeutic treatment approaches, argues strongly in favor of this being a manifestation of the cancer stem cell phenotype.

FIG. 2. CHARACTERISTICS OF FOXC1-OVEREXPRESSING PRO-METASTATIC CANCERS. 1. Plasticity whereby FOXC1+ cells undergo partial Epithelial-to-Mesenchymal Transition (EMT), and can revert back by undergoing a partial Mesenchymal-to-Epithelial Transition (MET). Detection of FOXC1+E/M hybrid biphenotypic Circulating Tumor Cells (CTCs) in the peripheral blood of breast cancer patients provides clinical evidence supporting the occurrence of this phenomenon in vivo. 2. Chemoresistance to a variety of chemotherapeutic agents driven by FOXC1 has been described in multiple cancer types with varied mechanisms. 3. Radiation-induced adaptation and subsequent resistance has been described in two different types of cancer cell line models to be characterized by FOXC1 overexpression. 4. Stem cell pathway activation, particularly of the non-canonical variety has been described for NFκB, Wnt, Hedgehog, P13K/AKT/mTOR and TGFβ signaling pathways in cancer. Moreover, they all converge on FOXC1. This has led to the suggestion that combination therapy with two or more pathway inhibitor drugs may be necessary to block FOXC1-driven cancer metastasis. 5. Superenhancer-driven transcriptional addiction to FOXC1 mediated by CDK7 has been described in breast cancer and was shown to be effectively thwarted using a CDK7 inhibitor drug. 6. FOXC1 contributes to an immunosuppressive microenvironment by upregulating multiple immunosuppressive factors including HIF1a, CXCR4, CXCR1 and LOX1. This helps explain how the cancer stem cell phenotype helps evade immune detection, and how FOXC1+ cancers are a valid target for immunotherapy approaches like immune checkpoint inhibitors.

FIG. 3. TARGETED NFκB, RAS/MAPK and P13K/AKT/mTOR THERAPY STRATEGIES FOR FOXC1-OVEREXPRESSING PROMETASTATIC CANCERS—1. Drugs that target the NFκB signaling pathway (Binding of transcriptional coactivator and RNA polymerase to p50/p65 heterodimer not shown). Indirect targeting opportunities are also depicted where targeted inhibition of alternate signaling pathways effectively blocks activation of the NFκB signaling pathway as well. These include targeting of the EGFR receptor, various members of the RAS/MAPK signaling pathway (including RAS, RAF, MEK and ERK), as well as the individual members of the P13K/AKT/mTOR signaling pathway.

FIG. 4. TARGETED TGF-β and HEDGEHOG THERAPY STRATEGIES FOR FOXC1-OVEREXPRESSING PRO-METASTATIC CANCERS-II. Drugs that target the TGF Beta and Hedgehog signaling pathways.

FIG. 5. TARGETED TME THERAPY STRATEGIES FOR TARGETING FOXC1-OVEREXPRESSING PRO-METASTATIC CANCERS—Ill. Drugs that target the IL8, CXCR1, CXCR4, FGFR1 and FGFR4 mediated cell-cell interactions in the tumor microenvironment.

FIGURE. 6. Collier-de-Perles of heavy chain variable region of AK 3295 antibody (SEQ ID NO: 21). Blue shaded circles represent hydrophobic (non-polar) residues that are hydrophobic in the majority of antibodies. Yellow shaded circles represent proline residues. Squares indicate key residues at start and end of CDR. Red amino acids are structurally conserved in the framework.

FIG. 7. Collier-de-Perles of light chain variable region of AK 3295 antibody (SEQ ID NO: 22). Blue shaded circles represent hydrophobic (non-polar) residues that are hydrophobic in the majority of antibodies. Yellow shaded circles represent proline residues. Squares indicate key residues at start and end of CDR. Red amino acids are structurally conserved in the framework.

FIG. 8. Collier-de-Perles of light chain variable region of AK 3295 antibody (SEQ ID NO: 23). Blue shaded circles represent hydrophobic (non-polar) residues that are hydrophobic in the majority of antibodies. Yellow shaded circles represent proline residues. Squares indicate key residues at start and end of CDR. Red amino acids are structurally conserved in the framework.

FIG. 9 is a CLUSTAL 0(1.2.4) multiple sequence alignment according to certain embodiments (SEQ ID NO: 24—SEQ ID NO: 34).

FIG. 10 is a CLUSTAL 0(1.2.4) multiple sequence alignment according to certain embodiments (SEQ ID NO: 35—SEQ ID NO: 40).

FIG. 11 is a CLUSTAL 0(1.2.4) multiple sequence alignment according to certain embodiments (SEQ ID NO: 41—SEQ ID NO: 43).

FIG. 12 shows the pathologic complete response rates from subjects that have triple negative breast cancer tumors with high or low FOXC1 expression and the sensitivity and specificity of FOXC1 expression as a predictor of NACT response.

FIG. 13 shows the sensitivity and specificity of FOXC1 expression as a predictor of NACT response measured as pathologic complete response rate vs. residual disease.

FIG. 14 shows the sensitivity and specificity of FOXC1 expression as a predictor of NACT response measured as pCR patients and patients with minimal cancer burden (residual cancer burden-I, RCB-I), compared with patients who had moderate or excessive burden (residual cancer burden-II and Ill, RCB-II and RCB-III) after treatment with taxane and platinum.

FIG. 15 shows the pathologic complete response rates from subjects that have triple negative breast cancer tumors with high or low FOXC1 and MK167 expression and the sensitivity and specificity of FOXC1 expression as a predictor of response after treatment with durvalumab.

FIG. 16 shows the pathologic complete response rates from subjects that have HER2 negative ER+ cancer tumors with high or low FOXC1 and MK167 expression and the sensitivity and specificity of FOXC1 expression as a predictor of response after treatment with durvalumab.

FIG. 17A shows the pathologic complete response rates from a first cohort of subjects that have triple negative breast cancer tumors with high FOXC1, MK167, and low PDL1 expression and the sensitivity and specificity of high FOXC1, MK167, and low PDL1 expression as a predictor of response after treatment with immune checkpoint inhibitors.

FIG. 17B shows the pathologic complete response rates from a second cohort of subjects that have triple negative breast cancer tumors with high FOXC1, MK167, and low PDL1 expression and the sensitivity and specificity of high FOXC1, MK167, and low PDL1 expression as a predictor of response after treatment with immune checkpoint inhibitors.

FIG. 17C shows the pathologic complete response rates from a third cohort of subjects that have triple negative breast cancer tumors with high FOXC1, MK167, and low PDL1 expression and the sensitivity and specificity of high FOXC1, MK167, and low PDL1 expression as a predictor of response after treatment with immune checkpoint inhibitors.

FIG. 18A shows a schematic of an example of the therapeutic response categories in the overall population of muscle-invasive bladder cancer (MIBC) patients treated with immune checkpoint inhibitors, if not segregated on the basis of a predicted biomarker strategy as well as a bar graph showing how the Predicted Responder (PR) and Predicted Non-responder (NR) categorization using our predictive strategy was independent of earlier described RNA Molecular Subtype categories of MIBC according to the Lund Classification System. NR=Predicted Non-responder. PR=Predicted Responder.

FIG. 18B shows the pathologic complete response rates from subjects that have invasive bladder tumors with high FOXC1 and MK167 expression and the sensitivity and specificity of high FOXC1 and MK167 expression as a predictor of response after treatment with immune checkpoint inhibitors.

FIGS. 19A and 19B show that RFS of Predicted Responders (PR) based on the MK167 and FOXC1 complementary diagnostic approach is superior to that of Predicted Non-Responders (NR).

FIG. 19C shows that CSS of Predicted Responders (PR) based on the MK167 and FOXC1 complementary diagnostic approach is superior to that of Predicted Non-Responders (NR) using the same method and is statistically significant.

FIG. 19D shows that OS of Predicted Responders (PR) based on the MK167 and FOXC1 complementary diagnostic approach is superior to that of Predicted Non-Responders (NR) using the same method and is statistically significant.

FIG. 20 shows ORR prediction accuracy in validation datasets in non-small cell lung cancer (NSCLC) AUC=0.96, OR=9.63 (0.98-94.54, 95% CI) p=0.03.

FIG. 21 shows that expression level of FOXC1 has prognostic significance comparable to multigene panels in being able to predict distinctly different rates of Overall Survival (OS) in multiple independent clinical datasets.

FIG. 22 shows that expression levels of FOXC1 was able to accurately discern between rates of brain metastasis-free survival (MFS)

FIG. 23 shows Kaplan-meier survival curves for breast cancer patients grouped according to molecular subtypes as assessed by standard immunohistochemistry.

FIG. 24 shows cox regression analysis of the prognostic significance of clinicopathologic and treatment variables on 5-year OS.

FIG. 25 shows Kaplan-Meier survival curves for breast cancer patients grouped according to 3-biomarker, 5-biomarker, or 4-biomarker models, each defining BLBC with a different combination of biomarkers.

FIG. 26 shows a comparison of the three different multivariate models of BLBC defined by IHC biomarker panels.

FIG. 27 shows the prognostic utility of VERESCA® FOXC1 IHC-defined recurrence risk in breast cancer: independent validation of risk stratification in terms of disease-specific mortality.

FIG. 28 shows univariate and multivariable analysis of 1992 patient dataset used to validate prognostic value of FOXC1 for 10-year follow-up.

FIG. 29 shows prognostic utility of VERESCA® FORXC1 IHC-defined recurrence risk in estrogen receptor positive breast cancer: risk stratification in terms of breast cancer-specific mortality.

FIG. 30 shows predictive utility of VERESCA® FOXC1 IHC-defined recurrence risk in ER+LN-breast cancer: stratification of recurrence risk despite treatment with adjuvant tamoxifen therapy.

FIG. 31 shows univariate and multivariable analysis of 411 patient compendium dataset of ER+LN-Tamoxifen treated chemotherapy untreated breast cancer patients used to validate predictive value of VERESCA® FOXC1 IHC based on 10-year recurrence-free survival.

FIG. 32 shows independent validation of predictive utility of VERESCA® FOXC1 IHC-defined mortality risk in ER+ HER2-LN-breast cancer: stratification of mortality risk despite treatment with adjuvant tamoxifen therapy.

FIG. 33 shows breast cancer metastasis risk stratification utilizing VERESCA® FOXC1: risk prediction to guide adjuvant chemotherapy recommendations.

FIG. 34 shows independent validation of breast cancer mortality risk stratification utilizing VERESCA® FOXC1: risk prediction to guide adjuvant chemotherapy recommendations.

FIG. 35 shows overall survival benefit of adjuvant chemotherapy in addition to adjuvant endocrine therapy in ER+ HER2-LN-breast cancer identified to have VERESCA® FOXC1 IHC-defined elevated mortality risk: clinical validation of predictive utility.

FIG. 36A shows the pathologic complete response rates from a TRIO-US-B07 trial of subjects that have HER2+ tumors with high FOXC1 expression and the sensitivity and specificity of high FOXC1 expression as a predictor of response after treatment with neoadjuvant taxane, Herceptin, and lapatinib. RD=Residual Disease, pCR=Pathologic Complete Response, ROC-AUC=Receiver Operator Characteristic Area Under Curve, OR=Odds Ratio, Cl=Confidence Interval, n=trial sample size.

FIG. 36B shows the pathologic complete response rates from a CHER-LOB trial of subjects that have HER2+ tumors with high FOXC1 expression and the sensitivity and specificity of high FOXC1 expression as a predictor of response after treatment with neoadjuvant taxane, Herceptin, and lapatinib. RD=Residual Disease, pCR=Pathologic Complete Response, ROC-AUC=Receiver Operator Characteristic Area Under Curve, OR=Odds Ratio, Cl=Confidence Interval, n=trial sample size.

FIG. 37 shows the pathologic complete response rates from subjects that have triple negative breast cancer tumors with high FOXC1 expression and the sensitivity and specificity of high FOXC1 expression as a predictor of response after treatment with neoadjuvant anthracycline monotherapy. RD=Residual Disease, pCR=Pathologic Complete Response, ROC-AUC=Receiver Operator Characteristic Area Under Curve, OR=Odds Ratio, Cl=Confidence Interval, n=trial sample size.

FIG. 38 shows the pathologic complete response rates from subjects that have triple negative breast cancer tumors with high FOXC1 expression and the sensitivity and specificity of high FOXC1 expression as a predictor of response after treatment with neoadjuvant taxane monotherapy. RD=Residual Disease, pCR=Pathologic Complete Response, ROC-AUC=Receiver Operator Characteristic Area Under Curve, OR=Odds Ratio, Cl=Confidence Interval, n=trial sample size.

FIG. 39 shows the pathologic complete response rates from subjects that have triple negative breast cancer tumors with high FOXC1 expression and the sensitivity and specificity of high FOXC1 expression as a predictor of response after treatment with neoadjuvant anthracycline plus taxane. RD=Residual Disease, pCR=Pathologic Complete Response, ROC-AUC=Receiver Operator Characteristic Area Under Curve, OR=Odds Ratio, Cl=Confidence Interval, n=trial sample size.

FIG. 40 shows the pathologic complete response rates from subjects that have triple negative breast cancer tumors with high FOXC1, Ki67, and PDL1 expression and the sensitivity and specificity of high FOXC1, Ki67, and PDL1 expression as a predictor of response after treatment with neoadjuvant taxane plus capecitabine. RD=Residual Disease, pCR=Pathologic Complete Response, ROC-AUC=Receiver Operator Characteristic Area Under Curve, OR=Odds Ratio, Cl=Confidence Interval, n=trial sample size.

FIG. 41 shows the pathologic complete response rates from subjects that have triple negative breast cancer tumors with high FOXC1, Ki67, and PDL1 expression and the sensitivity and specificity of high FOXC1, Ki67, and PDL1 expression as a predictor of response after treatment with neoadjuvant anthracycline plus taxane plus capecitabine. RD=Residual Disease, pCR=Pathologic Complete Response, ROC-AUC=Receiver Operator Characteristic Area Under Curve, OR=Odds Ratio, CI=Confidence Interval, n=trial sample size.

FIG. 42 shows the overall response rates from subjects that have advanced/metastatic melanoma with high FOXC1, MK167 and PDL1 expression and the sensitivity and specificity of high FOXC1, MK167 and PDL1 expression as a predictor of response after treatment with adjuvant nivolumab/pembrolizumab. RD=Residual Disease, pCR=Pathologic Complete Response, ROC-AUC=Receiver Operator Characteristic Area Under Curve, OR=Odds Ratio, Cl=Confidence Interval, n=trial sample size. Overall Response Rate (ORR)=Complete Response Rate (CRR)+ Partial Response Rate (PRR). ITT=Intention to Treat Population. NR=Non-responder. R=Responder. AUC=Area Under Curve. OR=Odds Ratio. Cl=Confidence Interval.

FIG. 43A shows the hyper-progressive disease incidence (%) from subjects that have advanced/metastatic melanoma with high FOXC1, MK167 and PDL1 expression and the sensitivity and specificity of high FOXC1, MK167 and PDL1 expression as a predictor of response after treatment with immune checkpoint inhibitors. ICI=Immune Checkpoint Inhibitor. Rx=Therapy. HPD=Hyper-Progressive Disease. AUC=Area Under Curve. OR=Odds Ratio. PD—Progressive Disease. HR=Hazard Ratio (Predicted Responder c/w Predicted Non-=resonder-HPD).

FIG. 43B shows Kaplan-Meier Survival curves of progression-free survival and overall survival for predicted responders, predicted non-responders with progressive disease (PD), and predicted non-responders with hyper-progressive disease (HPD) for patients with advanced/metastatic melanoma that were treated with immune checkpoint inhibitors. HPD=Hyper-Progressive Disease. AUC=Area Under Curve. OR=Odds Ratio. PD—Progressive Disease. HR=Hazard Ratio (Predicted Responder c/w Predicted Non-=resonder-HPD).

FIG. 44 shows the overall response rates from subjects that have Esophageal Squamous Cell Carcinoma with high FOXC1 expression and the sensitivity and specificity of high FOXC1 expression as a predictor of response after treatment with neoadjuvant chemoradiation therapy and atezolizumab. RD=Residual Disease, pCR=Pathologic Complete Response, ROC-AUC=Receiver Operator Characteristic Area Under Curve, OR=Odds Ratio, Cl=Confidence Interval, n=trial sample size. Overall Response Rate (ORR)=Complete Response Rate (CRR)+ Partial Response Rate (PRR). ITT=Intention to Treat Population. NR=Non-responder. R=Responder. AUC=Area Under Curve. OR=Odds Ratio. Cl=Confidence Interval.

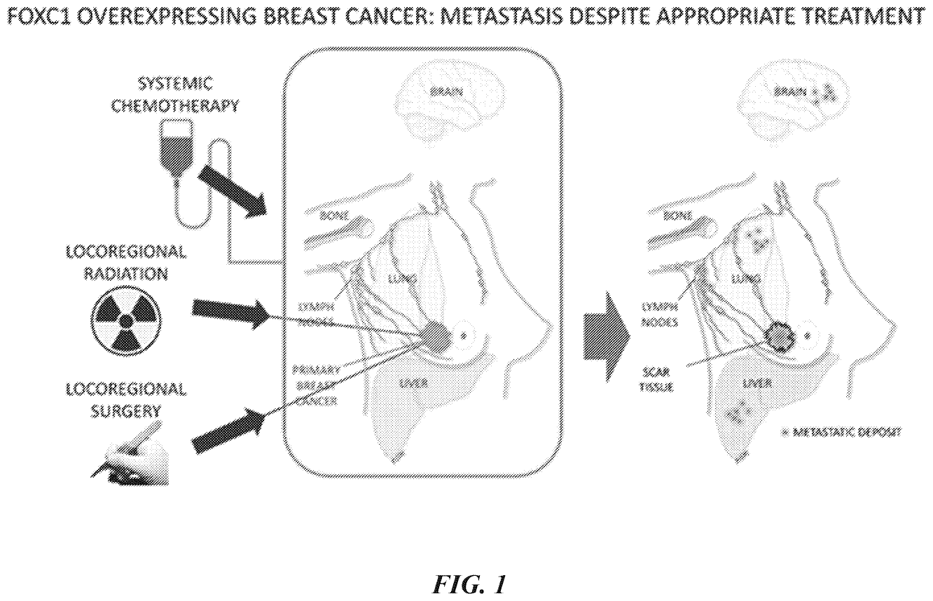

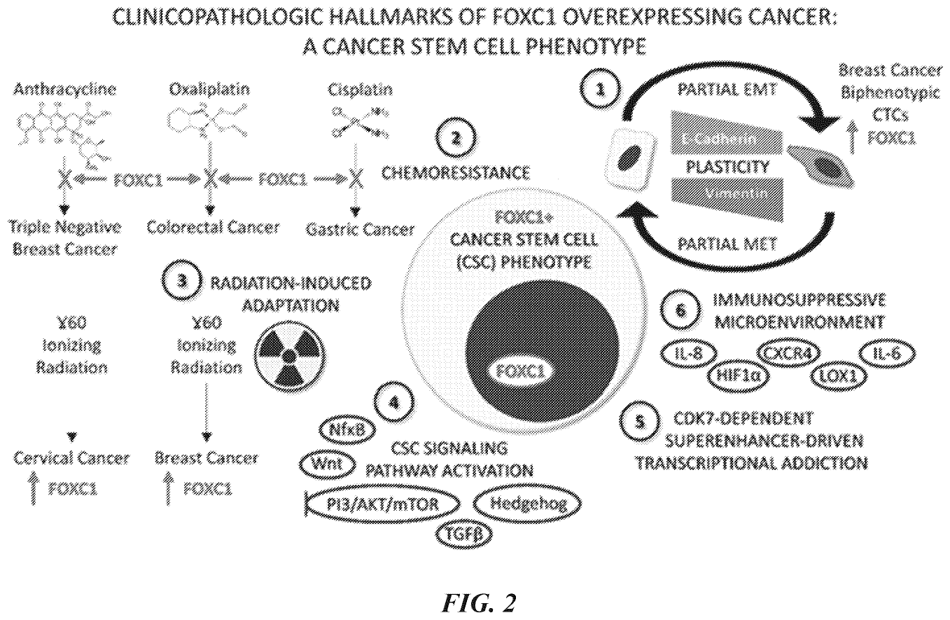

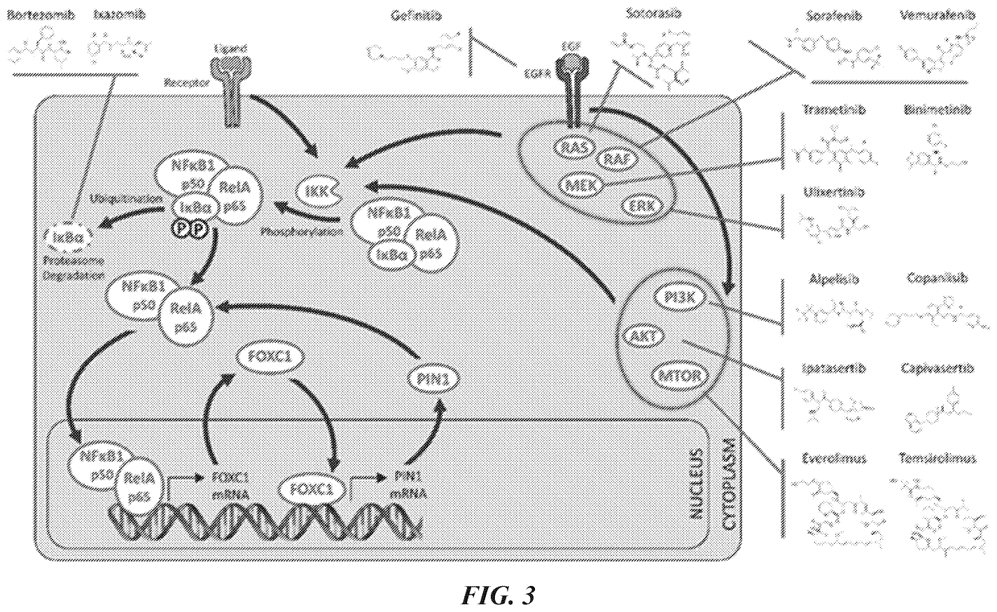

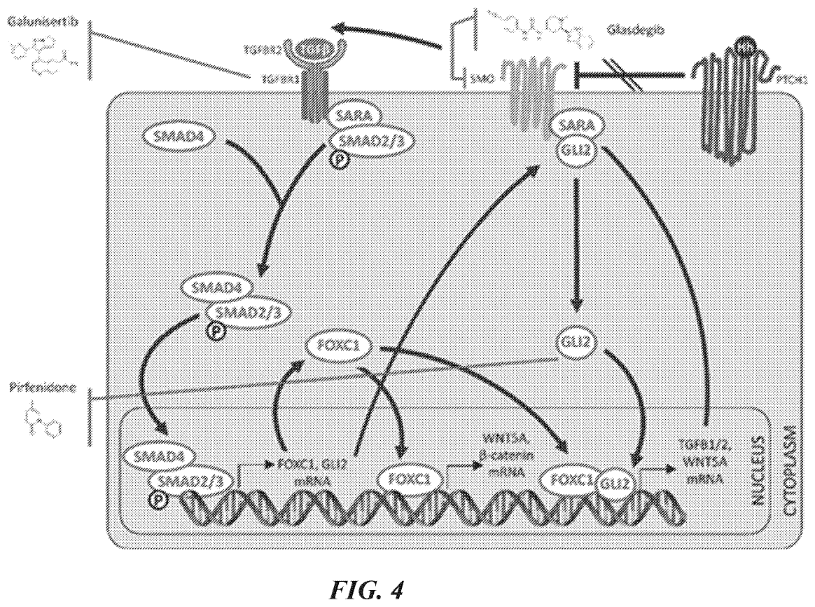

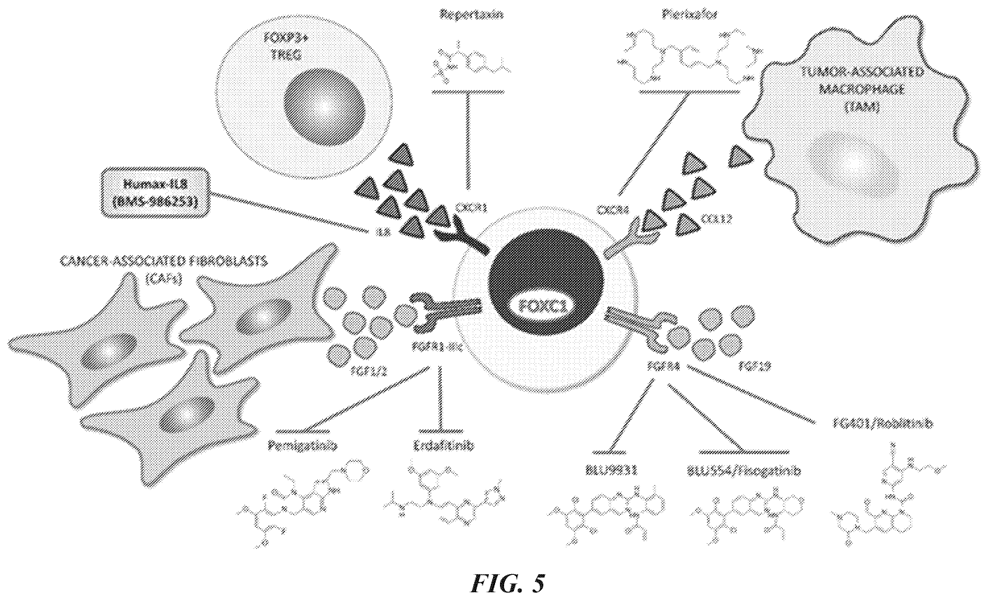

### DETAILED DESCRIPTION

The present technology includes antibodies and nucleotide sequences that encode antibodies that bind to the FOXC1 protein. The present technology also includes methods for identifying effective cancer therapies or predicting prognosis of a subject with cancer receiving or who may receive cancer therapies by determining the level of FOXC1 protein or nucleic acid in a sample obtained from the subject.

As used herein, a “subject” refers to a subject who has cancer according to the present technology. As used herein, the term “patient” is used interchangeably with “subject.”

The importance of cell plasticity, EMT, chemoresistance and the cancer stem cell phenotype to cancer progression and metastasis has come to be widely recognized in the field. However, because of the complexity of molecular regulatory mechanisms, attempts to target any individually promising approach have not had success. Many protein factors and biomarkers of metastasis have been implicated till date. Among them transcription factors (TFs) play a particularly impactful role in driving metastasis. This is because TFs are the central “coordination” hubs of large and extensive transcriptional networks. By virtue of influencing and fine tuning both the expression as well as repression of hundreds of genes at a time, TFs are capable of far reaching and pervasive action. TFs are, therefore, arguably one of the most powerful classes of metastasis biomarkers and merit in depth investigation with regard to direct or indirect therapeutic targeting as an effective means of thwarting metastatic spread. Several viewpoints on the role of the transcription factor FOXC1 playing an important regulatory role in mediating cell plasticity and partial EMT as important contributors to cancer progression and metastasis are presented herein. Also highlighted are therapeutic strategies that target cancers transcriptionally addicted to FOXC1 in order to help reduce recurrence risk, morbidity and mortality and improve quality of life.

In recent years, several excellent review articles on the role of the Forkhead box C1 (FOXC1) TF in cancer have been published, highlighting the increasing recognition of its importance as a clinically useful biomarker and potential therapeutic target (Han et al., 2017; Yang et al., 2017; Elian et al., 2018b; Wang et al., 2018a; Gilding and Somervaille, 2019). The focus of this review is to summarize and draw inferences from the body of literature that implicates FOXC1 as a transcriptional driver of cancer progression and metastasis. The current state of evidence supports the argument that FOXC1 plays this role by virtue of being essential for the emergence, maintenance and proliferation of cancer stem cells (CSCs). FOXC1 plays a functionally important role in mediating a wide variety of cancer traits all of which are, essentially, cancer stem cell traits. Such traits include cell proliferation, cell plasticity, partial EMT, cell migration, cell invasion, chemoresistance and radio-resistance. FOXC1 dependencies develop as a consequence of dysregulated programs in cancer cells and affect clinical progression, therapeutic responsiveness and outcome often emerging to a level of dependence referred to as “transcriptional addiction.” Collectively, the elucidated mechanisms by which FOXC1 modulates aggressive cancer cell traits support the contention that FOXC1 is predominantly a transcriptional driver of cellular plasticity and a cancer stem cell phenotype. Also highlighted are several potential targeted therapeutic strategies, based on utilizing already available FDA-approved oral anticancer drugs, that may help achieve improved clinical outcomes for patients diagnosed with FOXC1 pro-metastatic cancers. The therapeutic strategies described herein are potentially practice-changing in the field of oncology, and merit being tested in biomarker-driven, adaptive clinical trials.

The following description is merely exemplary in nature and is not intended to limit the present technology, its applications, or its uses. It should be understood that throughout the drawings, corresponding reference numerals indicate like or corresponding parts and features. The description of specific examples indicated in various embodiments of the present technology are intended for purposes of illustration only and are not intended to limit the scope of the present technology disclosed herein. Moreover, recitation of multiple embodiments having stated features is not intended to exclude other embodiments having additional features or other embodiments incorporating different combinations of the stated features.

Furthermore, the detailed description of various embodiments herein makes reference to the accompanying drawing/FIGS, which show various embodiments by way of illustration. While the embodiments are described in sufficient detail to enable those skilled in the art to practice the present technology, it should be understood that other embodiments may be realized and that logical and mechanical changes may be made without departing from the spirit and scope of the present technology. Thus, the detailed description herein is presented for purposes of illustration only and not of limitation. For example, steps or functions recited in descriptions, any method, system, or process, may be executed in any order and are not limited to the order presented. Moreover, any of the step or functions thereof may be outsourced to or performed by one or more third parties. FOXC1: detecting FOXC1 protein or nucleic acids

The present technology includes antibodies for detecting FOXC1 protein as well as methods that comprise detecting FOXC1 protein or nucleic acids in a sample obtained from a subject. In some aspects, the methods of the present technology include detecting FOXC1 protein and/or FOXC1 gene expression in the same. In some embodiments, the subject has cancer. In other embodiments, the subject may have cancer.

In some embodiments, the level of FOXC1 nucleic acid is determined in a tissue sample using quantitative reverse transcriptase polymerase chain reaction (qPCR) to detect the level of FOXC1 nucleic acid in the tissue sample. In some aspects, the level of FOXC1 mRNA is determined in a tissue. In some aspects, the FOXC1 nucleic acid that is detected is FOXC1 mRNA. In some aspects, the FOXC1 nucleic acid or mRNA is a nucleotide sequence having a sequence of SEQ ID NO: 44.

In some embodiments, FOXC1 protein is detected by any method known to one of skill in the art for detecting protein in a sample. In some aspects, FOXC1 protein is detecting using a protein that binds to a FOXC1 protein. In some aspects, the protein that binds to a FOXC1 protein is a FOXC1 antibody.

The present technology includes antibodies that bind to the FOXC1 protein. In some embodiments, the FOXC1 antibody includes a variable heavy (VH) domain having a complementarity-determining region (CDR) that binds to an antigen on a FOXC1 protein. In some aspects, the VH domain has a CDR having a sequence comprises the CDR regions of SEQ ID NO: 18. In some embodiments, the VH domain has a sequence comprising a first VH complementarity region (CDR) (VH CDR1) having an amino acid sequence comprising GFSITRDYA; a second VH CDR (VH CDR2) comprising INYSGTT; and a third VH CDR (VH CDR3) comprising VGWAVNYGLDY.

In some embodiments, the FOXC1 antibody includes a variable light (VL) domain having a complementarity-determining region (CDR) that binds to an antigen on a FOXC1 protein. In some aspects, the VL domain has a CDR having a sequence comprises the CDR regions of SEQ ID NO: 19. In some embodiments, the VL domain has a sequence comprising a first VL complementarity region (CDR) (VL CDR1) having an amino acid sequence comprising QSLLYSNGKTY or KSVSTSGYSY; a second VL CDR (VL CDR2) comprising LVS; and a third VL CDR (VL CDR3) comprising VQGTHFPHT or QHIRELTRSEGG.

In some aspects, the antibody is an antibody, an antibody fragment, an antibody conjugate, or an antibody fusion. In some aspects, the antibody is a monoclonal antibody. In some aspects, the antibody is a humanized antibody, a chimeric antibody, or a human antibody. In some aspects, the antibody is an scFv. In some aspects, the antibody comprises a sequence at least 80%, 85%, 90%, or 95% identical to one or more of SEQ ID NO:1-17. In some aspects, the antibody comprises one or more of SEQ ID NO:1-17.

The present technology also includes nucleotide sequences that encode a FOXC1 antibody. In some embodiments, the present technology includes a nucleotide sequence that encodes an antibody with (i) a VH region having at least 80% sequence identity to SEQ ID NO: 18, or (ii) both (i) and a VL region having at least 80% sequence identity to SEQ ID NO:19, or SEQ ID NO:20. In some embodiments, the present technology includes a nucleotide sequence that encodes an antibody with (i) a VH region having at least 85% sequence identity to SEQ ID NO: 18, or (ii) both (i) and a VL region having at least 85% sequence identity to SEQ ID NO:19, or SEQ ID NO:20. In some embodiments, the present technology includes a nucleotide sequence that encodes an antibody with (i) a VH region having at least 90% sequence identity to SEQ ID NO: 18, or (ii) both (i) and a VL region having at least 90% sequence identity to SEQ ID NO:19, or SEQ ID NO:20. In some embodiments, the present technology includes a nucleotide sequence that encodes an antibody with (i) a VH region having at least 95% sequence identity to SEQ ID NO: 18, or (ii) both (i) and a VL region having at least 95% sequence identity to SEQ ID NO:19, or SEQ ID NO:20. In some embodiments, the present technology includes a nucleotide sequence that encodes an antibody with (i) a VH region having at least 99% sequence identity to SEQ ID NO: 18, or (ii) both (i) and a VL region having at least 99% sequence identity to SEQ ID NO:19, or SEQ ID NO:20. In some embodiments, the present technology includes a nucleotide sequence that encodes an antibody with (i) a VH region having a sequence of SEQ ID NO: 18, or (ii) both (i) and a VL region having a sequence of SEQ ID NO:19, or SEQ ID NO:20. In some aspects, the nucleotide sequence encodes an amino acid sequence comprising one or more of SEQ ID NO:1-17.

In some aspects, the nucleotide sequence encodes an antibody, an antibody fragment, an antibody conjugate, or an antibody fusion. In some aspects, the nucleotide sequence encodes antibody that is a monoclonal antibody. In some aspects, the nucleotide sequence encodes an antibody that is a humanized antibody, a chimeric antibody, or a human antibody. In some aspects, the nucleotide sequence encodes an antibody that is an scFv. In some aspects, the nucleotide sequence encodes an antibody that is bispecific.

In some aspects, FOXC1 protein is detected using immunohistochemistry. In some aspects, the level of FOXC1 protein in a sample is scored using scoring techniques known to one of skill in the art. For example, a score of 0 may indicate no FOXC1 protein or substantially no FOXC1 protein detected in the sample. A score of 1 may indicate weakly positive staining. A score of 2 may indicate moderately positive staining. A score of 3 may indicate strongly positive staining.

### FOXC1: A Prognostic Biomarker of Cancer Progression and Metastasis

The present technology includes using FOXC1 as a biomarker of prognosis for cancer progression. The present technology includes methods of identifying effective cancer therapies in subjects with cancer by detecting FOXC1 protein or nucleic acid in a sample from the subject.

The earliest reports on the biologic role and function of the TF FOXC1 implicated it in abnormal pathologic conditions like glaucoma (Nishimura et al., 1998; Wang et al., 2001; Khan et al., 2008; Chakrabarti et al., 2009; Tanwar et al., 2010; Du et al., 2016; Medina-Trillo et al., 2016; Siggs et al., 2019), congenital hydrocephalus (Kume et al., 1998), congenital It defects (Nakano et al., 2003; Wu et al., 2020), congenital heart defects (Swiderski et al., 1999; Du et al., 2016; Sanchez-Castro et al., 2016; Khalil et al., 2017) and Axenfeld-Rieger Syndrome (Mears et al., 1998; Du et al., 2016; Micheal et al., 2016), a congenital disorder characterized by glaucoma and congenital heart defects. Contemporaneously, several reports detailing the role of FOXC1 in normal physiology were also reported with regard to development of the cornea and the anterior chamber of the eye (Kidson et al., 1999), renal development (Kume et al., 2000; Schwab et al., 2003) and cardiovascular development (Swiderski et al., 1999; Seo and Kume, 2006). Also elucidated was its role in coordinating the embryonic process of primitive mesoderm cell fate (Wilm et al., 2004) and migration of embryonic tissues (Mattiske et al., 2006).

The functional relevance and prognostic significance of FOXC1 in cancer was first reported in breast cancer in 2010 (Ray et al., 2010). Since that time, the pivotal role that FOXC1 plays in coordinating the aggressive biology underlying cancer progression and metastasis in multiple cancers has been shown (Tables I & II). Studies supporting the role of FOXC1 as a powerful prognostic biomarker are summarized in Table I and includes both “solid” as well as “liquid” cancers like acute myelogenous leukemia (AML). Indeed, an excellent and comprehensive systematic review and meta-analysis on the prognostic role of FOXC1 in cancer concluded that FOXC1 expression in cancer is indicative of poor survival outcome (Sabapathi et al., 2019). In support of FOXC1 playing a role in cancer progression, another meta-analysis reported that FOXC1 is 23.8% more likely to be expressed in late-stage cancers as opposed to early-stage cancers (Kume and Shackour, 2018). As discussed below, those studies demonstrate the role of FOXC1 in predicting an aggressive clinical course, specifically highlighting its role as a predictor of metastatic recurrence.

TABLE I
Clinical evidence supporting the role of FOXC1 in cancer progression and metastasis.

| | | FOXC1 in | | | | | | |
|---|---|---|---|---|---|---|---|---|
| | | Progression | | | | | | |
| | | and | | | | | | |
| | | Metastasis: | | Univariate Analysis | | Multivariate Analysis | | |
| Cancer | Sample | Clinical | Outcome | Hazard | | Hazard | | Reference |
| Type | Size | Evidence | Measured | ratio (CI) | p-value | ratio (CI) | p-value | PMID Number |
| AML | 458 | | OS | − | − | 1.784(1.29-2.46) | <0.001 | (Somerville et al., 2015) |
| | | | | | | | | 26373280 |
| AML | 452 | | OS | 1.592(1.263-2.007) | 0.0001 | 1.755(1.355-2.273) | <0.0001 | (Swaminathan et al., 2016) |
| | 313 | + | RFS | 1.539(1.208-1.961) | 0.0002 | 1.678(1.280-2.201) | 0.0001 | |
| Breast | 295 | | OS | − | 0.0001 | 1.25(1.02-1.52) | 0.02 | (Ray et al., 2010) 20406990 |
| | 286 | + | DMFS | − | <0.0001 | − | − | |
| | 232 | | OS | − | 0.0476 | − | − | |
| | 122 | | OS | − | 0.0098 | − | − | |
| | 159 | | OS | − | 0.0047 | − | − | |
| Breast | 724 | | OS | 3.364(1.758-6.438) | 0.0002 | 3.389(1.928-7.645) | 0.0001 | (Ray et al., 2011) 21424368 |
| Breast | 1975 | + | DSS | 1.71(1.31 to 2.23) | <0.001 | 1.55(1.17 to 2.06) | 0.003 | (Jensen et al., 2015) 26041837 |
| Breast | 1986 | + | DSS | 1.973(1.802-2.961) | <0.0001 | | | (Han et al., 2015) 26565916 |
| Breast | 120 | + | DFS | 2.62(1.05-6.50) | 0.038 | 2.58(1.04-6.42) | 0.041 | (Xu et al., 2017a) 28493031 |
| Cervical | 219 | | OS | − | − | 2.928(0.508-6.585) | 0.021 | (Huang et al., 2017b) 28386355 |
| | | + | RFS | − | − | 2.776(0.207-7.538) | 0.035 | |
| Cervical | 76 | | OS | | 0.0094 | | | (Wang et al., 2018b) 29328384 |
| Colon | 363 | | OS | 0.432(0.325-0.573) | <0.001 | 0.668(0.492-0.907) | 0.010 | (Liu et al., 2018a) 29884889 |
| | | + | DMFS | 0.422(0.319-0.558) | <0.001 | 0.617(0.457-0.834) | 0.002 | |
| Colon | 361 | | OS | 1.190(1.023-1.389) | 0.025 | | | (Li et al., 2019) 30171256 |
| | 185 | | OS | 3.371(1.745-6.513) | <0.001 | | | |
| | | + | DFS | 2.557(1.453-4.497) | 0.001 | | | |
| Colon | 361 | | OS | 1.20(1.03-1.40) | 0.002 | | | (Zhang et al., 2020c) 31650548 |
| | 86 | | OS | | 0.004 | | | |
| | 134 | | OS | | 0.002 | | | |
| Esophageal | 82 | | OS | − | 0.014 | − | − | (Pan et al., 2014) 25031703 |
| (SCC) | | | | | | | | |
| Esophageal | 147 | | OS | − | 0.023 | − | − | (Zhu et al., 2017) 28861321 |
| (SCC) | | + | DFS | − | 0.037 | − | − | |
| Gastric | 120 | | OS | 0.273(0.144-0.521) | <0.001 | 0.370(0.184-0.745) | 0.005 | (Xu et al., 2014) 24329718 |
| Gastric | 422 | | OS | 1.58(1.15-2.15) | 0.0038 | − | − | (Jiang et al., 2021) 33987183 |
| Hepatic | 406 | | OS | 0.587(0.453-0.760) | <0.0001 | 0.641(0.491-0.837) | 0.001 | (Xia et al., 2013) 22911555 |
| | | + | RFS | 0.566(0.434-0.738) | <0.0001 | 0.649(0.495-0.852) | 0.002 | |
| Lung | 125 | | OS | 1.324(0.657-2.175) | <0.001 | 1.328(0.625-2.021) | <0.001 | (Wei et al., 2013) 23264086 |
| (NSCLC) | | | | | | | | |
| Lung (LUAD) | 500 | | OS | − | 0.0484 | − | − | (Cao et al., 2018) 30548656 |
| Lung (LUSC) | 494 | | OS | − | 0.0363 | − | − | |
| Lung | 105 | | OS | 2.237(1.220-4.098) | 0.009 | 1.988(1.022-3.860) | 0.043 | (Gong et al., 2019) 31597217 |
| (NSCLC) | | | | | | | | |

“−” indicates that information was not mentioned in the text or supplementary material of the specific publication, AML acute myelogenous leukemia, CI confidence interval, DFS disease free survival, DMFS distant metastasis free survival, DSS disease-specific survival, FOXC1 Forkhead box C1, LUAD lung adenocarcinoma, LUSC lung squamous cell, NSCLC non-small cell lung cancer, OS overall survival, RFS-recurrence free survival

### Breast Cancer

The clinical evidence with regard to FOXC1 being a powerful prognostic indicator has been most extensively generated in breast cancer. FOXC1 mRNA and/or protein expression status has been demonstrated to be an independent, statistically significant, predictor of metastatic recurrence and poor survival. Multiple studies have now confirmed the clinical utility of FOXC1 expression, both at the mRNA as well as protein level, in predicting poor outcomes. Previous reports by us and others had shown that FOXC1 expression could accurately identify patients with the basal-like breast cancer (BLBC) molecular subtype, the most aggressive subtype of breast cancer (Ray et al., 2010; Ray et al., 2011; Sizemore and Keri, 2012; Cao et al., 2014; Jensen et al., 2015; Johnson et al., 2016; Kim et al., 2018; Zhao et al., 2020). BLBC has been shown to exist as a “hidden” diagnosis, irrespective of receptor profile status, and is not restricted or confined to only triple negative breast cancers (TNBCs) (Jensen et al., 2015). FOXC1 expression status further correlated with increased incidence of brain and lung metastases and decreased metastasis-free survival in patients without lymph node involvement (Ray et al., 2010). More recently, in a study involving unbiased bioinformatic screening of transcription factors, FOXC1 was found to display the highest correlation with an epithelial-to-mesenchymal-to-amoeboid (EMAT) cluster having the worst associated disease-specific survival in lymph node negative patients (Emad et al., 2020). A clinical grade, quantitatively robust, immunohistochemistry-based molecular diagnostic assay has also been developed which measures FOXC1 protein in FFPE tissue and has a high degree of correlation with measurement of FOXC1 mRNA using qRT-PCR from matched FFPE tissue samples (Jensen et al., 2015). The assay has undergone analytical and clinical validation, has achieved regulatory approval for breast cancer with the CE-mark designation and is available for in vitro diagnostic use in the clinic.

### Lung Cancer

The prognostic significance of FOXC1 in lung cancer was first reported in 2013 (Wei et al., 2013) and later confirmed by other independent investigators, both in lung adenocarcinoma as well as lung squamous cell carcinoma (Cao et al., 2018; Gong et al., 2019). Studies have shown that high FOXC1 expression is more frequently associated with adverse clinical parameters and poor overall survival independent of other clinicopathological prognostic factors, including lymph node status. The data are clear on elevated FOXC1 expression being a predictor of lung cancer progression, but are not yet available with regard to predicting spread outside of the primary organ. It is important to note that in the case of lung cancer, twice as many patients ultimately succumb to respiratory failure than as a consequence of distant metastasis to sites outside the lung (Nichols et al., 2012). Thus, lung cancer metastasis to locations or organs outside the lung does not appear to be the predominant cancer-related cause of death as in some other cancers that arise from the breast, colon, or skin (e.g. melanoma).

### Gastrointestinal Cancers

FOXC1 expression status has now also been shown to be a poor prognostic indicator in multiple gastrointestinal cancers including esophageal cancer, gastric cancer, liver cancer, pancreatic cancer and colon cancer. Two independent published reports provided initial insight into the potential prognostic significance of FOXC1 expression in esophageal squamous cell carcinoma with regard to overall survival. However, univariate and multivariate hazard ratio values were not available in the published reports (Pan et al., 2014; Zhu et al., 2017). One of these studies did however demonstrate that FOXC1 expression displayed a statistically significant association with disease-free survival as well (Zhu et al., 2017). Similarly, two independent studies in gastric cancer thus far have provided preliminary confirmation of the prognostic significance of FOXC1 expression with regard to overall survival (Xu et al., 2014; Jiang et al., 2021). Additional independent validation studies performed with greater statistical rigor are therefore required to ascertain the true prognostic value of FOXC1 expression in the case of both esophageal as well as gastric cancer.

A fairly large investigation of patients diagnosed with hepatocellular carcinoma (HCC) demonstrated that FOXC1 expression was a powerful, statistically significant prognostic indicator of worse overall survival as well as recurrence free survival, independent of other clinical variables (Xia et al., 2013). This result was confirmed on both univariate as well as multivariate analysis. While this is a retrospective, single institution study, the sample size and level of statistical significance suggest that FOXC1 expression status will likely be of clinical value in predicting the prognosis of patients diagnosed with HCC. With regard to pancreatic cancer, there is a single report investigating the potential prognostic relevance of FOXC1 expression in pancreatic cancer (Wang et al., 2013). While the sample size was not large, the study was successful in demonstrating that FOXC1 is a statistically significant and independent prognostic indicator of adverse outcomes in terms of poor overall survival. This result was strengthened by the fact that it was valid on both univariate as well as multivariate analysis.

Amongst the gastrointestinal cancers, the evidence in support of the prognostic utility of FOXC1 is perhaps most robust in colon cancer. Three independent studies have confirmed the fact that FOXC1 expression status can in fact predict worse overall survival in colon cancer (Liu et al., 2018a; Li et al., 2019; Zhang et al., 2020c). One of these studies' reports convincing data with regard to both overall survival and disease-free survival (Li et al., 2019). Yet another provides further confirmation of FOXC1 being a statistically significant independent predictor of shorter distant-metastasis-free survival on both univariate as well as multivariate analysis (Liu et al., 2018a). Thus, after breast cancer, clinical assessment of FOXC1 expression status for prognostic stratification of patients is most likely to be useful in colon cancer.

### Other Cancers

Within head and neck cancers, some functional and mechanistic studies have been published implicating FOXC1 in the biology of these tumors. However, investigations examining the prognostic role of FOXC1 are not yet available. A preliminary report on tongue cancer did demonstrate FOXC1 to be a statistically significant predictor of worse overall survival (Lin et al., 2014). However, the results of univariate and multivariate analysis were not accessible in the publication for review. Thus, information with regard to the prognostic role of FOXC1 in head and neck cancers is nascent at best, but does hold promise based on the findings of some initial studies on the functional significance of FOXC1. Studies investigating the prognostic significance of FOXC1 in nasopharyngeal carcinomas, laryngeal and oral squamous cell carcinomas are, therefore, needed. Similarly, with regard to melanoma, there is a single publication that reports FOXC1 to have prognostic significance in advanced Stage Ill or Stage IV melanoma in terms of distant metastasis-free survival. While highly insightful, the univariate and multivariate hazard ratios were not accessible in the publication for review. Thus, additional investigations are needed before we are able to draw any conclusions regarding the prognostic utility of FOXC1 expression in these cancers.

In contrast, data regarding the prognostic impact of FOXC1 expression in cervical cancer is more developed. Two independent studies have reported that FOXC1 possesses prognostic value with regard to predicting shorter overall survival (Huang et al., 2017b; Wang et al., 2018b). More importantly, one of these confirmed FOXC1 to be a predictor of shorter recurrence-free survival on multivariate analysis (Huang et al., 2017b). FOXC1 is a promising prognostic biomarker in cervical cancer and further studies should be performed to better delineate its clinical utility in this regard. In AML, high FOXC1 expression was found to be associated with adverse prognosis in comparison to low FOXC1 expression (Somerville et al., 2015). Subsequently, high FOXC1 expression was significantly correlated with both refractoriness to induction chemotherapy as well as an increased risk of relapse (Swaminathan et al., 2016). While further studies are needed in this regard, patients diagnosed with FOXC1+ AML should probably be recommended to enroll in clinical trials examining the efficacy of combination therapy protocols that combine various targeted therapies with standard induction chemotherapy. Such approaches may lead to their achieving minimal residual disease burden having no evidence of leukemic cancer stem cell markers, a known predictor of long-term remission following subsequent allogeneic bone marrow stem cell transplantation.

FOXC1 Expression Associated with Good Prognosis

It is important to note, however, that in two very specific cancer types, elevated FOXC1 expression, in contradistinction to the above evident trend, proved to be a predictor of favorable prognosis. These departures from the norm are unexpected exceptions to the seemingly apparent rule that is supported by the overwhelming majority of research findings in support of FOXC1 expression being an adverse prognostic indicator. Thus, while the preponderance of evidence supports elevated expression of FOXC1 being an accurate predictor of adverse clinical outcomes, the two notable exceptions include ovarian cancer and Luminal B molecular subtype of breast cancer where elevated expression of FOXC1 predicts good prognosis.

In the case of ovarian cancer, a retrospective study (Wang et al., 2016e) demonstrated that positive immunostaining for FOXC1 protein significantly decreased with advancing International Federation of Gynecology and Obstetrics Stage (1-II vs. Ill-IV) as well as pathologic subtypes from benign to borderline and malignant tumors trending towards good prognosis. However, it should be noted that the sample size was mall for cystadenocarcinomas (n=40) and pathologic subtype (n=80). Although nuclear and cytoplasmic FOXC1 staining was observed in cell lines, the above conclusion was based solely upon cytoplasmic FOXC1 as FOXC1 was not detected in the nucleus of the ovarian tumors.

The second report on FOXC1 expression in Luminal B breast cancer (Hirukawa et al., 2018) looks at FOXC1 as being predictive of favorable outcome and establishes the use of EZH2 methyltransferase inhibitors as a strategy to block metastasis. Specifically higher expression of FOXC1 was associated with increased relapse-free survival in Luminal B patients (HR=0.68 p=0.001), but not in BLBC patients (HR=1.01 p-0.94). This study demonstrated that FOXC1 activates certain anti-metastatic genes in the Luminal B breast cancer subset and that any pro-tumor effects of FOXC1 are overridden by the anti-metastatic functions of FOXC1 leading to an overall pro-survival effect in Luminal B patients expressing higher FOXC1 levels.

These highly exceptional findings merit further investigation into the context-specific subcellular localization of FOXC1, and factors that control and determine its post-translational stability and degradation. While initial insight into this aspect of FOXC1 regulation was provided in two published investigations of FOXC1 protein release, stability and degradation (Elian et al., 2018a; Li et al., 2018), it remains to be established whether these or similar mechanisms may help explain the above observations related to FOXC1 expression in ovarian cancer and Luminal B breast cancer.

### FOXC1: A Functional Driver of Cancer Progression and Metastasis

The insight into FOXC1 as a prognostic predictor of cancer progression and metastasis was accompanied by a growing body of work that also demonstrated its functional importance as a molecular driver of these processes utilizing both in vitro and in vivo models. The literature supporting this is summarized in Table 1l. Several other cancer types than are not featured in Table II have also been reported in which significant associations between the malignant phenotype and FOXC1 overexpression have been demonstrated. However, prognostic or functional roles of FOXC1 have yet to be elucidated in these cancers. These include carcinomas of the thyroid (Weinberger et al., 2017; Hossain et al., 2020), gallbladder (Li et al., 2013), kidney (Wang et al., 2016f; Yao et al., 2016), non-melanoma skin cancer (Singh et al., 2021) and synovial sarcoma (Fernebro et al., 2006). Further investigations are required to explore the potential prognostic and functional significance of these initial reports.

| | | | | EMT/ | | | | | |
|---|---|---|---|---|---|---|---|---|---|
| Cancer | | Cell | | pEMT/ | Circulating | | Chemo | Radio- | |
| Type | Transcriptional | Proliferation | Cellular | MET | Tumor | Endocrine | resistance | resistance | Cancer |
| Breast | | | X | X | | | | | |
| Breast | | | X | X | | | | | X |
| Breast | | | | | | | X | | |
| Breast | | | | | | | | X | |
| Breast | X | | | X | | | X | | |
| Breast | | | | X | X | | | | |
| Breast | | | | | | | | | X |
| Breast | X | | | | | | | | X |
| Breast | | | | X | | | | | X |
| Breast | | | | X | X | | | | X |
| Breast | | | X | | | | | | |
| Breast | X | | | | | | | | X |
| Breast | | | | | | | X | | |
| Breast | X | X | | | | | | | X |
| Breast | X | | X | | | | | | |
| Breast | X | | X | X | | | | | X |
| Breast | | | X | | | | X | | |
| Breast | X | | | | | | | | |
| Breast | | | | | | X | | | |
| Breast | | | | | | | | | X |
| Breast | | | | | | X | | | |
| Breast | | | | | | | X | | |
| Breast | | | | | | | | | |
| Breast | | | | X | | | | | X |
| Breast | | | X | | | | | | |
| Breast | | | | | | | | | X |
| Breast | | | | X | | | | | |
| Breast | | | X | | | | | | X |
| Breast | X | | | | | | | | X |
| Breast | | | | | | | X | | |
| Breast | | X | | | | | | | |
| Breast | X | | X | X | | | | | X |
| Breast | X | | X | | | | | | X |
| Breast | X | | X | | | | | | X |
| Prostate | | | | | | X | | | X |
| Prostate | | | | | | | | | X |
| Prostate | X | X | | | | | | | X |
| Prostate | X | X | | | | | | | X |
| Lung | | X | | X | | | | | X |
| Lung | | X | | X | | | | | X |
| Lung | | | X | | | | X | | |
| Lung | | X | | | | | | | X |
| Lung | | X | | | | | | | X |
| Lung | | X | | | | | | | X |
| Cervical | | | | X | | | | | X |
| Cervical | X | | | | | | | | X |
| Cervical | | | | | | | | X | X |
| Cervical | | | | | | | | | X |
| Endometrial | | X | | | | | | | X |
| Endometrial | | X | | | | | | | X |
| Esophageal | X | | | | | | | | |
| Esophageal | | X | X | X | | | | | |
| Gastric | | X | | | | | | | |
| Gastric | | X | X | | | | | | X |
| Gastric | | X | | | | | | | X |
| Gastric | | X | | | | | X | | X |
| Gastric | | | | | | | | | |
| Pancreatic | X | X | | | | | | | |
| Pancreatic | X | X | | X | | | | | X |
| Hepatic | X | X | | X | | | | | X |
| Hepatic | X | | | X | | | | | X |
| Hepatic | | | | | | | | | X |
| Hepatic | | | | | | | | | X |
| Colon | | | | | | | | | X |
| Colon | | X | X | | | | | | |
| Colon | | | | | | | X | | |
| Colon | X | | | | | | | | X |
| Colon | | | | | | | X | | |
| Oral SCC | X | X | | | | | | | X |
| Oral SCC | X | X | | | | | | | X |
| Oral SCC | | X | | | | | X | | X |
| Tongue | X | | | X | | | | | X |
| Salivary | X | X | | X | | | | | X |
| ACC | | | | | | | | | |
| Laryngeal | | | | | | | | | X |
| SCC | | | | | | | | | |
| Naso- | | | | X | | | | | |
| pharyngeal | | | | | | | | | |
| Naso- | X | | | X | | | | | X |
| pharyngeal | | | | | | | | | |
| Naso- | X | | | X | | | | | X |
| pharyngeal | | | | | | | | | |
| Naso- | X | | | X | | | | | X |
| pharyngeal | | | | | | | | | |
| Osteo- | X | X | | | | | | | X |
| sarcoma | | | | | | | | | |
| Osteo- | X | X | | | | | | | X |
| sarcoma | | | | | | | | | |
| Osteo- | | X | | | | | | | X |
| sarcoma | | | | | | | | | |
| Osteo- | X | X | | | | | | | X |
| sarcoma | | | | | | | | | |
| Melanoma | | | X | | | | | | X |
| Wilms | X | X | | | | | | | X |
| Tumor | | | | | | | | | |
| Glioblas- | X | | | | | | | | X |
| toma | | | | | | | | | |
| Glioblas- | X | | | X | | | | | X |
| toma | | | | | | | | | |
| Pituitary | | | | | | | | | X |
| AML | X | X | X | | | | | | |
| T-ALL | | | | | | | X | | |
| Lymphoma | X | X | | | | | | | |
| (HL) | | | | | | | | | |
| DLBCL | X | | | | | | | | X |

| | Molecular | | |
|---|---|---|---|
| | or | | |
| | Signal | | |
| Cancer | Transduction | | |
| Type | Pathway | Summary of Findings | Reference |
| Breast | | FOXC1 regulates CD44 + normal | (Bloushtain-Qimron |
| | | mammary progenitors | et al., 2008) |
| Breast | | 1st Report of FOXC1 as a | (Ray et al., 2010) |
| | | marker of aggressive traits | |
| Breast | | FOXC1 is associated with chemoresistance | (Dejeux et al., 2010) |
| Breast | | FOXC1 is associated with radioresistance | (Kuhmann et al., |
| | | | 2011) |
| Breast | ERα | BRCA1 and GATA3 transcriptionally represses | (Tkocz et al., 2012) |
| | | FOXC1 | |
| Breast | TGFβ | Elevated FOXC1 in breast CTCs | (Powell et al., 2012) |
| Breast | | Transient vs Stable | (Sizemore and Keri, |
| | | overexpression of FOXC1 | 2012) |
| Breast | NFκB | NFκB inhibitor blocks FOXC1 mediated M/I | (Wang et al., 2012) |
| Breast | Raf/ | FOXC1 downregulation associated with MET | (Leontovich et al., |
| | MEK/ | | 2012) |
| | MAPK | | |
| Breast | TGFβ | FOXC1 in breast CTCs | (Yu et al., 2013) |
| | | associated with progression | |
| Breast | | FOXC1 is enriched in | (Sizemore et al., |
| | | mammary luminal progenitors | 2013) |
| Breast | Ras/ | EGFR inhibitor blocks FOXC1 mediated M/I | (Jin et al., 2014) |
| | ERK, | | |
| | PI3K/ | | |
| | AKT | | |
| Breast | | FOXC1 is a marker of | (Klajic et al., 2014) |
| | | neoadjuvant chemoresistance | |
| Breast | FOXCUT | FOXC1-FOXCUT form mRNA-IncRNA pair | (Liu et al., 2015) |
| Breast | C-Wnt, | NFκB blocks stem cell escape following Wnt | (Ray et al., 2015b) |
| | NFκB | inhibition | |
| Breast | | FOXC1/FOXA1 transcriptional balance in breast | (Ray et al., 2015a) |
| | | cancer | |
| Breast | NC- | FOXC1 induces Gli2 activation | (Han et al., 2015) |
| | Hedge- | and NC Hedgehog | |
| | hog | | |
| Breast | CDK7 | CDK7-dependent SE-mediated FOXC1 addiction | (Wang et al., 2015) |
| Breast | ERα | FOXC1 transcriptionally represses ERα | (Yu-Rice et al., 2016) |
| Breast | | FOXC1 overexpression induces increased lung | (Zuo and Wu Yao, |
| | | metastasis | 2016) |
| Breast | ERα | FOXC1 transcriptionally represses ERα | (Wang et al., 2017a) |
| Breast | | FOXC1 is a marker of adjuvant Anthracycline | (Xu et al., 2017a) |
| | | resistance | |
| Breast | EGFR, | NFκB mediates EGF-induced FOXC1 expression | (Chung et al., 2017) |
| | NFκB | | |
| Breast | TGFβ, | TGFβ upregulates FOXC1, triggers | (Hopkins et al., 2017) |
| | FGFR1 | FGFR1 isoform switch | |
| Breast | | FOXC1 inhibits ELF5, | (Gao et al., 2017a) |
| | | lobuloalveolar development | |
| Breast | NC-Wnt, | FOXC1 mediates Wnt5A-NFκB-MMP7 induced | (Han et al., 2018) |
| | NFκB | invasion | |
| Breast | | Alternative splicing switch | (Li et al., 2018) |
| | | in FLNB induces FOXC1, EMT | |
| Breast | FAK- | ST8SIA1 regulates FOXC1 | (Nguyen et al., 2018) |
| | AKT- | | |
| | mTOR | | |
| Breast | CXCR4 | CXCR4-inhibitor blocks FOXC1 | (Pan et al., 2018) |
| | | mediated M/I, metastasis | |
| Breast | EZH2 | EZH2 epigenetically represses FOXC1 | (Zheng et al., 2020) |
| Breast | | FOXC1 upregulates LINC01123, ↓ miR663a | (Zhang et al., 2020b) |
| Breast | CDK7 | FOXC1 contributes to CDK7-inhibitor sensitivity | (Tang et al., 2020) |
| Breast | | FOXC1 is most significant EMAT/metastasis | (Emad et al., 2020) |
| | | activator | |
| Breast | CDK7 | FOXC1 is most significant invasion/metastasis | (Huang et al., 2021) |
| | | activator | |
| Prostate | | FOXC1 expression associated with AIPC | (Niewenhuijsen et |
| | | progression | al., 2009) |
| Prostate | EGFR | EGFR upregulates FOXC1 | (Peraldo-Neia et al., |
| | | | 2011) |
| Prostate | | MiR-138-5P inhibits FOXC1-mediated M/I | (Huang et al., 2020) |
| Prostate | | MiR-138-5P inhibits FOXC1-mediated M/I | (Zhang et al., 2020a) |
| Lung | | FOXC1 drives proliferation, EMT, invasion | (Chen et al., 2016) |
| Lung | HIF1α | HIF1a transcriptionally upregulates FOXC1 | (Lin et al., 2017) |
| Lung | β- | FOXC1 transcriptionally upregulates B-catenin | (Cao et al., 2018) |
| | catenin, | | |
| | C-Wnt | | |
| Lung | | IncRNA CCAT2 upregulates FOXC1 | (Hu et al., 2018) |
| Lung | LOX | FOXC1 transcriptionally upregulates LOX, ↑ | (Gong et al., 2019) |
| | | metastasis | |
| Lung | HIF1a | FOXC1 transcriptionally upregulates LINC00301, ↑ | (Sun et al., 2020) |
| | | HIF1α | |
| Cervical | PI3K/ | FOXC1 mediates PI3K/AKT induced EMT, M/I | (Huang et al., 2017b) |
| | AKT | | |
| Cervical | | miR-374c-5p inhibits FOXC1-mediated M/I | (Huang et al., 2017c) |
| Cervical | | FOXC1 is associated with radioresistance | (Khalilia, 2017) |
| Cervical | PI3K/ | FOXC1 mediates PI3K/AKT induced M/I | (Wang et al., 2018b) |
| | AKT | | |
| Endometrial | | miR-204 inhibits FOXC1- | (Chung et al., 2012) |
| | | mediated proliferation, M/I | |
| Endometrial | | miR-495 inhibits FOXC1- | (Xu et al., 2016) |
| | | mediated proliferation, M/I | |
| Esophageal | FOXCUT | FOXC1-FOXCUT form mRNA-IncRNA pair | (Pan et al., 2014) |
| Esophageal | | FOXC1 acts as transcriptional | (Zhu et al., 2017) |
| | | coactivator of, PBX1, ↑ ZEB2 | |
| Gastric | β- | FOXC1 transcriptionally downregulates DKK1, | (Jiang et al., 2021) |
| | catenin, | activating Wnt | |
| | C-Wnt | | |
| Gastric | Wnt | FOXC1 transcriptionally upregulates GPX8, | (Chen et al., 2020) |
| | | activating Wnt | |
| Gastric | | LINC00242 inhibits miR-141, upregulates FOXC1 | (Zhong et al., 2020) |
| Gastric | | MCM3AP-AS1 inhibits miR-148, upregulates | (Sun et al., 2021) |
| | | FOXC1 | |
| Gastric | EGFR | EGFR-FOXC1 upregulates histone H3C14 | (Rashid et al., 2021) |
| Pancreatic | | miR-138-5p inhibits FOXC1, ↓ proliferation, ↓ tumor | (Yu et al., 2015) |
| | | growth | |
| Pancreatic | PI3K/ | FOXC1 and IGF1R positively regulate each other | (Subramani et al., |
| | AKT/ | | 2018) |
| | mTOR | | |
| Hepatic | VEGF | FOXC1 | (Xu et al., 2012) |
| Hepatic | NEDD9 | FOXC1 transcriptionally upregulates NEDD9, ↑ | (Xia et al., 2013) |
| | | metastasis | |
| Hepatic | IL8, | IL8-dependent PI3K/AKT/HIF1α upregulates | (Huang et al., 2015) |
| | PI3K/ | FOXC1 | |
| | AKT, | | |
| | CXCR1 | | |
| Hepatic | HOTAIR | FOXC1 upregulates IncRNA HOTAIR, ↓ miRNA-1 | (Su et al., 2016) |
| Colon | FGFR4 | FOXC1 transcriptionally | (Liu et al., 2018a) |
| | | upregulates ITGA7, FGFR4 | |
| Colon | | FOXC1 transcriptionally upregulates FBP1, ↑ | (Li et al., 2019) |
| | | Warburg Effect | |
| Colon | | FOXC1 transcriptionally upregulates miR-31-5p, ↓ | (Hsu et al., 2019) |
| | | LATS2 | |
| Colon | | p38MAPK maintain FOXC1 protein stability, ↑ | (Zhang et al., 2020c) |
| | | metastasis | |
| Colon | | FOXC1 induces resistance to oxaliplatin, irinotecan | (Poorebrahim et al., |
| | | | 2020) |
| Oral SCC | FOXCUT | FOXC1-FOXCUT form mRNA-IncRNA pair | (Kong et al., 2014) |
| Oral SCC | | FOXC1 transcriptionally upregulates CCNB1/D1, | (Liu et al., 2018c) |
| | | MMP2/9 | |
| Oral SCC | | MCM3AP-AS1 inhibits miR-138, upregulates | (Li and Jiang, 2020) |
| | | FOXC1 | |
| Tongue | TGFβ | miR-639 inhibits FOXC1-mediated EMT, M/I | (Lin et al., 2014) |
| Salivary | | miR-582-5p inhibits FOXC1, ↓ M/I, ↓ metastasis | (Wang et al., 2017b) |
| ACC | | | |
| Laryngeal | | miR-204-5P inhibits FOXC1-mediated M/I | (Gao et al., 2017b) |
| SCC | | | |
| Naso- | | FOXC1 associated with | (Liu et al., 2014b) |
| pharyngeal | | low E-cadherin expression | |
| Naso- | | FOXC1 siRNA inhibited EMT, M/I | (Ou-Yang et al., |
| pharyngeal | | | 2015) |
| Naso- | | miR-4792 inhibits FOXC1-mediated EMT, M/I | (Li and Chen, 2015) |
| pharyngeal | | | |
| Naso- | FOXCUT | FOXCUT siRNA inhibited FOXC1-mediated | (Xu et al., 2017b) |
| pharyngeal | | MMP7/9 | |
| Osteo- | | miR-133b inhibits FOXC1- | (Deng et al., 2017) |
| sarcoma | | mediated proliferation, M/l | |
| Osteo- | EZH2 | FOXC1 transcriptionally upregulates EZH2 | (Qui, 2017) |
| sarcoma | | | |
| Osteo- | B- | Sp1/FOXC1/HOTTIP/LATS2/YAP/β-catenin | (Liu et al., 2020) |
| sarcoma | catenin, | cascade | |
| | C-Wnt | | |
| Osteo- | | miR-185-5p inhibits | (Zhao et al., 2021) |
| sarcoma | | FOXC1-mediated M/I | |
| Melanoma | PI3K/ | FOXC1 mediates MST1R mediated colony | (Wang et al., 2016c) |
| | AKT | formation, M/I | |
| Wilms | | HOXB2 and FOXC1 synergistically drive | (Jing et al., 2020) |
| Tumor | | progression | |
| Glioblas- | | miR-133 inhibits FOXC1-mediated M/I | (Liu et al., 2018b) |
| toma | | | |
| Glioblas- | B- | FOXC1 siRNA inhibited EMT, M/I | (Cao et al., 2019) |
| toma | catenin, | | |
| | C-Wnt | | |
| Pituitary | | miR-133 inhibits FOXC1, ↓ M/I | (Wang et al., 2016a) |
| AML | | FOXC1, HOXA9 ↑ clonogenic potential, ↓ | (Somerville et al., |
| | | differentiation | 2015) |
| T-ALL | NC- | FOXC1 stabilizes GLI2, drives SMO-inhibitor | (Tosello et al., 2020) |
| | Hedge- | resistance | |
| | hog | | |
| Lymphoma | FGF2 | OTX2 transcriptionally activates FOXC1 | (Nagel et al., 2015) |
| (HL) | | | |
| DLBCL | | JUN upregulates FOXC1, drives lymphoma | (Blonska et al., 2015) |
| | | dissemination | |

By virtue of being a transcription factor, and being a central hub gene controlling a network of hundreds of genes, it is not surprising that upregulation of FOXC1 in cancer does cast wide influence on a number of biologic processes critical for tumor survival and propagation. While this includes proliferation as reported in a number of studies, what has been a hallmark feature of FOXC1+ status is that it appears to predominantly be responsible for a pro-metastatic phenotype. This is evident in Table II based on the number of studies across a wide variety of cancers where this has been demonstrated to be the case. In addition to the clinical correlative studies outlined above, clear examples of the critical importance of FOXC1 in metastagenesis includes studies performed in breast cancer (Zuo and Wu Yao, 2016; Pan et al., 2018), lung cancer (Gong et al., 2019), HCC (Xia et al., 2013; Lin et al., 2021), colon cancer (Zhang et al., 2020c) and salivary gland cancer (Wang et al., 2017b). In these studies, overexpression led to the development of an increased number of metastatic lesions in preclinical animal models. Conversely, siRNA-mediated knockdown of FOXC1 practically abolished metastatic propensity (Xia et al., 2013; Pan et al., 2018; Zhang et al., 2020c) in most of these models, confirming the critical dependence of these cancers on FOXC1 to drive the metastatic program.

Several molecular mechanisms have also been elucidated in this regard. These include induction of partial EMT that promotes enhanced cancer cell migration (discussed below), increased production of matrix metalloproteinases that promotes cancer cell invasion (Table II), and interaction with other cell types in the tumor microenvironment by way of molecular crosstalk that further supplements and enhances these aggressive cancer cell characteristics (discussed below). Crosstalk with other cell types also leads to a state of pervasive immune suppression in the TME that significantly assists with cancer immune evasion. This also aids and abets the cancer cell in being able to continue on its aggressive pro-metastatic course. Cell crosstalk mechanisms pertinent to FOXC1+ cancers are discussed in more detail in a separate section below.

### FOXC1: A Driver of Aberrant Cellular Plasticity and Partial EMT

An epithelial-mesenchymal transition (EMT) enables cancer cells to depart from the primary tumor, invade surrounding tissue, and disseminate to distant organs. Several excellent reviews have implicated FOXC1's role in EMT. After multiple initial reports that linked FOXC1 with EMT in various cancers, some recent reports have further refined the understanding that FOXC1 is in fact associated with a partial EMT phenotype comprising of hybrid E/M cells.

Maheswaran and colleagues had found that mesenchymal cells expressing known EMT regulators, including TGF-β pathway components and the FOXC1 transcription factor, were highly enriched in circulating tumor cells (CTCs) and these mesenchymal CTCs were associated with disease progression (Yu et al., 2013). Similarly, Agelaki and colleagues found that EMT markers (Twist and Vimentin) are expressed in CTCs of patients with metastatic breast cancer (Kallergi et al., 2011). Additionally, Maheswaran and colleagues also noticed small populations of CTCs that were positive for both epithelial and mesenchymal markers by RNA-in situ hybridization, and these hybrid E/M CTCs were often enriched in patients with progressive disease after chemotherapy (Yu et al., 2013). In this same study, an index patient demonstrated dynamic switching between mesenchymal and epithelial CTCs upon each cycle of therapy, suggesting that CTCs maintain dynamic E/M plasticity (Yu et al., 2013; Hinohara and Polyak, 2019). This data supports the result from a study by Gupta and colleagues which utilized a DNA barcoding approach in the human breast cancer cell line MDA-MB-157 whereby a distinct clonal population of tumor cells was observed to fluctuate between epithelial and mesenchymal states, demonstrating intrinsic E/M plasticity (Mathis et al., 2017). Additionally, they further demonstrated that a single clonal population of tumor cells maintained stable co-expression of both epithelial-to-mesenchymal markers, suggesting the fact, that it is possible for cells to be plastic enough to maintain both epithelial and mesenchymal characteristics.

In an independent study performed by Blanpain and colleagues that utilized a model of squamous cell carcinoma, the clonal population of tumor cells which maintain a hybrid E/M state, has been demonstrated to possess greater metastatic potential than either complete E polarized or complete M polarized cancer cells (Pastushenko et al., 2018). In an HCC study, elevated FOXC1 expression was associated with enhanced trans-endothelial migration and microvascular invasion (Xu et al., 2012). However, siRNA-mediated knockdown of FOXC1 was only able to exert an incomplete reversion of EMT, characterized by decreased expression of mesenchymal markers (Vimentin, N-cadherin), but without an accompanying increase in a key epithelial marker (E-cadherin). Epithelial traits were only partially impacted in this condition, and E-cadherin remained unchanged in both expression level and distribution. In summary, this study provides evidence that metastasis may be more dependent on cells maintaining a high FOXC1 expression, which drives a partial EMT (having more of hybrid E/M characteristics) than it is on cells undergoing a complete EMT. This pool of highly plastic cells is more likely to survive in the bloodstream and represents the primary pool of cells from which metastatic lesions are seeded and arise. As such CTC FOXC1+ expression status may in fact help define those CTCs which are Metastasis Initiating Cells (MICs)(Baccelli et al., 2013).

FOXC1: Role in Cancer Cell Crosstalk with Other Cells in the Tumor Microenvironment

Tumor cells are surrounded by a heterogeneous and complex tumor microenvironment (TME). The TME consists of a tumor specific extracellular matrix, which recruits an abundance of non-cancer cells including epithelial cells, endothelial cells, mesenchymal cells, immune cells and fibroblasts, all of which interact with the primary tumor cell contributing to tumor progression and metastasis. TME-tumor signaling actively secrets chemokines, cytokines, growth factors, and other metabolites create a dynamic changing environment (Hu and Polyak, 2008; Balkwill et al., 2012). A chronic inflammatory, pro-angiogenic and immunosuppressive environment is created through ECM remodeling and through TME-tumor crosstalk, cancer progression and resistance to therapy (Tredan et al., 2007; Son et al., 2017). 2017). Infiltrating inflammatory cells can provide a chemotactic escape route for migrating cancer cells from the bulk tumor and modulate cell invasiveness (Aras and Zaidi, 2017; Qiu et al., 2018). Breast cancer cells and macrophages, through a reciprocal paracrine loop involving EGF, CSF-1, CSF-2 or CCL18, leads to EMT, increased cell motility, invasion and metastasis (Wyckoff et al., 2004; Su et al., 2014).

Akin to the above general trend, crosstalk between FOXC1+ cancer cells and other cells of the TME has also been a prominently noted feature. Upregulated FOXC1 in tumor cells induces production and release of cytokines, chemokines and growth factors which mediates recruitment of stromal cells to the TME. Proinflammatory cytokine Interleukin-8 (IL-8) is a member of the CXC chemokine family of angiogenesis/inflammation-related chemokines, secreted by stromal (endothelial cells and fibroblasts) and tumor cells. All biological effects of IL-8 are mediated by 2 receptors designated as CXCR1 (IL-8RA) and CXCR2 (IL-8RB). IL-8 induces FOXC1 upregulation by activation of the PI3K/AKT pathway and Hypoxia Inducible Factor 1a (HIF1a) (Huang et al., 2015). Consequently, activated FOXC1 transactivates CXC chemokine receptor 1 (CXCR1), a crucial promoter of cancer cell motility through activation of Rho-GTPases (Schraufstatter et al., 2001), that increases invasion and metastasis in HCC (Huang et al., 2015). CCL2 (monocyte chemoattractant protein-1, MCP-1) is a potent chemokine for monocytes, and a variety of other immune cells, known to activate JAK2/STAT23 signaling (Mellado et al., 1998). CCL2 was also transcriptionally upregulated by FOXC1 in a FOXC1 overexpressed HCC cell line, and appropriately repressed in a FOXC1 knockdown HCC cell line. This transactivation of CCL2 by FOXC1 significantly promoted macrophage infiltration and cancer metastasis in HCC mouse models. These findings were corroborated in human HCC tissues, where FOXC1 expression was found to correlate with levels of IL-8, CXCR1 and CCL2 expression, and infiltration of tumors by macrophages. What is important to note here is that while IL-8 induces FOXC1 transcriptional upregulation via a PI3K/AKT-HIF1α-driven mechanism, FOXC1 in turn transcriptionally upregulates the cognate receptor of IL-8 which is CXCR1, thereby establishing a self-sustaining positive feedback loop. With regard to a different cytokine, FOXC1 overexpression robustly increased NFκB-driven luciferase activity in breast cancer MDA-MB-231 and MCF-7 cells (Wang et al., 2012). Upregulated NFκB, in turn, induced interleukin-6 (IL-6) generation in MDA-MB-231 cells. The IL-6/STAT3/NFκB positive feedback loop is known to persistently activate breast stromal fibroblasts (Hendrayani et al., 2016). Pharmacologic targeting of the IL-8/FOXC1/CXCR1 and FOXC1/NFκB/IL-6 positive feedback loops hold promise for deriving clinical therapeutic benefit in FOXC1+ pro-metastatic cancers. The chemokine CXCL12 and its cognate receptor CXCR4, a transcriptional target of FOXC1 was shown to play central roles in cancer proliferation, angiogenesis, invasion, tumor microenvironment, as well as drug resistance induced by chemotherapy (Pan et al., 2018). CXCL12 affects tumor cell biology via 1) direct stimulation of signaling pathways that promote cancer cell growth, metastasis, and angiogenesis; 2) indirect effects, including the recruitment of CXCR4/CXCR7-positive cancer cells to CXCL12-expressing organs.

FGFR1 is a proven transcriptional target of FOXC1 in breast cancer, following its own transcriptional upregulation by TGFβ pathway activation (Hopkins et al., 2017). FGFR1 inhibition is known to promote infiltration of myeloid-derived suppressor cells (MDSCs), that exhibit strong immunosuppressive activity, into the breast cancer TME and promote both cancer progression and metastasis. Inhibition of FGFR1 markedly diminished the level of MDSC infiltration (Holdman et al., 2015) and efficiency of metastatic dissemination (Liu et al., 2014a). Crosstalk between FGFR1 and macrophage derived chemokines CXCL1 and CXCL5 promote tumor formation and progression (Bohrer and Schwertfeger, 2012). FGFR1 also promotes the release of inflammatory chemokine CX3CL1 which recruits macrophages to the TME and promotes angiogenesis, both processes being effectively blocked upon treatment with a CX3CL1 inhibitor (Reed et al., 2012). FOXC1 also transcriptionally upregulates FGFR4 in colon cancer (Liu et al., 2018a). FGFR4 upon ligation with FGF19 is known to promote drug resistance, cancer progression and metastasis. FGFR4 has also been demonstrated to be a poor prognostic indicator in colon cancer (Li et al., 2014). The activated FGF19-FGFR4 pathway enhances GSK3β-βcatenin signaling, consequently inducing EMT and resulting in increased HCC metastasis (Goetz and Mohammadi, 2013; Zhao et al., 2016) and CRC metastasis (Liu et al., 2018a).

FOXC1: Interplay with Signal Transduction Pathways in Cancer

FOXC1 has been shown to play a critical role in the development and progression of multiple malignancies. Aberrant FOXC1 expression is involved in diverse tumorigenic processes, such as abnormal cell proliferation, cancer stem cell maintenance, cancer migration, and angiogenesis. However, although FOXC1 overexpression often drives aggressive traits in a wide array of human carcinomas, the mechanisms of FOXC1 deregulation that influence the oncogenic and metastagenic processes seem specific to each tumor setting. An overview of FOXC1 and its correlation with various signal transduction pathways that have been reported in various cancers and highlight how the signal transduction-FOXC1 connection may provide an effective modality to therapeutically intervene and block the aggressive progression of FOXC1+ cancers.

FOXC1: Interconnected with EGFR, NF-KB, Ras/Raf/MEK/ERK and P13K/Akt/mTOR Signaling

FOXC1 and EGFR are both critical markers and functional regulators of BLBC. Cui and colleagues reported that EGFR activation regulates FOXC1 expression through ERK and AKT-mediated pathways in BLBC cells (Jin et al., 2014). In a separate study by the same group, NFκB transcription factor was found to regulate FOXC1 expression in BLBC cells through EGFR signaling (Chung et al., 2017). EGFR activation also promoted nuclear translocation of NFκB, which binds to the FOXC1 promoter. In the highly aggressive BLBC subtype of TNBC, FOXC1 regulated Pin1/NFκB signaling (Wang et al., 2012). Further, FOXC1 overexpression in basal-like MDA-MB-231 breast cancer cells markedly induced phosphorylation of NFκB p65 subunit at Ser-546 and its translocation into the nucleus. Battula and coworkers discovered that ganglioside GD2 expression defined breast cancer stem cells (BCSCs) and ST8SIA1 regulated GD2 expression and breast cancer stem cell (BCSC) function by activation of the FAK-AKT-mTOR signaling pathway. They also showed that in primary TNBC, ST8SIA1 was highly expressed and its expression positively correlated with the expression of FOXC1 (Nguyen et al., 2018). In HCC cell lines, IL-8 activated expression of FOXC1 via the phosphoinositide 3-kinase/AKT signaling and hypoxia-inducible factor la. FOXC1 transcriptionally activated CXCR1 and CCL2, which promoted inflammation, invasion and metastasis (Huang et al., 2015). In cervical cancers and melanoma, it was shown that FOXC1 increased MST1R and activated the PI3K/AKT pathway to drive invasion and migration in melanoma cells (Wang et al., 2016c). Overexpression of FOXC1 in MIA Pancreatic Cancer cells resulted in increasing the active forms of AKT, P13K, ERK, and p70s6k (Subramani et al., 2018). Taken together, these studies supported the finding that therapeutic targeting of the EGFR/FOXC1/NFκB pathway and PI3K/AKT/mTOR in BLBC, PI3K/AKT/HIF-1α/FOXC1 axis in HCC and MST1 R/PI3K/AKT in cervical cancers, melanoma and pancreatic cancers, may provide effective modalities for the treatments.

### FOXC1 And Wnt Signaling

Both WNT5A and FOXC1 are up-regulated in TNBC cells and play a significant role in invasion and metastasis. Han and colleagues showed that FOXC1 binds directly to the promoter of WNT5A and up-regulates WNT5A expression in TNBC cells via NFκB signaling (Han et al., 2018). Increased WNT5A expression in TNBC cells is also associated with increase in MMP7 expression. Collectively, FOXC1-WNT5A-MMP7-NFκB signaling axis plays an important role in the migration, invasion, and distant metastasis of TNBC cells. Further, FOXC1 negatively regulates DKK1 (a WNT inhibitor) expression by binding to its promoter region, thereby activating Wnt pathway in gastric cancer cells. FOXC1 also forms a complex with unphosphorylated β-catenin protein in the cytoplasm thereby promoting the entry of β-catenin into the nucleus. Once inside the nucleus, it dissociates from β-catenin, thus regulating transcription of c-MYC, which promotes the proliferation of gastric cancer cells (Jiang et al., 2021).

FOXC1 and Non-canonical Hedgehog Signaling

FOXC1 induces cancer stem cell (CSC) properties in BLBC cells via activation of Smoothened-independent Hedgehog (Hh) signaling (Han et al., 2015). This non-canonical activation of Hh signaling is specifically mediated by N-terminal domain of FOXC1 binding directly to Gli2, enhancing transcription-activating capacity of Gli2. Together with regulating non-canonical Hh signaling, FOXC1 overexpression also induces resistance to SMO-inhibitors targeting canonical Hedgehog signaling, thus further confirming that actions exerted by FOXC1 are in agreement with it being a marker of CSC function.

### FOXC1 And MAPK, IGF1/IGF1R and LOX Signaling Axis

Aberrant expression of FOXC1 and activation of the FOXC1-p38-MAPK loop promotes tumor metastasis in colorectal cancer (CRC) (Zhang et al., 2020c). IGF-1 R and FOXC1 regulate each other in pancreatic cancer and FOXC1 is a direct downstream signaling molecule of IGF-1/IGF-1 R axis. IGF-1 R upregulation in pancreatic cancer cells contributes to cancer progression and metastasis (Subramani et al., 2018). A positive correlation between FOXC1 expression and lysyl oxidase (LOX) expression was established in NSCLC patient samples wherein FOXC1 activated LOX transcription to drive cancer progression through the FOXC1-LOX axis (Gong et al., 2019). These results suggest that FOXC1 has oncogenic properties that favor metastasis of various cancers.

### FOXC1 And TGF-Beta Signaling

Exogenous exposure to TGFβ1 increased FOXC1 mRNA during TGFβ1-induced EMT via Smad2 and Smad3 transcription factors. Hopkins and colleagues demonstrated that FOXC1 expression was activated during TGF-β1-mediated EMT events through the binding of Smad3 proteins to a region in the FOXC1 promoter, −800 bp upstream of the transcription start site (Hopkins et al., 2017). In thus study, while FOXC1 was not essential for TGFβ-induced EMT, it was however critical for effecting an FGFR1 isoform switch that was critical for driving invasion and metastasis, that followed the TGFβ-induced EMT.

### FOXC1: Transcriptional Addiction in Triple Negative Breast Cancer

Transcription factors have been implicated in controlling extensive gene expression and regulating various cellular responses. Enhancers are regions of non-coding DNA, which mediate the transcription of adjacent genes, serving as a cis-regulatory element. Superenhancers (SEs) are a hyper active subset of enhancers, that recruit transcription factors, cofactors, and chromatin regulators to drive abundant expression of some significant genes (e.g., oncogenes) in the cancer context. FOXC1 is an SE-associated transcription factor that contributes to invasion, migration and metastasis in TNBC. Young and colleagues had found that TNBC cells and ER-negative cells are exceptionally dependent on the expression of at least a subset of the active genes that are transcriptionally regulated by cyclin-dependent kinase 7 (CDK7). Additionally, in this study it was demonstrated that TNBC cell proliferation is selectively sensitive to THZ1, a newly developed CDK7 inhibitor while ER and or PR-positive cells were largely unaffected by treatment of THZ1 (Wang et al., 2015). To seek potential biomarkers of THZ1 sensitivity, Tang and coworkers analyzed the mRNAs profile in breast cancer cells treated with THZ1 from the previous study and demonstrated that elevated expression of SOX9 was significantly associated with the sensitivity of THZ1 in TNBC (Tang et al., 2020). Furthermore, SOX9 and FOXC1 interacted with each other, to co-regulate the MYC signaling pathway in TNBC, while CDK7 inhibitor, THZ1 significantly disrupted the binding of SOX9 to FOXC1 and several enhancer and SE-associated transcription factors, increasing apoptotic cell death. In summary, these findings demonstrate that a collection of SE-associated TNBC genes (EGFR, FOSL1, FOXC1 and MYC) play a significant regulatory role in the proliferation, survival, invasion and metastasis of these CDK7-sensitive and TNBC-enriched cancers. As a logical conclusion, CDK7 inhibitors that block these targetable oncogenes could potentially serve as a rational targeted therapy option for patients diagnosed with TNBC.

### FOXC1: Regulator of Cancer Stem Cells, Quiescence and Tumor Escape Mechanism From Conventional Chemotherapy, Radiation Therapy and Targeted Therapy

FOXC1 plays an important role in mediating normal as well as cancer stem cell traits. In normal physiology, Yi and colleagues demonstrated that murine hair follicle stem cells induce FOXC1 to re-establish quiescence (Wang et al., 2016d). In this study, FOXC1 was shown to help preserve quiescent stem cell identity by activating NFATC1 and BMP signaling. In an independent study, hair follicle stem cells were demonstrated to have significantly higher FOXC1 expression, where it helps govern their proliferation and conserves their tissue-regenerating potential compared to downstream, more differentiated hair follicle cells (Lay et al., 2016). FOXC1 also helps reprogram murine epidermal cells to induced functional sweat gland-like cells, thus proving its potential to determine sweat gland fate in vitro (Yao et al., 2019). In murine reticular cells, FOXC1 expression is essential for maintenance of the niche where adult hematopoietic stem cells reside (Omatsu et al., 2014). These finding were later corroborated in the human system as well (Aoki et al., 2021). These studies collectively highlight the role FOXC1 as a key transcriptional regulator of normal stem cell activity.

Cancer stem cells (CSCs), are a small population of cancer cells that recapitulate most normal stem cell traits, but also play an essential role in tumor initiation, maintenance, progression, metastasis, drug resistance to anti-cancer drugs and metastatic recurrence. Recent studies have indicated that FOXC1, which is associated with a wide variety of cancers, is also strongly associated with mediating CSC activity. Cui and coworkers reported that FOXC1-overexpressed MDA-MB-231 cells, when injected orthotopically into the mammary glands of BALB/c nude mice, resulted in a marked increase in tumor formation efficiency compared to the control group (Han et al., 2015). On the contrary, when FOXC1-knockdown BT549 cells were injected into the mouse mammary glands, tumorigenesis was completely inhibited. In vitro, FOXC1 overexpressed SUM159 and MDA-MB-468 cells showed enhanced aldehyde dehydrogenase activity, the increase of which is used for characterizing breast CSC. The above results indicate that FOXC1 positively regulates CSC properties of BLBC cells in vivo and in vitro. In NSCLC, Xu and colleagues demonstrated that FOXC1 knockdown reduced CD133+ cell percentage, suppressed self-renewal ability, decreased expression of stemness-related genes (OCT4, NANOG, SOX2 and ABCG2) and inhibited NSCLC cell tumorigenicity in vivo (Cao et al., 2018). Battula and coworkers discovered that ganglioside GD2 expression defines breast cancer stem cells (BCSCs) (Nguyen et al., 2018). In thus study, ST8SIA1, which regulates GD2 expression was found to be positively correlated with FOXC1 upregulation.

Chemotherapeutic drug resistance is a well-established cancer stem cell property. Mullan and colleagues were the first to show that FOXC1 acts as a mediator of drug resistance to a chemotherapeutic drug regimen comprising 5-fluorouracil/epirubicin/mitomycin C (FEM) as well as to docetaxel (Tkocz et al., 2012). Basal levels of FOXC1 expression were increased in FEM-resistant clones compared to parental MDA-MB-468 cells. Additionally, knockdown of FOXC1 in MDA-MB-231 cells resulted in increased sensitivity to treatment with docetaxel. In NSCLC, FOXC1 knockdown increased cisplatin and docetaxel sensitivity and reduced gefitinib resistance, whereas FOXC1 overexpression enhanced CSC-like properties and resistance to cisplatin and docetaxel (Cao et al., 2018). Oxaliplatin (OXA) is currently used as first-line chemotherapy to treat stage Ill and stage IV metastatic colorectal cancer (CRC). Transcription factor FOXC1 binds to the miR-31 promoter to increase the expression of miR31-5p and regulate LATS2 expression in CRC, resulting in cancer cell resistance to OXA (Hsu et al., 2019). LincRNA MCM3AP-AS1 induced upregulation of FOXC1 expression, indicating cisplatin resistance in gastric cancer patients (Sun et al., 2021). Xu and coworkers investigated the effects of FOXC1 on chemosensitivity in TNBC patients and found that only a minority of TNBC patients have an excellent outcome after receiving standard chemotherapy (Xu et al., 2017a). Despite receiving standard cytotoxic chemotherapy, approximately 30-40% of patients with early-stage TNBC develop metastatic disease. They showed that a significant percentage of TNBC patients who had suboptimal outcomes with anthracycline-based standard chemotherapy were FOXC1 positive.

Therapeutic resistance in cancer also includes resistance to endocrine therapy with anti-estrogen drugs. In breast cancer, Cui and colleagues demonstrated that ectopic FOXC1 expression in ERα-positive MCF7 luminal breast cancer cells greatly diminished the effects of growth-stimulatory B-estradiol and growth-inhibitory antiestrogen treatment with Tamoxifen and Fulvestrant (Yu-Rice et al., 2016). Furthermore, in breast cancer patients with ER-positive primary tumors who received tamoxifen treatment, FOXC1 expression is associated with decreased or undetectable ER expression in recurrent tumors post endocrine treatment. In another independent study by Wang and coworkers, overexpression of FOXC1 decreased expression of ERα protein and reduced cellular responses to estradiol and tamoxifen, while knockdown of FOXC1 induced expression of ERα protein and improved cellular responses to estradiol and tamoxifen (Wang et al., 2017a).

### FOXC1: Role in Metabolic Programming in Cancer

Metabolic reprogramming is another essential hallmark of cancer (Hanahan and Weinberg, 2011). Specific metabolic processes can be directly involved in the transformation process or biological processes that support tumor growth. FOXC1, which has been shown to play an important role in the development and progression of multiple malignancies, also plays a pivotal role in metabolism. Xia and colleagues reported that FOXC1 could inhibit the cysteine metabolism-related genes, cystathionine γ-lyase (CTH) through upregulation of de novo DNA methylase 3B (DNMT3B) expression, which resulted in the decrease of cysteine levels and increase reactive oxygen species (ROS) levels (Lin et al., 2021). In human HCC cells FOXC1 was in turn upregulated by ROS-ERK1/2-β-ELK1 signaling axis. This positive feedback loop of OS-FOXC1-cysteine metabolism-ROS promotes liver cancer proliferation and metastasis, and this pathway may provide a prospective clinical treatment approach for HCC. Altered glycolysis metabolism is a well characterized signature of invasive cancers. Li and coworkers investigated the role of FOXC1 in regulating glycolysis in CRC cells and found that knockdown of FOXC1 expression in LoVo and RKO cells in vitro markedly reduced glucose uptake and lactate production, while ectopic expression of FOXC1 in HT29 and SW480 cells increased glucose consumption and lactate production. Further, FOXC1 promoted glycolysis and proliferation in CRC cells by inhibiting a key gluconeogenesis regulating enzyme, fructose-1,6-bisphosphatase 1 (FBP1) expression by binding directly to the promoter regions of the FBP1 gene and negatively regulating its transcription.

Methods for Identifying Effective Cancer Therapies and Subjects with Good Prognosis

The present technology includes methods for identifying an effective cancer therapy for a subject having cancer. In some aspects, the method comprises: (a) determining a level of FOXC1 protein or nucleic acid in a sample obtained from the subject; (b) determining whether the level of FOXC1 protein or nucleic acid is present in the sample at a level above a predetermined cutoff value; (c) predicting whether a cancer therapy will be clinically effective to reduce or treat the cancer in the subject based on the determining of step (b); (d) developing a treatment plan comprising (1) providing or continuing to provide the cancer therapy if the cancer therapy is determined to be the effective cancer therapy in (c), or (2) altering or stopping the cancer therapy if the cancer therapy is not determined to be the effective cancer therapy in (c).

In some embodiments, the cancer therapy useful with the methods of the present technology is an antihormonal therapy. In some embodiments, the cancer therapy is a chemotherapy. In some embodiments, the cancer therapy is an immunotherapy. In some embodiments, the cancer therapy comprises two or more anticancer agents, including, but not limited to, antihormonal therapy, chemotherapy, and immunotherapy.

In some embodiments, the cancer therapy is an agent that inhibits, blocks, or attenuates TGF-β signaling. In some aspects, the cancer therapy is galunisertib or pirfenidone.

In some embodiments, the cancer therapy is an agent that inhibits, blocks, or attenuates the nuclear factor kappa B (NFκB) signaling pathway. In some aspects, the cancer therapy is bortezomib, carfilzomib, or Ixazomib.

In some embodiments, the cancer therapy is an agent that inhibits, blocks, or attenuates the P13K, PTEN, AKT, or mTOR signaling pathways. In some aspects, the cancer therapy is an mTOR inhibitor. In some aspects the cancer therapy is ipatasertib, capivasertib, everolimus, or temsirolimus.

In some embodiments, the cancer therapy is an agent that inhibits, blocks, or attenuates the Wnt signaling pathway.

In some embodiments, the cancer therapy is an agent that inhibits, blocks, or attenuates the non-canonical hedgehog signaling pathway.

In some embodiments, the cancer therapy is a GLI2 inhibitor. In some aspects, the cancer therapy is glasdegib or pirfenidone.

In some embodiments, the cancer therapy is an agent that inhibits, blocks, or attenuates the non-canonical Notch signaling pathway.

In some embodiments, the cancer therapy is an agent that inhibits or blocks the PD1/PDL1 immune checkpoint. In some aspects, the cancer therapy is a PD1 inhibitor. In some aspects, the cancer therapy is a PDL1 inhibitor. In some aspects, the cancer therapy is Nivolumab, Pembrolizumab, Cemiplimab, Atezolizumab, Avelumab, or Durvalumab.

In some embodiments, the cancer therapy is an agent that inhibits or blocks the CTLA4 immune checkpoint. In some aspects, the cancer therapy is ipilimumab.

In some embodiments, the cancer therapy is an agent that inhibits or blocks the CDK4/6 class of cyclin-dependent kinase enzymes. In some aspects, the cancer therapy is Palbociclib, ribociclib, or abemaciclib.

In some embodiments, the cancer therapy is an agent that inhibits or blocks the epidermal growth factor receptor (EGFR). In some aspects, the cancer therapy is gefitinib.

In some embodiments, the cancer therapy is an agent that inhibits or blocks the RAS, RAF, MEK, or ERK kinase enzymes. In some aspects, the cancer therapy is a RAS, RAF, MEK, or ERK inhibitor. In some aspects, the cancer therapy is a sotorasib, sorafenib, vemurafenib, trametinib, binimetinib, or ulixertinib.

In some embodiments, the cancer therapy is an agent that inhibits, blocks, or attenuates the IL8 or CXCR1 pathway. In some aspects, the cancer therapy is an IL8 or CXCR1 inhibitor. In some aspects, the cancer therapy is Humax-IL8 (BMS-986253) or Repertaxi.

In some embodiments, the cancer therapy is an agent that inhibits, blocks, or attenuates the CCL12/CXCR4 pathway. In some aspects, the cancer therapy is a CXCR4 inhibitor. In some aspects, the cancer therapy is plerixafor.

In some embodiments, the cancer therapy is an agent that inhibits, blocks, or attenuates the FGF/FGFR1 pathway. In some aspects, the cancer therapy is a FGFR1 inhibitor. In some aspects, the cancer therapy is pemigatinib or erdafitinib.

In some embodiments, the cancer therapy is an agent that inhibits the FGF19 or FGFR4 pathway. In some aspects, the cancer therapy is a FGFR4 inhibitor. In some aspects, the cancer therapy is BLU9931, BLU554/Fisogatinib, FG401/Roblitinib.

In some embodiments, the cancer therapy is adjuvant chemotherapy and the cancer is ER+ breast cancer.

In some embodiments, the cancer therapy is neoadjuvant immunotherapy and the cancer is triple negative breast cancer of ER+ breast cancer.

In some embodiments, the cancer therapy is adjuvant immunotherapy or salvage immunotherapy and the cancer is Non-small cell lung cancer (NSCLC), melanoma, Head and Neck Squamous cell carcinoma (HNSCC), or Renal Cell Carcinoma.

In some embodiments, the cancer therapy is a neoadjuvant regimen comprising a PARP inhibitor, a taxane chemotherapy, or an PDL1 immune checkpoint inhibitor. In some aspects, the cancer therapy is neoadjuvant taxane and platinum. In some aspects, the cancer therapy is a neoadjuvant immune checkpoint inhibitor.

In some embodiments, the cancer therapy is adjuvant or salvage nivolumab plus pembrolizumab and the cancer is advanced or metastatic melanoma. In some aspects, the cancer therapy is an immune checkpoint inhibitor the cancer is advanced or metastatic melanoma.

In some embodiments, the cancer therapy is neoadjuvant chemoradiation therapy plus atezolizumab and the cancer is esophageal carcinoma.

In some embodiments, the cancer therapy is a hormonal or antihormonal treatment such as abiraterone, enzalutamide, anastrozole, aromatase inhibitors, bicalutamide, flutamide, nilutamide, dexamethasone, hydrocortisone, leuprorelin, goserelin, triptorelin, methylprednisolone, prednisolone, prednisone, or tamoxifen.

In some embodiments, the cancer therapy is a chemotherapy such as cisplatin, carboplatin, oxaliplatin, cyclophosphamide, altretamine, plicamydin, chlorambucil, chlormethine, ifosfamide, melphalan, carmustine, fotemustine, lomustine, streptozocin, busulfan, dacarbazine, mechlorethamine, procarbazine, temozolomide, thioTEPA, uramustine, paclitaxel, docetaxel, vinblastine, vincristine, vindesine, vinorelbine, hexamethylmelamine, etoposide, teniposide, methotrexate, pemetrexed, raltitrexed, cladribine, clofarabine, fludarabine, mercaptopurine, tioguanine, capecitabine, cytarabine, fluorouracil, fluxuridine, gemcitabine, daunorubicin, doxorubicin, epirubicin, idarubicin, mitoxantrone, valrubicin, bleomycin, hydroxyurea, mitomycin, topotecan, irinotecan, aminolevulinic acid, methyl aminolevulinate, porfimer sodium, verteporfin, alitretinoin, altretamine, amsacrine, anagrelide, arsenic trioxide, asparaginase, bexarotene, bortezomib, celecoxib, denileukin, diftitox, erlotinib, estramustine, gefitinib, hydroxycarbamide, imatinib, pentostatin, masoprocol, mitotane, pegaspargase, tretinoin, or combinations thereof.

In some embodiments, the cancer therapy is an immunotherapy, such as ilimumab, tremelimumab, pembrolizumab, nivolumab, durvalumab, anti-KIR, urelumab, anti-LAG-3, or combinations thereof.

In some embodiments, the cancer is breast cancer, lung cancer, or colon cancer. In some aspects, cancer is a triple-negative breast cancer or a HER2 negative ER+ cancer. In some aspects, the tumor is a bladder cancer tumor. In some aspects, the cancer is a triple-negative breast cancer tumor. In some aspects, the cancer is glioblastoma, breast cancer, colorectal cancer, renal cell carcinoma, chronic lymphocytic leukemia, hepatocellular carcinoma, non-small cell and small cell lung cancer, Non-Hodgkin lymphoma, acute myeloid leukemia, ovarian cancer, pancreatic cancer, prostate cancer, esophageal cancer including cancer of the gastric-esophageal junction, gallbladder cancer and cholangiocarcinoma, melanoma, gastric cancer, urinary bladder cancer, head and neck squamous cell carcinoma, or uterine cancer.

In some embodiments, the method includes determining a level of FOXC1 protein or nucleic acid in a sample obtained from the subject. In some aspects, the sample includes a cancer cell, cancerous tissue, or a tumor. The sample may be a solid or liquid biopsy obtained from the subject.

In some embodiments, the method includes determining the level of FOXC1 protein by applying the antibody of the present technology to the sample to detect FOXC1 protein.

In some embodiments, the method includes applying a FOXC1 antibody to a sample, where the antibody includes a VH domain having a CDR having a sequence comprises the CDR regions of SEQ ID NO: 18. In some embodiments, the antibody of the method comprises a VH domain with a sequence comprising a first VH complementarity region (CDR) (VH CDR1) having an amino acid sequence comprising GFSITRDYA; a second VH CDR (VH CDR2) comprising INYSGTT; and a third VH CDR (VH CDR3) comprising VGWAVNYGLDY.

In some embodiments, the method includes applying a FOXC1 antibody to a sample, where the antibody includes a variable light (VL) domain having a complementarity-determining region (CDR) that binds to an antigen on a FOXC1 protein. In some aspects, the method includes applying an antibody comprising a VL domain with a CDR having a sequence that comprises the CDR regions of SEQ ID NO: 19. In some embodiments, the antibody of the method comprises a sequence comprising a first VL complementarity region (CDR) (VL CDR1) having an amino acid sequence comprising QSLLYSNGKTY or KSVSTSGYSY; a second VL CDR (VL CDR2) comprising LVS; and a third VL CDR (VL CDR3) comprising VQGTHFPHT or QHIRELTRSEGG.

In some aspects, the method includes applying a FOXC1 antibody that is an antibody, an antibody fragment, an antibody conjugate, or an antibody fusion. In some aspects, the antibody of the method is a monoclonal antibody. In some aspects, the antibody of the method is a humanized antibody, a chimeric antibody, or a human antibody. In some aspects, the antibody is an scFv. In some aspects, the antibody of the method comprises a sequence at least 80%, 85%, 90%, or 95% identical to one or more of SEQ ID NO:1-17. In some aspects, the antibody of the method comprises one or more of SEQ ID NO:1-17.

In some embodiments, the method includes determining the level of FOXC1 protein in the tissue sample comprises performing immunohistochemistry (IHC) on the sample. In some aspects, the method includes using an antibody of the present technology to detect FOXC1. In some embodiments, the method includes scoring the level of FOXC1 protein using a scoring technique known to one of skill in the art.

In some embodiments, the method includes determining the level of FOXC1 nucleic acid in the sample using a quantitative reverse transcriptase polymerase chain reaction (qPCR) to detect the level of FOXC1 nucleic acid in the tissue sample. In some embodiments, the FOXC1 nucleic acid that is detected is FOXC1 mRNA. In some aspects, the method includes detecting a nucleic acid having a sequence of SEQ ID NO: 44.

In some embodiments, the method of the present technology includes predicting whether the therapy is clinically effective. In some aspects, the cancer therapy is clinically effective if treating the subject with the therapy results in a clinicopathologic outcome compared to before the cancer therapy was administered. In some aspects, clinical efficacy is measured as one or more clinicopathologic outcomes in the subject compared to the clinicopathologic outcome in a control subject. In some aspects, the control subject is a subject having a sample with no FOXC1 protein or nucleic acid detected, or FOXC1 protein or nucleic acid detected at a level below the predetermined cutoff value.

In some aspects, the clinicopathologic outcomes comprise or are selected from the group consisting of: (i) a decrease in tumor size or tumor number of the cancer in the subject, (ii) a decrease in cancer cell proliferation in the subject (iii) a decrease in cancer cell invasion or migration in the subject (iv) a decrease in incidence of recurrence of the cancer in the subject, or (v) a decrease in incidence of metastatic spread of the cancer in the subject, (vi) an increase or prolongation of disease-free survival, (vii) an increase or prolongation of recurrence-free survival, (viii) an increase/prolongation of distant metastasis-free survival, (ix) an increase or prolongation of event-free survival, (x) an increase or prolongation of progression-free survival, (xi) an increase or prolongation of disease-specific survival, (xii) an increase or prolongation of overall survival.

In some embodiments, determining whether the level of FOXC1 protein or nucleic acid is present at a level above a predetermined cutoff value comprises identifying the presence of FOXC1 in the tissue sample.

In some embodiments, the predetermined cutoff value is an IHC score of 1 or more. In some embodiments, the predetermined cutoff value is an IHC score of 2 or more.

In some embodiments, the predetermined cutoff value is determined by: (i) identifying the level of FOXC1 protein or nucleic acid in a plurality of retrospective subjects that were diagnosed with the cancer and were given the cancer therapy to treat the cancer; (ii) identifying the rate of observed pathologic complete response (pCR) for the plurality of retrospective subjects; (iii) correlating the level of FOXC1 protein or nucleic acid with the rate of observed pCR; and (iv) determining the predetermined cutoff value by calculating a Negative Predictive Value (NPV) and sensitivity for pCR prediction based on the predetermined cutoff value, wherein the predetermined cutoff value is selected to maximize the NPV and sensitivity for pCR.

In some embodiments, the method includes continuing to provide the cancer therapy to the subject. In some aspects, continuing to provide the cancer therapy comprises continuing to administer the same dose of the therapy at the same dose. In some aspects, continuing to provide the cancer therapy comprises continuing to administer the therapy with the same dosing regimen.

In some embodiments, the method includes altering the cancer therapy in the subject. In some aspects, altering the therapy comprises reducing or increasing the dose of the therapy. In some aspects, altering the therapy comprises changing the therapy to a different agent. In some aspects, altering the therapy comprises adding an additional therapeutic agent.

In some aspects, the methods of identifying an effective cancer therapy further comprises administering the therapy to the subject according to the treatment plan.

In some embodiments, the method further comprises determining the level of K167 protein or nucleic acid in the tissue sample. In some aspects, the method further comprises determining whether the level of K167 is present at a level above a second predetermined cutoff value, wherein the cancer therapy has elevated clinical efficacy if the level of FOXC1 is present at a level above a predetermined cutoff value and the level of K167 is present above a second predetermined cutoff value.

In some aspects, the second predetermined cutoff value is a Ki67 IHC score of 1 or greater. In some aspects, the second predetermined cutoff value is a Ki68 IHC score of 2 or greater. In some aspects, the second predetermined cutoff value is determined by (i) identifying the level of K167 protein or nucleic acid in a plurality of retrospective subjects that were diagnosed with the cancer and were given the cancer therapy to treat the cancer; (ii) identifying the rate of observed pathologic complete response (pCR) for the plurality of retrospective subjects; (iii) correlating the level of K167 protein or nucleic acid with the rate of observed pCR; and (iv) determining the predetermined cutoff value by calculating a Negative Predictive Value (NPV) and sensitivity for pCR prediction based on the predetermined cutoff value, wherein the predetermined cutoff value is selected to maximize the NPV and sensitivity for pCR.

In some embodiments, the method further comprises determining the level of PDL1 protein or nucleic acid in the tissue sample. In some aspects, the method further comprises determining whether the level of PDL1 is present at a level above a third predetermined cutoff value, wherein the cancer therapy has elevated clinical efficacy if the level of FOXC1 is present at a level above a predetermined cutoff value, the level of K167 is present above a second predetermined cutoff value, and the level of PDL1 is present above a third predetermined cutoff value.

In some embodiments, the third predetermined cutoff value is a PDL1 IHC score of 1 or greater. In some aspects, the third predetermined cutoff value is a PDL1 IHC score of 2 or greater. In some aspects, the third predetermined cutoff value is determined by: (i) identifying the level of PDL1 protein or nucleic acid in a plurality of retrospective subjects that were diagnosed with the cancer and were given the cancer therapy to treat the cancer; (ii) identifying the rate of observed pathologic complete response (pCR) for the plurality of retrospective subjects; (iii) correlating the level of PDL1 protein or nucleic acid with the rate of observed pCR; (iv) determining the predetermined cutoff value by calculating a Negative Predictive Value (NPV) and sensitivity for pCR prediction based on the predetermined cutoff value, wherein the predetermined cutoff value is selected to maximize the NPV and sensitivity for pCR.

The present technology also includes methods for predicting a prognosis of a cancer in a subject treated with a cancer therapy. In some aspects, the method comprises: (a) determining a level of FOXC1 protein by contacting in a sample obtained from the subject; (b) determining whether the level of FOXC1 protein is present in the sample at a level above a predetermined cutoff value; (c) predicting whether the subject receiving the cancer therapy has a good prognosis based on the determining step (b), wherein the subject has good prognosis if the expression level of FOXC1 protein is higher than the predetermined cutoff value.

In some embodiments, good prognosis is measured as one or more clinicopathologic outcomes in the subject compared to the clinicopathologic outcome in a control subject, wherein the clinicopathologic outcomes are selected from the group consisting of: (i) a decrease in tumor size or tumor number of the cancer in the subject, (ii) a decrease in cancer cell proliferation in the subject (iii) a decrease in cancer cell invasion, migration in the subject (iv) a decrease in incidence of recurrence of the cancer in the subject, (v) a decrease in incidence of metastatic spread of the cancer in the subject (vi) an increase or prolongation of disease-free survival, (vii) an increase or prolongation of recurrence-free survival, (viii) an increase/prolongation of distant metastasis-free survival, (ix) an increase or prolongation of event-free survival, (x) an increase or prolongation of progression-free survival, (xi) an increase or prolongation of disease-specific survival, (xii) an increase or prolongation of overall survival.

### Therapeutically Targeting FOXC1+ Prometastatic Cancers

It is evident from the above narrative that multiple signaling pathways converge upon and regulate the FOXC1 transcription factor. FOXC1, in turn, influences and coordinates multiple biological processes, again utilizing a variety of downstream signaling pathways, by which FOXC1 pro-metastatic cancers participate in the maturation of the aggressive migratory phenotype leading to metastasis. In some cases, self-sustaining positive-feedback loops are created which make their targeted interruption particularly attractive therapeutic strategies to test in the clinic. While further mechanistic elucidation of the upstream regulators and downstream mediators of FOXC1 activity are required, certain preliminary therapeutic strategies have begun to emerge based on the foundational investigations that have thus far been completed. Below we describe some of these potential strategies which hold promise and merit further consideration during the clinical trial design process.

### NfκB Signal Transduction Pathway

As described above, the NFκB signaling pathway and FOXC1 can in certain contexts reinforce one another's actions indefinitely, thereby constituting a self-perpetuating positive feedback loop that contributes to the maintenance of cancer stem cell traits. This suggests that it is a potential therapeutic vulnerability that could potentially be exploited to improve survival outcomes. Significant preliminary data in support of such an approach being therapeutically actionable in the clinic was provided by a preclinical study (Petrocca et al., 2013). Utilizing a genome-wide siRNA screen, proteasome addiction was identified as a vulnerability of basal-like TNBC cells. Basal-like TNBC cell lines, known to have elevated FOXC1 expression status, were selectively sensitive to proteasome inhibitor drugs, proportionate to their relative level of FOXC1 expression. Proteasome inhibition effectively blocked tumor-initiating cell function in vitro, and significantly reduced tumor growth and metastatic dissemination to the lungs in vivo. Further evidence to support such an approach was obtained in another preclinical study wherein the Wnt signaling pathway (a CSC-associated pathway) was therapeutically inhibited in an in vitro FOXC1+ cancer model (Ray et al., 2015b). While targeted inhibition of Wnt signaling was initially successful in markedly inhibiting mammosphere formation efficiency (a surrogate marker of cancer stem cell activity), resistant clones did emerge and mammosphere formation ability was regained. However, concurrent treatment with Bortezomib prevented the emergence of such resistant clones and effectively blocked the cancer stem cell escape mechanism.

Bortezomib and Ixazomib are two examples of FDA-approved drugs that are approved for use in the clinic to treat multiple myeloma. While Bortezomib is required to be administered intravenously (IV), Ixazomib is an oral drug with a superior toxicity profile, making it an ideal candidate for evaluating therapeutic efficacy against FOXC1+ pro-metastatic cancers by targeting the NFκB signaling pathway via proteasome inhibition. Based on this rationale, a Phase 1/II clinical trial (AGMT MBC-10) is currently underway to examine the efficacy of Ixazomib in combination with Carboplatin in previously treated advanced TNBC (Rinnerthaler et al., 2018). Marizomib, a next generation IV/oral, brain-penetrant, proteasome inhibitor which also displays dual oxidative phosphorylation inhibitory action, has been shown to possess excellent efficacy in an in vivo preclinical study of TNBC utilizing both nude mouse/syngeneic animal models, as well as patient-derived xenografts (Raninga et al., 2020). Marizomib not only caused a marked decline in tumor growth, it significantly reduced the development of lung and brain metastasis as well. Marizomib is currently being evaluated in multiple Phase 1/II and Ill clinical trials in a variety of cancer types. Thus, FOXC1 expression status may serve as a companion/complementary diagnostic for the proteasome inhibitor class of drugs by virtue of their ability to disrupt the NFκB-FOXC1 positive feedback loop.

### RAS/RAF/MEK/ERK/MAPK Signal Transduction Pathway

Utilizing a bioinformatic screening method, the breast cancer molecular subtype that was found to be most susceptible to treatment with small molecular MEK inhibitors was the basal-like subtype (Mirzoeva et al., 2009). The MAPK signal transduction cascade has been demonstrated to regulate and promote CSC traits in an in vivo mouse model of BLBC (Balko et al., 2013). Treatment with a MEK inhibitor in this model was successful in decreasing tumor growth. Pertinent to this review, RAS/MAPK pathway was demonstrated to regulate FOXC1 expression in breast cancer (Leontovich et al., 2012), suggesting that targeted inhibition of this pathway may offer therapeutic benefit in FOXC1+ pro-metastatic cancers. This and other preclinical evidence led to inhibitors of this pathway being tested as rational treatment strategy for TNBC (Giltnane and Balko, 2014). Subsequently it was demonstrated that RAS/MAPK activation was associated with reduced tumor infiltrating lymphocytes in TNBC and likely contributed towards immune evasion (Loi et al., 2016). This suggested the possibility that efficacy of immune checkpoint blockade might be further improved if combined with MEK inhibitor therapy. Since activation of this pathway was found to be elevated in patients diagnosed with TNBC who had already been treated with anthracycline-based chemotherapy regimens, the RAS/MAPK pathway may be an attractive therapeutic target in the subset of patients who relapse or have refractory disease (Eralp et al., 2008; Huang et al., 2017a). Emergence of resistance is a recognized phenomenon when undertaking targeted therapy of any signal transduction pathway in oncology, and the RAS/MAPK pathway proved to be no exception. Several groups reported a seemingly compensatory activation of the P13K/AKT pathway in response to RAS/MAPK inhibitor therapy, and therapeutic response was obtained upon employing P13K/AKT inhibitor therapy animal models of RAS/MAPK inhibitor-resistance (Hoeflich et al., 2009). This argued in favor of utilizing P13K/AKT inhibitors to address therapeutic resistance to RAS/MAPK inhibitor therapy, or to undertaking combination therapy with inhibitors of both the RAS/MAPK and P13K/AKT pathway (Saini et al., 2013). Another proposed approach to overcome therapeutic resistance encountered with MEK inhibitor therapy, or to achieve greater efficacy than could be achieved with MEK inhibitor therapy alone, was to undertake simultaneous blockade of multiple members of this signaling cascade. Thus, dual inhibition of both MEK and ERK (Hatzivassiliou et al., 2012) or of MEK and RAF (Nagaria et al., 2017), in preclinical models, proved to be successful in blocking the emergence of resistance as well as to overcome acquired resistance to MEK inhibitor therapy.

### P13K/AKT/mTOR Signal Transduction Pathway

P13K activating mutations and PTEN deactivating mutations are both known to increase and augment signal transduction through the P13K/AKT/mTOR pathway. And both of these pathway activating mutations are known to occur with greater frequency in TNBC (Cancer Genome Atlas, 2012). Furthermore, with regard to pathway activation status itself, the P13K/AKT/mTOR pathway is known to be significantly more upregulated in TNBC as compared to other receptor subtypes of breast cancer (Ueng et al., 2012; Walsh et al., 2012; Pelicano et al., 2014), and is known to contribute to both hormonal therapy resistance as well as chemotherapy resistance. Preclinical data was generated supporting the efficacy of P13K/AKT/mTOR inhibitors in in vitro and in vivo models of TNBC (Yunokawa et al., 2012). Based on these promising findings, a concerted effort was undertaken to examine the therapeutic efficacy of mTOR inhibitors in various clinical trials, in both ER+ breast cancer as well as TNBC. Everolimus was found to prolong progression free survival in patients diagnosed with ER+ breast cancer in the advanced/metastatic setting who had developed resistance to hormonal therapy (Kornblum et al., 2018). However, two separate Phase II trials failed to show any demonstrable efficacy in TNBC (Jovanovic et al., 2017; Park et al., 2018). However, an appropriate and suitable companion diagnostic was never established to better guide therapy in either ER+ breast cancer or TNBC. Therefore, at the present time, there is no way to predict which patients diagnosed with ER+ breast cancer, are most likely to respond to treatment with this class of targeted therapeutics. It is also therefore not known whether a specific subset within the TNBC patients tested in trials, did actually derive some clinical benefit, again because no correlation was found between therapeutic efficacy and the candidate companion diagnostic markers tested thus far.

From the clinical trial experience with targeted inhibition of both the RAS/RAF/MEK/ERK/MAPK and the PI3K/AKT/mTOR signal transduction pathways, we have learned that the responses have been variable and not consistent. Furthermore, undertaking a dual inhibitory approach of two different nodes (i.e. MEK and ERK, MEK and RAF, P13K and mTOR) or dual inhibition of both pathways is often fraught with severely limiting toxicity issues. One potential reason is that the bulk of the preclinical data that provided the initial rationale for these trials was obtained in models of BLBC. However, at the time of patient recruitment for these trials, the TNBC criterion was employed instead. As we have learned time and time again, the BLBC and TNBC definitions are definitely not synonymous and have a widely variable degree of overlap. The assumption that TNBC status is a close enough approximation and adequate surrogate equivalent of BLBC status is known to be incorrect. This might very well be an important factor contributing to potential miscalculation of the therapeutic efficacy in these trials as BLBC or high FOXC1+ status was not employed for patient enrichment, nor to assess potential companion diagnostic utility. A retrospective reassessment of the prospectively accrued patient samples from concluded trials on these lines may help to shed light on how trials evaluating the efficacy of this class of drugs might be better designed in the future.

### TGFβ Signal Transduction Pathway

Targeted inhibition of the TGFβ pathway to target tumor growth, progression and metastasis has been controversial. This is primarily because activation of this pathway was demonstrated by two independent studies to variably exert anti-tumorigenic effects on the one hand, but pro-metastagenic effects on the other (Siegel et al., 2003; Tang et al., 2003). Interest was however, rekindled in utilizing this approach for therapeutic benefit, when it was demonstrated that TGFβ signaling, in certain subsets of cancer patients like BLBC, contribute to immunosuppression in the tumor microenvironment and immune evasion by the tumor. This was first suggested by a study in which the therapeutic effects of a TGFβR1 inhibitor being evident in a syngeneic, immunocompetent mouse model with an intact immune system, but not demonstrable in an athymic nude mouse model that lacks the ability to mount an immune response (Ge et al., 2006). Independent validation followed in another report wherein use of an anti-TGFβ antibody successfully suppressed metastasis mainly by inducing a highly significant enhancement of the CD8+ T-cell-mediated antitumor immune response(Nam et al., 2008). A subsequent independent study highlighted how such an approach held promise in the therapy of BLBC (Ganapathy et al., 2010). Since then, multiple independent studies have reported that targeting TGFB with a therapeutic antibody (Mariathasan et al., 2018), with a small molecule inhibitor (Holmgaard et al., 2018) or with a bifunctional fusion protein (Lan et al., 2018) was able to overcome therapeutic resistance to immune checkpoint blockade, resulting in complete and durable therapeutic responses in otherwise poorly immunogenic tumors. Recently, another elegant and comprehensive study provided independent confirmation that therapeutic approaches based on TGFβ inhibition in breast cancer were most likely to be successful in patients with high grade, ER-negative disease of the claudin-low and basal subtypes (Yang et al., 2020). In summary, targeted inhibition of the TGFβ signaling pathway merits investigation in clinical trials to examine whether efficacy thereof can be predicted based on FOXC1 expression status.

### GLI2 Non-Canonical Hedgehog Signal Transduction Pathway

In a study by Cui and colleagues, FOXC1 was demonstrated to regulate CSC maintenance through activation of Smoothened-independent hedgehog signaling and binding to GLI2 in BLBC cells (Han et al., 2015). Furthermore, FOXC1 over-expression also induced resistance to SMO-inhibitors targeting canonical Hedgehog signaling Therefore, blockade of the non-canonical pathway described above through targeted inactivation of Gli2 might be a potential strategy to overcome acquired resistance to canonical Hedgehog SMO-inhibitors, and attenuate FOXC1 induced tumorigenicity and metastatic dissemination. Glasdegib is an oral SMO-inhibitor which is FDA-approved for the treatment of newly-diagnosed AML in adults older than 75 years or those who have comorbidities that preclude use of intensive induction chemotherapy (Cortes et al., 2019). In a preclinical study, Glasdegib was demonstrated to successfully target leukemia stem cells (Fukushima et al., 2016). Interestingly, although Glasdegib is a SMO-inhibitor, its effects downstream of its SMO-inhibitory action include GLI2 inhibition which abrogates leukemia stem cell dormancy (Sadarangani et al., 2015). As such, efficacy of Glasdegib in AML and/or other relevant cancers may be predicted by FOXC1 expression status.

Perfenidone is an oral drug approved for treatment of Idiopathic Pulmonary Fibrosis (Noble et al., 2011; King et al., 2014). Perfenidone has been proven to decrease fibrosis by decreasing TGFβ1 through a GLI2 inhibitory mechanism (Didiasova et al., 2017). However, by virtue of this same mechanism, Pirfenidone was recently demonstrated to be capable of inhibiting fibroblast activation and tumor-fibroblast crosstalk in the TME (Fujiwara et al., 2020). Another study reported that treatment with Perfenidone was successful in reducing immunosuppressive capacity of cancer-associated fibroblasts in the TME (Aboulkheyr Es et al., 2020). Most importantly, treatment with Pirfenidone was recently reported to augment the observed therapeutic benefit of PD1/PDL1 immune checkpoint blockade in syngeneic, immunocompetent mouse models of lung, liver and colon cancer (Qin et al., 2020). Collectively, these findings present a unique opportunity for testing in cancer clinical trials where FOXC1 expression status may be found to be useful in predicting efficacy of Perfenidone in ameliorating tumor growth and/or metastatic dissemination.

### CXCR1 Driven Cell-Cell Interaction

FOXC1 transcriptionally upregulates CXCR1 in breast cancer (Huang et al., 2015). CXCR1 is a breast CSC marker(Charafe-Jauffret et al., 2009), and upregulation has been reported to increase recruitment of FOXP3+ T regulatory cells to the TME and contribute to an immunosuppressive state (Eikawa et al., 2010). Repartaxin is a small molecule inhibitor of CXCR1 and has been demonstrated to have therapeutic efficacy in preclinical models of breast cancer (Ginestier et al., 2010) and gastric cancer (Wang et al., 2016b). In another study utilizing preclinical models of HER2-positive breast cancer, Repartaxin successfully reduced breast CSC activity and demonstrated enhanced therapeutic efficacy when combined with Lapatinib (Singh et al., 2013). In a window of opportunity neoadjuvant trial, Repartaxin was successful in decreasing the breast CSC count in by more than 20% in preoperative tumors (Goldstein et al., 2020). Newer CXCR1 inhibitors have also been reported to have efficacy in preclinical models of RCC and HNSCC (Dufies et al., 2019). Utilizing CXCR1 inhibitors to target CSCs is a promising therapeutic approach with sufficient pre-clinical data to merit examination of their therapeutic efficacy in FOXC1+ pro-metastatic cancers, especially in the setting of advanced/metastatic and/or recurrent cancers which are predicted to be enriched for FOXC1+ CSCs.

### CXCR4 Driven Cell-Cell Interaction

CXCR4 is a direct transcriptional target of FOXC1 in breast cancer that helps mediate increased invasion and metastasis in a preclinical model (Pan et al., 2018). In this model, treatment with Plerixafor, a CXCR4 antagonist was successful in reversing the CXCR4-mediated invasion and metastasis. Plerixafor is an oral inhibitor of CXCR4 currently FDA-approved as an HPSC mobilizer. The antitumor efficacy of Plerixafor in combination with a standard radio-chemotherapy protocol was evaluated in a murine model of cervical cancer (Chaudary et al., 2017). In another study, Plerixafor and another CXCR4 antagonist were observed to successfully reduce the incidence of bone metastasis in an animal model of prostate cancer (Gravina et al., 2015). More recently, Plerixafor was found to mobilize cancer stem cells from their niche in both AML as well as glioblastoma (Hira et al., 2020). Beyond CXCR4 expression status itself, FOXC1 expression status may provide additional information for more accurately predicting the therapeutic efficacy of CXCR4 inhibitors in various cancers and merits examination in clinical trials.

### FGFR1 Signal Transduction Pathway

As discussed above, FGFR1 is a proven transcriptional target of FOXC1 in breast cancer. Erdafitinib is a pan-FGFR oral inhibitor currently FDA-approved for the treatment of advanced or metastatic urothelial carcinoma (Loriot et al., 2019). Pemigatinib is another oral inhibitor of FGFR1/2/3 currently FDA-approved for the treatment of locally advanced or metastatic cholangiocarcinoma (Abou-Alfa et al., 2020). FOXC1 expression status may help predict therapeutic efficacy and improve patient selection for treatment with this class of drugs.

### FGFR4 Signal Transduction Pathway

As earlier discussed, FOXC1 transcriptionally upregulates FGFR4 in colon cancer (Liu et al., 2018a). Several selective small molecule inhibitors of FGFR4 have been developed (Hagel et al., 2015; Hatlen et al., 2019; Kim et al., 2019; Weiss et al., 2019; Rezende Miranda et al., 2020) whose efficacy in HCC and various other cancers is currently being evaluated in early phase clinical trials. FGFR4 activation is closely associated with its specific ligand FGF19. Beyond FGF19 expression status, FOXC1 expression status may provide additional information for more accurately predicting the therapeutic efficacy of FGFR4 inhibitors.

### CONCLUSION

Over the course of the past decade, much has been learned regarding the important role played by the transcription factor FOXC1 in cancer. It is now well accepted that FOXC1 regulates a diverse set of biologically aggressive traits in cancer. Beyond studies of association, FOXC1 has been demonstrated to play a causative role in cancer stem cell biology that contributes not only to an aggressive phenotype but to an aggressive clinical course as well, often culminating in metastatic dissemination and death. From the first report of its central role in aggressive BLBCs more than 10 years ago, we have come to understand that FOXC1 plays a pivotal functional role in more than 16 different cancer types. This list is likely to keep growing. During this interval, our understanding has advanced significantly. From an ever-expanding understanding of the functional and mechanistic role of FOXC1 in cancer, we have also developed an appreciation of its potential application as a powerful prognostic biomarker. FOXC1 expression in cancer tissues, has been demonstrated to be capable of identifying those patients who are at heightened risk of suffering metastatic recurrence. This is of particular relevance in those patient subsets (e.g. lymph node negative patients) where, based on traditional clinical factors, a heightened recurrence risk would have been least suspected. The fact that FOXC1 expression can now be measured quite easily using an inexpensive, clinically validated and commercially available assay (Jensen et al., 2015)means that testing can truly be accomplished on a global scale, even in resource-challenged settings. This is a huge step from a global oncology care perspective. By accurately identifying those patients with FOXC1+ pro-metastatic cancer who are at greatest risk, such a test serves to refine and improve resource utilization, to optimize delivery of life-saving chemotherapy and targeted therapy to the patients who need it the most. Testing to allow identification of metastasis risk early in the disease process is crucial in the fight to reduce glaring inequalities in global cancer control. Even by conservative estimates, a lack of such testing, if not remedied very quickly, will allow cancer to assume global pandemic proportions within the next decade (Lopez-Gomez et al., 2013).

While identification of elevated metastasis risk is important, it serves another purpose than the one delineated above. Not only would it allow for potentially life-extending systemic treatment (chemotherapy) to be administered to identified high risk patients, it would also allow for their targeted enrollment in the latest clinical trials. Both of these FOXC1 biomarker status-driven interventions have the potential to exert a significant decrease in overall cancer related morbidity and mortality. Beyond the above outlined points of clinical utility, however, sufficient evidence has now accrued to suggest that FOXC1 expression status might also find use to predict the efficacy of certain classes of targeted therapeutics in oncology practice. We have presented some such potentially therapeutic rationales in this review with the intention of spurring debate and stimulating discussion amongst the global oncology community. This of course will need many years of focused effort to first identify the most promising therapeutic approaches to target FOXC1+ pro-metastatic cancers, followed by carefully conducted clinical trials to test and prove their effectiveness. But if successful, such practice-changing advances hold the promise of diminishing the devastating prognostic impact of metastasis, currently the single largest contributor to cancer-related mortality, and extend the lives of cancer survivors.

According to the embodiments disclosed herein, complementary diagnostic protocols or assays are provided, which are based on detection of FOXC1 protein or mRNA, MK167/Ki67 protein or mRNA, CD274/PD-L1 protein or mRNA, alone or in specific combinations, in a relevant biologic/clinical tissue or cell sample (e.g., tumor tissue, circulating tumor cells, lymphocytes, etc.), to predict clinical efficacy of one or more classes of oncology drugs or drug combination regimens wherein one or more classes of oncology drugs include (i) a chemotherapeutic agent, (ii) a targeted therapeutic agent (biologic or non-biologic), (iii) an immunotherapeutic agent (biologic or non-biologic), or (iv) any combination of (i), (ii), and/or (iii) above.

In some embodiments, where a complementary diagnostic assay is used to predict clinical efficacy of a chemotherapeutic agent, the chemotherapeutic agent may include (a) an anthracycline class of chemotherapeutic drug including but not limited to doxorubicin, epirubicin, etc.; (b). a vinca alkaloid class of chemotherapeutic drug including but not limited to vincristine, vinblastine, vinorelbine, vindesine, etc.; (c) a taxane class of chemotherapeutic drug including but not limited to paclitaxel, nab-paclitaxel, docetaxel, nab-docetaxel, etc., (d) A nucleoside analog class of chemotherapeutic drug including but not limited to gemcitabine, etc.; (e) an antimetabolite class of chemotherapeutic drug including but not limited to 5-FU, capecitabine, etc.

In some embodiments, where a complementary diagnostic assay is used to predict clinical efficacy of a biologic agent, the biologic agent may include (a) an inhibitor of the Tumor Growth Factor Beta (TGF-β) signalling pathway including but not limited to Galunisertib, Perfenidone, etc.; (b) an inhibitor of the Nuclear Factor Kappa B (NF-kB) signalling pathway including but not limited to Bortezomib, Carfilzomib, Ixazomib, etc.; (c) an inhibitor of the P13K/PTEN/AKT/mTOR signalling pathway including but not limited to P13K inhibitors like Alpelisib, Copanlisib, AKT Inhibitors like Ipatasertib, Capivasertib, MTOR Inhibitors like Everolimus, Temsirolimus, etc.; (d) an inhibitor of the non-canonical Wnt signalling pathway; (e) an inhibitor of the non-canonical Hedgehog signalling pathway including but not limited to GL12 inhibitors like Glasdegib, Pirfenidone, etc.; (f) an inhibitor of the non-canonical Notch signalling pathway; (g) an inhibitor of the Epidermal Growth Factor Receptor (EGFR) signalling pathway including but not limited to Gefitinib, etc.; (h) an inhibitor of the RAS/RAF/MEK/ERK kinase enzymes including but not limited to RAS Inhibitors like Sotorasib, RAF Inhibitors like Sorafenib, Vemurafenib, MEK Inhibitors like Trametinib, Binimetinib, Selumetinib, ERK Inhibitors like Ulixertinib, etc.; (i) an inhibitor of the IL8/CXCR1 pathway including but not limited to IL8 inhibitors like Humax-IL8 (BMS-986253), CXCR1 inhibitors like Repertaxin, etc.; (j) an inhibitor of the CCL12/CXCR4 pathway including but not limited to CXCR4 inhibitors like Plerixafor, etc.; (k) an inhibitor of the FGF/FGFR1 pathway including but not limited to FGFR1 inhibitors like Pemigatinib, Erdafitinib, etc.; (1) an inhibitor of the the FGF19/FGFR4 pathway including but not limited to FGFR4 inhibitors like BLU9931, BLU554/Fisogatinib, FG401/Roblitinib, etc.; (m) an inhibitor of the CDK4/6 class of cyclin-dependent kinase enzymes including but not limited to Palbociclib, Ribociclib, Abemaciclib, etc.; (n) an inhibitor of the CDK7 class of cyclin-dependent kinase enzymes including but not limited to THZ1, THZ2, SY5609, CT7001/ICEC0942 (Samuraciclib), XL102, YKL-5-124, etc.; and/or (o) an inhibitor of KRAS(G12C) including but not limited to AMG510 (Sotorasib), Trametinib, etc.

In some embodiments, where a complementary diagnostic assay is used to predict clinical efficacy of a immunotherapeutic agent, the immunotherapeutic agent may include (a) an inhibitor of the PD1/PDL1 immune checkpoint including but not limited to PD1 inhibitors like Nivolumab, Pembrolizumab, Cemiplimab, etc.; (b) an inhibitor of the PD1/PDL1 immune checkpoint including but not limited to PDL1 inhibitors like Atezolizumab, Avelumab, Durvalumab, etc.; (c) an inhibitor of the CTLA4 immune checkpoint including but not limited to Ipilimumab, etc.; (d) An inhibitor of the LAG-3 immune checkpoint; (e) an inhibitor of the TIM-3 immune checkpoint; (f) an inhibitor of the B7H3/B7H4 immune checkpoint; (g) an inhibitor of the A2aR/A2bR/CD73 immune checkpoint; (h) an inhibitor of the NKG2A immune checkpoint; and/or (i) an inhibitor of the PVRIG/PVRL2 immune checkpoint.

In some embodiments where a complementary diagnostic assay is used to predict clinical efficacy of an oncology drug or drug combination regimen, the oncology drug or drug combination regimen may include but are not limited to (a) an anthracycline monotherapy regimen; (b) a taxane monotherapy regimen; (c) a platinum agent monotherapy regimen; (d) an anthracycline and taxane combination regimen including but not limited to doxorubicin and paclitaxel, epirubicin and paclitaxel, doxorubicin and docetaxel, epirubicin and docetaxel, etc.; (e) an anthracycline, platinum and anti-metabolite combination regimen including but not limited to doxorubicin, cisplatin and 5-FU, epirubucin, cisplatin and 5-FU, etc.; (f) a taxane and platinum combination regimen including but not limited to paclitaxel and cisplatin, paclitaxel and carboplatin, nab-paclitaxel and cisplatin, nab-paclitaxel and carboplatin, docetaxel and cisplatin, docetaxel and carboplatin, etc.; (g) a PARP inhibitor, platinum compound and PDL1 inhibitor combination regimen including but not limited to olaparib, paclitaxel and durvalumab, etc.; and/or (h) a taxane, platinum compound and PD1 inhibitor combination regimen including but not limited to docetaxel, carboplatin and pembrolizumab, etc.

In some embodiments, a method of using of a pre-specified cutoff value of FOXC1 protein detected using an anti-FOXC1 antibody in a biological or clinical sample obtained from a patient diagnosed with a cancer (e.g., breast cancer, lung cancer, colon cancer, endometrial cancer, cervical cancer, melanoma, head and neck cancer, leukemia, lymphoma) is provided to predict the efficacy or an enhanced efficacy of the above listed drugs or drug combination regimens wherein said efficacy is any or all of the following clinicopathologic outcome measures: (1) a decrease in size/number of the primary/metastatic tumor; (2) a decrease in cancer cell proliferation in the primary/metastatic tumor; (3) a decrease in cancer cell invasion, migration in the primary/metastatic tumor; (4) a decrease in incidence of recurrence of the cancer; (5) a decrease in incidence of metastatic spread of the cancer; (6) an increase/prolongation of disease-free survival of patients diagnosed with the cancer; (7) an increase/prolongation of recurrence-free survival of patients diagnosed with the cancer; (8) an increase/prolongation of distant metastasis-free survival of patients diagnosed with the cancer; (9) an increase/prolongation of event-free survival of patients diagnosed with the cancer; (10) an increase/prolongation of progression-free survival of patients diagnosed with the cancer; (11) an increase/prolongation of disease-specific survival of patients diagnosed with the cancer; and/or (12) an increase/prolongation of overall survival of patients diagnosed with the cancer.

The biologic or clinical sample may be any relevant tissue or cell sample including, but not limited to, tumor tissue, blood, plasma, bone marrow, circulating tumor cells, lymphocytes, etc.

The FOXC1 protein may be detected using any suitable method including, but not limited to, assays that use a FOXC1 antibody to target the FOXC1 protein or fragment thereof. In certain embodiments, the FOXC1 antibody is an anti-human FOXC1 mouse monoclonal antibody or mAb. In other embodiments, the FOXC1 antibody is the anti-human FOXC1 mouse monoclonal antibody described herein.

In some embodiments, a method of using of a pre-specified cutoff value of FOXC1 mRNA detected using one or more FOXC1 qRTPCR probes in a biological or clinical sample obtained from a patient diagnosed with a cancer (e.g., breast cancer, lung cancer, colon cancer, endometrial cancer, cervical cancer, melanoma, head and neck cancer, leukemia, lymphoma) is provided to predict the efficacy or an enhanced efficacy of the above listed drugs or drug combination regimens wherein said efficacy is any or all of the following clinicopathologic outcome measures: (1) a decrease in size/number of the primary/metastatic tumor; (2) a decrease in cancer cell proliferation in the primary/metastatic tumor; (3) a decrease in cancer cell invasion, migration in the primary/metastatic tumor; (4) a decrease in incidence of recurrence of the cancer; (5) a decrease in incidence of metastatic spread of the cancer; (6) an increase/prolongation of disease-free survival of patients diagnosed with the cancer; (7) an increase/prolongation of recurrence-free survival of patients diagnosed with the cancer; (8) an increase/prolongation of distant metastasis-free survival of patients diagnosed with the cancer; (9) an increase/prolongation of event-free survival of patients diagnosed with the cancer; (10) an increase/prolongation of progression-free survival of patients diagnosed with the cancer; (11) an increase/prolongation of disease-specific survival of patients diagnosed with the cancer; and/or (12) an increase/prolongation of overall survival of patients diagnosed with the cancer.

The biologic or clinical sample may be any relevant tissue or cell sample including, but not limited to, tumor tissue, blood, plasma, bone marrow, circulating tumor cells, lymphocytes, etc.

The FOXC1 qRTPCR probe or probes may be selected from those disclosed in International Application No. PCT/US10/44817 (which is hereby incorporated by reference as fully set forth herein), or any other probe designed to target Ki67 mRNA (e.g., oligonucleotides, siRNA or other RNAi molecule), and may be detected using any suitable method including, but not limited to, assays that use qRTPCR probes to target the FOXC1 mRNA or fragment thereof.

In some embodiments, a method of using of a pre-specified cutoff value of MK167/Ki67 protein detected using an MK167/Ki67 antibody in a biological or clinical sample obtained from a patient diagnosed with a cancer (e.g., breast cancer, lung cancer, colon cancer, endometrial cancer, cervical cancer, melanoma, head and neck cancer, leukemia, lymphoma) is provided to predict the efficacy or an enhanced efficacy of the above listed drugs or drug combination regimens wherein said efficacy is any or all of the following clinicopathologic outcome measures: (1) a decrease in size/number of the primary/metastatic tumor; (2) a decrease in cancer cell proliferation in the primary/metastatic tumor; (3) a decrease in cancer cell invasion, migration in the primary/metastatic tumor; (4) a decrease in incidence of recurrence of the cancer; (5) a decrease in incidence of metastatic spread of the cancer; (6) an increase/prolongation of disease-free survival of patients diagnosed with the cancer; (7) an increase/prolongation of recurrence-free survival of patients diagnosed with the cancer; (8) an increase/prolongation of distant metastasis-free survival of patients diagnosed with the cancer; (9) an increase/prolongation of event-free survival of patients diagnosed with the cancer; (10) an increase/prolongation of progression-free survival of patients diagnosed with the cancer; (11) an increase/prolongation of disease-specific survival of patients diagnosed with the cancer; and/or (12) an increase/prolongation of overall survival of patients diagnosed with the cancer.

The biologic or clinical sample may be any relevant tissue or cell sample including, but not limited to, tumor tissue, blood, plasma, bone marrow, circulating tumor cells, lymphocytes, etc.

The MK167/Ki67 protein may be detected using any suitable method including, but not limited to, assays that use an anti-MK167/Ki67 antibody to target the MK167/Ki67 protein or fragment thereof. In certain embodiments, the MK167/Ki67 antibody is an anti-human MK167/Ki67 mouse monoclonal antibody or mAb.

In some embodiments, a method of using of a pre-specified cutoff value of MK167/Ki67 mRNA detected using one or more MK167/Ki67 qRTPCR probes in a biological or clinical sample obtained from a patient diagnosed with a cancer (e.g., breast cancer, lung cancer, colon cancer, endometrial cancer, cervical cancer, melanoma, head and neck cancer, leukemia, lymphoma) is provided to predict the efficacy or an enhanced efficacy of the above listed drugs or drug combination regimens wherein said efficacy is any or all of the following clinicopathologic outcome measures: (1) a decrease in size/number of the primary/metastatic tumor; (2) a decrease in cancer cell proliferation in the primary/metastatic tumor; (3) a decrease in cancer cell invasion, migration in the primary/metastatic tumor; (4) a decrease in incidence of recurrence of the cancer; (5) a decrease in incidence of metastatic spread of the cancer; (6) an increase/prolongation of disease-free survival of patients diagnosed with the cancer; (7) an increase/prolongation of recurrence-free survival of patients diagnosed with the cancer; (8) an increase/prolongation of distant metastasis-free survival of patients diagnosed with the cancer; (9) an increase/prolongation of event-free survival of patients diagnosed with the cancer; (10) an increase/prolongation of progression-free survival of patients diagnosed with the cancer; (11) an increase/prolongation of disease-specific survival of patients diagnosed with the cancer; and/or (12) an increase/prolongation of overall survival of patients diagnosed with the cancer.

The biologic or clinical sample may be any relevant tissue or cell sample including, but not limited to, tumor tissue, blood, plasma, bone marrow, circulating tumor cells, lymphocytes, etc.

The MK167/Ki67 qRTPCR probe or probes can be any probe (e.g., oligonucleotide or the like) designed to target Ki67 mRNA and may be detected using any suitable method including, but not limited to, assays that use qRTPCR probes to target the MK167/Ki67 mRNA or fragment thereof.

In some embodiments, a method of using of a pre-specified cutoff value of CD274/PD-L1 protein detected using an CD274/PD-L1 antibody in a biological or clinical sample obtained from a patient diagnosed with a cancer (e.g., breast cancer, lung cancer, colon cancer, endometrial cancer, cervical cancer, melanoma, head and neck cancer, leukemia, lymphoma) is provided to predict the efficacy or an enhanced efficacy of the above listed drugs or drug combination regimens wherein said efficacy is any or all of the following clinicopathologic outcome measures: (1) a decrease in size/number of the primary/metastatic tumor; (2) a decrease in cancer cell proliferation in the primary/metastatic tumor; (3) a decrease in cancer cell invasion, migration in the primary/metastatic tumor; (4) a decrease in incidence of recurrence of the cancer; (5) a decrease in incidence of metastatic spread of the cancer; (6) an increase/prolongation of disease-free survival of patients diagnosed with the cancer; (7) an increase/prolongation of recurrence-free survival of patients diagnosed with the cancer; (8) an increase/prolongation of distant metastasis-free survival of patients diagnosed with the cancer; (9) an increase/prolongation of event-free survival of patients diagnosed with the cancer; (10) an increase/prolongation of progression-free survival of patients diagnosed with the cancer; (11) an increase/prolongation of disease-specific survival of patients diagnosed with the cancer; and/or (12) an increase/prolongation of overall survival of patients diagnosed with the cancer.

The biologic or clinical sample may be any relevant tissue or cell sample including, but not limited to, tumor tissue, blood, plasma, bone marrow, circulating tumor cells, lymphocytes, etc.

The CD274/PD-L1 protein may be detected using any suitable method including, but not limited to, assays that use an anti-CD274/PD-L1 antibody to target the CD274/PD-L1 protein or fragment thereof. In certain embodiments, the CD274/PD-L1 antibody is an anti-human CD274/PD-L1 mouse monoclonal antibody or mAb.

In some embodiments, a method of using of a pre-specified cutoff value of CD274/PD-L1 mRNA detected using one or more CD274/PD-L1 qRTPCR probes in a biological or clinical sample obtained from a patient diagnosed with a cancer (e.g., breast cancer, lung cancer, colon cancer, endometrial cancer, cervical cancer, melanoma, head and neck cancer, leukemia, lymphoma) is provided to predict the efficacy or an enhanced efficacy of the above listed drugs or drug combination regimens wherein said efficacy is any or all of the following clinicopathologic outcome measures: (1) a decrease in size/number of the primary/metastatic tumor; (2) a decrease in cancer cell proliferation in the primary/metastatic tumor; (3) a decrease in cancer cell invasion, migration in the primary/metastatic tumor; (4) a decrease in incidence of recurrence of the cancer; (5) a decrease in incidence of metastatic spread of the cancer; (6) an increase/prolongation of disease-free survival of patients diagnosed with the cancer; (7) an increase/prolongation of recurrence-free survival of patients diagnosed with the cancer; (8) an increase/prolongation of distant metastasis-free survival of patients diagnosed with the cancer; (9) an increase/prolongation of event-free survival of patients diagnosed with the cancer; (10) an increase/prolongation of progression-free survival of patients diagnosed with the cancer; (11) an increase/prolongation of disease-specific survival of patients diagnosed with the cancer; and/or (12) an increase/prolongation of overall survival of patients diagnosed with the cancer.

The biologic or clinical sample may be any relevant tissue or cell sample including, but not limited to, tumor tissue, blood, plasma, bone marrow, circulating tumor cells, lymphocytes, etc.

The CD274/PD-L1 qRTPCR probe or probes may be selected from those any probe designed to target CD274/PD-L1 mRNA and may be detected using any suitable method including, but not limited to, assays that use qRTPCR probes to target the CD274/PD-L1 mRNA or fragment thereof.

In some embodiments, a method of using of a pre-specified cutoff value of FOXC1 and MK167/Ki67 proteins detected using an anti-FOXC1 and an anti-MK167/Ki67 antibody in a biological or clinical sample obtained from a patient diagnosed with a cancer (e.g., breast cancer, lung cancer, colon cancer, endometrial cancer, cervical cancer, melanoma, head and neck cancer, leukemia, lymphoma) is provided to predict the efficacy or an enhanced efficacy of the above listed drugs or drug combination regimens wherein said efficacy is any or all of the following clinicopathologic outcome measures: (1) a decrease in size/number of the primary/metastatic tumor; (2) a decrease in cancer cell proliferation in the primary/metastatic tumor; (3) a decrease in cancer cell invasion, migration in the primary/metastatic tumor; (4) a decrease in incidence of recurrence of the cancer; (5) a decrease in incidence of metastatic spread of the cancer; (6) an increase/prolongation of disease-free survival of patients diagnosed with the cancer; (7) an increase/prolongation of recurrence-free survival of patients diagnosed with the cancer; (8) an increase/prolongation of distant metastasis-free survival of patients diagnosed with the cancer; (9) an increase/prolongation of event-free survival of patients diagnosed with the cancer; (10) an increase/prolongation of progression-free survival of patients diagnosed with the cancer; (11) an increase/prolongation of disease-specific survival of patients diagnosed with the cancer; and/or (12) an increase/prolongation of overall survival of patients diagnosed with the cancer.

The biologic or clinical sample may be any relevant tissue or cell sample including, but not limited to, tumor tissue, blood, plasma, bone marrow, circulating tumor cells, lymphocytes, etc.

The FOXC1 and MK167/Ki67 proteins may be detected using any suitable method including, but not limited to, assays that use anti-FOXC1 and anti-MK167/Ki67 antibodies to target the FOXC1 and MK167/Ki67 proteins or fragments thereof. In certain embodiments, the anti-FOXC1 and anti-MK167/Ki67 antibodies are an anti-human anti-FOXC1 and anti-MK167/Ki67 mouse monoclonal antibodies or mAbs.

In some embodiments, a method of using of a pre-specified cutoff value of FOXC1 and MK167/Ki67 mRNA detected using one or more FOXC1 qRTPCR probes and one or more MK167/Ki67 qRTPCR probes in a biological or clinical sample obtained from a patient diagnosed with a cancer (e.g., breast cancer, lung cancer, colon cancer, endometrial cancer, cervical cancer, melanoma, head and neck cancer, leukemia, lymphoma) is provided to predict the efficacy or an enhanced efficacy of the above listed drugs or drug combination regimens wherein said efficacy is any or all of the following clinicopathologic outcome measures: (1) a decrease in size/number of the primary/metastatic tumor; (2) a decrease in cancer cell proliferation in the primary/metastatic tumor; (3) a decrease in cancer cell invasion, migration in the primary/metastatic tumor; (4) a decrease in incidence of recurrence of the cancer; (5) a decrease in incidence of metastatic spread of the cancer; (6) an increase/prolongation of disease-free survival of patients diagnosed with the cancer; (7) an increase/prolongation of recurrence-free survival of patients diagnosed with the cancer; (8) an increase/prolongation of distant metastasis-free survival of patients diagnosed with the cancer; (9) an increase/prolongation of event-free survival of patients diagnosed with the cancer; (10) an increase/prolongation of progression-free survival of patients diagnosed with the cancer; (11) an increase/prolongation of disease-specific survival of patients diagnosed with the cancer; and/or (12) an increase/prolongation of overall survival of patients diagnosed with the cancer.

The biologic or clinical sample may be any relevant tissue or cell sample including, but not limited to, tumor tissue, blood, plasma, bone marrow, circulating tumor cells, lymphocytes, etc.

The FOXC1 qRTPCR probes and MK167/Ki67 qRTPCR probes may be selected from those any probe designed to target FOXC1 and MK167/Ki67 mRNA and may be detected using any suitable method including, but not limited to, assays that use qRTPCR probes to target the FOXC1 and MK167/Ki67 mRNA or fragments thereof.

IX. Use of pre-specified cutoff values of FOXC1, MK167/Ki67 and CD274/PD-L1 proteins detected in a relevant biologic/clinical tissue/cell sample (tumor tissue, circulating tumor cells, lymphocyte, etc.), using the below described anti-human FOXC1, MK167/Ki67 and CD274/PD-L1 mouse monoclonal antibody or mAb, obtained from patients diagnosed with breast cancer, lung cancer, colon cancer, endometrial cancer, cervical cancer, melanoma, head and neck cancer, leukemia, lymphoma, or a different cancer, to predict efficacy/enhanced efficacy of the above listed drugs or drug combination regimens wherein said efficacy is any or all of the following clinicopathologic outcome measures:

In some embodiments, a method of using of a pre-specified cutoff value of FOXC1, MK167/Ki67 and CD274/PD-L1 proteins detected using an anti-FOXC1, an anti-MK167/Ki67 antibody, and an anti-CD27/PD-L1 antibody in a biological or clinical sample obtained from a patient diagnosed with a cancer (e.g., breast cancer, lung cancer, colon cancer, endometrial cancer, cervical cancer, melanoma, head and neck cancer, leukemia, lymphoma) is provided to predict the efficacy or an enhanced efficacy of the above listed drugs or drug combination regimens wherein said efficacy is any or all of the following clinicopathologic outcome measures: (1) a decrease in size/number of the primary/metastatic tumor; (2) a decrease in cancer cell proliferation in the primary/metastatic tumor; (3) a decrease in cancer cell invasion, migration in the primary/metastatic tumor; (4) a decrease in incidence of recurrence of the cancer; (5) a decrease in incidence of metastatic spread of the cancer; (6) an increase/prolongation of disease-free survival of patients diagnosed with the cancer; (7) an increase/prolongation of recurrence-free survival of patients diagnosed with the cancer; (8) an increase/prolongation of distant metastasis-free survival of patients diagnosed with the cancer; (9) an increase/prolongation of event-free survival of patients diagnosed with the cancer; (10) an increase/prolongation of progression-free survival of patients diagnosed with the cancer; (11) an increase/prolongation of disease-specific survival of patients diagnosed with the cancer; and/or (12) an increase/prolongation of overall survival of patients diagnosed with the cancer.

The biologic or clinical sample may be any relevant tissue or cell sample including, but not limited to, tumor tissue, blood, plasma, bone marrow, circulating tumor cells, lymphocytes, etc.

The FOXC1, MK167/Ki67 and CD274/PD-L1 proteins may be detected using any suitable method including, but not limited to, assays that use anti-FOXC1, anti-MK167/Ki67, and anti-CD27/PD-L1 antibodies to target the FOXC1, MK167/Ki67 and CD274/PD-L1 proteins or fragments thereof. In certain embodiments, the anti-FOXC1, anti-MK167/Ki67, and anti-CD27/PD-L1 antibodies are an anti-human anti-FOXC1, anti-MK167/Ki67, and anti-CD27/PD-L1 mouse monoclonal antibodies or mAbs.

In some embodiments, a method of using of a pre-specified cutoff value of FOXC1, MK167/Ki67 and CD274/PD-L1 mRNA detected using one or more FOXC1 qRTPCR probes, one or more MK167/Ki67 qRTPCR probes, and one or more CD27/PD-L1 qRTPCR probes in a biological or clinical sample obtained from a patient diagnosed with a cancer (e.g., breast cancer, lung cancer, colon cancer, endometrial cancer, cervical cancer, melanoma, head and neck cancer, leukemia, lymphoma) is provided to predict the efficacy or an enhanced efficacy of the above listed drugs or drug combination regimens wherein said efficacy is any or all of the following clinicopathologic outcome measures: (1) a decrease in size/number of the primary/metastatic tumor; (2) a decrease in cancer cell proliferation in the primary/metastatic tumor; (3) a decrease in cancer cell invasion, migration in the primary/metastatic tumor; (4) a decrease in incidence of recurrence of the cancer; (5) a decrease in incidence of metastatic spread of the cancer; (6) an increase/prolongation of disease-free survival of patients diagnosed with the cancer; (7) an increase/prolongation of recurrence-free survival of patients diagnosed with the cancer; (8) an increase/prolongation of distant metastasis-free survival of patients diagnosed with the cancer; (9) an increase/prolongation of event-free survival of patients diagnosed with the cancer; (10) an increase/prolongation of progression-free survival of patients diagnosed with the cancer; (11) an increase/prolongation of disease-specific survival of patients diagnosed with the cancer; and/or (12) an increase/prolongation of overall survival of patients diagnosed with the cancer.

The biologic or clinical sample may be any relevant tissue or cell sample including, but not limited to, tumor tissue, blood, plasma, bone marrow, circulating tumor cells, lymphocytes, etc.

The FOXC1 qRTPCR probes, MK167/Ki67 qRTPCR probes, and CD27/PD-L1 qRTPCR probes may be selected from those any probe designed to target FOXC1, MK167/Ki67, and CD27/PD-L1 mRNA and may be detected using any suitable method including, but not limited to, assays that use qRTPCR probes to target the FOXC1, MK167/Ki67 and CD274/PD-L1 mRNA or fragments thereof.

Biomarkers that predict efficacy of specific chemotherapy or immunotherapy regimens in cancer are useful for guiding therapy. If a specific chemotherapy or immunotherapy that is intended to be used can be accurately predicted to have a high probability of having therapeutic efficacy in a patient, then that therapeutic approach can be prescribed with confidence of having a high likelihood of exerting a positive clinical benefit. If however, the intended approach is accurately predicted to have a high probability of lacking therapeutic efficacy, then the therapeutic approach should probably be changed to an alternative approach in rode rot have a higher likelihood of exerting a positive clinical benefit.

“Pre-treatment” biomarkers that can be measured in pre-treatment biopsy tissue samples are preferable to “on-treatment” biomarkers that typically require repeat biopsies and tissue sampling. The described method falls under the category of being a pre-treatment predictive biomarker to predict therapeutic efficacy of a wide range of chemotherapeutic regimens and/or immunotherapeutic regimens in cancer and as such represents a pragmatic solution to achieving personalized cancer care.

The method involves obtaining a pre-treatment tumor tissue biopsy sample processed with formalin fixation and paraffin embedding (FFPE) using standard protocols, and then subjected to measurement of the said biomarkers (MK167, FOXC1 or PDL1/CD274) at the mRNA level using the quantitative reverse transcriptase polymerase chain reaction (qRTPCR) method or the ribonucleic acid sequencing (RNA-Seq) method. Alternatively, the said biomarkers can be measured at the protein level using standard immunohistochemistry (IHC) protocols using the specific antibodies named in this application.

Once measured values for each biomarker are obtained, specific cutoff values are applied and the information combined to obtain the specific therapeutic efficacy prediction result. Data in support of the various clinical utilities of this approach in various cancers are summarized below.

### Examples

The following Examples provide an additional description of the present technology for illustrative purposes only and should not be construed to limit the scope of the present technology in any way.

### Example 1: Complementary Diagnostic Assays to predict clinical

efficacy of the class of oncology products (drugs, biologics, radiologics, or any combination thereof) that inhibit/block the Tumor Growth Factor Beta (TGF-β) signaling pathway in a variety of cancers. These include but are not limited to Galunisertib, Pirfenidone, etc.

Use of a pre-specified cutoff value of FOXC1 protein detected in tumor tissue (using the below described anti-human FOXC1 mouse monoclonal antibody or mAb) to predict efficacy/enhanced efficacy of the class of oncology products (drugs and/or biologics) that inhibit/block/attenuate TGF-β signaling pathway activation/activity, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) to predict efficacy/enhanced efficacy of the class of oncology products (drugs and/or biologics) that inhibit/block/attenuate TGF-β signaling pathway activation/activity, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, to predict efficacy/enhanced efficacy of the class of oncology products (drugs and/or biologics) that decrease/attenuate/inhibit/block TGF-β signaling pathway activation/activity, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 2: Complementary Diagnostic Assays to Predict Clinical

efficacy of the class of oncology products (drugs, biologics, radiologics, or any combination thereof) that inhibit/block the Nuclear Factor Kappa B (NFκB) signaling pathway in a variety of cancers. These include but are not limited to Bortezomib, Carfilzomib, Ixazomib, etc.

Use of a pre-specified cutoff value of FOXC1 protein detected in tumor tissue (using the below described anti-human FOXC1 mouse monoclonal antibody or mAb) to predict efficacy/enhanced efficacy of the class of oncology products (drugs and/or biologics) that inhibit/block/attenuate NFκB signaling pathway activation/activity, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) to predict efficacy/enhanced efficacy of the class of oncology products (drugs and/or biologics) that inhibit/block/attenuate NFκB signaling pathway activation/activity, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, to predict efficacy/enhanced efficacy of the class of oncology products (drugs and/or biologics) that decrease/attenuate/inhibit/block NFκB signaling pathway activation/activity, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 3: Complementary Diagnostic Assays to predict clinical

efficacy of the class of oncology products (drugs, biologics, radiologics, or any combination thereof) that inhibit/block the P13K/PTEN/AKT/mTOR signaling pathway in a variety of cancers. These include but are not limited to P13K inhibitors like Alpelisib, Copanlisib, AKT Inhibitors like Ipatasertib, Capivasertib, MTOR Inhibitors like Everolimus, Temsirolimus, etc.

Use of a pre-specified cutoff value of FOXC1 protein detected in tumor tissue (using the below described anti-human FOXC1 mouse monoclonal antibody or mAb) to predict efficacy/enhanced efficacy of the class of oncology products (drugs and/or biologics) that inhibit/block/attenuate P13K/PTEN/AKT/mTOR signaling pathway activation/activity, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) to predict efficacy/enhanced efficacy of the class of oncology products (drugs and/or biologics) that inhibit/block/attenuate P13K/PTEN/AKT/mTOR signaling pathway activation/activity, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, to predict efficacy/enhanced efficacy of the class of oncology products (drugs and/or biologics) that decrease/attenuate/inhibit/block P13K/PTEN/AKT/mTOR signaling pathway activation/activity, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 4: Complementary Diagnostic Assays to Predict Clinical

efficacy of the class of oncology products (drugs, biologics, radiologics, or any combination thereof) that inhibit/block the non-canonical Wnt signaling pathway in a variety of cancers.

Use of a pre-specified cutoff value of FOXC1 protein detected in tumor tissue (using the below described anti-human FOXC1 mouse monoclonal antibody or mAb) to predict efficacy/enhanced efficacy of the class of oncology products (drugs and/or biologics) that inhibit/block/attenuate non-canonical Wnt signaling pathway activation/activity, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) to predict efficacy/enhanced efficacy of the class of oncology products (drugs and/or biologics) that inhibit/block/attenuate non-canonical Wnt signaling pathway activation/activity, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, to predict efficacy/enhanced efficacy of the class of oncology products (drugs and/or biologics) that decrease/attenuate/inhibit/block non-canonical Wnt signaling pathway activation/activity, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 5: Complementary Diagnostic Assays to predict clinical

efficacy of the class of oncology products (drugs, biologics, radiologics, or any combination thereof) that inhibit/block the non-canonical Hedgehog signaling pathway in a variety of cancers. This includes but is not limited to GLI2 inhibitors like Glasdegib, Pirfenidone, etc.

Use of a pre-specified cutoff value of FOXC1 protein detected in tumor tissue (using the below described anti-human FOXC1 mouse monoclonal antibody or mAb) to predict efficacy/enhanced efficacy of the class of oncology products (drugs and/or biologics) that inhibit/block/attenuate non-canonical Hedgehog signaling pathway activation/activity, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) to predict efficacy/enhanced efficacy of the class of oncology products (drugs and/or biologics) that inhibit/block/attenuate non-canonical Hedgehog pathway activation/activity, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, to predict efficacy/enhanced efficacy of the class of oncology products (drugs and/or biologics) that decrease/attenuate/inhibit/block non-canonical Hedgehog signaling pathway activation/activity, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 6: Complementary Diagnostic Assays to Predict Clinical

efficacy of the class of oncology products (drugs, biologics, radiologics, or any combination thereof) that inhibit/block the non-canonical Notch signaling pathway in a variety of cancers.

Use of a pre-specified cutoff value of FOXC1 protein detected in tumor tissue (using the below described anti-human FOXC1 mouse monoclonal antibody or mAb) to predict efficacy/enhanced efficacy of the class of oncology products (drugs and/or biologics) that inhibit/block/attenuate non-canonical Notch signaling pathway activation/activity, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) to predict efficacy/enhanced efficacy of the class of oncology products (drugs and/or biologics) that inhibit/block/attenuate non-canonical Notch signaling pathway activation/activity, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, to predict efficacy/enhanced efficacy of the class of oncology products (drugs and/or biologics) that decrease/attenuate/inhibit/block non-canonical Notch signaling pathway activation/activity, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 7: Complementary Diagnostic Assays to Predict Clinical

efficacy of the class of oncology products (drugs, biologics, radiologics, or any combination thereof) that inhibit/block the PD1/PDL1 immune checkpoint in a variety of cancers. These include but are not limited to PD1 inhibitors like Nivolumab, Pembrolizumab, Cemiplimab, PDL1 inhibitors like Atezolizumab, Avelumab, Durvalumab, etc.

Use of a pre-specified cutoff value of FOXC1 protein detected in tumor tissue (using the below described anti-human FOXC1 mouse monoclonal antibody or mAb) to predict efficacy/enhanced efficacy of the class of oncology products (drugs and/or biologics) that inhibit/block/attenuate the PD1/PDL1 immune checkpoint, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) to predict efficacy/enhanced efficacy of the class of oncology products (drugs and/or biologics) that inhibit/block/attenuate the PD1/PDL1 immune checkpoint, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that decrease/attenuate/inhibit/block the PD1/PDL1 immune checkpoint, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 8: Complementary Diagnostic Assays to Predict Clinical

efficacy of the class of oncology products (drugs, biologics, radiologics, or any combination thereof) that inhibit/block the CTLA4 immune checkpoint in a variety of cancers. This includes but is not limited to Ipilimumab, etc.

Use of a pre-specified cutoff value of FOXC1 protein detected in tumor tissue (using the below described anti-human FOXC1 mouse monoclonal antibody or mAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the CTLA4 immune checkpoint, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the CTLA4 immune checkpoint, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that decrease/attenuate/inhibit/block the CTLA4 immune checkpoint, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 9: Complementary Diagnostic Assays to Predict Clinical

efficacy of the class of oncology products (drugs, biologics, radiologics, or any combination thereof) that inhibit/block the CDK4/6 class of cyclin-dependent kinase enzymes in a variety of cancers. This includes but is not limited to Palbociclib, Ribociclib, Abemaciclib, etc.

Use of a pre-specified cutoff value of FOXC1 protein detected in tumor tissue (using the below described anti-human FOXC1 mouse monoclonal antibody or mAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the CDK4/6 class of cyclin-dependent kinase enzymes in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the CDK4/6 class of cyclin-dependent kinase enzymes in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that decrease/attenuate/inhibit/block the CDK4/6 class of cyclin-dependent kinase enzymes in a variety of cancers, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 10: Complementary Diagnostic Assays to Predict Clinical

efficacy of the class of oncology products (drugs, biologics, radiologics, or any combination thereof) that inhibit/block EGFR in a variety of cancers. This includes but is not limited to Gefitinib, etc.

Use of a pre-specified cutoff value of FOXC1 protein detected in tumor tissue (using the below described anti-human FOXC1 mouse monoclonal antibody or mAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate EGFR in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate EGFR in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that decrease/attenuate/inhibit/block EGFR in a variety of cancers, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 11: Complementary Diagnostic Assays to Predict Clinical

efficacy of the class of oncology products (drugs, biologics, radiologics, or any combination thereof) that inhibit/block the RAS/RAF/MEK/ERK kinase enzymes in a variety of cancers. This includes but is not limited to RAS Inhibitors like Sotorasib, RAF Inhibitors like Sorafenib, Vemurafenib, MEK Inhibitors like Trametinib, Binimetinib, ERK Inhibitors like Ulixertinib, etc.

Use of a pre-specified cutoff value of FOXC1 protein detected in tumor tissue (using the below described anti-human FOXC1 mouse monoclonal antibody or mAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the RAS/RAF/MEK/ERK kinase enzymes in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the RAS/RAF/MEK/ERK kinase enzymes in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that decrease/attenuate/inhibit/block the RAS/RAF/MEK/ERK kinase enzymes in a variety of cancers, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 12: Complementary Diagnostic Assays to Predict Clinical

efficacy of the class of oncology products (drugs, biologics, radiologics, or any combination thereof) that inhibit/block the IL8/CXCR1 pathway in a variety of cancers. This includes but is not limited to IL8 inhibitors like Humax-IL8 (BMS-986253), CXCR1 inhibitors like Repertaxin, etc.

Use of a pre-specified cutoff value of FOXC1 protein detected in tumor tissue (using the below described anti-human FOXC1 mouse monoclonal antibody or mAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the IL8/CXCR1 pathway in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the IL8/CXCR1 pathway in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that decrease/attenuate/inhibit/block the IL8/CXCR1 pathway in a variety of cancers, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 13: Complementary Diagnostic Assays to Predict Clinical

efficacy of the class of oncology products (drugs, biologics, radiologics, or any combination thereof) that inhibit/block the CCL12/CXCR4 pathway in a variety of cancers. This includes but is not limited to CXCR4 inhibitors like Plerixafor, etc.

Use of a pre-specified cutoff value of FOXC1 protein detected in tumor tissue (using the below described anti-human FOXC1 mouse monoclonal antibody or mAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the CCL12/CXCR4 pathway in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the CCL12/CXCR4 pathway in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that decrease/attenuate/inhibit/block the CCL12/CXCR4 pathway in a variety of cancers, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 14: Complementary Diagnostic Assays to Predict Clinical

efficacy of the class of oncology products (drugs, biologics, radiologics, or any combination thereof) that inhibit/block the FGF/FGFR1 pathway in a variety of cancers. This includes but is not limited to FGFR1 inhibitors like Pemigatinib, Erdafitinib, etc.

Use of a pre-specified cutoff value of FOXC1 protein detected in tumor tissue (using the below described anti-human FOXC1 mouse monoclonal antibody or mAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the FGF/FGFR1 pathway in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the FGF/FGFR1 pathway in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that decrease/attenuate/inhibit/block the FGF/FGFR1 pathway in a variety of cancers, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 15: Complementary Diagnostic Assays to Predict Clinical

efficacy of the class of oncology products (drugs, biologics, radiologics, or any combination thereof) that inhibit/block the FGF19/FGFR4 pathway in a variety of cancers. This includes but is not limited to FGFR4 inhibitors like BLU9931, BLU554/Fisogatinib, FG401/Roblitinib, etc.

Use of a pre-specified cutoff value of FOXC1 protein detected in tumor tissue (using the below described anti-human FOXC1 mouse monoclonal antibody or mAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the FGF19/FGFR4 pathway in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a pre-specified cutoff value of FOXC1 protein detected in tumor tissue using the below described anti-human FOXC1 recombinant monoclonal antibody or rAb) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that inhibit/block/attenuate the FGF19/FGFR4 pathway in a variety of cancers, wherein said efficacy is any or all of includes the following clinicopathologic outcome measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

Use of a prespecified cutoff value of FOXC1 mRNA detected in tumor tissue (using the below described quantitative reverse transcriptase polymerase chain reaction or q-RTPCR primer probes) obtained from patients diagnosed with breast cancer, lung cancer, colon cancer, endometrial cancer, cervical cancer, melanoma, head and neck cancer, or a different cancer, to predict efficacy/enhanced efficacy of the class of oncology products (drugs and/or biologics) that decrease/attenuate/inhibit/block the FGF19/FGFR4 pathway in a variety of cancers, wherein said efficacy is any or all of includes the following clinicopathologic measures:
decrease in size/number of the primary/metastatic tumor
decrease in cancer cell proliferation in the primary/metastatic tumor
decrease in cancer cell invasion, migration in the primary/metastatic tumor
decrease in incidence of recurrence of the cancer
decrease in incidence of metastatic spread of the cancer
increase/prolongation of disease-free survival of patients diagnosed with the cancer
increase/prolongation of recurrence-free survival of patients diagnosed with the cancer
increase/prolongation of distant metastasis-free survival of patients diagnosed with the cancer
increase/prolongation of event-free survival of patients diagnosed with the cancer
increase/prolongation of progression-free survival of patients diagnosed with the cancer
increase/prolongation of disease-specific survival of patients diagnosed with the cancer
increase/prolongation of overall survival of patients diagnosed with the cancer

### Example 16: FOXC1 and MK167 Expression as a Complementary Diagnostic Assay for Predicting Efficacy of Oncology Drugs and Drug Combination Regimens and Methods for their Use

Immune checkpoint inhibitors (ICIs) have shown clinical efficacy when administered in combination with neoadjuvant chemotherapy (NACT) for the treatment of triple negative breast cancer (TNBC) and HER2 negative estrogen receptor (ER)+ breast cancer. However, suitable complementary diagnostics to help guide and tailor treatment recommendations are still lacking. Ki67 is a well-accepted and routinely used marker that tracks proliferation and has been shown to predict efficacy of neoadjuvant chemotherapy. Forkhead Box C1 (FOXC1), a transcriptional driver of cell plasticity/partial EMT/metastasis/immune evasion has proven prognostic value, but remains of uncertain predictive value. We sought to evaluate the potential of a Ki67 and FOXC1-based response predictor as a possible complementary diagnostic for a neoadjuvant regimen comprising of a PARP inhibitor (Olaparib), a Taxane chemotherapeutic (Paclitaxel) and an ICI of the PDL1 class (Durvalumab) in patients diagnosed with primary TNBC or HER2 negative ER+ breast cancer.

Methods. 41 Pre-NACT tumor biopsy MK167 and FOXC1 mRNA expression values were retrospectively obtained from TNBC patients and HER2 negative ER+ breast cancer patients who had been enrolled and treated in the I-SPY2 clinical trial (Durvalumab arm: 21, Control arm: 20) and correlated with the rate of observed pathologic complete response (pCR). The area under the curve (AUC) of each model was calculated and used to determine suitable cutoff values to maximize Negative Predictive Value (NPV) and Sensitivity for pCR prediction.

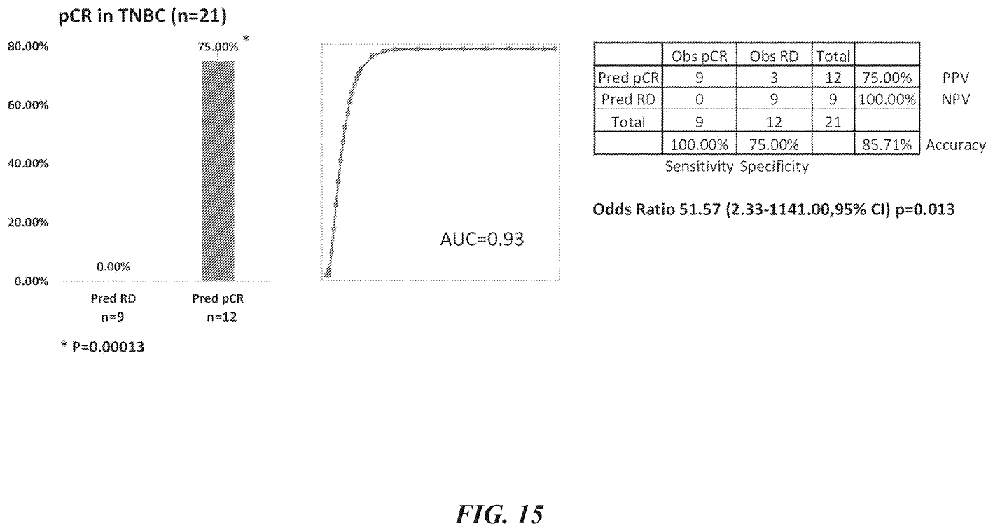

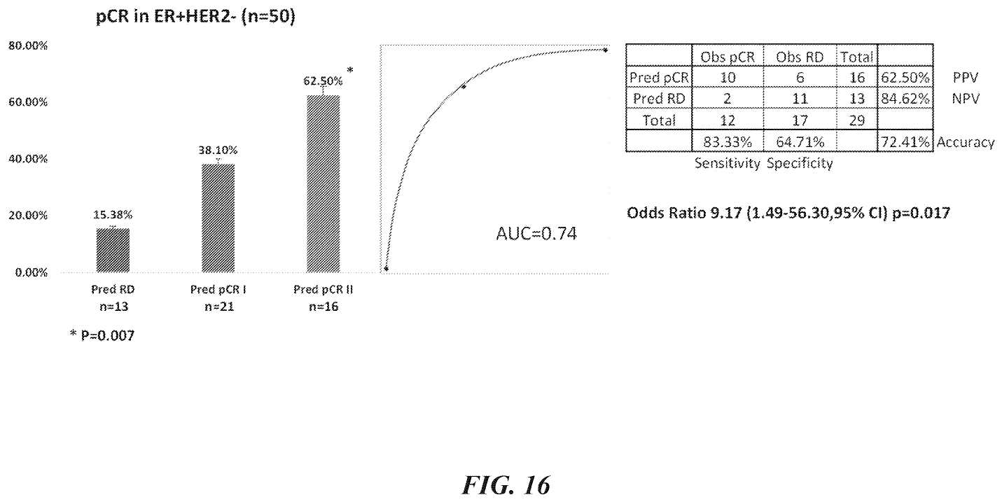

Results. As shown in FIG. 15, predicted responders in the Durvalumab Arm had a pCR rate of 75% vs 0% in predicted non-responders, (p=0.00013) with NPV and sensitivity of 100%, accuracy of 85.7%, Odds Ratio 51.57 (2.33-1141.00,95% CI). The strategy was not predictive in the Control Arm. Multiple logistic regression pCR-predictive models may further improve predictive accuracy. As shown in FIG. 16, predicted responders in the Durvalumab Arm had a pCR I rate of 38.1% and a pCR II rate of 62.5% vs 15.38% in predicted non-responders, (p=0.007) with NPV and sensitivity of 83.33%, accuracy of 72.41%, Odds Ratio 9.17 (1.49-56.30, 95% CI). The strategy was not predictive in the Control Arm. Multiple logistic regression pCR-predictive models may further improve predictive accuracy.

Conclusions. Complementary diagnostic role of pre-NACT MK167+ FOXC1 expression merits prospective clinical trial evaluation in TNBC and HER2 negative ER+ breast cancer treated with neoadjuvant combination regimens that include ICIs. This may help to optimize achieved pCR rates and extend disease-free survival in patients diagnosed with TNBC and HER2 negative ER+ breast cancer.

### Example 17: Assessment of FOXC1 Expression as a Predictor of Response to Neoadjuvant Taxane Plus Platinum Regimens in Primary Triple-Negative Breast Cancer: Retrospective Analysis of Three Clinical Trial Cohorts

Taxane and platinum (TP) NAC regimens, e.g. Carboplatin and Docetaxel (CbD), in TNBC are currently of great interest, having good pathologic complete response (pCR) rates but with a significantly more manageable toxicity profile compared to anthracycline-based NAC regimens. Forkhead Box C1 (FOXC1), a transcriptional driver of cell plasticity/partial EMT/metastasis is an es. tablished mesenchymal marker diagnostic of basal-like breast cancer having proven prognostic value, but of uncertain predictive value. We sought to evaluate the potential of FOXC1 in predicting pCR to neoadjuvant TP regimens in patients diagnosed with TNBC.

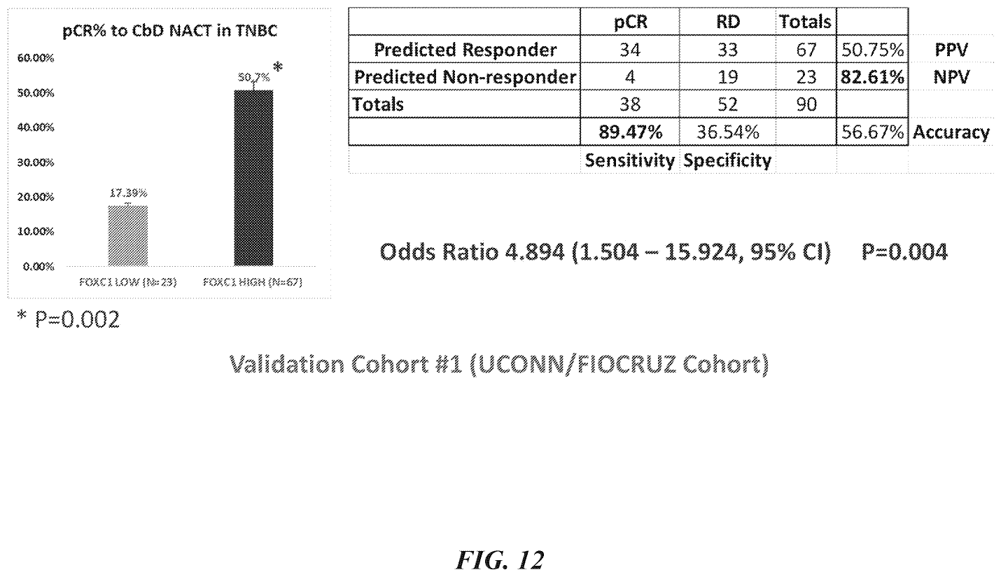

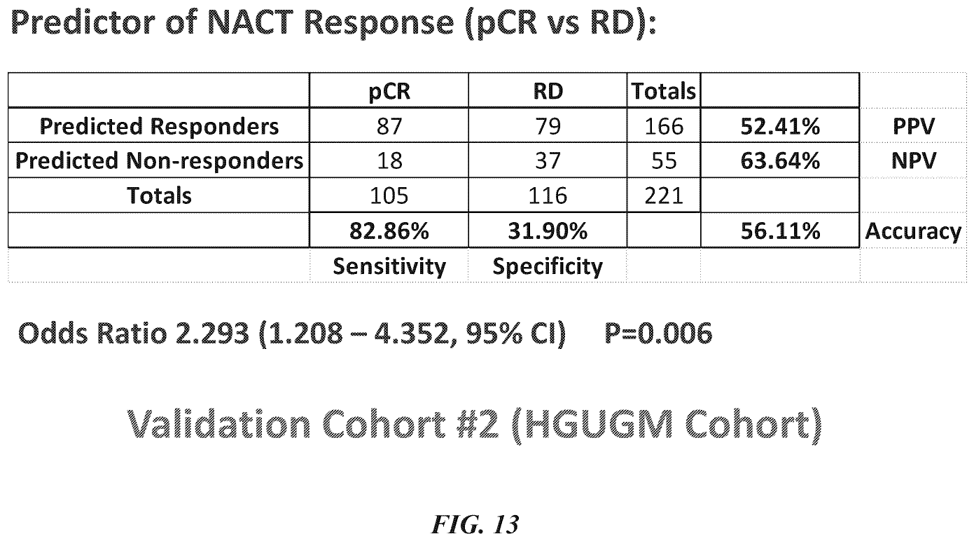

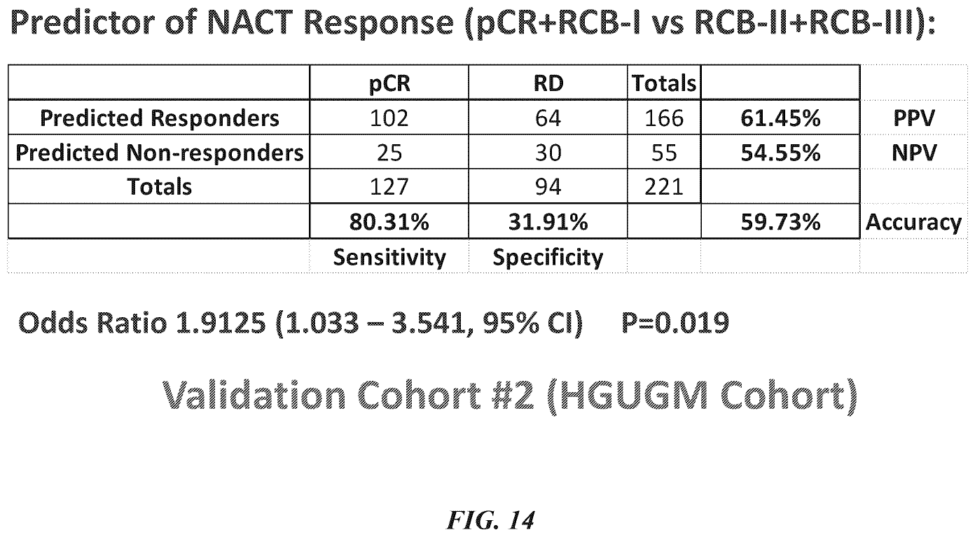

Methods. Pre-NAC tumor biopsy FOXC1 mRNA expression status was correlated with rate of pCR in a pooled, ambispective cohort (prospective cohort GEICAM/2006-03, NCT00432172 pooled with multi-institutional retrospective cohort, n=119). A specific FOXC1 mRNA expression cutoff value was derived to maximize Negative Predictive Value (NPV) and Sensitivity for pCR prediction. The pCR-predictive ability of FOXC1 mRNA expression was then assessed in two validation cohorts of evaluable patients who had been enrolled in prospective clinical trials (UCONN/FIOCRUZ, n=222, HGUGM, NCT01560663, n=221), comparing pCR patients versus patients with residual disease (RD), as shown in FIGS. 12 and 13, or comparing pCR patients and patients with minimal cancer burden (residual cancer burden-I, RCB-I), compared with patients who had moderate or excessive burden (residual cancer burden-II and Ill, RCB-II and RCB-Ill), as shown in FIG. 14. All evaluated patients had been diagnosed with TNBC and had received a Taxane plus Platinum-based NAC regimen.

Results: FOXC1 expression cutoff value was the 25^{th} percentile of expression of FOXC1 mRNA on qRTPCR, 25^{th} percentile of expression of FOXC1 mRNA on RNA-Seq, or FOXC1 Score of 2 using VERESCA® FOXC1 IHC kit. FOXC1 expression below the 25th percentile (FOXC1<25TH % ILE) mRNA EXPRESSION or VERESCA® FOXC1 IHC Score of 0 predicts efficacy of alternative NAC regimen. FOXC1 expression above the 25th percentile (FOXC1>25th % ILE) by mRNA EXPRESSION or VERESCA® FOXC1 IHC Score of 2—Predicts efficacy of Taxane plus platinum NAC regimen.

FOXC1 mRNA expression was associated with pCR in CbD/TP treated TNBC patients with pCR rates of 43.48%, 47.89% and 52.73% observed in the discovery and two validation cohorts (two tailed T-test β-values of 0.0005, 0.002, 0.009, respectively). As shown in FIGS. 12 and 13, FOXC1 expression above the pre-determined cutoff value was associated with pCR to CbD/TP NAC in patients diagnosed with TNBC in both validation cohorts (UCONN/FIOCRUZ cohort in FIG. 12, HGUGM cohort in FIG. 13) (OR 4.894, 1.504-15.924; p=0.004 and OR 2.293, 1.208-4.352; p=0.006). As shown in FIG. 14, FOXC1 expression was also associated with pCR and RCB-I patients (OR 1.9125, 1.033-3.541, p=0.019).

Conclusions. We report the retrospective validation of pre-NAC breast cancer biopsy FOXC1 mRNA expression for predicting efficacy of CbD/TP NAC in two independent, prospectively accrued TNBC patient cohorts. The described strategy may be acceptable for patient stratification to guide CbD/TP NAC recommendations in TNBC. FOXC1 mRNA or protein expression, assessed using qRT-PCR or routine immunohistochemistry (IHC), respectively, could potentially be utilized in future fixed-arm/adaptive clinical trials to further optimize NAC efficacy, in terms of achieved pCR rates, and to extend disease-free survival in patients diagnosed with TNBC.

### Example 18: Production of Anti-FOXC1 Antibody

This example is related to the determination of the variable domain sequences from an antibody (AK 3295) expressed by a monoclonal murine hybridoma cell line.

Total RNA-extraction from hybridoma cells. Primary material: living cell culture from murine hybridoma cell line AK 3295. Total RNA was isolated out of ˜1×10⁶ cells and concentration was determined using a NanoPhotometer.

cDNA-Synthesis. Reverse-transcription (RT) PCR: creation of cDNA from RNA by reverse-transcription using an optimized blend of oligo-(dT) and random primers. Transcription of 1.0 μg total RNA using a standard protocol. Genomic DNA was eliminated. cDNA quality was determined by successful amplification of murine GAPDH cDNA.

Sequence specific amplification by PCR and subcloning. Amplification of Fv coding sequences: PCR reaction using murine Fv specific primers. A combination of murine Fv specific primers was used to amplify VH and VL regions of the monoclonal antibody.

Subcloning of Fv coding sequences: cloning of amplification products for sequencing. VH and VL amplification products were cloned into a cloning and sequencing vector and transformed in E. coli DH5a. Plasmid DNA was prepared from selected clones. Analysis of Fv coding sequences of selected clones by DNA sequencing using vector specific standard primers. All processes described herein were performed according to ISO 9001:2015 guidelines.

Coding sequences of VH amplification products from selected clones are shown in Table 1 (Cloning vector sequences are clipped).

TABLE 1

| Clone ID | Sequence | SEQ ID NO |
|---|---|---|
| HC1_K1 | GGTGAAGCTGCAGGAGTCAGGACCTAGCCTGGTGAAGCCTTCTCAGTCTC | 1 |
| | TGTCCCTCACCTGCACTGTCACTGGCTTCTCAATCACCCGTGATTATGCCT | |
| | GGAACTGGATCCGGCAGTTTCCAGGAAACAAAGTGGAGTGGATGGGTAAC | |
| | ATAAATTACAGTGGTACCACTAACTACAACCCATCTCTCGAAAGTCGAATCT | |
| | CTATCACTCGAGACACATCCAAGAACCAGTTCTTCCTGCAGTTGAATTCTGT | |
| | GACTACTGAGGACACAGCCACATATTACTGTGTAGGATGGGCGGTAAATTA | |
| | TGGTTTGGACTACTGGGGTCCAGGAACCTCAGTCACCGTCTCCTCAGCCAA | |
| | AACGACACCCAAGCTTGTCTATCCACTGGCCCCTGG | |
| HC1_K2 | CCAGGGGCCAGTGGATAGACAAGCTTGGGTGTCGTTTTGGCTGAGGAGAC | 2 |
| | GGTGACTGAGGTTCCTGGACCCCAGTAGTCCAAACCATAATTTACCGCCCA | |
| | TCCTACACAGTAATATGTGGCTGTGTCCTCAGTAGTCACAGAATTCAACTGC | |
| | AGGAAGAACTGGTTCTTGGATGTGTCTCGAGTGATAGAGATTCGACTTTCG | |
| | AGAGATGGGTTGTAGTTAGTGGTACCACTGTAATTTATGTTACCCATCCACT | |
| | CCACTTTGTTTCCTGGAAACTGCCGGATCCAGTTCCAGGCATAATCACGGG | |
| | TGATTGAGAAGCCAGTGACAGTGCAGGTGAGGGACAGAGACTGAGAAGGC | |
| | TTCACCAGGCTAGGTCCTGACTCCTGCAGCTTCACCTC | |
| HC1_K3 | GAGGTGAAGCTGCAGGAGTCAGGACCTAGCCTGGTGAAGCCTTCTCAGTC | 3 |
| | TCTGTCCCTCACCTGCACTGTCACTGGCTTCTCAATCACCCGTGATTATGCC | |
| | TGGAACTGGATCCGGCAGTTTCCAGGAAACAAAGTGGAGTGGATGGGTAA | |
| | CATAAATTACAGTGGTACCACTAACTACAACCCATCTCTCGAAAGTCGAATC | |
| | TCTATCACTCGAGACACATCCAAGAACCAGTTCTTCCTGCAGTTGAATTCTG | |
| | TGACTACTGAGGACACAGCCACATATTACTGTGTAGGATGGGGGTAAATT | |
| | ATGGTTTGGACTACTGGGGTCCAGGAACCTCAGTCACCGTCTCCTCAGCCA | |
| | AAACGACACCCAAGCTTGTCTATCCACTGGCCCCTGG | |
| HC3_K1 | CCAGGGGCCAGTGGATAGACAAGCTTGGGTGTCGTTTTGGCTGAGGAGAC | 4 |
| | GGTGACTGAGGTTCCTGGACCCCAGTAGTCCAAACCATAATTTACCGCCCA | |
| | TCCTACACAGTAATATGTGGCTGTGTCCTCAGTAGTCACAGAATTCAACTGC | |
| | AGGAAGAACTGGTTCTTGGATGTGTCTCGAGTGATAGAGATTCGACTTTCG | |
| | AGAGATGGGTTGTAGTTAGTGGTACCACTGTAATTTATGTTACCCATCCACT | |
| | CCACTTTGTTTCCTGGAAACTGCCGGATCCAGTTCCAGGCATAATCACGGG | |
| | TGATTGAGAAGCCAGTGACAGTGCAGGTGAGGGACAGAGACTGAGAAGGC | |
| | TTCACCAGGCCAGGTCCAGACTCCTGCAGCTGCACCT | |
| HC3_K2 | AGCTGCAGGAGTCTGGACCTGGCCTGGTGAAGCCTTCTCAGTCTCTGTCC | 5 |
| | CTCACCTGCACTGTCACTGGCTTCTCAATCACCCGTGATTATGCCTGGAAC | |
| | TGGATCCGGCAGTTTCCAGGAAACAAAGTGGAGTGGATGGGTAACATAAAT | |
| | TACAGTGGTACCACTAACTACAACCCATCTCTCGAAAGTCGAATCTCTATCA | |
| | CTCGAGACACATCCAAGAACCAGTTCTTCCTGCAGTTGAATTCTGTGACTAC | |
| | TGAGGACACAGCCACATATTACTGTGTAGGATGGGCGGTAAATTATGGTTT | |
| | GGACTACTGGGGTCCAGGAACCTCAGTCACCGTCTCCTCAGCCAAAACGA | |
| | CACCCAAGCTTGTCTATCCACTGGCCCCTGG | |
| HC3_K3 | CCAGGGGCCAGTGGATAGACAAGCTTGGGTGTCGTTTTGGCTGAGGAGAC | 6 |
| | GGTGACTGAGGTTCCTGGACCCCAGTAGTCCAAACCATAATTTACCGCCCA | |
| | TCCTACACAGTAATATGTGGCTGTGTCCTCAGTAGTCACAGAATTCAACTGC | |
| | AGGAAGAACTGGTTCTTGGATGTGTCTCGAGTGATAGAGATTCGACTTTCG | |
| | AGAGATGGGTTGTAGTTAGTGGTACCACTGTAATTTATGTTACCCATCCACT | |
| | CCACTTTGTTTCCTGGAAACTGCCGGATCCAGTTCCAGGCATAATCACGGG | |
| | TGATTGAGAAGCCAGTGACAGTGCAGGTGAGGGACAGAGACTGAGAAGGC | |
| | TTCACCAGGCCAGGTCCAGACTCCTGCAGCTTGACCT | |
| HC_D1_K1 | CAGATGCAGCTTCAGGAGTCGGGACCTGGCCTGGTGAAGCCTTCTCAGTC | 7 |
| | TCTGTCCCTCACCTGCACTGTCACTGGCTTCTCAATCACCCGTGATTATGCC | |
| | TGGAACTGGATCCGGCAGTTTCCAGGAAACAAAGTGGAGTGGATGGGTAA | |
| | CATAAATTACAGTGGTACCACTAACTACAACCCATCTCTCGAAAGTCGAATC | |
| | TCTATCACTCGAGACACATCCAAGAACCAGTTCTTCCTGCAGTTGAATTCTG | |
| | TGACTACTGAGGACACAGCCACATATTACTGTGTAGGATGGGCGGTAAATT | |
| | ATGGTTTGGACTACTGGGGTCCAGGAACCTCAGTCACCGTCTCCTCAAACT | |
| | TAATTAACG | |
| HC_D4_K1 | CGTTAATTAAGTTTGAGGAGACGGTGACTGAGGTTCCTGGACCCCAGTAGT | 8 |
| | CCAAACCATAATTTACCGCCCATCCTACACAGTAATATGTGGCTGTGTCCTC | |
| | AGTAGTCACAGAATTCAACTGCAGGAAGAACTGGTTCTTGGATGTGTCTCG | |
| | AGTGATAGAGATTCGACTTTCGAGAGATGGGTTGTAGTTAGTGGTACCACT | |
| | GTAATTTATGTTACCCATCCACTCCACTTTGTTTCCTGGAAACTGCCGGATC | |
| | CAGTTCCAGGCATAATCACGGGTGATTGAGAAGCCAGTGACAGTGCAGGT | |
| | GAGGGACAGAGACTGAGAAGGCTTCACCAGGCCAGGTCCCGACTCCTGAA | |
| | GCTGCACATC | |
| HC_E1_K1 | GAGGTGCAGCTGAAGGAGTCGGGACCTGGCCTGGTGAAGCCTTCTCAGTC | 9 |
| | TCTGTCCCTCACCTGCACTGTCACTGGCTTCTCAATCACCCGTGATTATGCC | |
| | TGGAACTGGATCCGGCAGTTTCCAGGAAACAAAGTGGAGTGGATGGGTAA | |
| | CATAAATTACAGTGGTACCACTAACTACAACCCATCTCTCGAAAGTCGAATC | |
| | TCTATCACTCGAGACACATCCAAGAACCAGTTCTTCCTGCAGTTGAATTCTG | |
| | TGACTACTGAGGACACAGCCACATATTACTGTGTAGGATGGGCGGTAAATT | |
| | ATGGTTTGGACTACTGGGGTCCAGGAACCTCAGTCACCGTCTCCTCAAACT | |
| | TAATTAACG | |
| HC_E4_K1 | GAGGTGCAGCTGAAGGAGTCGGGACCTGGCCTGGTGAAGCCTTCTCAGTC | 10 |
| | TCTGTCCCTCACCTGCACTGTCACTGGCTTCTCAATCACCCGTGATTATGCC | |
| | TGGAACTGGATCCGGCAGTTTCCAGGAAACAAAGTGGAGTGGATGGGTAA | |
| | CATAAATTACAGTGGTACCACTAACTACAACCCATCTCTCGAAAGTCGAATC | |
| | TCTATCACTCGAGACACATCCAAGAACCAGTTCTTCCTGCAGTTGAATTCTG | |
| | TGACTACTGAGGACACAGCCACATATTACTGTGTAGGATGGGCGGTAAATT | |
| | ATGGTTTGGACTACTGGGGTCCAGGAACCTCAGTCACCGTCTCCTCAAACT | |
| | TAATTAACG | |

Coding sequences of VL amplification products from selected clones are shown in Table 2 (Cloning vector sequences are clipped).

TABLE 2

| Clone ID | Sequence | SEQ ID NO |
|---|---|---|
| LC1_K1 | AGATGGATCCAGTTGGTGCAGCATCAGCCCGTTTTATTTCCAGCTTGGTCC | 11 |
| | CCCCTCCGAACGTGTGAGGAAAATGTGTACCTTGCACGCAGTAATAAACTC | |
| | CCAAATCCTCAGCCTCCACTCTGCTGATTTTCAGTGTAAAATCTGTTCCTGA | |
| | TCCACTGCCAATGAACCTGTCAGGGACTCCAGAGTCCAGTTTAGACACCAG | |
| | ATAGATTAGGCGCTTTGGAGACTGGCCTGGCCTCTGTAATAACCAATTCAA | |
| | ATAGGTTTTTCCATTACTATATAAGAGGCTCTGACTTGACCTGCAAGAGATA | |
| | GAGGCTGGTTGTCCAAAGGTAACCGACAAAGTGAGTTCATCCTGTGTTATC | |
| | ACGATATCACC | |
| LC1_K2 | GGGAAGATGGATCCAGTTGGTGCAGCATCAGCCCGTTTTATTTCCAGCTTG | 12 |
| | GTCCCCCCTCCGAACGTGTGAGGAAAATGTGTACCTTGCACGCAGTAATAA | |
| | ACTCCCAAATCCTCAGCCTCCACTCTGCTGATTTTCAGTGTAAAATCTGTTC | |
| | CTGATCCACTGCCAATGAACCTGTCAGGGACTCCAGAGTCCAGTTTAGACA | |
| | CCAGATAGATTAGGCGCTTTGGAGACTGGCCTGGCCTCTGTAATAACCAAT | |
| | TCAAATAGGTTTTTCCATTACTATATAAGAGGCTCTGACTTGACCTGCAAGA | |
| | GATAGAGGCTGGTTGTCCAAAGGTAACCGACAAAGTGAGTTCATCTTGTGT | |
| | CATCACGATATCACC | |
| LC1_K3 | GGGAAGATGGATCCAGTTGGTGCAGCATCAGCCCGTTTTATTTCCAGCTTG | 13 |
| | GTCCCCCCTCCGAACGTGTGAGGAAAATGTGTACCTTGCACGCAGTAATAA | |
| | ACTCCCAAATCCTCAGCCTCCACTCTGCTGATTTTCAGTGTAAAATCTGTTC | |
| | CTGATCCACTGCCAATGAACCTGTCAGGGACTCCAGAGTCCAGTTTAGACA | |
| | CCAGATAGATTAGGCGCTTTGGAGACTGGCCTGGCCTCTGTAATAACCAAT | |
| | TCAAATAGGTTTTTCCATTACTATATAAGAGGCTCTGACTTGACCTGCAAGA | |
| | GATAGAGGCTGGTTGTCCAAAGGTAACCGACAAAGTGAGTTCATCCTGTGT | |
| | TATCACGATATCACC | |
| LC_D5_K1 | GATGTTGTGATGACCCAGAATCCACTCACTTTGTCGGTTACCTTTGGACAAC | 14 |
| | CAGCCTCTATCTCTTGCAGGTCAAGTCAGAGCCTCTTATATAGTAATGGAAA | |
| | AACCTATTTGAATTGGTTATTACAGAGGCCAGGCCAGTCTCCAAAGCGCCT | |
| | AATCTATCTGGTGTCTAAACTGGACTCTGGAGTCCCTGACAGGTTCATTGG | |
| | CAGTGGATCAGGAACAGATTTTACACTGAAAATCAGCAGAGTGGAGGCTGA | |
| | GGATTTGGGAGTTTATTACTGCGTGCAAGGTACACATTTTCCTCACACGTTC | |
| | GGAGGGGGGACCAAGCTGGAAATCAAACGTTCGGCCGTCG | |
| LC_D6_K1 | CGACGGCCGAACGTTTTATTTCCAGCTTGGTCCCCCCTCCGAACGTGTGAG | 15 |
| | GAAAATGTGTACCTTGCACGCAGTAATAAACTCCCAAATCCTCAGCCTCCAC | |
| | TCTGCTGATTTTCAGTGTAAAATCTGTTCCTGATCCACTGCCAATGAACCTG | |
| | TCAGGGACTCCAGAGTCCAGTTTAGACACCAGATAGATTAGGCGCTTTGGA | |
| | GACTGGCCTGGCCTCTGTAATAACCAATTCAAATAGGTTTTTCCATTACTAT | |
| | ATAAGAGGCTCTGACTTGACCTGCAAGAGATAGAGGCTGGTTGTCCAAAGG | |
| | TAACCGACAAAGTGAGTGGATTCTGGGTCATCACAACATC | |
| LC_B5_K1 | CGACGGCCGAACGTTTGATTTCCAGCTTGGTCCCCCCTCCGAACGTGTAAG | 16 |
| | CTCCCTAATGTGCTGACAGTAATAGGTTGCAGCATCCTCCTCCTCCACAGG | |
| | ATGGATGTTGAGGGTGAAGTCTGTCCCAGACCCACTGCCACTGAACCTGG | |
| | CAGGGACCCCAGATTCTAGGTTGGATACAAGATAGATGAGGAGTCTGGGT | |
| | GGCTGTCCTGGTTTCTGTTGGTTCCAGTGCATATAACTATAGCCAGATGTAC | |
| | TGACACTTTTGCTGGCCCTGTATGAGATGGTGGCCCTCTGCCCCAGAGATA | |
| | CAGCTAAGGAAGCAGGAGACTGTGACATCACAATGTC | |
| LC_F5_K1 | CGACGGCCGAACGTTTGATTTCCAGCTTGGTCCCCCCTCCGAACGTGTAAG | 17 |
| | CTCCCTAATGTGCTGACAGTAATAGGTTGCAGCATCCTCCTCCTCCACAGG | |
| | ATGGATGTTGAGGGTGAAGTCTGTCCCAGACCCACTGCCACTGAACCTGG | |
| | CAGGGACCCCAGATTCTAGGTTGGATACAAGATAGATGAGGAGTCTGGGT | |
| | GGCTGTCCTGGTTTCTGTTGGTTCCAGTGCATATAACTATAGCCAGATGTAC | |
| | TGACACTTTTGCTGGCCCTGTATGAGATGGTGGCCCTCTGCCCCAGAGATA | |
| | CAGCTAAGGAAGCAGGAGACTGTGTCAGCACAATGTCD | |

### Sequencing.

For heavy chain variable domain (VH) from AK 3295 antibody, one consensus sequence was determined and CDRs identified.

For light chain variable domain (VL) from AK 3295, 2 different consensus sequences were determined, named VL_1 and VL_2. VL_1 is more abundant in the performed analysis and represents murine Ig-kappa V1 type. A consensus sequence was determined and CDRs identified. VL_2 is less abundant in performed analysis and represents murine Ig-kappa V3 type. CDRs were determined, but analysis of putative regions/s and domain/s by gene alignment reveal no conclusive and distinct results for VL_2 by comparison with the closest genes and alleles in Mus musculus.

### Results

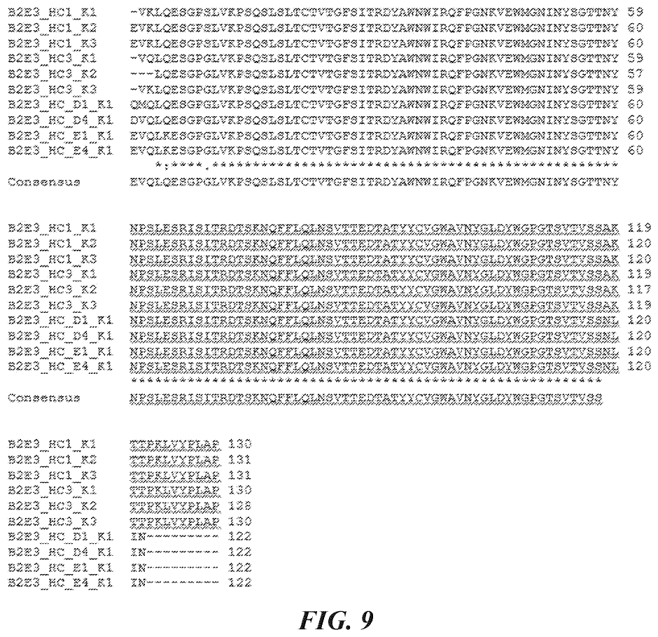

Heavy chain variable domain (VH) from AK 3295 antibody. A VH amino acid sequence alignment is shown in FIG. 9. Differences are highlighted; the consensus sequence is shown below.

VH consensus amino acid sequence: Complementary determining regions (CDR) are underlined.

(SEQ ID NO: 18)
EVQLQESGPGLVKPSQSLSLTCTVTGFSITRDYAWNWIRQFPGNKVEWM
GNINYSGTTNYNPSLESRISITRDTSKNQFFLQLNSVTTEDTATYYCVG
WAVNYGLDYWGPGTSVTVSS

CDR according to IMGT, the international ImMunoGeneTics database. Ref.: Lefranc M P, et al., Nucleic Acids Res. 1999 Jan. 1; 27(1):209-12.

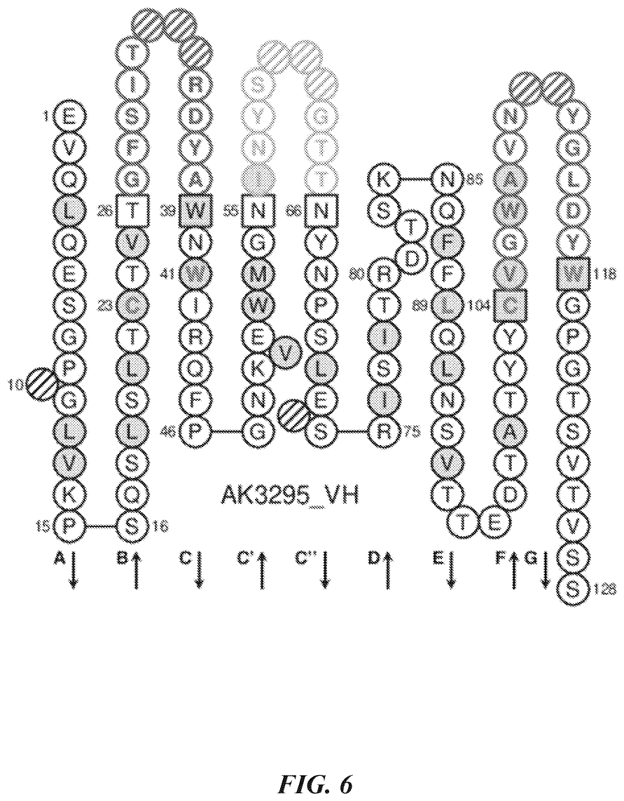

A graphic presentation (VH Collier-de-Perles) of complementary determining regions (CDR) is shown in FIG. 6. Region(s) and domain(s) have been identified in the sequence by comparison with the closest genes and alleles in Mus musculus. (Ref.: IMGT/3Dstructure-DB and IMGT/DomainGapAlign. Ehrenmann F, et al., Nucleic Acids Res. 2010 January;38:D301-7.)

Light chain variable domain (VL) from AK 3295 antibody.

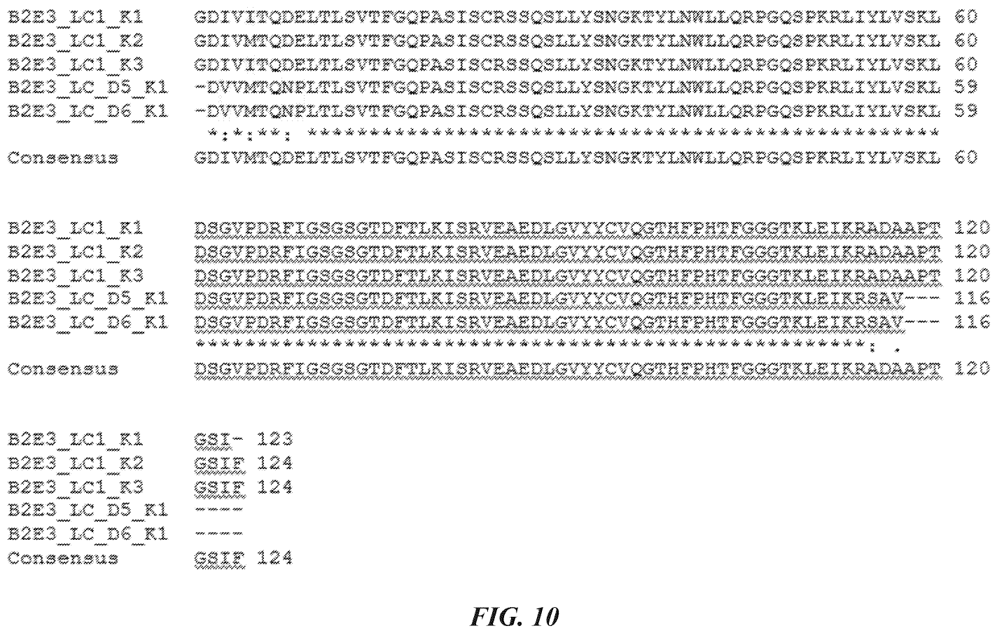

VL_1. VL amino acid sequence alignment is shown in FIG. 10. Differences are highlighted; the consensus sequence is shown below.

VL consensus amino acid sequence: Complementary determining regions (CDR) are underlined.

(SEQ ID NO: 19)
GDIVMTQDELTLSVTFGQPASISCRSSQSLLYSNGKTYLNWLLQRPGQS
PKRLIYLVSKLDSGVPDRFIGSGSGTDFTLKISRVEAEDLGVYYCVQGT
HFPHTFGGGTKLEIKRADAAPTGSIF

CDR according to IMGT, the international ImMunoGeneTics database. Ref.: Lefranc MP, et al., Nucleic Acids Res. 1999 Jan. 1; 27(1):209-12.

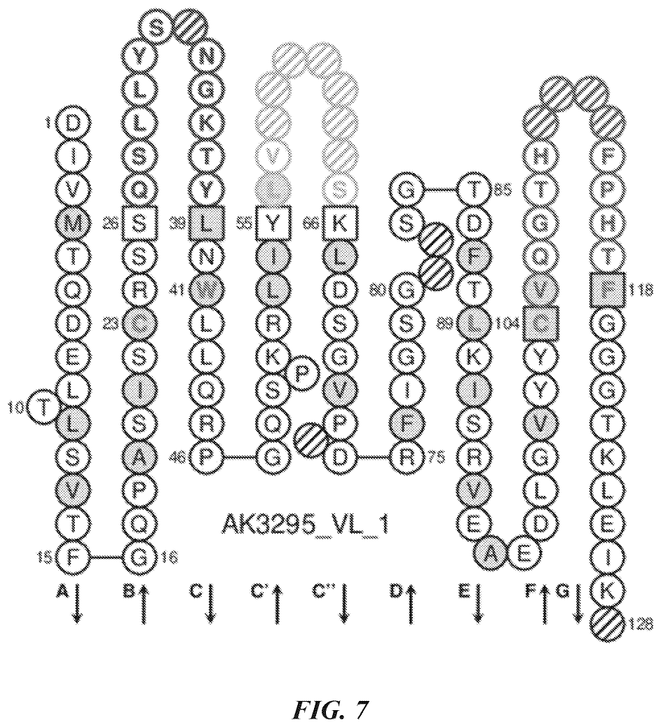

A graphic depiction (VL Collier-de-Perles) of complementary determining regions (CDR) is shown in FIG. 7. Region(s) and domain(s) have been identified in the sequence by comparison with the closest genes and alleles in Mus musculus. Ref.: IMGT/3Dstructure-DB and IMGT/DomainGapAlign. Ehrenmann F, et al., Nucleic Acids Res. 2010 January;38:D301-7.

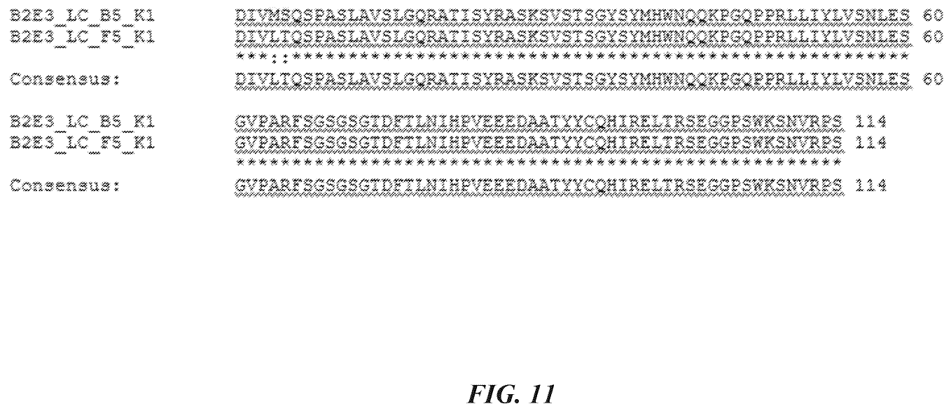

VL_2. A VL amino acid sequence alignment is shown in FIG. 11. Differences are highlighted; the consensus sequence is shown below.

VL consensus amino acid sequence: Complementary determining regions (CDR) are underlined.

(SEQ ID NO: 20)
DIVLTQSPASLAVSLGQRATISYRASKSVSTSGYSYMHWNQQKPGQPPR
LLIYLVSNLESGVPARFSGSGSGTDFTLNIHPVEEEDAATYYCQHIREL
TRSEGGPSWKSNVRPS

CDR according to IMGT, the international ImMunoGeneTics database. Ref.: Lefranc MP, et al., Nucleic Acids Res. 1999 Jan. 1; 27(1):209-12.

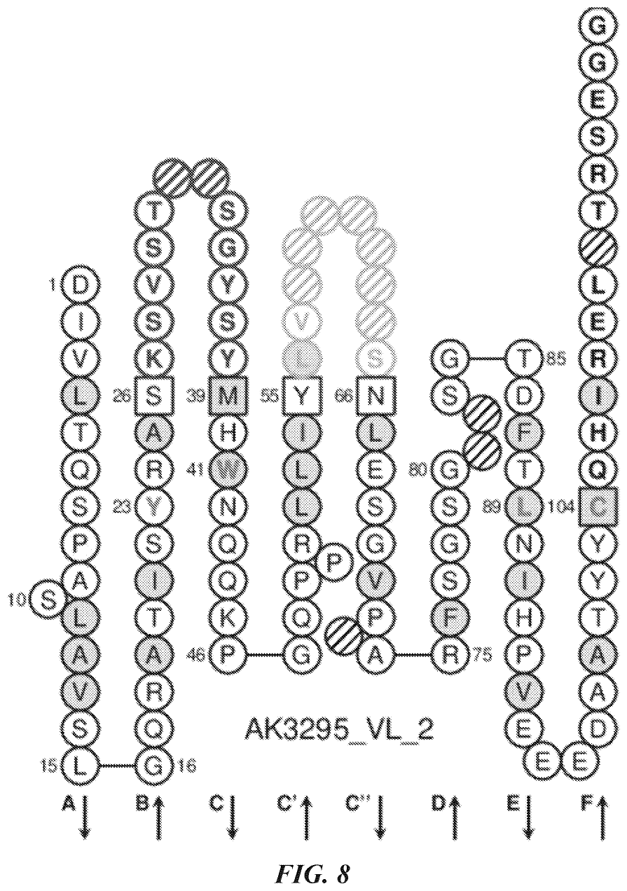

A graphic depiction (VL Collier-de-Perles) of complementary determining regions (CDR) is shown in FIG. 8. Region(s) and domain(s) have been identified in the sequence by comparison with the closest genes and alleles in Mus musculus. Ref.: IMGT/3Dstructure-DB and IMGT/DomainGapAlign. Ehrenmann F, et al., Nucleic Acids Res. 2010 January;38:D301-7

### Example 19: Examples of Anti-FOXC1 Antibodies

Embodiment 1. A nucleotide sequence that encodes an antibody with (i) a VH region having at least 80% sequence identity to SEQ ID NO:18, or (ii) both (i) and a VL region having at least 80% sequence identity to SEQ ID NO:19, or SEQ ID NO:20.
Embodiment 2. The nucleotide sequence of embodiment 1, wherein the antibody is an antibody, an antibody fragment, an antibody conjugate, or an antibody fusion.
Embodiment 3. The nucleotide sequence of embodiment 2 or 3, wherein the antibody is a monoclonal antibody.
Embodiment 4. The nucleotide sequence of any one of embodiments 1 to 3, wherein the antibody is a humanized antibody, a chimeric antibody, or a human antibody.
Embodiment 5. The nucleotide sequence of any one of embodiments 1 to 4, wherein the antibody is an scFv
Embodiment 6. The nucleotide sequence of any one of embodiments 1 to 5, wherein the antibody is bispecific.
Embodiment 7. The nucleotide sequence of any one of embodiments 1 to 6, wherein the nucleotide sequence encodes a VH region having at least 85% sequence identity to SEQ ID NO:18; or (ii) both (i) and a VL region having at least 85% sequence identity to SEQ ID NO:19 or SEQ ID NO:20.
Embodiment 8. The nucleotide sequence of any one of embodiments 1 to 6, wherein the nucleotide sequence encodes a VH region having at least 90% sequence identity to SEQ ID NO:18; or (ii) both (i) and a VL region having at least 90% sequence identity to SEQ ID NO:19 or SEQ ID NO:20.
Embodiment 9. The nucleotide sequence of any one of embodiments 1 to 6, wherein the nucleotide sequence encodes a VH region having at least 95% sequence identity to SEQ ID NO:18; or (ii) both (i) and a VL region having at least 95% sequence identity to SEQ ID NO:19 or SEQ ID NO:20.
Embodiment 10. The nucleotide sequence of any one of embodiments 1 to 6, wherein the nucleotide sequence encodes a VH region having at least 99% sequence identity to SEQ ID NO:18; or (ii) both (i) and a VL region having at least 99% sequence identity to SEQ ID NO:19 or SEQ ID NO:20.
Embodiment 11. The nucleotide sequence of any one of embodiments 1 to 6, wherein the antibody comprises one or more of SEQ ID NO:1-17.
Embodiment 12. An anti-FOXC1 antibody comprising
   (i) a variable heavy chain (VH) region comprising a first VH complementarity region (CDR) (VH CDR1) having an amino acid sequence comprising GFSITRDYA; a second VH CDR (VH CDR2) comprising INYSGTT; and a third VH CDR (VH CDR3) comprising VGWAVNYGLDY; or
   (ii) both (i) and a variable light chain region comprising a first VL complementarity region (CDR) (VL CDR1) having an amino acid sequence comprising QSLLYSNGKTY or KSVSTSGYSY; a second VL CDR (VL CDR2) comprising LVS; and a third VL CDR (VL CDR3) comprising VQGTHFPHT or QHIRELTRSEGG.
Embodiment 13 The antibody of embodiment 12, wherein the antibody is an antibody, an antibody fragment, an antibody conjugate, or an antibody fusion.
Embodiment 14 The antibody of embodiment 11 or 12, wherein the antibody is a monoclonal antibody.
Embodiment 15 The antibody of any one of embodiments 12 to 14, wherein the antibody is a humanized antibody, a chimeric antibody, or a human antibody.
Embodiment 16 The antibody of any one of embodiments 12 to 15, wherein the antibody is an scFv
Embodiment 17 The antibody of any one of embodiments 12 to 16, wherein the antibody comprises one or more of SEQ ID NO:1-17.

### Example 20: FOXC1, MK167, and PDL1 Expression as a Complementary Diagnostic Assay for Predicting Efficacy of Oncology Drugs and Drug Combination Regimens and Methods for their Use

Background Immune checkpoint inhibitors (ICIs) have clinical efficacy in the neoadjuvant setting in multiple solid cancer types. However, suitable tissue-agnostic complementary diagnostics to help guide and tailor treatment recommendations are still lacking. Ki67, a routinely used proliferation marker predicts efficacy of chemotherapeutics but not ICIs. Forkhead Box C1 (FOXC1), a transcriptional driver of cell plasticity/partialEMT/metastasis was recently demonstrated to have potential value in predicting therapeutic efficacy of chemotherapy+ immunotherapy in TNBC. PDL1, a marker of immune evasion, is a proven companion diagnostic for ICI therapy in some but not all situations. We sought to evaluate a Ki67+ FOXC1+ PDL1-expression based response predictor as a possible complementary diagnostic for ICI therapy in the neoadjuvant setting across different cancer types.

Methods. Pre-treatment tumor biopsy MK167, FOXC1 and PDL1 mRNA expression values were retrospectively obtained from patients who had been enrolled and treated in independent clinical trial cohorts (1. I-SPY2 TNBC: AC+ taxane+ pembrolizumab; 2. I-SPY2 TNBC: AC+ olaparib+ paclitaxel+ durvalumab; 3. HNSCC: Nivolumab/Pembrolizumab). Optimized biomarker cutoff values based on model area-under-curve were leave-one-out cross validated in the first dataset for Predicted Responder (PR) and Predicted Non-responder (NR) prediction. The unmodified strategy was then validated in the other datasets.

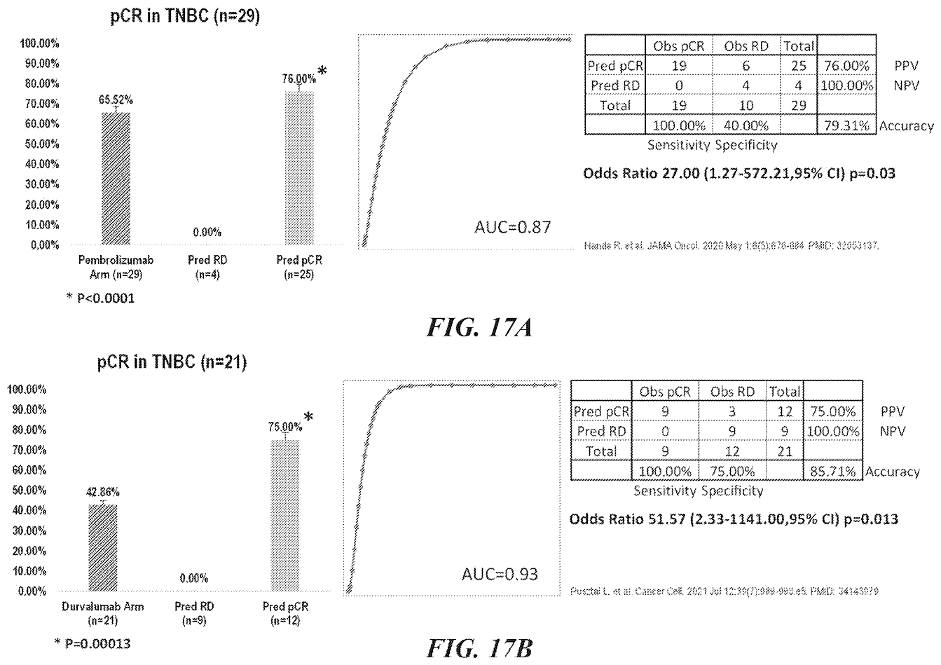

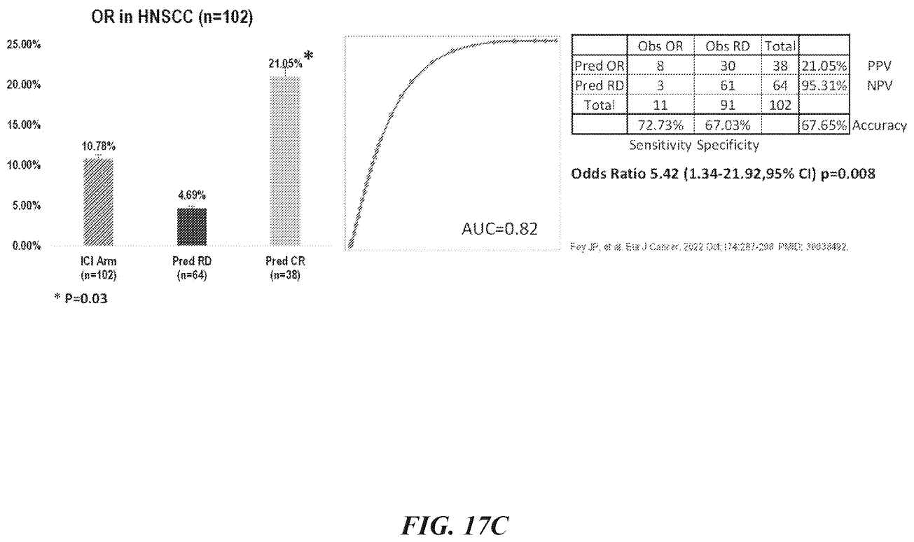

Results. As shown in FIGS. 17A-17C, observed response rates were 66% (n=29) in cohort 1 (FIG. 17A), 43% (n=21) in cohort 2 (FIG. 17B), and and 11% (n=102) in cohort 3 (FIG. 17C). In the biomarker-defined PR groups (n=22, n=12 and n=38) response rates were 76%, 75% and 21% in cohorts 1-3 (FIGS. 17A-17C, respectively). In the biomarker-defined NR groups (n=7, n=9 and n=64) response rates were 0%, 0% and 4% in datasets 1-3(OR=27, 2.5-291.2 95% CI, p=0.003; OR=52, 2.3-1141.0 95% CI, p=0.01; OR=5, 1.3-21.9, 95% CI, p=0.008, respectively). Multiple logistic regression models may further improve predictive accuracy.

Conclusion. Complementary diagnostic role of pre-treatment MK167+ FOXC1+ PDL1 expression merits prospective clinical trial evaluation in multiple cancer types treated with neoadjuvant ICIs alone or in combination with chemotherapeutics.

### Example 21: Ki67 and FOXC1-Based Complementary Diagnostic for Neoadjuvant Cisplatin-Based Chemotherapy Predicts Response and Improved Survival in Muscle-Invasive Bladder Cancer

Background Neoadjuvant Cisplatin-based chemotherapy (NACCT) regimens are widely used in the treatment of muscle-invasive bladder cancer (MIBC). However, suitable complementary diagnostics to help guide and tailor treatment recommendations are still lacking. Ki67 is a well-accepted and routinely used marker that tracks proliferation and has been shown to predict efficacy of neoadjuvant chemotherapy. Forkhead Box C1 (FOXC1), a transcriptional driver of cell plasticity/partial EMT/metastasis/immune evasion has proven prognostic value, and predictive value in breast cancer. Recently FOXC1 was demonstrated to bind enhancers and promote cisplatin resistance in bladder cancer. In this study, FOXC1 contributed to phenotypic plasticity by binding enhancers and promoted a mutation-independent shift towards cisplatin-resistance in bladder cancer.

Methods. 125 pre-treatment tumor biopsy MK167 and FOXC1 mRNA expression values were retrospectively obtained from MIBC patients who had received either NACCT (MVAC regimen-Methotrexate, Vinblastin, Adriamycin and Cisplatin or GemCis regimen—Gemcitabine, Cisplatin) prior to surgical resection (radical cystectomy) of their tumors, and correlated with the rate of observed partial/complete pathologic response, or persistent residual disease5. One patient did not have clinical followup data and was excluded form the analysis. 24 pre-treatment tumor biopsies were included in the study from patients who underwent Induction Chemotherapy for either N+or T4b disease prior to surgical resection (radical cystectomy). The area under the curve (AUC) of each model was calculated and used to determine suitable cutoff values to maximize Negative Predictive Value (NPV) and Sensitivity for Predicted Responder (PR) and Predicted Non-responder (NR) prediction. Differences in recurrence-free survival (RFS), cause-specific survival (CSS) and overall survival (OS) outcomes were also assessed between the predicted PR and predicted RD groups.

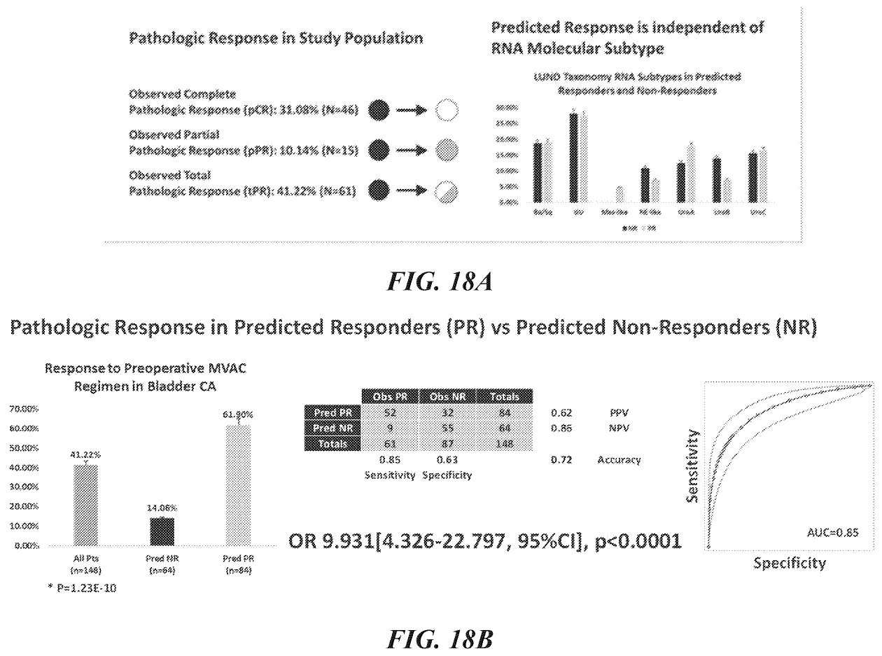

As shown in FIG. 18A, the therapeutic responses were divided into three categories: Complete Pathologic Response (pCR), Partial Pathologic Response (pPR) and the 2 together comprised Total Pathologic Response (tPR). Predicted Responders (PR) and Predicted Non-responders (NR) were categorized using the predictive strategy independently of earlier described RNA Molecular Subtype categories of MIBC according to the Lund Classification System. As such, the methods of the present technology possess therapeutic response predictive value independent of the earlier reported molecular subtyping classification in the Lund system.

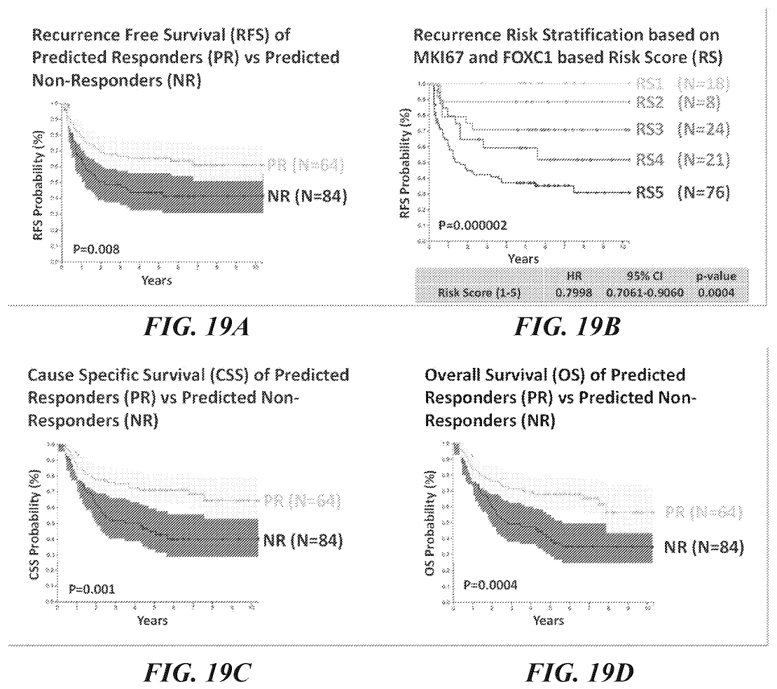

Results. As shown in FIG. 18B, Ki67 and FOXC1 expression based complementary diagnostic predicts response to neoadjuvant cisplatin-based chemotherapy that is independent of molecular subtype and highly comparable to that of gene expression panels. RFS (FIGS. 19A-19B), CSS (FIG. 19C) and OS (FIG. 19D) of Predicted Responders (PR) based on the MK167 and FOXC1 complementary diagnostic approach is superior to that of Predicted Non-Responders (NR) using the same method and is statistically significant. Detection of Ki67 and FOXC1 protein using clinically validated immunohistochemistry kits is quantitatively robust and comparable to detection of Ki67 and FOXC1 mRNA in tumor tissues in the range required to distinguish between PR and NR. MK167 and FOXC1 based complementary diagnostic thus represents a potentially pragmatic approach for routine risk stratification of MIBC in the clinic to help guide neoadjuvant chemotherapy recommendations.

### Example 22: Predicting Therapeutic Response, Durable Clinical Benefit And Risk for Hyperprogression with PD1/PDL1 Inhibitors in Advanced/Metastatic Cancers with Combined Expression of MK167, FOXC1 and PDL1: Pan-Tumor Validation in Clinical Trial and Real-World Cohorts

Background Accurate prediction of therapeutic response (TR) to treatment with immune checkpoint blockade (ICB) as well as any resultant durable clinical benefit in terms of improved progression-free survival (PFS) and overall survival (OS) is not consistently achieved with existing biomarkers like PDL1 or tumor mutational burden (TMB). We hypothesized that a multi-marker predictive biomarker strategy that tracks plasticity using FOXC1 expression, in parallel to tumor proliferation and immune evasion, using expression of MK167 and PDL1, respectively, may demonstrate superior TR prediction, and also enable accurate prediction of risk for hyperprogressive disease (HPD).

Methods. Pre-treatment tumor RNA-Seq data obtained from training/validation clinical trial cohorts and real-world patients diagnosed with advanced/metastatic non-small cell lung cancer (NSCLC, n=82/28) and melanoma (n=154/121) were analyzed for FOXC1, MK167 and PDL1 expression, and correlated with overall response rate (ORR), PFS, OS and HPD, the latter defined as time-to-treatment-failure<=2 months post-treatment initiation. Optimized biomarker cutoff values based on model area-under-curve were leave-one-out cross validated and cancer-type specific (CTS) prediction algorithms derived. The unmodified strategy was then validated in the independent, CTS validation datasets.

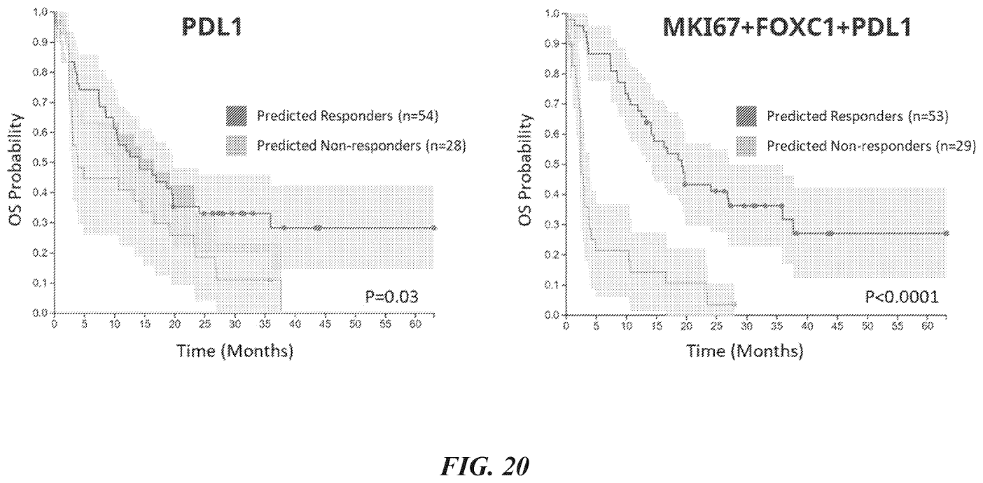

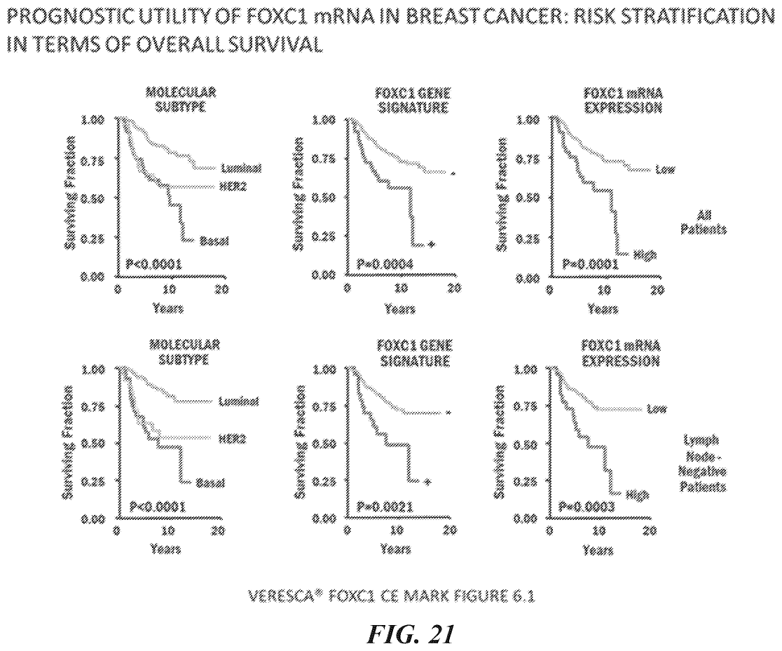

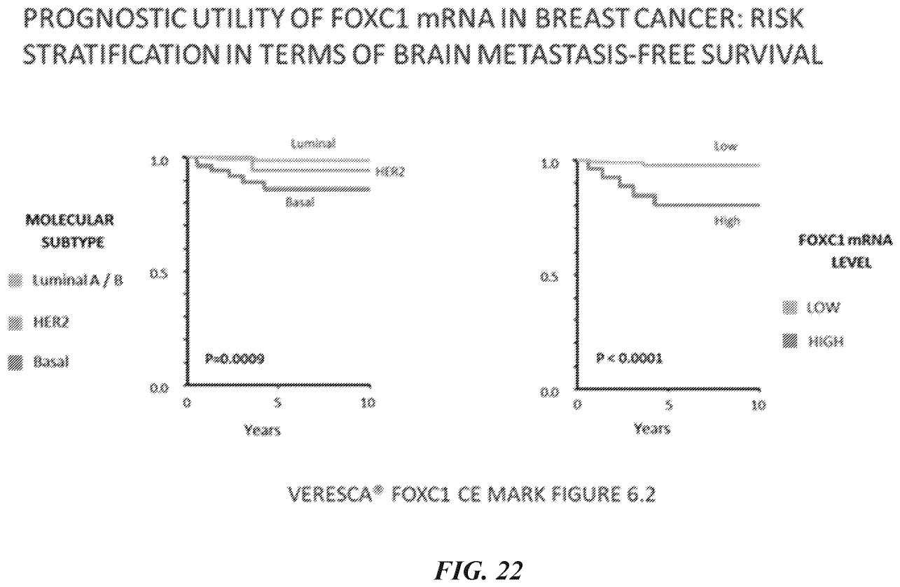

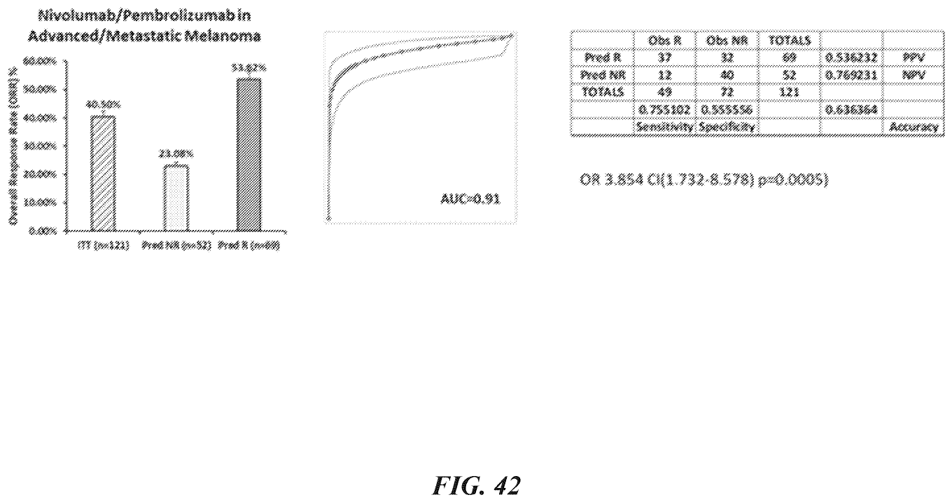

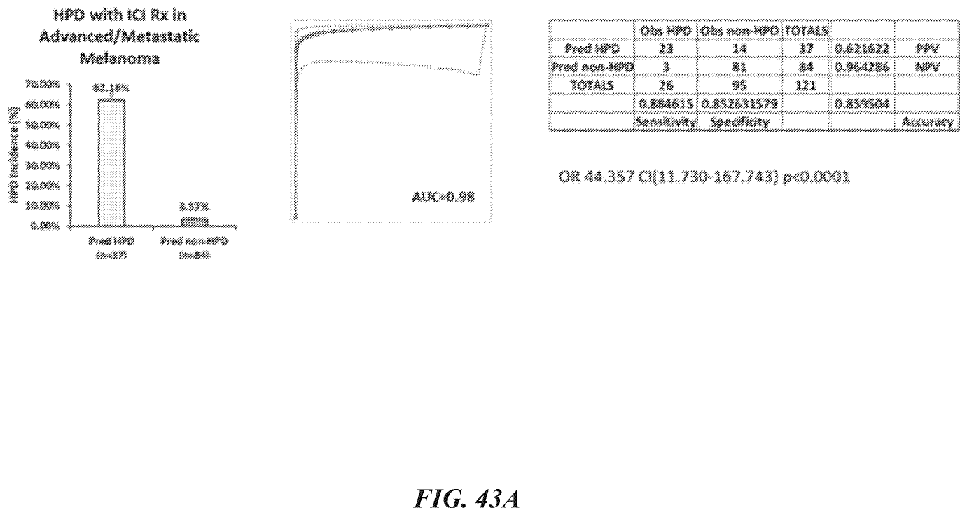

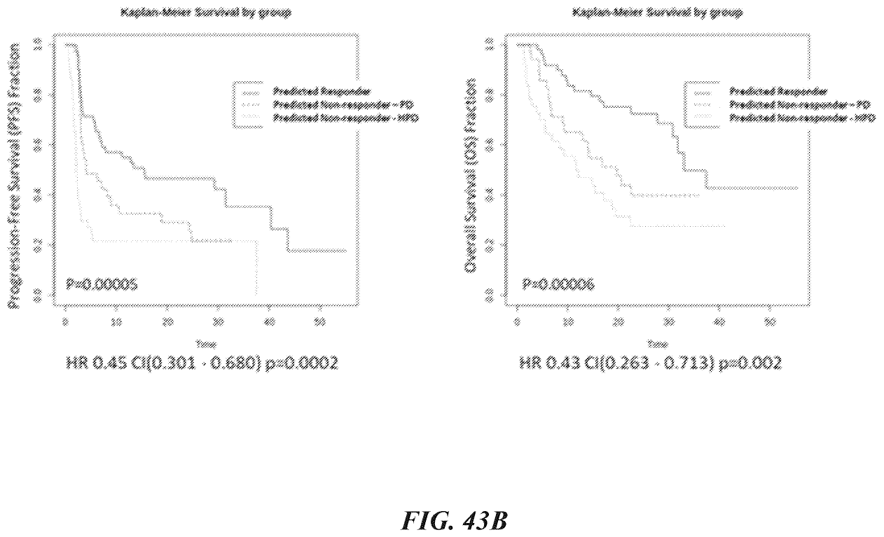

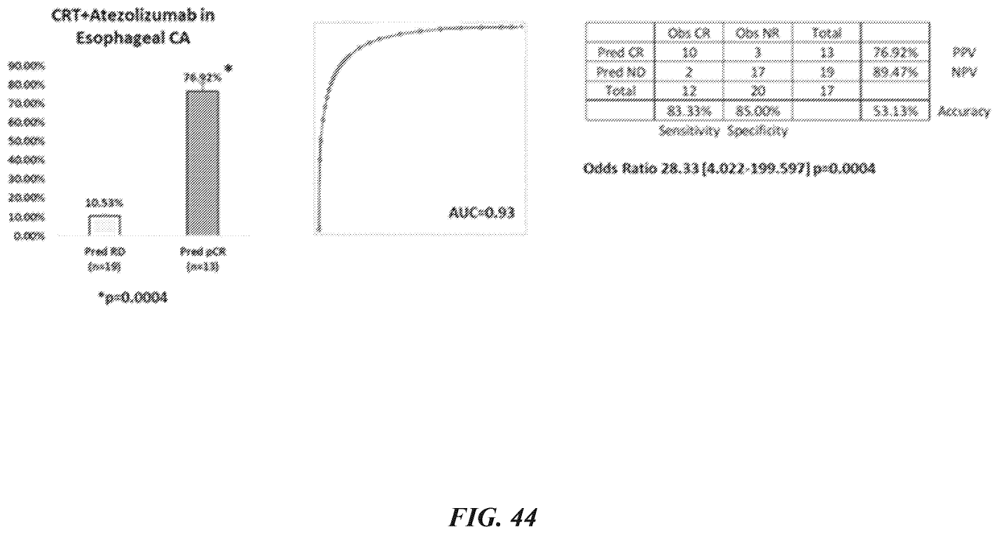

Results. ORR prediction accuracy was confirmed in validation datasets with high accuracy: NSCLC AUC=0.96, OR=9.63 (0.98-94.54, 95% CI) p=0.03 (FIG. 20) and melanoma AUC=0.91, OR=3.85 (1.73-8.58, 95% CI), p=0.0005 (FIG. 42). Predicted Responders consistently displayed superior PFS and OS compared to predicted Non-Responders: NSCLC PFS HR=0.51 (0.32-0.82, 95% CI) p=0.007, OS HR=0.44 (0.27-0.73, 95% CI) p=0.003; melanoma PFS HR=0.45 (0.30-0.68, 95% CI) p=0.002, OS HR=0.43 (0.26-0.71, 95% CI) p=0.002 (FIG. 43B). Patients at risk for HPD were identified with high accuracy: NSCLC AUC=0.96, OR=9.67 (2.95-31.73) p<0.0001; melanoma AUC=0.98, OR=44.36 (11.73-167.74, 95% CI) p<0.0001 (FIG. 43A).

Conclusion. Tracking multiple dimensions of cancer biology including plasticity (using FOXC1), as opposed to tracking immune evasion alone, proved to be a superior and accurate pan-cancer, tissue-agnostic predictor of TR to ICB therapy in advanced/metastatic tumors in terms of ORR, PFS, OS as well as HPD risk prediction. This approach merits further testing in prospective clinical trials.

### Example 23: Prognostic Utility of VERESCA® FOXC1 IHC in Breast Cancer

BACKGROUND INFORMATION-PROGNOSTIC SIGNIFICANCE OF FOXC1 mRNA AND IHC-DETECTED FOXC1 PROTEIN IN BREAST CANCER: SUMMARY OF FINDINGS AND LITERATURE REVIEW

CLINICAL UTILITY: Assessing expression levels of FOXC1 mRNA or protein in breast cancer tissue helps predict long-term prognosis. This helps inform the discussion between oncologist and patient regarding prognostic expectations following an initial diagnosis of cancer.

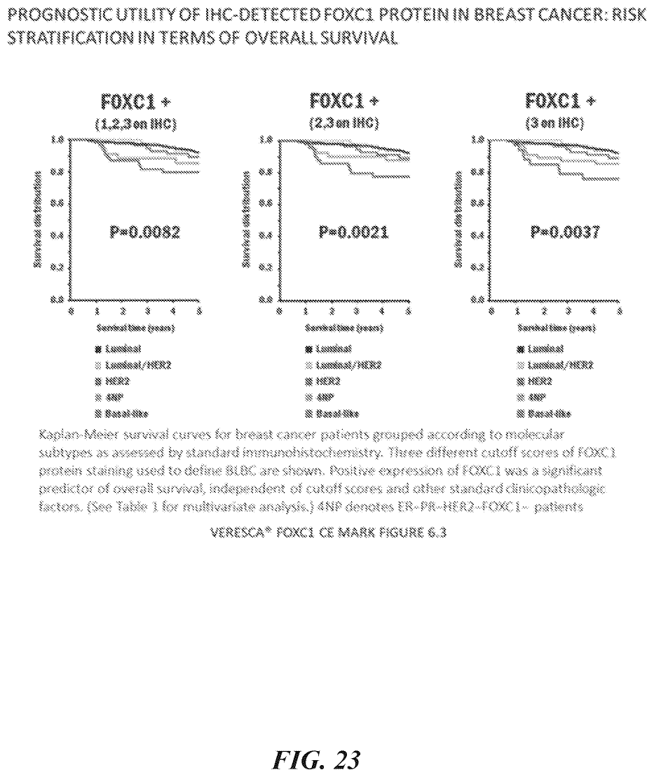

The prognostic significance of FOXC1 in cancer was first reported in 2010 in breast cancer [REF Cancer Research Ray et al., 2010]. Data from this report demonstrated for the first time that the expression level of this single gene had prognostic significance comparable to multigene panels in being able to predict distinctly different rates of Overall Survival (OS) in multiple independent clinical datasets (FIG. 21). Similarly, expression levels of the single gene FOXC1 was able to accurately discern between rates of brain metastasis-free survival (MFS, FIG. 22).

Following the above seminal publication, worldwide investigation into the role of FOXC1 in cancer has burgeoned into an area of intense research. Our understanding of the biologic and mechanistic role played by FOXC1 in cancer has grown tremendously since that time. However, the most immediate clinically actionable aspect of this growth in scientific knowledge has been in our understanding of FOXC1 as a theranostic biomarker—one that has diagnostic, prognostic as well as predictive significance in cancer. To provide proper background and context for the discussion that appears in the later sections of this document, we briefly discuss and summarize pertinent findings and provide a brief overview of the pertinent literature.

SUPERIORITY OF IHC-DETECTED FOXC1 PROTEIN OVER IHC-DETECTED EGFR OR IHC-DETECTED CK5/6 OR CK14 AS AN AID IN DIAGNOSIS OF BASAL-LIKE BREAST CANCER

CLINICAL UTILITY: Assessing expression levels of FOXC1 mRNA or protein in breast cancer tissue is superior to other conventional methods (TNBC, EGFR, CK5/6 or CK14) as an aid in the diagnosis of basal-like breast cancer (BLBC).

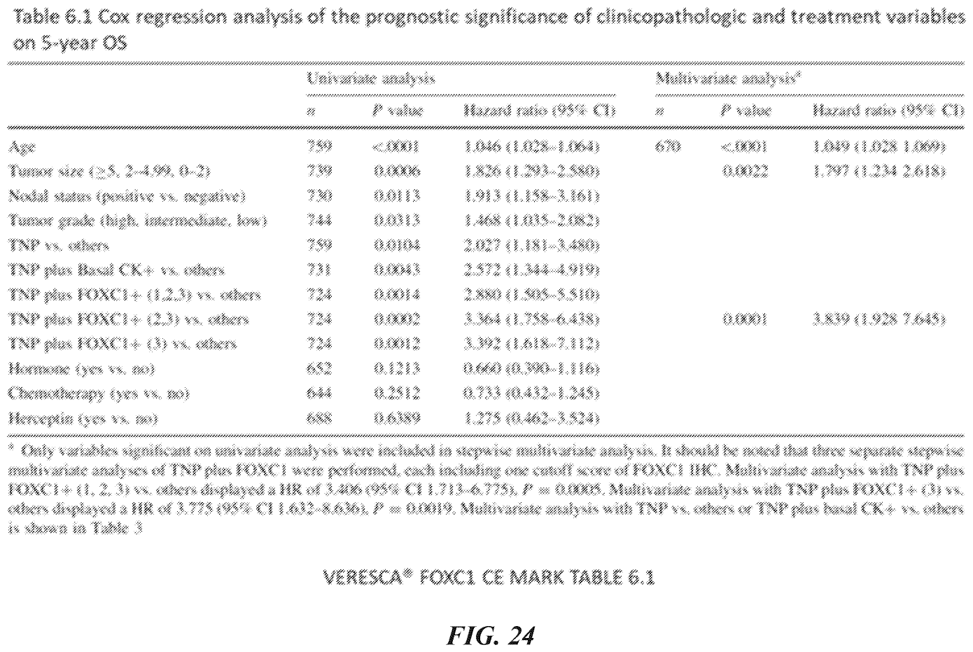

The study involved retrospective analysis of 759 patients diagnosed with breast cancer at a single institution [ASO, Ray et al., 2011]. Archived TNP specimens of primary invasive ductal breast cancer from 759 patients were examined by immunohistochemical staining for FOXC1, CK5/6, and CK14; prognostic significance was assessed using multivariate analyses. Positive expression of FOXC1 protein was a significant predictor of OS over 5 years and 10 years on univariate as well as multivariable analysis (FIGS. 23 and 24).

CONCLUSION: Immunohistochemical detection of FOXC1 expression in TNP invasive breast cancer is an independent prognostic indicator.

PROGNOSTIC SUPERIORITY OF BIOMARKER PANEL INCLUSIVE OF IHC-DETECTED FOXC1 PROTEIN COMPARED TO OTHER BIOMARKER PANELS IN BREAST CANCER

CLINICAL UTILITY: Assessing expression levels of FOXC1 mRNA or protein in breast cancer tissue is superior to other methods (TNBC, CK5/6 or CK14) in predicting long-term prognosis in breast cancer.

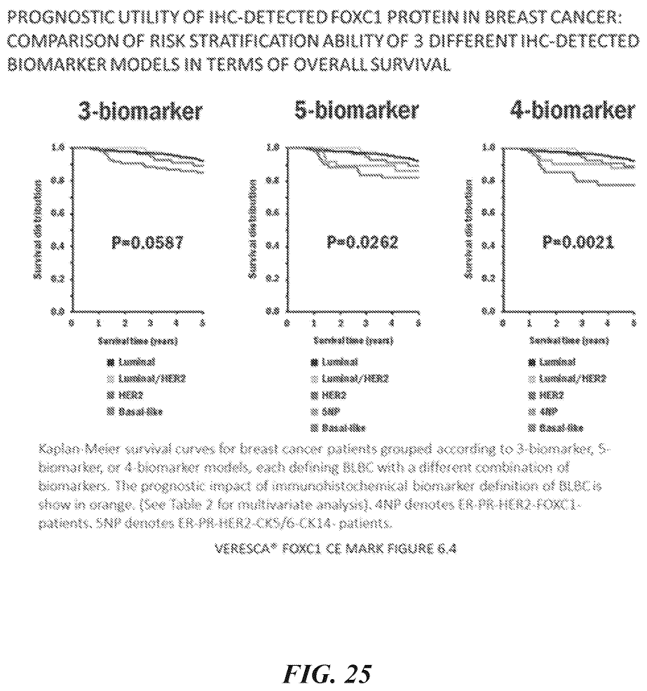

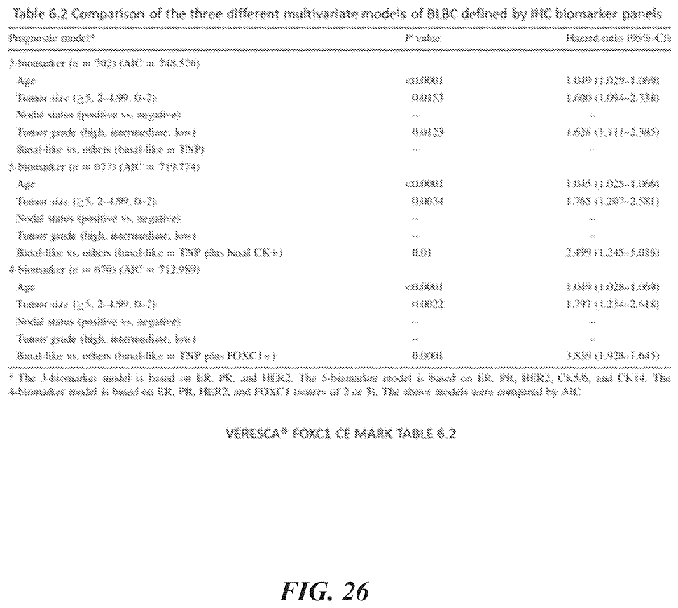

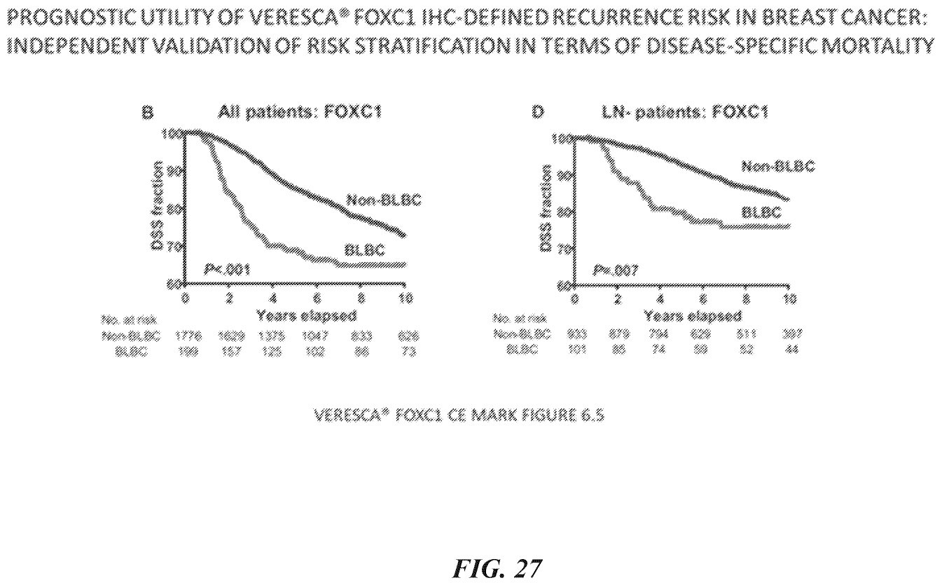

In the above discussed study [ASO, Ray et al., 2011], the impact of adding FOXC1 versus basal CKs to TNP-based BLBC assessment was examined. BLBC defined by triple negative phenotype (TNP) plus FOXC1 demonstrated superior prognostic relevance compared to BLBC defined by TNP alone or TNP plus basal cytokeratins (CKs, FIGS. 25 and 26).

CONCLUSION: Immunohistochemical detection of FOXC1 expression in TNP invasive breast cancer is superior to conventional immunohisto-chemical surrogates of BLBC.

VALIDATION OF PROGNOSTIC UTILITY OF VERESCA FOXC1 (mRNA-equivalent research assay) IN LN-BREAST CANCER PATIENTS

CLINICAL UTILITY: Assessing expression levels of FOXC1 mRNA or protein in LN-breast cancer tissue helps predict long-term prognosis. This is particularly important as LN-status typically is associated with good prognosis. Elevated expression of FOXC1 in LN-breast cancer tissue portends a worse than expected clinical outcome in these patients who might otherwise mistakenly be stratified to have low recurrence risk (on account of their LN-status). This helps inform the discussion between oncologist and patient regarding prognostic expectations following an initial diagnosis of cancer.

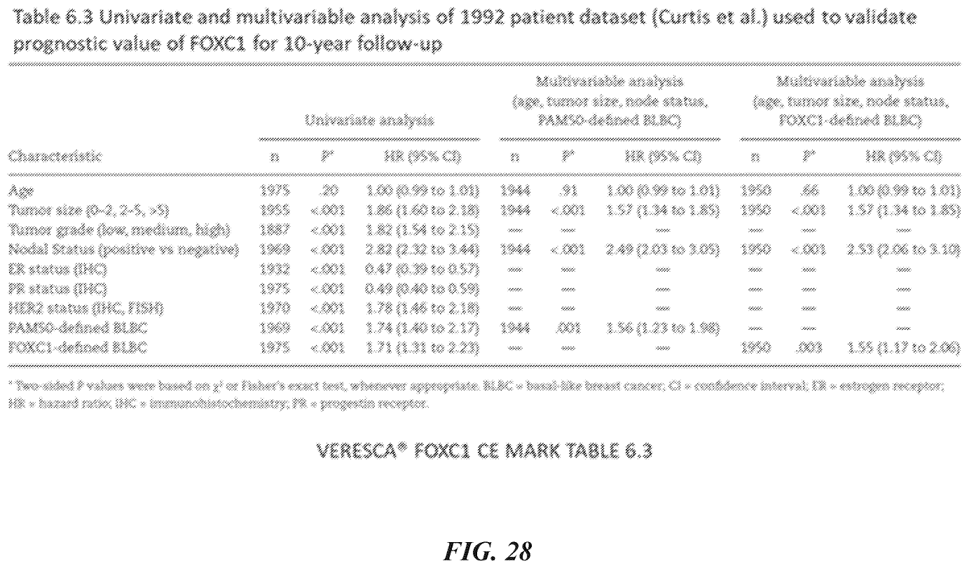

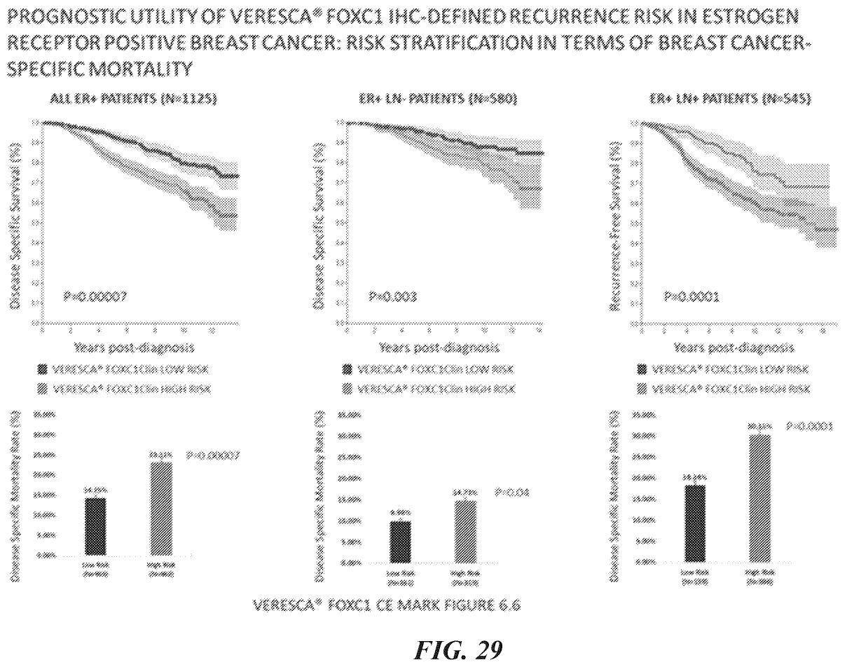

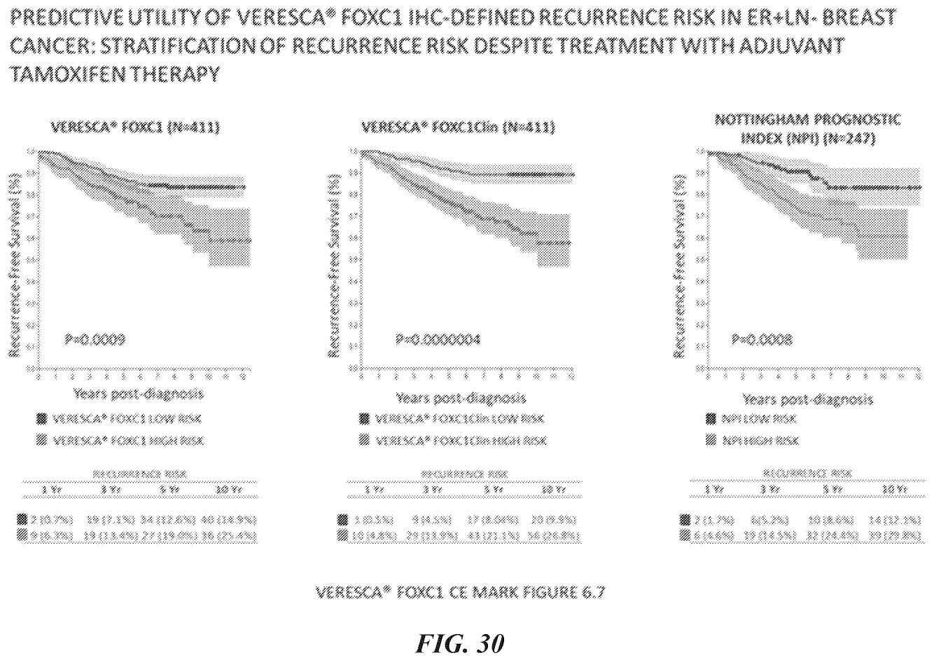

With the goal of validating the earlier reported prognostic significance of FOXC1 in breast cancer by accurately detecting the BLBC molecular subtype, we examined the 1992-sample Curtis et al. dataset with respect to disease-specific survival (DSS) (FIGS. 27 and 28, Jensen et al., JNCI 2015). In multivariate analysis, FOXC1 expression again proved to be an independent prognostic indicator of DSS after adjusting for clinical/pathologic variables such as age, tumor size, and lymph node status. The cutoff used in this validation dataset was based on predetermined cutoff value from earlier training datasets. The FOXC1-defined Basal-like designation was comparable to the multigene PAM50 panel-defined Basal-like designation in terms of prognostic prediction. Furthermore, the FOXC1-defined Basal-like designation allowed prognostic stratification of lymph node-negative breast cancer patients. This prognostic stratification was comparable to that obtained with the PAM50 basal-like designation.

CONCLUSION: Elevated FOXC1 mRNA expression in LN-breast cancer tissue is an independent prognostic indicator in LN-breast cancer patients. Of note, the FOXC1 mRNA expression level cutoff point utilized in this study was proven to be equivalent to a level of FOXC1 protein expression easily and accurately detectable using VERESCA® FOXC1 IHC, through synchronous FOXC1 mRNA and protein profiling on matched tumor tissues.

VALIDATION OF PROGNOSTIC UTILITY OF VERESCA FOXC1 (mRNA-equivalent research assay) IN ER+ BREAST CANCER: RISK STRATIFICATION IN TERMS OF BREAST CANCER-SPECIFIC SURVIVAL

CLINICAL UTILITY: Assessing expression levels of FOXC1 mRNA or protein, in combination with specific clinicopathologic factors (VERESCA® FOXC1Clin), in ER+ breast cancer tissue helps predict long-term prognosis. This is particularly important as ER+ status typically is associated with good prognosis. Elevated expression of FOXC1 in ER+ breast cancer tissue portends a worse than expected clinical outcome in these patients who might otherwise mistakenly be stratified to have low recurrence risk. This helps inform the discussion between oncologist and patient regarding prognostic expectations following an initial diagnosis of cancer.

With the goal of validating and extending the earlier reported prognostic significance of FOXC1 in breast cancer by accurately detecting the BLBC molecular subtype, we again examined the 1992-sample Curtis et al. dataset with respect to disease-specific survival (DSS) but restricted the analysis to ER+ patients (FIG. 29).

CONCLUSION: Elevated FOXC1 mRNA or protein expression, in combination with specific clinicopathologic factors (VERESCA® FOXC1Clin) in ER+ breast cancer tissue is an independent prognostic indicator in ER+ breast cancer patients. Of note, the FOXC1 mRNA expression level cutoff point utilized in this study was proven to be equivalent to a level of FOXC1 protein expression easily and accurately detectable using VERESCA® FOXC1 IHC.

VALIDATION OF PROGNOSTIC UTILITY OF VERESCA FOXC1 (mRNA-equivalent research assay) IN ER+LN-BREAST CANCER: RISK STRATIFICATION IN TERMS OF RECURRENCE-FREE SURVIVAL

CLINICAL UTILITY: Assessing expression levels of FOXC1 mRNA or protein, in combination with specific clinicopathologic factors (VERESCA® FOXC1Clin), in ER+LN-breast cancer tissue helps predict long-term prognosis. This is particularly important as ER+LN-status typically is associated with good prognosis. Elevated expression of FOXC1 in ER+LN-breast cancer tissue portends a worse than expected clinical outcome in these patients who might otherwise mistakenly be stratified to have low recurrence risk. This helps inform the discussion between oncologist and patient regarding prognostic expectations following an initial diagnosis of cancer.

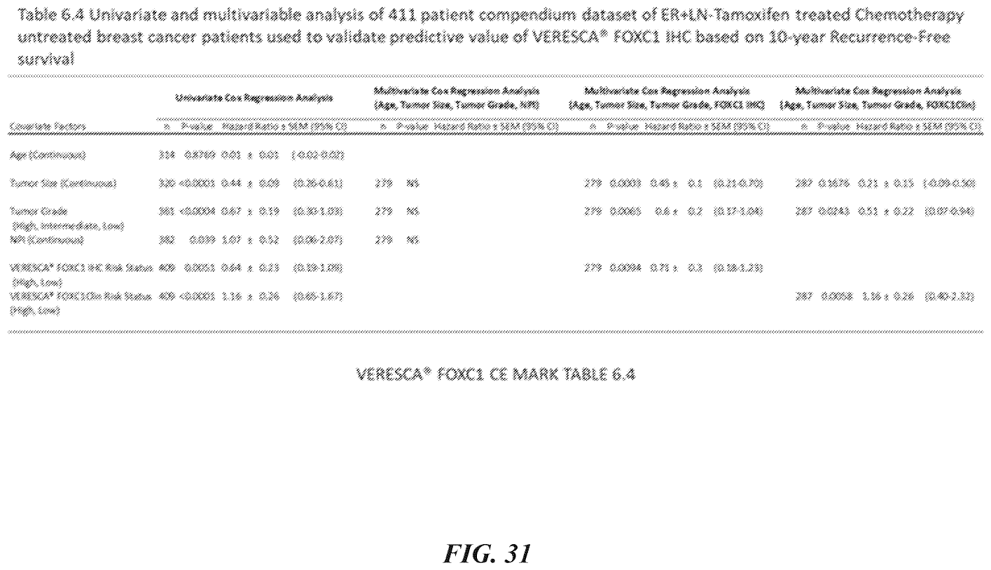

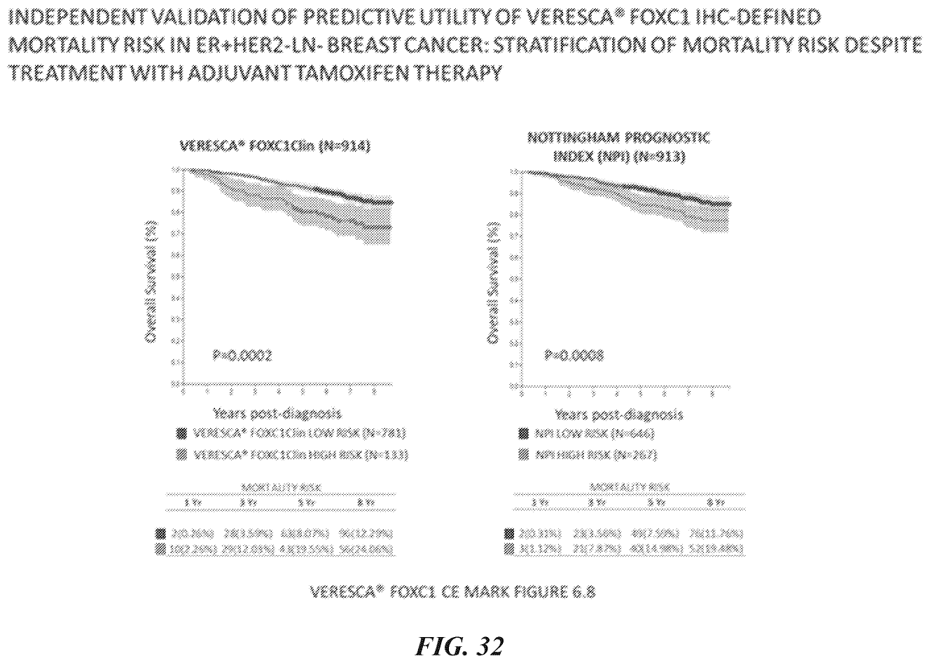

The VERESCA® FOXC1 IHC assay (or its mRNA equivalent assay) and the VERESCA® FOXC1Clin criteria have prognostic utility in ER+LN-breast cancer despite these patients receiving adjuvant Tamoxifen therapy. The VERESCA® FOXC1 IHC assay (or its mRNA-equivalent assay) and the VERESCA® FOXC1Clin criteria can effectively stratify patients according to their recurrence risk, despite receiving adjuvant Tamoxifen therapy (FIGS. 30 and 31).

The ability of VERESCA® FOXC1 IHC to discriminate between patients who have a low recurrence risk versus those who have a high recurrence risk, when combined with standard pathologic criteria (VERESCA® FOXC1Clin) is superior to that of the Nottingham Prognostic Index (NPI) on univariate and multivariable analyses (FIG. 31). As with other predictive assays, if a breast cancer patient is identified to harbor an elevated recurrence risk, then they are typically advised by their treating physician/medical oncologist to receive adjuvant chemotherapy, in addition to adjuvant Tamoxifen therapy, to help mitigate such risk.

The prognostic utility of VERESCA® FOXC1 IHC as well as that of VERESCA® FOXC1 Clin criteria were validated in a compendium dataset comprised of 411 ER+LN-breast cancer who received adjuvant Tamoxifen therapy for 5 years post-diagnosis but did not receive adjuvant chemotherapy, and for whom clinical, treatment and gene expression data were available.

The VERESCA® FOXC1 IHC score to RNA expression level proprietary conversion factor (described in the prior section) was utilized to assign a VERESCA® FOXC1 IHC score to each patient in the compendium dataset. Based on the validation results obtained, a VERESCA® FOXC1 IHC score of 0 indicates a low recurrence risk while a score of 2 or greater indicates an elevated recurrence risk. Furthermore, a VERESCA FOXC1 Clin criteria recurrence risk score of 0 indicates a low risk of disease recurrence (metastasis) while a VERESCA FOXC1 Clin criteria recurrence score of 1 indicates an elevated risk of disease recurrence (metastasis).

The dataset used to derive the VERESCA® FOXC1 IHC score to RNA expression level proprietary conversion factor (described in the prior section) comprised of 350 out of 1000 evaluated human breast cancer patients for whom VERESCA® FOXC1 IHC scores as well as FOXC1 mRNA expression on qRT-PCR data (on matched tumor tissue) were obtained (unpublished results, Onconostic Technologies, Inc.). The dataset (METABRIC) that was used to define the predetermined FOXC1 expression cutoff value (used to distinguish between low and high recurrence risk) was independent of the compendium datasets used for validation in order to minimize study bias. Finally standard pathologic criteria were combined with the VERESCA® FOXC1 IHC score to derive VERESCA® FOXC1Clin criteria recurrence risk scores of either 0 or 1.

ADDITIONAL VALIDATION OF PROGNOSTIC UTILITY OF VERESCA FOXC1 (mRNA-equivalent research assay) IN ER+LN-BREAST CANCER: STRATIFICATION OF MORTALITY RISK DESPITE ADJUVANT TAMOXIFEN THERAPY X 5 YEARS

CLINICAL UTILITY: Which patients diagnosed with ER+LN-breast cancer will benefit from adjuvant Tamoxifen therapy and display decreased future mortality risk due to decreased recurrence risk/incidence of distant metastasis. Which patients diagnosed with ER+LN-breast cancer will not benefit from adjuvant Tamoxifen therapy and display increased future mortality risk due to increased recurrence risk/incidence of distant metastasis.

The prognostic utility of the VERESCA® FOXC1 IHC assay and the VERESCA® FOXC1Clin Criteria recurrence risk score was additionally validated in a second independent clinical trial dataset (SCAN-B) comprising 3207 breast cancer patients for whom clinical, treatment, gene expression data and outcomes data (Overall Survival) were available.

As with other prognostic assays, if a patient is identified to harbor a persistently elevated recurrence risk beyond 5 years of having received adjuvant Tamoxifen therapy, then they are typically advised by their treating physician/medical oncologist to continue receiving adjuvant hormonal therapy (typically a Tamoxifen alternative) up to 10 years post-diagnosis, to help reduce such risk.

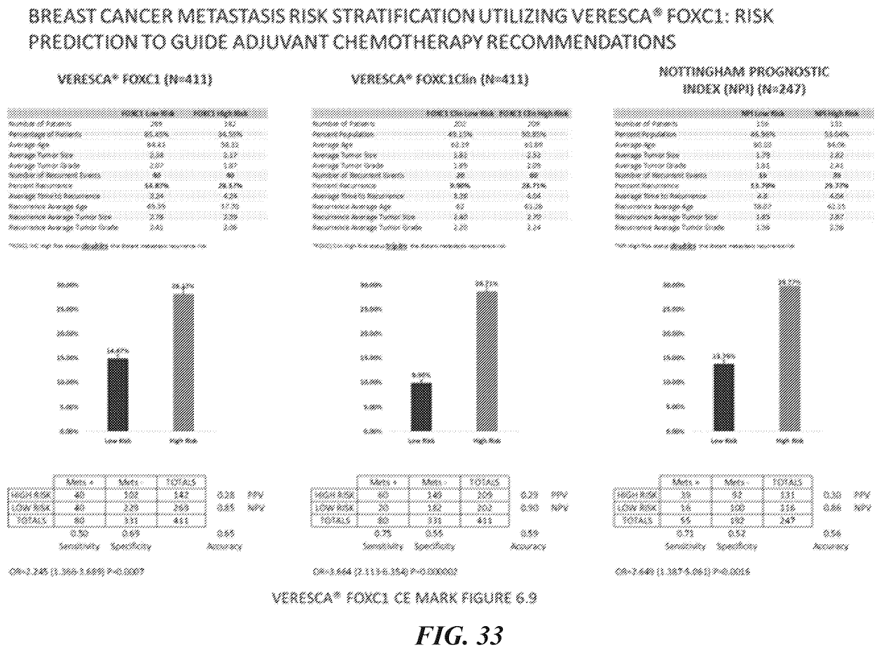

The ability of VERESCA® FOXC1Clin criteria recurrence risk score to predict all-cause mortality rate in the 8-year period post-diagnosis was statistically superior (Odds Ratio 5.189 [Cl 3.461-5.796]p<0.0001) to that observed with NPI criteria (Odds Ratio 2.000[CI 1.356-2.949]p=0.0002) as shown in FIG. 32.

CONCLUSION: The VERESCA® FOXC1 IHC assay has demonstrable prognostic clinical utility in ER+LN-breast cancer patients for recurrence risk stratification and of the consequently resultant mortality risk stratification. Use of the VERESCA® FOXC1 IHC assay can aid in clinical decision making and in formulating rational treatment recommendations with regard to adjuvant chemotherapy, as well as extended hormonal therapy beyond 5 years, to mitigate recurrence risk.

VALIDATION OF PREDICTIVE UTILITY OF VERESCA FOXC1 (mRNA-equivalent research assay) IN ER+LN-BREAST CANCER: STRATIFICATION OF METASTASIS RISK DESPITE ADJUVANT TAMOXIFEN THERAPY X 5 YEARS

CLINICAL UTILITY: Which patients diagnosed with ER+LN-breast cancer will benefit from adjuvant Tamoxifen therapy and display decreased future recurrence risk/incidence of distant metastasis. Which patients diagnosed with ER+LN-breast cancer will not benefit from adjuvant Tamoxifen therapy and display increased future recurrence risk/incidence of distant metastasis.

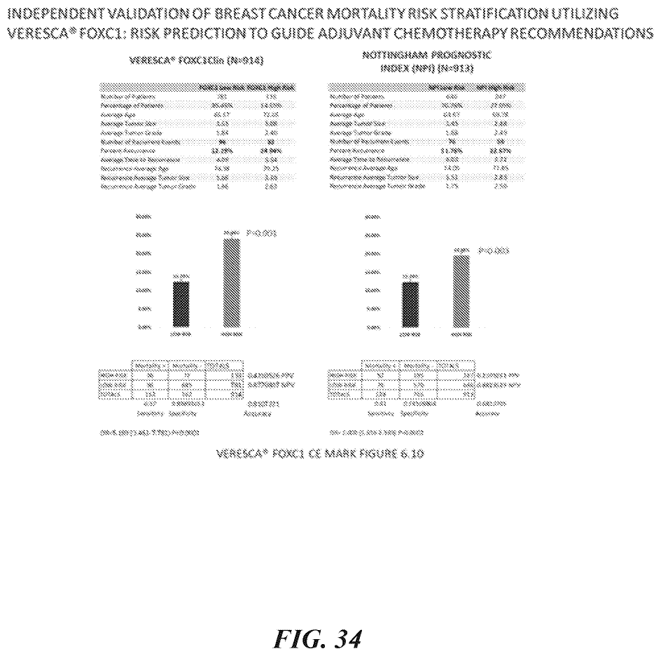

Long-term clinical follow up data, of patients who had been assigned a VERESCA® FOXC1Clin recurrence risk score of 0 or Low Risk, confirmed a significantly lower rate of metastatic disease recurrence (FIG. 32). In contrast, the long-term clinical follow up data, of patients who had been assigned a VERESCA® FOXC1Clin recurrence risk score of 1 or High Risk, confirmed a significantly greater rate of metastatic disease recurrence. This data is summarized in tabular format and recurrence rate graphs in FIG. 33. Also shown, for comparison, are the comparison data of patients assigned either a low or high risk of recurrence based on the NPI.

The ability of the VERESCA® FOXC1 IHC assay and the VERESCA FOXC1 Clin recurrence risk score to predict risk of metastatic disease recurrence is not restricted to the early post-diagnosis period (i.e. 5 years) but persists and extends beyond the early post-diagnosis period (i.e. at least to 10 years post-diagnosis). This is shown in the tables of observed recurrences in the assigned low risk and assigned high risk groups at years 1, 3, 5 and 10 post-diagnosis, at the bottom of FIG. 30. As with other prognostic assays, if a patient is identified to harbor a persistently elevated recurrence risk beyond 5 years of having received adjuvant Tamoxifen therapy, then they are typically advised by their treating physician/medical oncologist to continue receiving adjuvant hormonal therapy (typically a Tamoxifen alternative) up to 10 years post-diagnosis, to help reduce such risk.

The ability of VERESCA® FOXC1Clin criteria recurrence risk score to predict onset of metastatic disease in the 10-year period post-diagnosis was statistically superior (Odds Ratio 3.331 [Cl 1.914-5.796]p=0.00001) to that observed with NPI criteria (Odds Ratio 2.649[CI 1.387-5.061]p=0.0016).

CONCLUSION: The VERESCA® FOXC1 IHC assay has demonstrable prognostic clinical utility in ER+LN-breast cancer patients for recurrence risk stratification. Use of the VERESCA FOXC1 IHC assay can aid in clinical decision making and in formulating rational treatment recommendations with regard to adjuvant chemotherapy to mitigate recurrence risk.

ADDITIONAL VALIDATION OF PREDICTIVE UTILITY OF VERESCA FOXC1 (mRNA-equivalent research assay) IN ER+LN-BREAST CANCER: STRATIFICATION OF MORTALITY RISK DESPITE ADJUVANT TAMOXIFEN THERAPY X 5 YEARS

CLINICAL UTILITY: Which patients diagnosed with ER+LN-breast cancer will benefit from adjuvant Tamoxifen therapy and display decreased future mortality risk due to decreased recurrence risk/incidence of distant metastasis.

Which patients diagnosed with ER+LN-breast cancer will not benefit from adjuvant Tamoxifen therapy and display increased future mortality risk due to increased recurrence risk/incidence of distant metastasis.

The ability of VERESCA® FOXC1Clin criteria recurrence risk score to predict all-cause mortality rate in the 8-year period post-diagnosis was statistically superior (Odds Ratio 5.189 [Cl 3.461-5.796]p<0.0001) to that observed with NPI criteria (Odds Ratio 2.000[CI 1.356-2.949]p=0.0002) as shown in FIG. 34.

CONCLUSION: The VERESCA® FOXC1 IHC assay has demonstrable prognostic clinical utility in ER+LN-breast cancer patients for recurrence risk stratification and of the consequently resultant mortality risk stratification. Use of the VERESCA® FOXC1 IHC assay can aid in clinical decision making and in formulating rational treatment recommendations with regard to adjuvant chemotherapy, as well as extended hormonal therapy beyond 5 years, to mitigate recurrence risk.

VALIDATION OF PREDICTIVE UTILITY OF VERESCA FOXC1 (mRNA-equivalent research assay) IN ER+LN-BREAST CANCER: MORTALITY RISK REDUCTION BY ADDING ADJUVANT CHEMOTHERAPY TO ADJUVANT TAMOXIFEN THERAPY

CLINICAL UTILITY: Which patients diagnosed with ER+LN-breast cancer will benefit from the additional administration of standard chemotherapy anthracycline+ taxane regimen) to 5 years of adjuvant Tamoxifen therapy and display decreased future mortality risk (due to decreased recurrence risk/incidence of distant metastasis. Which patients diagnosed with ER+LN-breast cancer will not benefit from the additional administration of standard chemotherapy (anthracycline+ taxane regimen) to 5 years of adjuvant Tamoxifen therapy and will not display decreased future mortality risk due to decreased recurrence risk/incidence of distant metastasis.

FOXC1 expression cutoff value was 60th percentile of expression of FOXC1 mRNA on qRTPCR, 60th percentile of expression of FOXC1 mRNA on RNA-Seq, or FOXC1 Score of 2 using VERESCA® FOXC1 IHC kit. FOXC1 expression less than the 60th percentile (FOXC1<60TH % ILE) by mRNA EXPRESSION or <VERESCA® FOXC1 IHC Score of 2 predicts LOW RISK and efficacy of the current standard of care which is adjuvant Tamoxifen X 5 years) in successfully reducing disease recurrence risk, and its resultant impact on all-cause mortality, without the need for additional/adjuvant chemotherapy.

FOXC1 expression greater than the 60th percentile (FOXC1 60th % ILE) by mRNA EXPRESSION or VERESCA® FOXC1 IHC Score of 2 predicts therapeutic efficacy of adjuvant chemotherapy (Anthracycline plus Taxane combination regimen) (administered in conjunction with the current standard of care which is adjuvant Tamoxifen X 5 years) in this specific clinical scenario, in successfully reducing disease recurrence risk, and its resultant impact on all-cause mortality or Overall Survival—from greater than 24% risk of death without adjuvant chemotherapy, down to less than 5% risk of death with adjuvant chemotherapy.

Patients whose ER+ breast tumors express FOXC1 greater than the prespecified level described above would, therefore, likely derive significant therapeutic benefit if administered adjuvant chemotherapy in this setting, in addition to adjuvant chemotherapy.

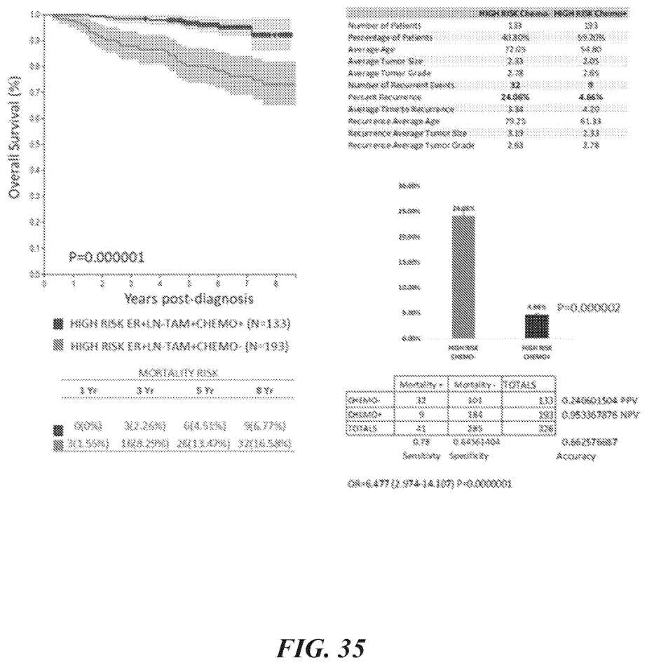

Long-term clinical follow up data, of patients who had been assigned an elevated recurrence risk score based on VERESCA® FOXC1Clin criteria, confirmed a significantly lower rate of all-cause mortality, when treated with adjuvant chemotherapy in addition to adjuvant Tamoxifen therapy. In contrast, the long-term clinical follow up data, of patients who had been assigned an eevated recurrence risk score based on VERESCA® FOXC1Clin criteria, confirmed a significantly higher rate of all-cause mortality when they received only adjuvant Tamoxifen therapy without adjuvant chemotherapy. This is shown in the Kaplan Meier curves of Overall Survival shown in FIG. 35.

The ability of the VERESCA® FOXC1Clin criteria recurrence risk score to predict a statistically significant decrease in all-cause mortality rate in the 8-year period post-diagnosis in response to having received adjuvant chemotherapy in addition to adjuvant Tamoxifen therapy was validated in SCAN-B clinical trial dataset (Odds Ratio 6.477 [Cl 2.974-14.107], p=0.0000001).

CONCLUSION: The VERESCA® FOXC1 IHC assay has demonstrable predictive clinical utility in ER+LN-breast cancer patients and can accurately predict therapeutic benefit of adjuvant chemotherapy when administered in addition to adjuvant Tamoxifen therapy in patients designated ot have elevsted risk of recurrence based on VERESCA® FOXC1Clin criteria. The VERESCA® FOXC1 IHC assay can therefore be used to guide clinical decision making and in formulating rational treatment recommendations with regard to adjuvant chemotherapy to mitigate recurrence risk when present despite treatment with adjuvant Tamoxifen x 5 years in patients diagnosed with ER+LN-breast cancer.

COMPLEMENTARY DIAGNOSTIC UTILITY OF VERESCA® FOXC1 IHC IN BREAST CANCER

Triple Negative Breast Cancer Neoadjuvant Taxane+ Platinum chemotherapy regimen

Clinical Utility: FOXC1 expression is a predictor of response to neoadjuvant taxane plus platinum regimens in primary triple-negative breast cancer: Results from 3 clinical trials.

Background Taxane and platinum (TP) NAC regimens, e.g. Carboplatin and Docetaxel (CbD), in TNBC are currently of great interest, having good pathologic complete response (pCR) rates but with a significantly more manageable toxicity profile compared to anthracycline-based NAC regimens. Forkhead Box C1 (FOXC1), a transcriptional driver of cell plasticity/partial EMT/metastasis is an established mesenchymal marker diagnostic of basal-like breast cancer having proven prognostic value, but of uncertain predictive value. We sought to evaluate the potential of FOXC1 in predicting pCR to neoadjuvant TP regimens in patients diagnosed with TNBC.

Methods. Pre-NAC tumor biopsy FOXC1 mRNA expression status was correlated with rate of pCR in a pooled, ambispective cohort (prospective cohort GEICAM/2006-03, NCT00432172 pooled with multi-institutional retrospective cohort, n=119). A specific FOXC1 mRNA expression cutoff value was derived to maximize Negative Predictive Value (NPV) and Sensitivity for pCR prediction. The pCR-predictive ability of FOXC1 mRNA expression was then assessed in two validation cohorts of evaluable patients who had been enrolled in prospective clinical trials (UCONN/FIOCRUZ, n=222, HGUGM, NCT01560663, n=221). All evaluated patients had been diagnosed with TNBC and had received a Taxane plus Platinum-based NAC regimen.

Results. FOXC1 mRNA expression was associated with pCR in CbD/TP treated TNBC patients with pCR rates of 43.48%, 47.89% and 52.73% observed in the discovery and two validation cohorts (two tailed T-test β-values of 0.0005, 0.002, 0.009, respectively). FOXC1 expression above the pre-determined cutoff value was associated with pCR to CbD/TP NAC in patients diagnosed with TNBC in both validation cohorts (OR 4.894, 1.504-15.924; p=0.004 and OR 2.293, 1.208-4.352; p=0.006).

Conclusions. We report the retrospective validation of pre-NAC breast cancer biopsy FOXC1 mRNA expression for predicting efficacy of CbD/TP NAC in two independent, prospectively accrued TNBC patient cohorts. The described strategy may be acceptable for patient stratification to guide CbD/TP NAC recommendations in TNBC. FOXC1 mRNA or protein expression, assessed using qRT-PCR or routine immunohistochemistry (IHC), respectively, could potentially be utilized in future fixed-arm/adaptive clinical trials to further optimize NAC efficacy, in terms of achieved pCR rates, and to extend disease-free survival in patients diagnosed with TNBC.

Neoadjuvant Olaparib+ Taxane+ Durvalumab

Clinical Utility: A complementary diagnostic strategy based on FOXC1 expression is a predictor of response to neoadjuvant Olaparib, Taxane and Durvalumab in primary triple-negative breast cancer: Results from a subarm of the I-SPY2 clinical trial.

Background Immune checkpoint inhibitors (ICIs) have shown clinical efficacy when administered in combination with neoadjuvant chemotherapy (NACT) for the treatment of triple negative breast cancer (TNBC). However, suitable complementary diagnostics to help guide and tailor treatment recommendations are still lacking. Ki67 is a well-accepted and routinely used marker that tracks proliferation and has been shown to predict efficacy of neoadjuvant chemotherapy. Forkhead Box C1 (FOXC1), a transcriptional driver of cell plasticity/partial EMT/metastasis/immune evasion has proven prognostic value, but remains of uncertain predictive value. We sought to evaluate the potential of a Ki67 and FOXC1-based response predictor as a possible complementary diagnostic for a neoadjuvant regimen comprising of a PARP inhibitor (Olaparib), a Taxane chemotherapeutic (Paclitaxel) and an ICI of the PDL1 class (Durvalumab) in patients diagnosed with primary TNBC.

Methods. 41 Pre-NACT tumor biopsy MK167 and FOXC1 mRNA expression values were retrospectively obtained from TNBC patients who had been enrolled and treated in the I-SPY2 clinical trial (Durvalumab arm: 21, Control arm: 20) and correlated with the rate of observed pathologic complete response (pCR). The area under the curve (AUC) of each model was calculated and used to determine suitable cutoff values to maximize Negative Predictive Value (NPV) and Sensitivity for pCR prediction.

Results. Predicted responders in the Durvalumab Arm had a pCR rate of 75% vs 0% in predicted non-responders, p=0.00013) with NPV and sensitivity of 100%, accuracy of 85.7%, Odds Ratio 51.57 (2.33-1141.00,95% CI). The strategy was not predictive in the Control Arm.

Conclusions. Pre-NACT MK167+ FOXC1 expression can predict efficacy of neoadjuvant combination regimens that include ICIs. This may help to optimize achieved pCR rates and extend disease-free survival in patients diagnosed with TNBC.

HER2+ Breast Cancer. Neoadjuvant Herceptin+ Lapatinib

Clinical Utility: A complementary diagnostic strategy based on FOXC1 expression is a predictor of response to neoadjuvant chemotherapy+ Trastuzumab+ Lapatinib in primary ER-HER2+ breast cancer: Results of the CHER-LOB and TRIO-US B07 clinical trials

Background: Neoadjuvant chemotherapy combined with dual HER2-blockade (Herceptin+ Lapatinib, CHL) has been shown to display greater efficacy compared to neoadjuvant chemotherapy (NACT) combined with Herceptin alone for the treatment of HER2+ breast cancer. However, suitable complementary diagnostics to help guide and tailor treatment recommendations are still lacking. Ki67 is a well-accepted and routinely used marker that tracks proliferation and has been shown to predict efficacy of neoadjuvant chemotherapy. Forkhead Box C1 (FOXC1), a transcriptional driver of cell plasticity/partial EMT/metastasis/immune evasion has proven prognostic value, but remains of uncertain predictive value. We sought to evaluate the potential of a Ki67 and FOXC1-based response predictor as a possible complementary diagnostic for such neoadjuvant regimens in patients diagnosed with primary ER-HER2+ breast cancer.

Methods: Pre-NACT tumor biopsy MK167 and FOXC1 mRNA expression values were retrospectively obtained from ER-HER2+ patients who had been enrolled and treated with CHL in the CHER-LOB (CHL Arm: 34, Control Arm: X) and TRIO-US B07 (CHL Arm: 50, Control Arm: X) clinical trials and correlated with the rate of observed pathologic complete response (pCR). The area under the curve (AUC) of each model was calculated and used to determine suitable cutoff values to maximize Negative Predictive Value (NPV) and Sensitivity for pCR prediction.

Results: In the CHER-LOB cohort, predicted responders had a pCR rate of 63.2% vs 6.7% in predicted non-responders, p=0.0059) with NPV, sensitivity and accuracy of 85.7%, 81.8% and 75% respectively (Odds Ratio 24 [2.574-223.790, 95% Cl]p=0.0026). In the TRIO-US B07 cohort, predicted responders had a pCR rate of 75% vs 0% in predicted non-responders, p=0.0059) with NPV, sensitivity and accuracy of 85.7%, 81.8% and 75% respectively (Odds Ratio 51.57 (2.33-1141.00,95% CI). The strategy was not predictive in either Control Arm. Multiple logistic regression pCR-predictive models may further improve predictive accuracy. Conclusions: Pre-NACT MK167+ FOXC1 expression can predict efficacy of neoadjuvant therapy with CHL in patients diagnosed with ER-HER2+ breast cancer. This may help to optimize achieved pCR rates and extend disease-free survival in patients diagnosed with primary ER-HER2+ breast cancer.

### Example 23: FOXC1 Expression as a Predictor of Response to Neoadjuvant Trastuzumab Plus Lapatinib in Primary HER2+ Breast Cancer

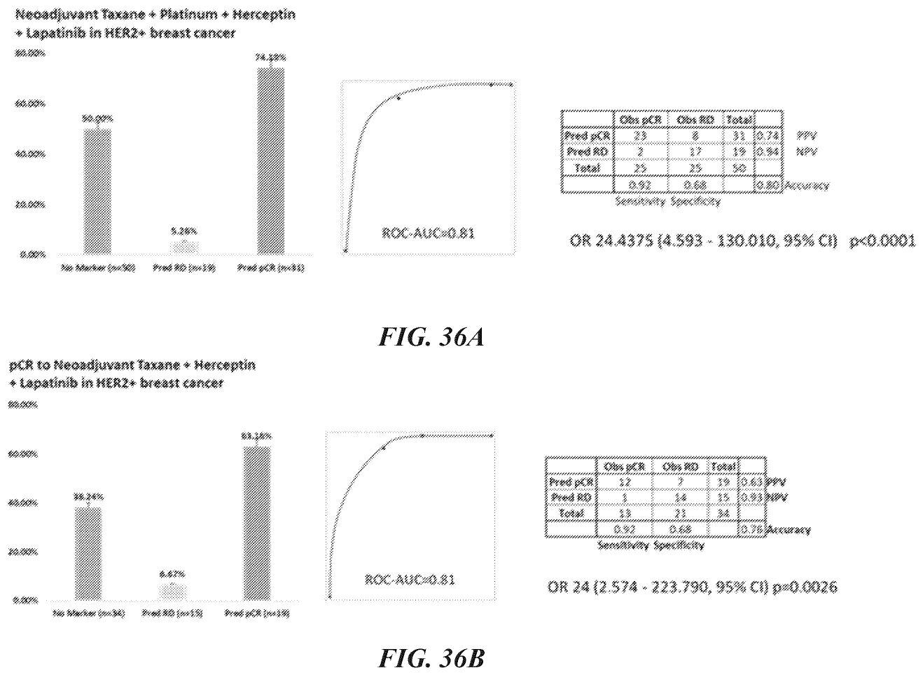

As shown in FIGS. 36A and 36B, FOXC1 expression based complementary diagnostic predicts response to neoadjuvant taxane, platinum, Herceptin, and lapatinib chemotherapy. FOXC1 expression cutoff value was 25th percentile of expression of FOXC1 mRNA on qRTPCR, 25th percentile of expression of FOXC1 mRNA on RNA-Seq, or FOXC1 Score of 2 using VERESCA® FOXC1 IHC kit.

FOXC1 expression below the cutoff value (FOXC1<25TH % ILE) by mRNA EXPRESSION or VERESCA® FOXC1 IHC Score of 0 predicts efficacy of alternative NAC regimen. FOXC1 expression above the cutoff value (FOXC1>25th % ILE) by mRNA EXPRESSION or VERESCA® FOXC1 IHC Score of 2 or greater predicts efficacy of Trastuzumab plus Lapatinib NAC regimen.

### Example 24: FOXC1 Expression as a Predictor of Response to Neoadjuvant Anthracycline Monotherapy in Triple-Negative Breast Cancer

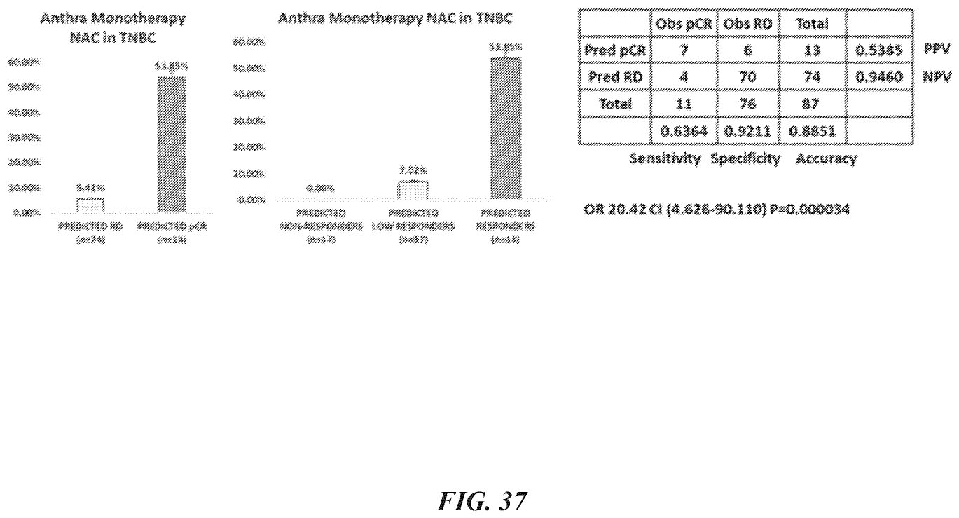

As shown in FIG. 37, FOXC1 expression based complementary diagnostic predicts response to neoadjuvant anthracycline monotherapy in subjects with triple negative breast cancer. FOXC1 expression cutoff value was the 25^{th} percentile of expression of FOXC1 mRNA on qRTPCR, 25^{th} percentile of expression of FOXC1 mRNA on RNA-Seq, or FOXC1 Score of 2 using VERESCA® FOXC1 IHC kit.

FOXC1 expression below the cutoff value (FOXC1<25TH % ILE) by mRNA EXPRESSION or VERESCA® FOXC1 IHC Score of 0 predicts efficacy of Anthracycline monotherapy NAC regimen. FOXC1 expression above the cutoff value (FOXC1 25th % ILE) by mRNA EXPRESSION or VERESCA® FOXC1 IHC Score of 2 or greater predicts efficacy of alternative NAC regimen.

### Example 25: FOXC1 Expression as a Predictor of Response to Neoadjuvant Taxane Monotherapy in Primary Triple-Negative Breast Cancer

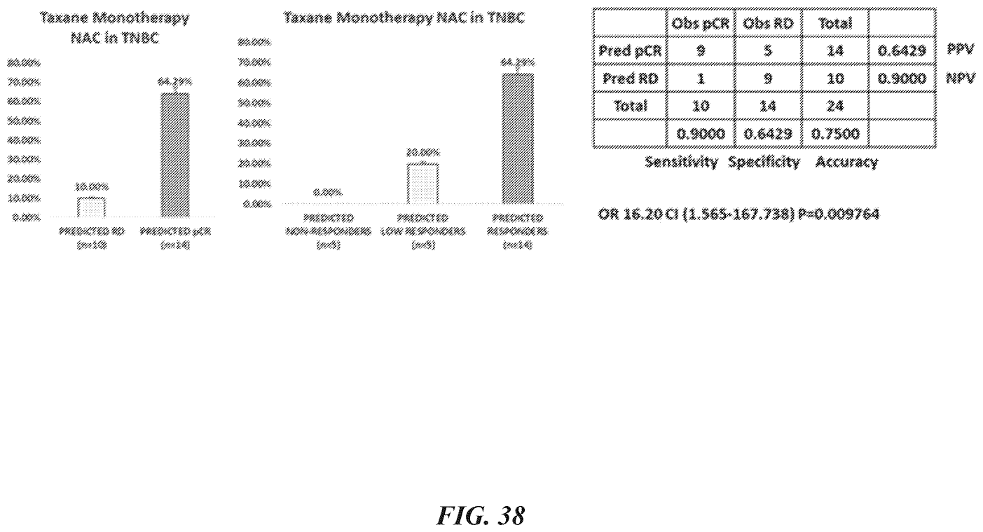

As shown in FIG. 38, FOXC1 expression based complementary diagnostic predicts response to neoadjuvant taxane monotherapy in subjects with triple negative breast cancer. FOXC1 expression cutoff value was the 25th percentile of expression of FOXC1 mRNA on qRTPCR, 25th percentile of expression of FOXC1 mRNA on RNA-Seq, or FOXC1 Score of 2 using VERESCA® FOXC1 IHC kit.

FOXC1 expression below the 25th percentile (FOXC1<25TH % ILE) by mRNA EXPRESSION or VERESCA® FOXC1 IHC Score of 0 predicts efficacy of alternative NAC regimen. FOXC1 expression above the 25th percentile (FOXC1 25th % ILE) by mRNA EXPRESSION or VERESCA® FOXC1 IHC Score of 2 or greater predicts efficacy of Taxane monotherapy NAC regimen.

### Example 26: FOXC1 Expression as a Predictor of Response to Neoadjuvant Anthracycline Plus Taxane Regimens (TFAC/TFEC) in Primary Triple-Negative Breast Cancer

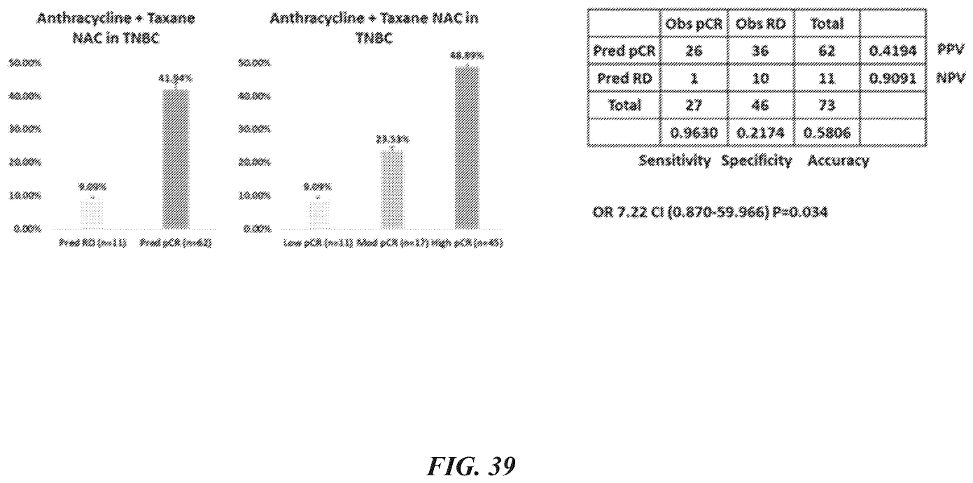

As shown in FIG. 39, FOXC1 expression based complementary diagnostic predicts response to neoadjuvant taxane monotherapy in subjects with triple negative breast cancer. FOXC1 expression cutoff value was the 25th percentile of expression of FOXC1 mRNA on qRTPCR, 25th percentile of expression of FOXC1 mRNA on RNA-Seq, or FOXC1 Score of 2 using VERESCA® FOXC1 IHC kit.

FOXC1 expression below the 25th percentile (FOXC1<25TH % ILE) by mRNA EXPRESSION or VERESCA® FOXC1 IHC Score of 0 predicts efficacy of alternative NAC regimen. FOXC1 expression above the 25th percentile (FOXC1 25th % ILE) by mRNA EXPRESSION or VERESCA® FOXC1 IHC Score of 2 predicts efficacy of TFAC/TFEC NAC regimen Example 27: Combined MK167, FOXC1 and PDL1 expression as a predictor of response to neoadjuvant taxane plus capecitabine (TX) regimens in primary triple negative breast cancer

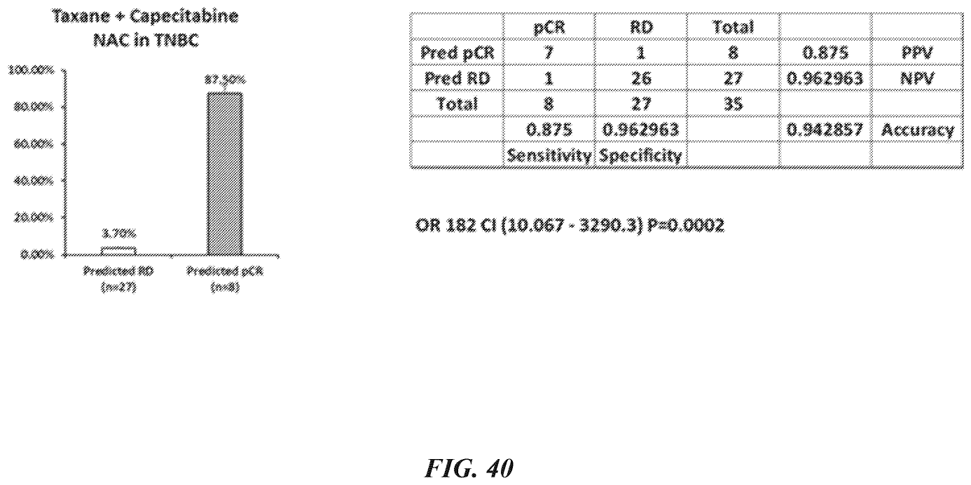

As shown in FIG. 40, FOXC1, Ki67, and PDL1 expression based complementary diagnostic predicts response to neoadjuvant taxane plus capecitabine in subjects with triple negative breast cancer.

MK167 expression cutoff value was 70^{th} percentile of expression of MK167 mRNA on qRTPCR, 70^{th} percentile of expression of MK167 mRNA on RNA-Seq, or MK167 score on IHC of 3 using MIB1 antibody. FOXC1 expression cutoff value was 85^{th} percentile of expression of FOXC1 mRNA on qRTPCR, 85^{th} percentile of expression of FOXC1 mRNA on RNA-Seq, or FOXC1 Score of 6 using VERESCA® FOXC1 IHC kit. PDL1 (CD274) expression cutoff value was 15^{th} percentile of expression of PDL1 (CD274) mRNA on qRTPCR, 15^{th} percentile of expression of PDL1 (CD274) mRNA on RNA-Seq, or PDL1 score of 1 using PDL1 IHC kit.

MK167 less than the 70^{th} percentile (MK167<70^{th}% ILE) by mRNA EXPRESSION or <MK167 IHC Score of 3 predicts efficacy of alternative NAC regimen. MK167 expression greater than the 70^{th} percentile (MK167 70^{th}% ILE) mRNA EXPRESSION or MK167 IHC Score of 3 predicts efficacy of TX NAC regimen

FOXC1 expression less than the 25^{th} percentile (FOXC1<25TH % ILE) by mRNA EXPRESSION or VERESCA® FOXC1 IHC Score of 0 predicts efficacy of alternative NAC regimen. FOXC1 expression greater than the 25^{th} percentile (FOXC1 25^{th}% ILE) mRNA EXPRESSION or VERESCA® FOXC1 IHC Score of 2—Predicts efficacy of TX NAC regimen.

PDL1 expression less than the 15^{th} percentile (PDL1 (CD274)<15^{th}% ILE by mRNA EXPRESSION or <PDL1 IHC Score of 1 predicts efficacy of alternative NAC regimen. PDL1 expression greater than the 15^{th} percentile (PDL1 (CD274) 15^{th}% ILE) by mRNA EXPRESSION or ≥PDL1 IHC Score of 1 predicts efficacy of TX NAC regimen Example 28: Combined MK167, FOXC1 and PDL1 expression as a predictor of response to neoadjuvant anthracycline plus taxane plus capecitabine (FEC-TX) regimens in primary triple negative breast cancer

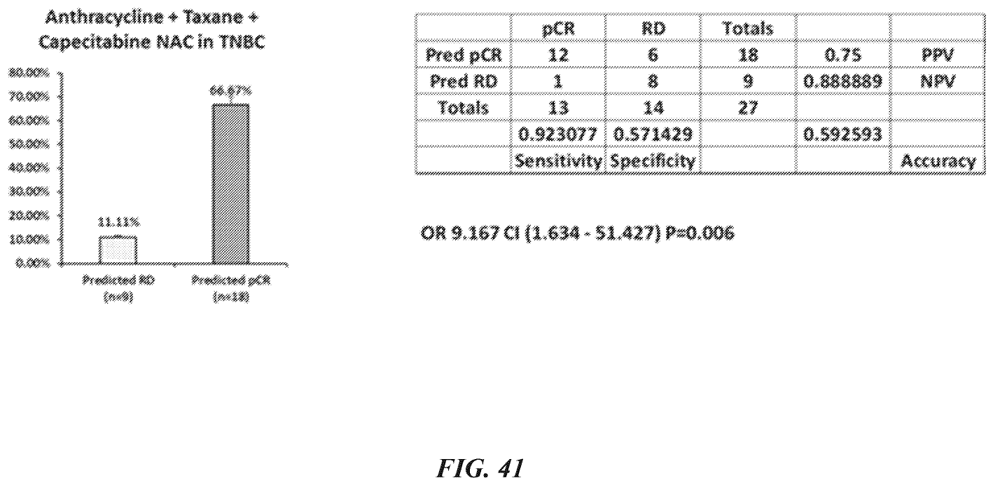

As shown in FIG. 41, FOXC1, Ki67, and PDL1 expression based complementary diagnostic predicts response to neoadjuvant anthracycline plus taxane plus capecitabine in subjects with triple negative breast cancer.

MK167 expression cutoff value was 70^{th} percentile of expression of MK167 mRNA on qRTPCR, 70^{th} percentile of expression of MK167 mRNA on RNA-Seq, or MK167 score on IHC of 3 using MIB1 antibody. FOXC1 expression cutoff value was 85^{th} percentile of expression of FOXC1 mRNA on qRTPCR, 85^{th} percentile of expression of FOXC1 mRNA on RNA-Seq, or FOXC1 Score of 6 using VERESCA® FOXC1 IHC kit. PDL1 (CD274) expression cutoff value was 15^{th} percentile of expression of PDL1 (CD274) mRNA on qRTPCR, 15^{th} percentile of expression of PDL1 (CD274) mRNA on RNA-Seq, or PDL1 score of 1 using PDL1 IHC kit.

MK167 less than the 70^{th} percentile (MK167<70^{th}% ILE) by mRNA EXPRESSION or <MK167 IHC Score of 3 predicts efficacy of alternative NAC regimen. MK167 expression greater than the 70^{th} percentile (MK167 70^{th}% ILE) mRNA EXPRESSION or MK167 IHC Score of 3 predicts efficacy of TX NAC regimen

FOXC1 expression less than the 85^{th} percentile (FOXC1<25TH % ILE) by mRNA EXPRESSION or VERESCA® FOXC1 IHC Score of 0 predicts efficacy of alternative NAC regimen. FOXC1 expression greater than the 85^{th} percentile (FOXC1 25^{th}% ILE) mRNA EXPRESSION or VERESCA® FOXC1 IHC Score of 2—Predicts efficacy of TX NAC regimen.

PDL1 expression less than the 15^{th} percentile (PDL1 (CD274)<15^{th}% ILE by mRNA EXPRESSION or <PDL1 IHC Score of 1 predicts efficacy of alternative NAC regimen. PDL1 expression greater than the 15^{th} percentile (PDL1 (CD274) 15^{th}% ILE) by mRNA EXPRESSION or ≥PDL1 IHC Score of 1 predicts efficacy of TX NAC regimen

From the foregoing, it will be appreciated that specific embodiments of the present technology have been described herein for purposes of illustration, but that various modifications may be made without deviating from the scope of the present technology. Accordingly, the present technology is not limited except as by the appended claims.

### Example 29: FOXC1 Expression as a Predictor of Therapeutic Response to Neoadjuvant Chemoradiation Therapy+ Atezolizumab in Esophageal Carcinoma

Methods: Tumor biopsy FOXC1 mRNA expression values were retrospectively obtained from patients with esophageal squamous cell carcinoma who had been enrolled and treated with chemoradiation therapy and atezolizumab in clinical trials and correlated with the rate of observed pathologic complete response (pCR). The area under the curve (AUC) of each model was calculated and used to determine suitable cutoff values to maximize Negative Predictive Value (NPV) and Sensitivity for pCR prediction.

Results: Predicted responders had a pCR rate of 76.92% vs 10.53% in predicted non-responders with residual disease (RD), (p=0.0004), sensitivity and sensitivity of 83.33% and 85% respectively (Odds Ratio 28.33 [4.022-199.597 Cl]p=0.0004).

### REFERENCES

Abou-Alfa, G. K., Sahai, V., Hollebecque, A., Vaccaro, G., Melisi, D., AI-Rajabi, R., et al. (2020). Pemigatinib for previously treated, locally advanced or metastatic cholangiocarcinoma: a multicentre, openlabel, phase 2 study. Lancet Oncol 21(5), 671-684. doi: 10.1016/S1470-2045(20)30109-1.
Aboulkheyr Es, H., Zhand, S., Thiery, J. P., and Warkiani, M. E. (2020). Pirfenidone reduces immunesuppressive capacity of cancer-associated fibroblasts through targeting CCL17 and TNF-beta. IntegrBiol (Camb) 12(7), 188-197. doi: 10.1093/intbio/zyaa014.
Andrae, B., Andersson, T. M., Lambert, P. C., Kemetli, L., Silfverdal, L., Strander, B., et al. (2012). Screening and cervical cancer cure: population based cohort study. BMJ 344, e900. doi: 10.1136/bmj.e900.
Aoki, K., Kurashige, M., Ichii, M., Higaki, K., Sugiyama, T., Kaito, T., et al. (2021). Identification of CXCL12-abundant reticular cells in human adult bone marrow. Br J Haematol 193(3), 659-668. doi:10.1111/bjh.17396.
Aras, S., and Zaidi, M. R. (2017). TAMeless traitors: macrophages in cancer progression and metastasis. BrJ Cancer 117(11), 1583-1591. doi: 10.1038/bjc.2017.356.
Baccelli, I., Schneeweiss, A., Riethdorf, S., Stenzinger, A., Schillert, A., Vogel, V., et al. (2013). Identification of a population of blood circulating tumor cells from breast cancer patients that initiates metastasis in a xenograft assay. Nat Biotechnol 31(6), 539-544. doi: 10.1038/nbt.2576.
Balko, J. M., Schwarz, L. J., Bhola, N.E., Kurupi, R., Owens, P., Miller, T. W., et al. (2013). Activation of MAPK pathways due to DUSP4 loss promotes cancer stem cell-like phenotypes in basal-like breast cancer. Cancer Res 73(20), 6346-6358. doi: 10.1158/0008-5472.CAN-13-1385.
Balkwill, F. R., Capasso, M., and Hagemann, T. (2012). The tumor microenvironment at a glance. J Cell Sci 125(Pt 23), 5591-5596. doi: 10.1242/jcs.116392.
Blonska, M., Zhu, Y., Chuang, H. H., You, M. J., Kunkalla, K., Vega, F., et al. (2015). Jun-regulated genes promote interaction of diffuse large B-cell lymphoma with the microenvironment. Blood 125(6), 981-991. doi: 10.1182/blood-2014-04-568188.
Bloushtain-Qimron, N., Yao, J., Snyder, E. L., Shipitsin, M., Campbell, L. L., Mani, S. A., et al. (2008). Cell type-specific DNA methylation patterns in the human breast. Proc Natl Acad Sci USA 105(37), 14076-14081. doi: 10.1073/pnas.0805206105.
Bohrer, L. R., and Schwertfeger, K. L. (2012). Macrophages promote fibroblast growth factor receptor-driven tumor cell migration and invasion in a CXCR2-dependent manner. Mol CancerRes 10(10), 1294-1305. doi: 10.1158/1541-7786.MCR-12-0275.
Burke, H. B. (2004). Outcome prediction and the future of the TNM staging system. J Natl Cancer Inst 96(19), 1408-1409. doi: 10.1093/jnci/djh293. Cancer Genome Atlas, N. (2012). Comprehensive molecular portraits of human breast tumours. Nature 490(7418), 61-70. doi: 10.1038/nature11412.
Cao, P. L., Wang, K., Wang, C. B., Li, X. F., Yang, Z., Yang, Z., et al. (2014). [Expressions of FOXC1 and MMP-7 in molecular subtypes of breast cancer and their association with clinicopathological characteristics]. Zhejiang Da Xue Bao YiXue Ban 43(4), 406-412.
Cao, Q., Wang, X., Shi, Y., Zhang, M., Yang, J., Dong, M., et al. (2019). FOXC1 silencing inhibits the epithelialtomesenchymal transition of glioma cells: Involvement of betacatenin signaling. Mol Med Rep 19(1), 251-261. doi: 10.3892/mmr.2018.9650.
Cao, S., Wang, Z., Gao, X., He, W., Cai, Y., Chen, H., et al. (2018). FOXC1 induces cancer stem cell-like properties through upregulation of beta-catenin in NSCLC. J Exp Clin Cancer Res 37(1), 220. doi: 10.1186/s13046-018-0894-0.
Chakrabarti, S., Kaur, K., Rao, K. N., Mandal, A. K., Kaur, I., Parikh, R. S., et al. (2009). The transcription factor gene FOXC1 exhibits a limited role in primary congenital glaucoma. Invest Ophthalmol Vis Sci 50(1), 75-83. doi: 10.1167/iovs.08-2253.
Charafe-Jauffret, E., Ginestier, C., lovino, F., Wicinski, J., Cervera, N., Finetti, P., et al. (2009). Breast cancer cell lines contain functional cancer stem cells with metastatic capacity and a distinct molecular signature. Cancer Res 69(4), 1302-1313. doi: 10.1158/0008-5472.CAN-08-2741.
Chaudary, N., Pintilie, M., Jelveh, S., Lindsay, P., Hill, R. P., and Milosevic, M. (2017). Plerixafor Improves Primary Tumor Response and Reduces Metastases in Cervical Cancer Treated with Radio-Chemotherapy. Clin Cancer Res 23(5), 1242-1249. doi: 10.1158/1078-0432.CCR-16-1730.
Chen, H., Xu, L., Shan, Z. L., Chen, S., and Hu, H. (2020). GPX8 is transcriptionally regulated by FOXC1 and promotes the growth of gastric cancer cells through activating the Wnt signaling pathway. Cancer Cell Int 20(1), 596. doi: 10.1186/s12935-020-01692-z.
Chen, S., Jiao, S., Jia, Y., and Li, Y. (2016). Effects of targeted silencing of FOXC1 gene on proliferation and in vitro migration of human non-small-cell lung carcinoma cells. Am J Trans/Res 8(8), 3309-3318.
Chung, S., Jin, Y., Han, B., Qu, Y., Gao, B., Giuliano, A. E., et al. (2017). Identification of EGF-NF-kappaBFOXC1 signaling axis in basal-like breast cancer. Cell Commun Signal 15(1), 22. doi: 10.1186/s12964-017-0180-3.
Chung, T. K., Lau, T. S., Cheung, T. H., Yim, S. F., Lo, K. W., Siu, N. S., et al. (2012). Dysregulation of microRNA-204 mediates migration and invasion of endometrial cancer by regulating FOXC1. Int J Cancer 130(5), 1036-1045. doi: 10.1002/ijc.26060.
Cortes, J. E., Heidel, F. H., Hellmann, A., Fiedler, W., Smith, B. D., Robak, T., et al. (2019). Randomized comparison of low dose cytarabine with or without glasdegib in patients with newly diagnosed acute myeloid leukemia or high-risk myelodysplastic syndrome. Leukemia 33(2), 379-389. doi: 10.1038/s41375-018-0312-9.
Dejeux, E., Ronneberg, J. A., Solvang, H., Bukholm, I., Geisler, S., Aas, T., et al. (2010). DNA methylation profiling in doxorubicin treated primary locally advanced breast tumours identifies novel genes associated with survival and treatment response. Mol Cancer 9, 68. doi: 10.1186/1476-4598-9-68.
Deng, L., Liu, T., Zhang, B., Wu, H., Zhao, J., and Chen, J. (2017). Forkhead box C1 is targeted by microRNA-133b and promotes cell proliferation and migration in osteosarcoma. Exp Ther Med 14(4), 2823-2830. doi: 10.3892/etm.2017.4870.
Didiasova, M., Singh, R., Wilhelm, J., Kwapiszewska, G., Wujak, L., Zakrzewicz, D., et al. (2017). Pirfenidone exerts antifibrotic effects through inhibition of GLI transcription factors. FASEB J 31(5), 1916-1928. doi: 10.1096/fj.201600892RR.
Du, R. F., Huang, H., Fan, L. L., Li, X. P., Xia, K., and Xiang, R. (2016). A Novel Mutation of FOXC1 (R127L) in an Axenfeld-Rieger Syndrome Family with Glaucoma and Multiple Congenital Heart Diseases. Ophthalmic Genet 37(1), 111-115. doi: 10.3109/13816810.2014.924016.
Dufies, M., Grytsai, O., Ronco, C., Camara, O., Ambrosetti, D., Hagege, A., et al. (2019). New CXCR1/CXCR2 inhibitors represent an effective treatment for kidney or head and neck cancers sensitive or refractory to reference treatments. Theranostics 9(18), 5332-5346. doi: 10.7150/thno.34681.
Eikawa, S., Ohue, Y., Kitaoka, K., Aji, T., Uenaka, A., Oka, M., et al. (2010). Enrichment of Foxp3+CD4 regulatory T cells in migrated T cells to IL-6—and IL-8-expressing tumors through predominant induction of CXCR1 by IL-6. J Immunol 185(11), 6734-6740. doi: 10.4049/jimmunol.1000225.
Elian, F. A., McMullen, T. P., Brindley, D. N., and Walter, M. A. (2018a). FOXC1 is Over-expressed and is More Stable in Triple Negative/Basal-Like Breast Cancer. FASEB J 32(S1), S648.646. doi: https://doi.org/10.1096/fasebj.2018.32.1_supplenent,648.6.
Elian, F. A., Yan, E., and Walter, M. A. (2018b). FOXC1, the new player in the cancer sandbox. Oncotarget 9(8), 8165-8178. doi: 10.18632/oncotarget.22742.
Emad, A., Ray, T., Jensen, T. W., Parat, M., Natrajan, R., Sinha, S., et al. (2020). Superior breast cancer metastasis risk stratification using an epithelial-mesenchymal-amoeboid transition gene signature. Breast Cancer Res 22(1), 74. doi: 10.1186/s13058-020-01304-8.
Eralp, Y., Derin, D., Ozluk, Y., Yavuz, E., Guney, N., Saip, P., et al. (2008). MAPK overexpression is associated with anthracycline resistance and increased risk for recurrence in patients with triple-negative breast cancer. Ann Oncol 19(4), 669-674. doi: 10.1093/annonc/mdm522.
Fernebro, J., Francis, P., Eden, P., Borg, A., Panagopoulos, I., Mertens, F., et al. (2006). Gene expression profiles relate to SS18/SSX fusion type in synovial sarcoma. Int J Cancer 118(5), 1165-1172. doi: 10.1002/ijc.21475.
Fujiwara, A., Funaki, S., Fukui, E., Kimura, K., Kanou, T., Ose, N., et al. (2020). Effects of pirfenidone targeting the tumor microenvironment and tumor-stroma interaction as a novel treatment for non-small cell lung cancer. Sci Rep 10(1), 10900. doi: 10.1038/s41598-020-67904-8.
Fukushima, N., Minami, Y., Kakiuchi, S., Kuwatsuka, Y., Hayakawa, F., Jamieson, C., et al. (2016). Small-molecule Hedgehog inhibitor attenuates the leukemia-initiation potential of acute myeloid leukemia cells. Cancer Sci 107(10), 1422-1429. doi: 10.1111/cas.13019.
Ganapathy, V., Ge, R., Grazioli, A., Xie, W., Banach-Petrosky, W., Kang, Y., et al. (2010). Targeting the Transforming Growth Factor-beta pathway inhibits human basal-like breast cancer metastasis. Mol Cancer 9, 122. doi: 10.1186/1476-4598-9-122.
Gao, B., Qu, Y., Han, B., Nagaoka, Y., Katsumata, M., Deng, N., et al. (2017a). Inhibition of lobuloalveolar development by FOXC1 overexpression in the mouse mammary gland. Sci Rep 7(1), 14017. doi: 10.1038/s41598-017-14342-8.
Gao, W., Wu, Y., He, X., Zhang, C., Zhu, M., Chen, B., et al. (2017b). MicroRNA-204-5p inhibits invasion and metastasis of laryngeal squamous cell carcinoma by suppressing forkhead box C1. J Cancer 8(12), 2356-2368. doi: 10.7150/jca.19470.
Ge, R., Rajeev, V., Ray, P., Lattime, E., Rittling, S., Medicherla, S., et al. (2006). Inhibition of growth and metastasis of mouse mammary carcinoma by selective inhibitor of transforming growth factor-beta type I receptor kinase in vivo. Clin Cancer Res 12(14 Pt 1), 4315-4330. doi: 10.1158/1078-0432.CCR-06-0162.
Gilding, L. N., and Somervaille, T. C. P. (2019). The Diverse Consequences of FOXC1 Deregulation in Cancer. Cancers (Basel) 11(2). doi: 10.3390/cancers11020184.
Giltnane, J. M., and Balko, J. M. (2014). Rationale for targeting the Ras/MAPK pathway in triple-negative breast cancer. Discov Med 17(95), 275-283.
Ginestier, C., Liu, S., Diebel, M. E., Korkaya, H., Luo, M., Brown, M., et al. (2010). CXCR1 blockade selectively targets human breast cancer stem cells in vitro and in xenografts. J Clin Invest 120(2), 485-497. doi: 10.1172/JC139397.
Goetz, R., and Mohammadi, M. (2013). Exploring mechanisms of FGF signalling through the lens of structural biology. Nat Rev Mol Cell Biol 14(3), 166-180. doi: 10.1038/nrm3528.
Goldstein, L. J., Perez, R.P., Yardley, D., Han, L. K., Reuben, J. M., Gao, H., et al. (2020). A window-of-opportunity trial of the CXCR1/2 inhibitor reparixin in operable HER-2-negative breast cancer. Breast Cancer Res 22(1), 4. doi: 10.1186/s13058-019-1243-8.
Gong, R., Lin, W., Gao, A., Liu, Y., Li, J., Sun, M., et al. (2019). Forkhead box C1 promotes metastasis and invasion of non-small cell lung cancer by binding directly to the lysyl oxidase promoter. Cancer Sci 110(12), 3663-3676. doi: 10.1111/cas.14213.
Gravina, G. L., Mancini, A., Muzi, P., Ventura, L., Biordi, L., Ricevuto, E., et al. (2015). CXCR4 pharmacogical inhibition reduces bone and soft tissue metastatic burden by affecting tumor growth and tumorigenic potential in prostate cancer preclinical models. Prostate 75(12), 1227-1246. doi: 10.1002/pros.23007.
Hagel, M., Miduturu, C., Sheets, M., Rubin, N., Weng, W., Stransky, N., et al. (2015). First Selective Small Molecule Inhibitor of FGFR4 for the Treatment of Hepatocellular Carcinomas with an Activated FGFR4 Signaling Pathway. Cancer Discov 5(4), 424-437. doi: 10.1158/2159-8290.CD-14-1029.
Han, B., Bhowmick, N., Qu, Y., Chung, S., Giuliano, A. E., and Cui, X. (2017). FOXC1: an emerging marker and therapeutic target for cancer. Oncogene 36(28), 3957-3963. doi: 10.1038/onc.2017.48.
Han, B., Qu, Y., Jin, Y., Yu, Y., Deng, N., Wawrowsky, K., et al. (2015). FOXC1 Activates Smoothened-Independent Hedgehog Signaling in Basal-like Breast Cancer. Cell Rep 13(5), 1046-1058. doi: 10.1016/j.celrep.2015.09.063.
Han, B., Zhou, B., Qu, Y., Gao, B., Xu, Y., Chung, S., et al. (2018). FOXC1-induced non-canonical WNT5AMMP7 signaling regulates invasiveness in triple-negative breast cancer. Oncogene 37(10), 1399-1408. doi: 10.1038/s41388-017-0021-2.
Hanahan, D., and Weinberg, R. A. (2011). Hallmarks of cancer: the next generation. Cell 144(5), 646-674. doi: 10.1016/j.cell.2011.02.013.
Hatlen, M. A., Schmidt-Kittler, O., Sherwin, C. A., Rozsahegyi, E., Rubin, N., Sheets, M. P., et al. (2019). Acquired On-Target Clinical Resistance Validates FGFR4 as a Driver of Hepatocellular Carcinoma. Cancer Discov 9(12), 1686-1695. doi: 10.1158/2159-8290.CD-19-0367.
Hatzivassiliou, G., Liu, B., O'Brien, C., Spoerke, J. M., Hoeflich, K. P., Haverty, P. M., et al. (2012). ERK inhibition overcomes acquired resistance to MEK inhibitors. Mol Cancer Ther 11(5), 1143-1154. doi: 10.1158/1535-7163.MCT-11-1010.
Hendrayani, S. F., AI-Harbi, B., AI-Ansari, M. M., Silva, G., and Aboussekhra, A. (2016). The inflammatory/cancer-related IL-6/STAT3/NF-kappaB positive feedback loop includes AUF1 and maintains the active state of breast myofibroblasts. Oncotarget 7(27), 41974-41985. doi: 10.18632/oncotarget.9633.
Hinohara, K., and Polyak, K. (2019). Intratumoral Heterogeneity: More Than Just Mutations. Trends Cell Biol 29(7), 569-579. doi: 10.1016/j.tcb.2019.03.003.
Hira, V. V. V., Van Noorden, C. J. F., and Molenaar, R.J. (2020). CXCR4 Antagonists as Stem Cell Mobilizers and Therapy Sensitizers for Acute Myeloid Leukemia and Glioblastoma?Biology (Basel) 9(2). doi: 10.3390/biology9020031.
Hirukawa, A., Smith, H. W., Zuo, D., Dufour, C. R., Savage, P., Bertos, N., et al. (2018). Targeting EZH2 reactivates a breast cancer subtype-specific anti-metastatic transcriptional program. Nat Commun 9(1), 2547. doi: 10.1038/s41467-018-04864-8.
Hoeflich, K. P., O'Brien, C., Boyd, Z., Cavet, G., Guerrero, S., Jung, K., et al. (2009). In vivo antitumor activity of MEK and phosphatidylinositol 3-kinase inhibitors in basal-like breast cancer models. Clin Cancer Res 15(14), 4649-4664. doi: 10.1158/1078-0432.CCR-09-0317.
Holdman, X. B., Welte, T., Rajapakshe, K., Pond, A., Coarfa, C., Mo, Q., et al. (2015). Upregulation of EGFR signaling is correlated with tumor stroma remodeling and tumor recurrence in FGFR1-driven breast cancer. Breast Cancer Res 17, 141. doi: 10.1186/s13058-015-0649-1.
Holmgaard, R. B., Schaer, D. A., Li, Y., Castaneda, S. P., Murphy, M. Y., Xu, X., et al. (2018). Targeting the TGFbeta pathway with galunisertib, a TGFbetaRl small molecule inhibitor, promotes anti-tumor immunity leading to durable, complete responses, as monotherapy and in combination with checkpoint blockade. J Immunother Cancer 6(1), 47. doi: 10.1186/s40425-018-0356-4.
Hopkins, A., Coatham, M. L., and Berry, F. B. (2017). FOXC1 Regulates FGFR1 Isoform Switching to Promote Invasion Following TGFbeta-Induced EMT. Mol Cancer Res 15(10), 1341-1353. doi: 10.1158/1541-7786.MCR-17-0185.
Hossain, M. A., Asa, T. A., Rahman, M. M., Uddin, S., Moustafa, A. A., Quinn, J. M. W., et al. (2020). Network-Based Genetic Profiling Reveals Cellular Pathway Differences Between Follicular Thyroid Carcinoma and Follicular Thyroid Adenoma. Int J Environ Res Public Health 17(4). doi: 10.3390/ijerph17041373.
Hsu, H. H., Kuo, W. W., Shih, H. N., Cheng, S. F., Yang, C. K., Chen, M. C., et al. (2019). FOXC1 Regulation of miR-31-5p Confers Oxaliplatin Resistance by Targeting LATS2 in Colorectal Cancer. Cancers (Basel) 11(10). doi: 10.3390/cancers 11101576.
Hu, G. D., Wang, C. X., Wang, H. Y., Wang, Y. Q., Hu, S., Cao, Z. W., et al. (2018). Long noncoding RNA CCAT2 functions as a competitive endogenous RNA to regulate FOXC1 expression by sponging miR-23b-5p in lung adenocarcinoma. J Cell Biochem. doi: 10.1002/jcb.28077.
Hu, M., and Polyak, K. (2008). Microenvironmental regulation of cancer development. Curr Opin Genet Dev 18(1), 27-34. doi: 10.1016/j.gde.2007.12.006.
Huang, H., Hu, J., Maryam, A., Huang, Q., Zhang, Y., Ramakrishnan, S., et al. (2021). Defining super-enhancer landscape in triple-negative breast cancer by multiomic profiling. Nat Commun 12(1), 2242. doi: 10.1038/s41467-021-22445-0.
Huang, H., Xiong, Y., Wu, Z., He, Y., Gao, X., Zhou, Z., et al. (2020). MIR-138-5P inhibits the progression of prostate cancer by targeting FOXC1. Mol Genet Genomic Med 8(4), el 193. doi: 10.1002/mgg3.1193.
Huang, J., Luo, Q., Xiao, Y., Li, H., Kong, L., and Ren, G. (2017a). The implication from RAS/RAF/ERK signaling pathway increased activation in epirubicin treated triple negative breast cancer. Oncotarget 8(64), 108249-108260. doi: 10.18632/oncotarget.22604.
Huang, L., Huang, Z., Fan, Y., He, L., Ye, M., Shi, K., et al. (2017b). FOXC1 promotes proliferation and epithelial-mesenchymal transition in cervical carcinoma through the PI3K-AKT signal pathway. Am J Trans/Res 9(3), 1297-1306.
Huang, W., Chen, Z., Zhang, L., Tian, D., Wang, D., Fan, D., et al. (2015). Interleukin-8 Induces Expression of FOXC1 to Promote Transactivation of CXCR1 and CCL2 in Hepatocellular Carcinoma Cell Lines and Formation of Metastases in Mice. Gastroenterology 149(4), 1053-1067 e1014. doi: 10.1053/j.gastro.2015.05.058.
Huang, Y., Huang, H., Li, M., Zhang, X., Liu, Y., and Wang, Y. (2017c). MicroRNA-374c-5p regulates the invasion and migration of cervical cancer by acting on the Foxc1/snail pathway. Biomed Pharmacother 94, 1038-1047. doi: 10.1016/j.biopha.2017.07.150.
Jensen, T. W., Ray, T., Wang, J., Li, X., Naritoku, W. Y., Han, B., et al. (2015). Diagnosis of Basal-Like Breast Cancer Using a FOXC1-Based Assay. J Natl Cancer Inst 107(8). doi: 10.1093/jnci/djv148.
Jiang, J., Li, J., Yao, W., Wang, W., Shi, B., Yuan, F., et al. (2021). FOXC1 Negatively Regulates DKK1 Expression to Promote Gastric Cancer Cell Proliferation Through Activation of Wnt Signaling Pathway. Front Cell Dev Biol 9, 662624. doi: 10.3389/fcell.2021.662624.
Jin, Y., Han, B., Chen, J., Wiedemeyer, R., Orsulic, S., Bose, S., et al. (2014). FOXC1 is a critical mediator of EGFR function in human basal-like breast cancer. Ann Surg Oncol 21 Suppl 4, S758-766. doi: 10.1245/s10434-014-3980-3.
Jing, P., Zou, J., Zhang, L., Wang, C., Yang, Y., Deng, L., et al. (2020). HOXB2 and FOXC1 synergistically drive the progression of Wilms tumor. Exp Mol Pathol 115, 104469. doi: 10.1016/j.yexmp.2020.104469.
Johnson, J., Choi, M., Dadmanesh, F., Han, B., Qu, Y., Yu-Rice, Y., et al. (2016). FOXC1 identifies basal-like breast cancer in a hereditary breast cancer cohort. Oncotarget 7(46), 75729-75738. doi: 10.18632/oncotarget.12370.
Jovanovic, B., Mayer, I. A., Mayer, E. L., Abramson, V. G., Bardia, A., Sanders, M. E., et al. (2017). A Randomized Phase II Neoadjuvant Study of Cisplatin, Paclitaxel With or Without Everolimus in Patients with Stage Il/IIl Triple-Negative Breast Cancer (TNBC): Responses and Long-term Outcome Correlated with Increased Frequency of DNA Damage Response Gene Mutations, TNBC Subtype, AR Status, and Ki67. Clin Cancer Res 23(15), 4035-4045. doi: 10.1158/1078-0432.CCR-16-3055.
Kallergi, G., Papadaki, M. A., Politaki, E., Mavroudis, D., Georgoulias, V., and Agelaki, S. (2011). Epithelial to mesenchymal transition markers expressed in circulating tumour cells of early and metastatic breast cancer patients. Breast Cancer Res 13(3), R59. doi: 10.1186/bcr2896.
Keup, C., Suryaprakash, V., Storbeck, M., Hoffmann, O., Kimmig, R., and Kasimir-Bauer, S. (2021). Longitudinal Multi-Parametric Liquid Biopsy Approach Identifies Unique Features of Circulating Tumor Cell, Extracellular Vesicle, and Cell-Free DNA Characterization for Disease Monitoring in Metastatic Breast Cancer Patients. Cells 10(2). doi: 10.3390/cells10020212.
Khalil, A., AI-Haddad, C., Hariri, H., Shibbani, K., Bitar, F., Kurban, M., et al. (2017). A Novel Mutation in FOXC1 in a Lebanese Family with Congenital Heart Disease and Anterior Segment Dysgenesis: Potential Roles for NFATC1 and DPT in the Phenotypic Variations. Front Cardiovasc Med 4, 58. doi: 10.3389/fcvm.2017.00058.
Khalilia, W. M. O., Gul; Karacam, Songul (2017). Gene Expression and Pathway Analysis of Radiation-Induced Apoptosis in C-4 I Cervical Cancer Cells. Journal of Autoimmunity and Research 4(1), 1014.
Khan, A. O., Aldahmesh, M. A., and AI-Amri, A. (2008). Heterozygous FOXC1 mutation (M161K) associated with congenital glaucoma and aniridia in an infant and a milder phenotype in her mother. Ophthalmic Genet 29(2), 67-71. doi: 10.1080/13816810801908152.
Kidson, S. H., Kume, T., Deng, K., Winfrey, V., and Hogan, B. L. (1999). The forkhead/winged-helix gene, Mf1, is necessary for the normal development of the cornea and formation of the anterior chamber in the mouse eye. Dev Biol 211(2), 306-322. doi: 10.1006/dbio.1999.9314.
Kim, R. D., Sarker, D., Meyer, T., Yau, T., Macarulla, T., Park, J. W., et al. (2019). First-in-Human Phase I Study of Fisogatinib (BLU-554) Validates Aberrant FGF19 Signaling as a Driver Event in Hepatocellular Carcinoma. Cancer Discov 9(12), 1696-1707. doi: 10.1158/2159-8290.CD-19-0555.
Kim, S., Moon, B. I., Lim, W., Park, S., Cho, M. S., and Sung, S. H. (2018). Feasibility of Classification of Triple Negative Breast Cancer by Immunohistochemical Surrogate Markers. Clin Breast Cancer 18(5), el 123-e1132. doi: 10.1016/j.clbc.2018.03.012.
King, T. E., Jr., Bradford, W. Z., Castro-Bernardini, S., Fagan, E. A., Glaspole, I., Glassberg, M. K., et al. (2014). A phase 3 trial of pirfenidone in patients with idiopathic pulmonary fibrosis. N Eng/J Med 370(22), 2083-2092. doi: 10.1056/NEJMoa1402582.
Klajic, J., Busato, F., Edvardsen, H., Touleimat, N., Fleischer, T., Bukholm, I., et al. (2014). DNA methylation status of key cell-cycle regulators such as CDKNA2/p16 and CCNA1 correlates with treatment response to doxorubicin and 5-fluorouracil in locally advanced breast tumors. Clin Cancer Res 20(24), 6357-6366. doi: 10.1158/1078-0432.CCR-14-0297.
Kong, X. P., Yao, J., Luo, W., Feng, F. K., Ma, J. T., Ren, Y. P., et al. (2014). The expression and functional role of a FOXC1 related mRNA-lncRNA pair in oral squamous cell carcinoma. Mol Cell Biochem 394(1-2), 177-186. doi: 10.1007/s 11010-014-2093-4.
Kornblum, N., Zhao, F., Manola, J., Klein, P., Ramaswamy, B., Brufsky, A., et al. (2018). Randomized Phase II Trial of Fulvestrant Plus Everolimus or Placebo in Postmenopausal Women With Hormone Receptor-Positive, Human Epidermal Growth Factor Receptor 2-Negative Metastatic Breast Cancer Resistant to Aromatase Inhibitor Therapy: Results of PrE0102. J Clin Oncol 36(16), 1556-1563. doi: 10.1200/JCO.2017.76.9331.
Kuhmann, C., Weichenhan, D., Rehli, M., Plass, C., Schmezer, P., and Popanda, O. (2011). DNA methylation changes in cells regrowing after fractioned ionizing radiation. RadiotherOncol 101(1), 116-121. doi: 10.1016/j.radonc.2011.05.048.
Kume, T., Deng, K., and Hogan, B. L. (2000). Murine forkhead/winged helix genes Foxc1 (Mf1) and Foxc2 (Mfh1) are required for the early organogenesis of the kidney and urinary tract. Development 127(7), 1387-1395.
Kume, T., Deng, K. Y., Winfrey, V., Gould, D. B., Walter, M. A., and Hogan, B. L. (1998). The forkhead/winged helix gene Mf1 is disrupted in the pleiotropic mouse mutation congenital hydrocephalus. Cel/93(6), 985-996. doi: 10.1016/s0092-8674(00)81204-0.
Kume, T., and Shackour, T. (2018). Meta-analysis of the likelihood of FOXC1 expression in early- and late-stage tumors. Oncotarget 9(93), 36625-36630. doi: 10.18632/oncotarget.26358.
Labrie, F., Candas, B., Dupont, A., Cusan, L., Gomez, J. L., Suburu, R. E., et al. (1999). Screening decreases prostate cancer death: first analysis of the 1988 Quebec prospective randomized controlled trial. Prostate 38(2), 83-91. doi: 10.1002/(sici)1097-0045(19990201)38:2<83:aid-pros1>3.0.co;2-b.
Lan, Y., Zhang, D., Xu, C., Hance, K. W., Marelli, B., Qi, J., et al. (2018). Enhanced preclinical antitumor activity of M7824, a bifunctional fusion protein simultaneously targeting PD-L1 and TGF-beta. Sci Transl Med 10(424). doi: 10.1 126/scitranslmed.aan5488.
Lay, K., Kume, T., and Fuchs, E. (2016). FOXC1 maintains the hair follicle stem cell niche and governs stem cell quiescence to preserve long-term tissue-regenerating potential. Proc Natl Acad Sci USA 113(11), E1506-1515. doi: 10.1073/pnas.1601569113.
Leontovich, A. A., Zhang, S., Quatraro, C., lankov, I., Veroux, P. F., Gambino, M. W., et al. (2012). Raf-1 oncogenic signaling is linked to activation of mesenchymal to epithelial transition pathway in metastatic breast cancer cells. Int J Oncol 40(6), 1858-1864. doi: 10.3892/ijo.2012.1407.
Li, C., Shen, W., Shen, S., and Ai, Z. (2013). Gene expression patterns combined with bioinformatics analysis identify genes associated with cholangiocarcinoma. Comput Biol Chem 47, 192-197. doi: 10.1016/j.compbiolchem.2013.08.010.
Li, C. S., Zhang, S. X., Liu, H. J., Shi, Y. L., Li, L.P., Guo, X. B., et al. (2014). Fibroblast growth factor receptor 4 as a potential prognostic and therapeutic marker in colorectal cancer. Biomarkers 19(1), 81-85. doi: 10.3109/1354750X.2013.876555.
Li, H., and Jiang, J. (2020). LncRNA MCM3AP-AS1 promotes proliferation, migration and invasion of oral squamous cell carcinoma cells via regulating miR-204-5p/FOXC1. J Investig Med 68(7), 1282-1288. doi: 10.1136/jim-2020-001415.
Li, J., Choi, P. S., Chaffer, C. L., Labella, K., Hwang, J. H., Giacomelli, A. O., et al. (2018). An alternative splicing switch in FLNB promotes the mesenchymal cell state in human breast cancer. Elife 7. doi: 10.7554/eLife.37184.
Li, Q., Wei, P., Wu, J., Zhang, M., Li, G., Li, Y., et al. (2019). The FOXC1/FBP1 signaling axis promotes colorectal cancer proliferation by enhancing the Warburg effect. Oncogene 38(4), 483-496. doi: 10.1038/s41388-018-0469-8.
Li, Y., and Chen, X. (2015). miR-4792 inhibits epithelial-mesenchymal transition and invasion in nasopharyngeal carcinoma by targeting FOXC1. Biochem Biophys Res Commun 468(4), 863-869. doi: 10.1016/j.bbrc.2015.11.045.
Lin, Y. J., Shyu, W.C., Chang, C. W., Wang, C. C., Wu, C. P., Lee, H. T., et al. (2017). Tumor Hypoxia Regulates Forkhead Box C1 to Promote Lung Cancer Progression. Theranostics 7(5), 1177-1191. doi: 10.7150/thno.17895.
Lin, Z., Huang, W., He, Q., Li, D., Wang, Z., Feng, Y., et al. (2021). FOXC1 promotes HCC proliferation and metastasis by Upregulating DNMT3B to induce DNA Hypermethylation of CTH promoter. J Exp Clin Cancer Res 40(1), 50. doi: 10.1186/s13046-021-01829-6.
Lin, Z., Sun, L., Chen, W., Liu, B., Wang, Y., Fan, S., et al. (2014). miR-639 regulates transforming growth factor beta-induced epithelial-mesenchymal transition in human tongue cancer cells by targeting FOXC1. Cancer Sci 105(10), 1288-1298. doi: 10.1111/cas.12499.
Liu, J., Shen, L., Yao, J., Li, Y., Wang, Y., Chen, H., et al. (2015). Forkhead box C1 promoter upstream transcript, a novel long non-coding RNA, regulates proliferation and migration in basal-like breast cancer. Mol Med Rep 11(4), 3155-3159. doi: 10.3892/mmr.2014.3089.
Liu, J., Zhang, Z., Li, X., Chen, J., Wang, G., Tian, Z., et al. (2018a). Forkhead box C1 promotes colorectal cancer metastasis through transactivating ITGA7 and FGFR4 expression. Oncogene 37(41), 5477-5491. doi: 10.1038/s41388-018-0355-4.
Liu, K., Ni, J. D., Li, W. Z., Pan, B. Q., Yang, Y. T., Xia, Q., et al. (2020). The Sp1/FOXC1/HOTTIP/LATS2/YAP/beta-catenin cascade promotes malignant and metastatic progression of osteosarcoma. Mol Oncol. doi: 10.1002/1878-0261.12760.
Liu, L., Ye, T. H., Han, Y. P., Song, H., Zhang, Y. K., Xia, Y., et al. (2014a). Reductions in myeloid-derived suppressor cells and lung metastases using AZD4547 treatment of a metastatic murine breast tumor model. Cell Physiol Biochem 33(3), 633-645. doi: 10.1159/000358640.
Liu, P., Tan, S., Xiao, S., Yi, S., Ouyang, L., Hou, Q., et al. (2014b). [Expression of FOXC1 and its relationship with E-cadherin in nasopharyngeal carcinoma tissues]. Lin Chung Er Bi Yan Hou Tou Jing Wai Ke Za Zhi 28(15), 1109-1112.
Liu, Y., Han, L., Bai, Y., Du, W., and Yang, B. (2018b). Down-regulation of MicroRNA-133 predicts poor overall survival and regulates the growth and invasive abilities in glioma. Artif Cells Nanomed Biotechnol 46(1), 206-210. doi: 10.1080/21691401.2017.1304551.
Liu, Z., Xu, S., Chu, H., Lu, Y., Yuan, P., and Zeng, X. (2018c). Silencing FOXC1 inhibits growth and migration of human oral squamous cell carcinoma cells. Exp Ther Med 16(4), 3369-3376. doi: 10.3892/etm.2018.6627.
Loi, S., Dushyanthen, S., Beavis, P. A., Salgado, R., Denkert, C., Savas, P., et al. (2016). RAS/MAPK Activation Is Associated with Reduced Tumor-Infiltrating Lymphocytes in Triple-Negative Breast Cancer: Therapeutic Cooperation Between MEK and PD-1/PD-L1 Immune Checkpoint Inhibitors. Clin CancerRes 22(6), 1499-1509. doi: 10.1158/1078-0432.CCR-15-1125.
Lopez-Gomez, M., Malmierca, E., de Gorgolas, M., and Casado, E. (2013). Cancer in developing countries: the next most preventable pandemic. The global problem of cancer. Crit Rev Oncol Hematol 88(1), 117-122. doi: 10.1016/j.critrevonc.2013.03.011.
Loriot, Y., Necchi, A., Park, S. H., Garcia-Donas, J., Huddart, R., Burgess, E., et al. (2019). Erdafitinib in Locally Advanced or Metastatic Urothelial Carcinoma. N Eng/J Med 381(4), 338-348. doi: 10.1056/NEJMoa1817323.
Mapp, T. J., Hardcastle, J. D., Moss, S. M., and Robinson, M. H. (1999). Survival of patients with colorectal cancer diagnosed in a randomized controlled trial of faecal occult blood screening. BrJ Surg 86(10), 1286-1291. doi: 10.1046/j.1365-2168.1999.01229.x.
Mariathasan, S., Turley, S. J., Nickles, D., Castiglioni, A., Yuen, K., Wang, Y., et al. (2018). TGF-beta attenuates tumour response to PD-L1 blockade by contributing to exclusion of T cells. Nature 554(7693), 544-548. doi: 10.1038/nature25501.
Mathis, R. A., Sokol, E. S., and Gupta, P. B. (2017). Cancer cells exhibit clonal diversity in phenotypic plasticity. Open Biol7(2). doi: 10.1098/rsob.160283.
Mattiske, D., Kume, T., and Hogan, B. L. (2006). The mouse forkhead gene Foxc1 is required for primordial germ cell migration and antral follicle development. Dev Biol 290(2), 447-458. doi: 10.1016/j.ydbio.2005.12.007.
Mears, A. J., Jordan, T., Mirzayans, F., Dubois, S., Kume, T., Parlee, M., et al. (1998). Mutations of the forkhead/winged-helix gene, FKHL7, in patients with Axenfeld-Rieger anomaly. Am J Hum Genet 63(5), 1316-1328. doi: 10.1086/302109.
Medina-Trillo, C., Aroca-Aguilar, J.D., Mendez-Hernandez, C.D., Morales, L., Garcia-Anton, M., Garcia-Feijoo, J., et al. (2016). Rare FOXC1 variants in congenital glaucoma: identification of translation regulatory sequences. Eur J Hum Genet 24(5), 672-680. doi: 10.1038/ejhg.2015.169.
Mellado, M., Rodriguez-Frade, J. M., Aragay, A., del Real, G., Martin, A.M., Vila-Coro, A. J., et al. (1998). The chemokine monocyte chemotactic protein 1 triggers Janus kinase 2 activation and tyrosine phosphorylation of the CCR2B receptor. J Immunol 161(2), 805-813.
Micheal, S., Siddiqui, S. N., Zafar, S. N., Villanueva-Mendoza, C., Cortes-Gonzalez, V., Khan, M. I., et al. (2016). A Novel Homozygous Mutation in FOXC1 Causes Axenfeld Rieger Syndrome with Congenital Glaucoma. PLoS One 11(7), e0160016. doi: 10.1371/journal.pone.0160016.
Mirzoeva, O. K., Das, D., Heiser, L. M., Bhattacharya, S., Siwak, D., Gendelman, R., et al. (2009). Basal subtype and MAPK/ERK kinase (MEK)-phosphoinositide 3-kinase feedback signaling determine susceptibility of breast cancer cells to MEK inhibition. Cancer Res 69(2), 565-572. doi: 10.1158/0008-5472.CAN-08-3389.
Nagaria, T. S., Shi, C., Leduc, C., Hoskin, V., Sikdar, S., Sangrar, W., et al. (2017). Combined targeting of Raf and Mek synergistically inhibits tumorigenesis in triple negative breast cancer model systems. Oncotarget 8(46), 80804-80819. doi: 10.18632/oncotarget.20534.
Nagel, S., Ehrentraut, S., Meyer, C., Kaufmann, M., Drexler, H. G., and MacLeod, R. A. (2015). Aberrantly Expressed OTX Homeobox Genes Deregulate B-Cell Differentiation in Hodgkin Lymphoma. PLoS One 10(9), e0138416. doi: 10.1371/journal.pone.0138416.
Nakano, T., Niimura, F., Hohenfellner, K., Miyakita, E., and Ichikawa, I. (2003). Screening for mutations in BMP4 and FOXC1 genes in congenital anomalies of the kidney and urinary tract in humans. Tokai J Exp Clin Med 28(3), 121-126.
Nam, J. S., Terabe, M., Mamura, M., Kang, M. J., Chae, H., Stuelten, C., et al. (2008). An anti-transforming growth factor beta antibody suppresses metastasis via cooperative effects on multiple cell compartments. Cancer Res 68(10), 3835-3843. doi: 10.1158/0008-5472.CAN-08-0215.
Nguyen, K., Yan, Y., Yuan, B., Dasgupta, A., Sun, J., Mu, H., et al. (2018). ST8SIA1 Regulates Tumor Growth and Metastasis in TNBC by Activating the FAK-AKT-mTOR Signaling Pathway. Mol Cancer Ther 17(12), 2689-2701. doi: 10.1158/1535-7163.MCT-18-0399.
Nichols, L., Saunders, R., and Knollmann, F. D. (2012). Causes of death of patients with lung cancer. Arch Pathol Lab Med 136(12), 1552-1557. doi: 10.5858/arpa.2011-0521-OA.
Nishimura, D. Y., Swiderski, R. E., Alward, W. L., Searby, C. C., Patil, S. R., Bennet, S. R., et al. (1998). The forkhead transcription factor gene FKHL7 is responsible for glaucoma phenotypes which map to 6p25. Nat Genet 19(2), 140-147. doi: 10.1038/493.
Noble, P. W., Albera, C., Bradford, W. Z., Costabel, U., Glassberg, M. K., Kardatzke, D., et al. (2011). Pirfenidone in patients with idiopathic pulmonary fibrosis (CAPACITY): two randomised trials. Lancet 377(9779), 1760-1769. doi: 10.1016/S0140-6736(11)60405-4.
Omatsu, Y., Seike, M., Sugiyama, T., Kume, T., and Nagasawa, T. (2014). Foxc1 is a critical regulator of haematopoietic stem/progenitor cell niche formation. Nature 508(7497), 536-540. doi: 10.1038/nature13071.
Ou-Yang, L., Xiao, S. J., Liu, P., Yi, S. J., Zhang, X. L., Ou-Yang, S., et al. (2015). Forkhead box C1 induces epithelialmesenchymal transition and is a potential therapeutic target in nasopharyngeal carcinoma. Mol Med Rep 12(6), 8003-8009. doi: 10.3892/mmr.2015.4427.
Pan, F., Yao, J., Chen, Y., Zhou, C., Geng, P., Mao, H., et al. (2014). A novel long non-coding RNA FOXCUT and mRNA FOXC1 pair promote progression and predict poor prognosis in esophageal squamous cell carcinoma. Int J Clin Exp Pathol 7(6), 2838-2849.
Pan, H., Peng, Z., Lin, J., Ren, X., Zhang, G., and Cui, Y. (2018). Forkhead box C1 boosts triple-negative breast cancer metastasis through activating the transcription of chemokine receptor-4. Cancer Sci 109(12), 3794-3804. doi: 10.1111/cas.13823.
Park, I. H., Kong, S. Y., Kwon, Y., Kim, M. K., Sim, S. H., Joo, J., et al. (2018). Phase 1/II clinical trial of everolimus combined with gemcitabine/cisplatin for metastatic triple-negative breast cancer. J Cancer 9(7), 1145-1151. doi: 10.7150/jca.24035.
Pastushenko, I., Brisebarre, A., Sifrim, A., Fioramonti, M., Revenco, T., Boumahdi, S., et al. (2018). Identification of the tumour transition states occurring during EMT. Nature 556(7702), 463-468. doi: 10.1038/s41586-018-0040-3.
Pelicano, H., Zhang, W., Liu, J., Hammoudi, N., Dai, J., Xu, R. H., et al. (2014). Mitochondrial dysfunction in some triple-negative breast cancer cell lines: role of mTOR pathway and therapeutic potential. Breast Cancer Res 16(5), 434. doi: 10.1186/s13058-014-0434-6.
Peraldo-Neia, C., Migliardi, G., Mello-Grand, M., Montemurro, F., Segir, R., Pignochino, Y., et al. (2011). Epidermal Growth Factor Receptor (EGFR) mutation analysis, gene expression profiling and EGFR protein expression in primary prostate cancer. BMC Cancer 11, 31. doi: 10.1186/1471-2407-11-31.
Petrocca, F., Altschuler, G., Tan, S. M., Mendillo, M. L., Yan, H., Jerry, D. J., et al. (2013). A genome-wide siRNA screen identifies proteasome addiction as a vulnerability of basal-like triple-negative breast cancer cells. Cancer Cell 24(2), 182-196. doi: 10.1016/j.ccr.2013.07.008.
Poorebrahim, M., Sadeghi, S., Ghanbarian, M., Kalhor, H., Mehrtash, A., and Teimoori-Toolabi, L. (2020). Identification of candidate genes and miRNAs for sensitizing resistant colorectal cancer cells to oxaliplatin and irinotecan. Cancer Chemother Pharmacol 85(1), 153-171. doi: 10.1007/s00280-019-03975-3.
Powell, A. A., Talasaz, A.H., Zhang, H., Coram, M. A., Reddy, A., Deng, G., et al. (2012). Single cell profiling of circulating tumor cells: transcriptional heterogeneity and diversity from breast cancer cell lines. PLoS One 7(5), e33788. doi: 10.1371/journal.pone.0033788.
Qin, W., Zou, J., Huang, Y., Liu, C., Kang, Y., Han, H., et al. (2020). Pirfenidone facilitates immune infiltration and enhances the antitumor efficacy of PD-L1 blockade in mice. Oncoimmunology9(1), 1824631. doi: 10.1080/2162402X.2020.1824631.
Qiu, S. Q., Waaijer, S. J. H., Zwager, M. C., de Vries, E. G. E., van der Vegt, B., and Schroder, C.P. (2018). Tumor-associated macrophages in breast cancer: Innocent bystander or important player?Cancer Treat Rev 70, 178-189. doi: 10.1016/j.ctrv.2018.08.010.
Qiu, Z. D., Zhongshu; Che, Xiaoming (2017). FoxC1 promotes osteosarcoma cell proliferation and metastasis through the activation of EZH2. Int J Clin Exp Med 10(1), 376-384.
Raninga, P. V., Lee, A., Sinha, D., Dong, L. F., Datta, K. K., Lu, X., et al. (2020). Marizomib suppresses triple-negative breast cancer via proteasome and oxidative phosphorylation inhibition. Theranostics 10(12), 5259-5275. doi: 10.7150/thno.42705.
Rashid, M., Shah, S. G., Verma, T., Chaudhary, N., Rauniyar, S., Patel, V. B., et al. (2021). Tumor-specific overexpression of histone gene, H3C14 in gastric cancer is mediated through EGFR-FOXC1 axis. Biochim Biophys Acta Gene Regul Mech 1864(4-5), 194703. doi: 10.1016/j.bbagrm.2021.194703.
Ray, P. S., Bagaria, S. P., Wang, J., Shamonki, J. M., Ye, X., Sim, M. S., et al. (2011). Basal-like breast cancer defined by FOXC1 expression offers superior prognostic value: a retrospective immunohistochemical study. Ann Surg Oncol 18(13), 3839-3847. doi: 10.1245/s10434-011-1657-8.
Ray, P. S., Jensen, T. W., Ray, T., Gholamalamdari, O., Tsai, C. Y., Kim, B., et al. (2015a). Abstract P1-07-30: FOXC1/FOXA1 transcriptional balance in breast cancer: From acquisition of mesenchymal and stem cell traits to occult lymph node independent breast cancer metastasis. Cancer Research 75(9 Supplement), P1-07-30. doi: 10.1158/1538-7445.SABCS14-P1-07-30.
Ray, P. S., Tsai, C. Y., Ray, T., Kim, B., and Jensen, T. W. (2015b). Abstract P2-06-04: Proteosome inhibitor bortezomib inhibits NFκB and effectively overcomes cancer stem cell escape triggered by Wnt inhibitor therapy in FOXC1+ basal-Like/claudin-low breast cancer. Cancer Research 75(9 Supplement), P2-06-04-P02-06-04. doi: 10.1158/1538-7445.Sabcs14-p2-06-04.
Ray, P. S., Wang, J., Qu, Y., Sim, M. S., Shamonki, J., Bagaria, S. P., et al. (2010). FOXC1 is a potential prognostic biomarker with functional significance in basal-like breast cancer. Cancer Res 70(10), 3870-3876. doi: 10.1158/0008-5472.CAN-09-4120.
Reed, J. R., Stone, M. D., Beadnell, T. C., Ryu, Y., Griffin, T. J., and Schwertfeger, K. L. (2012). Fibroblast growth factor receptor 1 activation in mammary tumor cells promotes macrophage recruitment in a CX3CL1-dependent manner. PLoS One 7(9), e45877. doi: 10.1371/journal.pone.0045877.
Rezende Miranda, R., Fu, Y., Chen, X., Perino, J., Cao, P., Carpten, J., et al. (2020). Development of a Potent and Specific FGFR4 Inhibitor for the Treatment of Hepatocellular Carcinoma. J Med Chem 63(20), 11484-11497. doi: 10.1021/acs.jmedchem.0c00044.
Rinnerthaler, G., Gampenrieder, S. P., Petzer, A., Burgstaller, S., Fuchs, D., Rossmann, D., et al. (2018). Ixazomib in combination with carboplatin in pretreated women with advanced triple-negative breast cancer, a phase I/II trial of the AGMT (AGMT MBC-10 trial). BMC Cancer 18(1), 1074. doi: 10.1186/s12885-018-4979-0.
Sabapathi, N., Sabarimurugan, S., Madurantakam Royam, M., Kumarasamy, C., Xu, X., Xu, G., et al. (2019). Prognostic Significance of FOXC1 in Various Cancers: A Systematic Review and Meta-Analysis. Mol Diagn Ther 23(6), 695-706. doi: 10.1007/s40291-019-00416-y.
Sadarangani, A., Pineda, G., Lennon, K. M., Chun, H.J., Shih, A., Schairer, A. E., et al. (2015). GLI2 inhibition abrogates human leukemia stem cell dormancy. J Trans/Med 13, 98. doi: 10.1186/s12967-015-0453-9.
Saini, K. S., Loi, S., de Azambuja, E., Metzger-Filho, O., Saini, M. L., Ignatiadis, M., et al. (2013). Targeting the PI3K/AKT/mTOR and Raf/MEK/ERK pathways in the treatment of breast cancer. Cancer Treat Rev 39(8), 935-946. doi: 10.1016/j.ctrv.2013.03.009.
Sanchez-Castro, M., Eldjouzi, H., Charpentier, E., Busson, P. F., Hauet, Q., Lindenbaum, P., et al. (2016). Search for Rare Copy-Number Variants in Congenital Heart Defects Identifies Novel Candidate Genes and a Potential Role for FOXC1 in Patients With Coarctation of the Aorta. Circ Cardiovasc Genet 9(1), 86-94. doi: 10.1161/CIRCGENETICS.115.001213.
Schraufstatter, I. U., Chung, J., and Burger, M. (2001). IL-8 activates endothelial cell CXCR1 and CXCR2 through Rho and Rac signaling pathways. Am J Physiol Lung Cell Mol Physiol 280(6), L1094-1103. doi: 10.1 152/ajplung.2001.280.6.L1094.
Schwab, K., Patterson, L. T., Aronow, B. J., Luckas, R., Liang, H. C., and Potter, S. S. (2003). A catalogue of gene expression in the developing kidney. Kidney Int 64(5), 1588-1604. doi: 10.1046/j.1523-1755.2003.00276.x.
Seo, S., and Kume, T. (2006). Forkhead transcription factors, Foxc1 and Foxc2, are required for the morphogenesis of the cardiac outflow tract. Dev Biol 296(2), 421-436. doi:10.1016/j.ydbio.2006.06.012.
Sheffield, B. S. (2016). Immunohistochemistry as a Practical Tool in Molecular Pathology. Arch Pathol Lab Med 140(8), 766-769. doi: 10.5858/arpa.2015-0453-RA.
Siegel, P. M., Shu, W., Cardiff, R. D., Muller, W. J., and Massague, J. (2003). Transforming growth factor beta signaling impairs Neu-induced mammary tumorigenesis while promoting pulmonary metastasis. Proc Nat!Acad Sci USA 100(14), 8430-8435. doi: 10.1073/pnas.0932636100.
Siggs, O. M., Souzeau, E., Pasutto, F., Dubowsky, A., Smith, J. E. H., Taranath, D., et al. (2019). Prevalence of FOXC1 Variants in Individuals With a Suspected Diagnosis of Primary Congenital Glaucoma. JAMA Ophthalmol 137(4), 348-355. doi: 10.1001/jamaophthalmol.2018.5646.
Singh, A., Gupta, A., Chowdhary, M., and Brahmbhatt, H. D. (2021). Integrated analysis of miRNA-mRNA networks reveals a strong anti-skin cancer signature in vitiligo epidermis. Exp Dermatol. doi: 10.1111/exd.14317.
Singh, J. K., Farnie, G., Bundred, N. J., Simoes, B. M., Shergill, A., Landberg, G., et al. (2013). Targeting CXCR1/2 significantly reduces breast cancer stem cell activity and increases the efficacy of inhibiting HER2 via HER2-dependent and -independent mechanisms. Clin Cancer Res 19(3), 643-656. doi: 10.1158/1078-0432.CCR-12-1063.
Sizemore, G. M., Sizemore, S. T., Pal, B., Booth, C. N., Seachrist, D. D., Abdul-Karim, F. W., et al. (2013). FOXC1 is enriched in the mammary luminal progenitor population, but is not necessary for mouse mammary ductal morphogenesis. Biol Reprod 89(1), 10. doi: 10.1095/biolreprod.113.108001.
Sizemore, S. T., and Keri, R. A. (2012). The forkhead box transcription factor FOXC1 promotes breast cancer invasion by inducing matrix metalloprotease 7 (MMP7) expression. J Biol Chem 287(29), 24631-24640. doi: 10.1074/jbc.M112.375865.
Somerville, T. D., Wiseman, D. H., Spencer, G. J., Huang, X., Lynch, J. T., Leong, H. S., et al. (2015). Frequent Derepression of the Mesenchymal Transcription Factor Gene FOXC1 in Acute Myeloid Leukemia. Cancer Cell 28(3), 329-342. doi: 10.1016/j.ccell.2015.07.017.
Son, B., Lee, S., Youn, H., Kim, E., Kim, W., and Youn, B. (2017). The role of tumor microenvironment in therapeutic resistance. Oncotarget 8(3), 3933-3945. doi: 10.18632/oncotarget.13907.
Su, D. N., Wu, S. P., Chen, H. T., and He, J. H. (2016). HOTAIR, a long non-coding RNA driver of malignancy whose expression is activated by FOXC1, negatively regulates miRNA-1 in hepatocellular carcinoma. Oncol Lett 12(5), 4061-4067. doi: 10.3892/01.2016.5127.
Su, S., Liu, Q., Chen, J., Chen, J., Chen, F., He, C., et al. (2014). A positive feedback loop between mesenchymal-like cancer cells and macrophages is essential to breast cancer metastasis. Cancer Cell25(5), 605-620. doi: 10.1016/j.ccr.2014.03.021.
Subramani, R., Camacho, F. A., Levin, C. I., Flores, K., Clift, A., Galvez, A., et al. (2018). FOXC1 plays a crucial role in the growth of pancreatic cancer. Oncogenesis 7(7), 52. doi: 10.1038/s41389-018-0061-7.
Sun, C.C., Zhu, W., Li, S. J., Hu, W., Zhang, J., Zhuo, Y., et al. (2020). FOXC1-mediated LINC00301 facilitates tumor progression and triggers an immune-suppressing microenvironment in non-small cell lung cancer by regulating the HIF1alpha pathway. Genome Med 12(1), 77. doi: 10.1186/s13073-020-00773-y.
Sun, H., Wu, P., Zhang, B., Wu, X., and Chen, W. (2021). MCM3AP-AS1 promotes cisplatin resistance in gastric cancer cells via the miR-138/FOXC1 axis. Oncol Lett 21(3), 211. doi: 10.3892/01.2021.12472.
Sung, H., Ferlay, J., Siegel, R. L., Laversanne, M., Soerjomataram, I., Jemal, A., et al. (2021). Global Cancer Statistics 2020: GLOBOCAN Estimates of Incidence and Mortality Worldwide for 36 Cancers in 185 Countries. CA Cancer J Clin 71(3), 209-249. doi: 10.3322/caac.21660.
Swaminathan, M., W, J. T., Tania, R., D, A. N., Anit, S., Egner, J. R., et al. (2016). ABSTRACT 617: FOXC1 Expression in Acute Myeloid Leukemia: Potential Predictor of Disease Relapse and/or Refractory Disease. Blood 128(22), 5260. doi: 10.1182/blood.V128.22.5260.5260.
Swanson, P.E. (2015). Immunohistochemistry as a surrogate for molecular testing: a review. App//mmunohistochem Mol Morphol 23(2), 81-96. doi: 10.1097/PAI.0000000000000181.
Swiderski, R. E., Reiter, R. S., Nishimura, D. Y., Alward, W. L., Kalenak, J. W., Searby, C. S., et al. (1999). Expression of the Mf1 gene in developing mouse hearts: implication in the development of human congenital heart defects. Dev Dyn 216(1), 16-27. doi: 10.1002/(SICI)1097-0177(199909)216:1<16:AID-DVDY4>3.0.CO;2-1.
Tabar, L., Fagerberg, C. J., Gad, A., Baldetorp, L., Holmberg, L. H., Grontoft, O., et al. (1985). Reduction in mortality from breast cancer after mass screening with mammography. Randomised trial from the Breast Cancer Screening Working Group of the Swedish National Board of Health and Welfare. Lancet 1(8433), 829-832. doi: 10.1016/s0140-6736(85)92204-4.
Tang, B., Vu, M., Booker, T., Santner, S. J., Miller, F. R., Anver, M. R., et al. (2003). TGF-beta switches from tumor suppressor to prometastatic factor in a model of breast cancer progression. J Clin Invest 112(7), 1116-1124. doi: 10.1172/JC118899.
Tang, L., Jin, J., Xu, K., Wang, X., Tang, J., and Guan, X. (2020). SOX9 interacts with FOXC1 to activate MYC and regulate CDK7 inhibitor sensitivity in triple-negative breast cancer. Oncogenesis 9(5), 47. doi: 10.1038/s41389-020-0232-1.
Tanwar, M., Kumar, M., Dada, T., Sihota, R., and Dada, R. (2010). MYOC and FOXC1 gene analysis in primary congenital glaucoma. Mol Vis 16, 1996-2006.
Tkocz, D., Crawford, N. T., Buckley, N. E., Berry, F. B., Kennedy, R.D., Gorski, J. J., et al. (2012). BRCA1 and GATA3 corepress FOXC1 to inhibit the pathogenesis of basal-like breast cancers. Oncogene 31(32), 3667-3678. doi: 10.1038/onc.2011.531.
Tosello, V., Bongiovanni, D., Liu, J., Pan, Q., Yan, K. K., Saccomani, V., et al. (2020). Cross-talk between GLI transcription factors and FOXC1 promotes T-cell acute lymphoblastic leukemia dissemination. Leukemia. doi: 10.1038/s41375-020-0999-2.
Tredan, O., Galmarini, C. M., Patel, K., and Tannock, I. F. (2007). Drug resistance and the solid tumor microenvironment. J Natl Cancer Inst 99(19), 1441-1454. doi: 10.1093/jnci/djm135.
Ueng, S. H., Chen, S. C., Chang, Y. S., Hsueh, S., Lin, Y. C., Chien, H. P., et al. (2012). Phosphorylated mTOR expression correlates with poor outcome in early-stage triple negative breast carcinomas. Int J Clin Exp Pathol 5(8), 806-813.
van der Heul-Nieuwenhuijsen, L., Dits, N. F., and Jenster, G. (2009). Gene expression of forkhead transcription factors in the normal and diseased human prostate. BJU Int 103(11), 1574-1580. doi: 10.1111/j.1464-410X.2009.08351.x.
Walsh, S., Flanagan, L., Quinn, C., Evoy, D., McDermott, E.W., Pierce, A., et al. (2012). mTOR in breast cancer: differential expression in triple-negative and non-triple-negative tumors. Breast 21(2), 178-182. doi: 10.1016/j.breast.2011.09.008.
Wang, D.S., Zhang, H. Q., Zhang, B., Yuan, Z. B., Yu, Z. K., Yang, T., et al. (2016a). miR-133 inhibits pituitary tumor cell migration and invasion via down-regulating FOXC1 expression. Genet Mol Res 15(1). doi: 10.4238/gmr.15017453.
Wang, J., Hu, W., Wang, K., Yu, J., Luo, B., Luo, G., et al. (2016b). Repertaxin, an inhibitor of the chemokine receptors CXCR1 and CXCR2, inhibits malignant behavior of human gastric cancer MKN45 cells in vitro and in vivo and enhances efficacy of 5-fluorouracil. Int J Oncol 48(4), 1341-1352. doi: 10.3892/ijo.2016.3371.
Wang, J., Li, L., Liu, S., Zhao, Y., Wang, L., and Du, G. (2016c). FOXC1 promotes melanoma by activating MST1 R/P13K/AKT. Oncotarget 7(51), 84375-84387. doi: 10.18632/oncotarget.11224.
Wang, J., Li, W., Zheng, X., Pang, X., and Du, G. (2018a). Research progress on the forkhead box C1. Oncotarget 9(15), 12471-12478. doi: 10.18632/oncotarget.22527.
Wang, J., Ray, P. S., Sim, M. S., Zhou, X. Z., Lu, K. P., Lee, A. V., et al. (2012). FOXC1 regulates the functions of human basal-like breast cancer cells by activating NF-kappaB signaling. Oncogene 31(45), 4798-4802. doi: 10.1038/onc.2011.635.
Wang, J., Xu, Y., Li, L., Wang, L., Yao, R., Sun, Q., et al. (2017a). FOXC1 is associated with estrogen receptor alpha and affects sensitivity of tamoxifen treatment in breast cancer. Cancer Med 6(1), 275-287. doi: 10.1002/cam4.990.
Wang, L., Chai, L., Ji, Q., Cheng, R., Wang, J., and Han, S. (2018b). Forkhead box protein C1 promotes cell proliferation and invasion in human cervical cancer. Mol Med Rep 17(3), 4392-4398. doi: 10.3892/mmr.2018.8423.
Wang, L., Gu, F., Liu, C. Y., Wang, R. J., Li, J., and Xu, J. Y. (2013). High level of FOXC1 expression is associated with poor prognosis in pancreatic ductal adenocarcinoma. TumourBiol 34(2), 853-858. doi: 10.1007/s13277-012-0617-7.
Wang, L., Siegenthaler, J. A., Dowell, R. D., and Yi, R. (2016d). Foxc1 reinforces quiescence in self-renewing hair follicle stem cells. Science 351(6273), 613-617. doi: 10.1126/science.aad5440.
Wang, L. Y., Li, L. S., and Yang, Z. (2016e). Correlation of FOXC1 protein with clinicopathological features in serous ovarian tumors. Oncol Lett 11(2), 933-938. doi: 10.3892/01.2015.3996.
Wang, S. J., Hung, H. M., and O'Neill, R. T. (2009). Adaptive patient enrichment designs in therapeutic trials. Biom J 51(2), 358-374. doi: 10.1002/bimj.200900003.
Wang, W. H., McNatt, L. G., Shepard, A. R., Jacobson, N., Nishimura, D. Y., Stone, E. M., et al. (2001). Optimal procedure for extracting RNA from human ocular tissues and expression profiling of the congenital glaucoma gene FOXC1 using quantitative RT-PCR. Mol Vis 7, 89-94.
Wang, W. W., Chen, B., Lei, C. B., Liu, G. X., Wang, Y. G., Yi, C., et al. (2017b). miR-582-5p inhibits invasion and migration of salivary adenoid cystic carcinoma cells by targeting FOXC1. Jpn J Clin Oncol 47(8), 690-698. doi: 10.1093/jjco/hyx073.
Wang, Y., Guo, X., Bray, M. J., Ding, Z., and Zhao, Z. (2016f). An integrative genomics approach for identifying novel functional consequences of PBRM1 truncated mutations in clear cell renal cell carcinoma (ccRCC). BMC Genomics 17 Suppl 7, 515. doi: 10.1186/s12864-016-2906-9.
Wang, Y., Zhang, T., Kwiatkowski, N., Abraham, B. J., Lee, T. I., Xie, S., et al. (2015). CDK7-dependent transcriptional addiction in triple-negative breast cancer. Cell 163(1), 174-186. doi: 10.1016/j.cell.2015.08.063.
Wei, L. X., Zhou, R. S., Xu, H. F., Wang, J. Y., and Yuan, M. H. (2013). High expression of FOXC1 is associated with poor clinical outcome in non-small cell lung cancer patients. Tumour Biol 34(2), 941-946. doi: 10.1007/s13277-012-0629-3.
Weinberger, P., Ponny, S.R., Xu, H., Bai, S., Smallridge, R., Copland, J., et al. (2017). Cell Cycle M-Phase Genes Are Highly Upregulated in Anaplastic Thyroid Carcinoma. Thyroid 27(2), 236-252. doi: 10.1089/thy.2016.0285.
Weiss, A., Adler, F., Buhles, A., Stamm, C., Fairhurst, R. A., Kiffe, M., et al. (2019). FGF401, A First-In-Class Highly Selective and Potent FGFR4 Inhibitor for the Treatment of FGF19-Driven Hepatocellular Cancer. Mol Cancer Ther 18(12), 2194-2206. doi: 10.1158/1535-7163.MCT-18-1291.
Wilm, B., James, R. G., Schultheiss, T. M., and Hogan, B. L. (2004). The forkhead genes, Foxc1 and Foxc2, regulate paraxial versus intermediate mesoderm cell fate. Dev Biol 271(1), 176-189. doi: 10.1016/j.ydbio.2004.03.034.
Wu, C. W., Mann, N., Nakayama, M., Connaughton, D. M., Dai, R., Kolvenbach, C. M., et al. (2020). Phenotype expansion of heterozygous FOXC1 pathogenic variants toward involvement of congenital anomalies of the kidneys and urinary tract (CAKUT). Genet Med 22(10), 1673-1681. doi: 10.1038/s41436-020-0844-z.
Wyckoff, J., Wang, W., Lin, E. Y., Wang, Y., Pixley, F., Stanley, E. R., et al. (2004). A paracrine loop between tumor cells and macrophages is required for tumor cell migration in mammary tumors. Cancer Res 64(19), 7022-7029. doi: 10.1158/0008-5472.CAN-04-1449.
Xia, L., Huang, W., Tian, D., Zhu, H., Qi, X., Chen, Z., et al. (2013). Overexpression of forkhead box C1 promotes tumor metastasis and indicates poor prognosis in hepatocellular carcinoma. Hepatology 57(2), 610-624. doi: 10.1002/hep.26029.
Xu, Y., Shao, Q. S., Yao, H. B., Jin, Y., Ma, Y. Y., and Jia, L. H. (2014). Overexpression of FOXC1 correlates with poor prognosis in gastric cancer patients. Histopathology 64(7), 963-970. doi: 10.1111/his.12347.
Xu, Y. L., Yao, R., Li, J., Zhou, Y. D., Mao, F., Pan, B., et al. (2017a). FOXC1 overexpression is a marker of poor response to anthracycline-based adjuvant chemotherapy in sporadic triple-negative breast cancer. Cancer Chemother Pharmacol 79(6), 1205-1213. doi: 10.1007/s00280-017-3319-4.
Xu, Y. Y., Tian, J., Hao, Q., and Yin, L. R. (2016). MicroRNA-495 downregulates FOXC1 expression to suppress cell growth and migration in endometrial cancer. Tumour Biol 37(1), 239-251. doi: 10.1007/s13277-015-3686-6.
Xu, Y. Z., Chen, F. F., Zhang, Y., Zhao, Q. F., Guan, X. L., Wang, H. Y., et al. (2017b). The long noncoding RNA FOXCUT promotes proliferation and migration by targeting FOXC1 in nasopharyngeal carcinoma. Tumour Biol 39(6), 1010428317706054. doi: 10.1177/1010428317706054.
Xu, Z. Y., Ding, S. M., Zhou, L., Xie, H. Y., Chen, K. J., Zhang, W., et al. (2012). FOXC1 contributes to microvascular invasion in primary hepatocellular carcinoma via regulating epithelial-mesenchymal transition. Int J Biol Sci 8(8), 1130-1141. doi: 10.7150/ijbs.4769.
Yang, Y., Yang, H. H., Tang, B., Wu, A. M. L., Flanders, K. C., Moshkovich, N., et al. (2020). The Outcome of TGF-beta Antagonism in Metastatic Breast Cancer Models In Vivo Reflects a Complex Balance between Tumor-Suppressive and Proprogression Activities of TGF-beta. Clin Cancer Res 26(3), 643-656. doi: 10.1158/1078-0432.CCR-19-2370.
Yang, Z., Jiang, S., Cheng, Y., Li, T., Hu, W., Ma, Z., et al. (2017). FOXC1 in cancer development and therapy: deciphering its emerging and divergent roles. Ther Adv Med Oncol 9(12), 797-816. doi: 10.1177/1758834017742576.
Yao, B., Xie, J., Liu, N., Hu, T., Song, W., Huang, S., et al. (2019). Direct reprogramming of epidermal cells toward sweat gland-like cells by defined factors. Cell Death Dis 10(4), 272. doi: 10.1038/s41419-019-1503-7.
Yao, T., Wang, Q., Zhang, W., Bian, A., and Zhang, J. (2016). Identification of genes associated with renal cell carcinoma using gene expression profiling analysis. Oncol Lett 12(1), 73-78. doi: 10.3892/ol.2016.4573.
Yu-Rice, Y., Jin, Y., Han, B., Qu, Y., Johnson, J., Watanabe, T., et al. (2016). FOXC1 is involved in ER-alpha silencing by counteracting GATA3 binding and is implicated in endocrine resistance. Oncogene 35(41), 5400-5411. doi: 10.1038/onc.2016.78.
Yu, C., Wang, M., Li, Z., Xiao, J., Peng, F., Guo, X., et al. (2015). MicroRNA-138-5p regulates pancreatic cancer cell growth through targeting FOXC1. Cell Oncol (Dordr) 38(3), 173-181. doi: 10.1007/s13402-014-0200-x.
Yu, M., Bardia, A., Wittner, B. S., Stott, S. L., Smas, M.E., Ting, D. T., et al. (2013). Circulating breast tumor cells exhibit dynamic changes in epithelial and mesenchymal composition. Science 339(6119), 580-584. doi: 10.1 126/science.1228522.
Yunokawa, M., Koizumi, F., Kitamura, Y., Katanasaka, Y., Okamoto, N., Kodaira, M., et al. (2012). Efficacy of everolimus, a novel mTOR inhibitor, against basal-like triple-negative breast cancer cells. Cancer Sci 103(9), 1665-1671. doi: 10.1111/j.1349-7006.2012.02359.x.
Zhang, D., Liu, X., Zhang, Q., and Chen, X. (2020a). miR-138-5p inhibits the malignant progression of prostate cancer by targeting FOXC1. Cancer Cell Int 20, 297. doi: 10.1186/s12935-020-01386-6.
Zhang, P., Long, Q., Zeng, S., Wen, M., and Lu, Q. (2020b). FOXC1-induced LINC01123 acts as a mediator in triple negative breast cancer. Cancer Cell lnt 20, 199. doi: 10.1186/s12935-020-01258-z.
Zhang, Y., Liao, Y., Chen, C., Sun, W., Sun, X., Liu, Y., et al. (2020c). p38-regulated FOXC1 stability is required for colorectal cancer metastasis. J Pathol 250(2), 217-230. doi: 10.1002/path.5362.
Zhao, H., Lv, F., Liang, G., Huang, X., Wu, G., Zhang, W., et al. (2016). FGF19 promotes epithelial-mesenchymal transition in hepatocellular carcinoma cells by modulating the GSK3beta/beta-catenin signaling cascade via FGFR4 activation. Oncotarget 7(12), 13575-13586. doi: 10.18632/oncotarget.6185.
Zhao, S., Ma, D., Xiao, Y., Li, X. M., Ma, J. L., Zhang, H., et al. (2020). Molecular Subtyping of Triple-Negative Breast Cancers by Immunohistochemistry: Molecular Basis and Clinical Relevance. Oncologist 25(10), e1481-e1491. doi: 10.1634/theoncologist.2019-0982.
Zhao, Z., Chen, J., and Xia, D. (2021). Knockdown of HCG18 Inhibits Cell Viability, Migration and Invasion in Pediatric Osteosarcoma by Targeting miR-188-5p/FOXC1 Axis. Mol Biotechnol. doi: 10.1007/s12033-021-00343-6.
Zheng, X. J., Li, W., Yi, J., Liu, J. Y., Ren, L. W., Zhu, X. M., et al. (2020). EZH2 regulates expression of FOXC1 by mediating H3K27me3 in breast cancers. Acta Pharmacol Sin. doi: 10.1038/s41401-020-00543-x.
Zhong, X., Yu, X., Wen, X., Chen, L., and Gu, N. (2020). Activation of the LINC00242/miR-141/FOXC1 axis underpins the development of gastric cancer. Cancer Cell Int 20, 272. doi: 10.1186/s12935-020-01369-7.
Zhu, X., Wei, L., Bai, Y., Wu, S., and Han, S. (2017). FoxC1 promotes epithelial-mesenchymal transition through PBX1 dependent transactivation of ZEB2 in esophageal cancer. Am J Cancer Res 7(8), 1642-1653.
Zuo, H. D., and Wu Yao, W. (2016). The role and the potential regulatory pathways of high expression of forkhead box C1 in promoting tumor growth and metastasis of basal-like breast cancer. J BUON 21(4), 818-825.

We claim:

1. A nucleotide sequence that encodes an antibody with (i) a VH region having at least 80% sequence identity to SEQ ID NO:18, or (ii) both (i) and a VL region having at least 80% sequence identity to SEQ ID NO:19, or SEQ ID NO:20.

2. The nucleotide sequence of claim 1, wherein the antibody is an antibody, an antibody fragment, an antibody conjugate, or an antibody fusion.

3. The nucleotide sequence of claim 2 or 3, wherein the antibody is a monoclonal antibody.

4. The nucleotide sequence of any one of claims 1 to 3, wherein the antibody is a humanized antibody, a chimeric antibody, or a human antibody.

5. The nucleotide sequence of any one of claims 1 to 4, wherein the antibody is an scFv

6. The nucleotide sequence of any one of claims 1 to 5, wherein the antibody is bispecific.

7. The nucleotide sequence of any one of claims 1 to 6, wherein the nucleotide sequence encodes a VH region having at least 85% sequence identity to SEQ ID NO:18; or (ii) both (i) and a VL region having at least 85% sequence identity to SEQ ID NO:19 or SEQ ID NO:20.

8. The nucleotide sequence of any one of claims 1 to 6, wherein the nucleotide sequence encodes a VH region having at least 90% sequence identity to SEQ ID NO:18; or (ii) both (i) and a VL region having at least 90% sequence identity to SEQ ID NO:19 or SEQ ID NO:20.

9. The nucleotide sequence of any one of claims 1 to 6, wherein the nucleotide sequence encodes a VH region having at least 95% sequence identity to SEQ ID NO:18; or (ii) both (i) and a VL region having at least 95% sequence identity to SEQ ID NO:19 or SEQ ID NO:20.

10. The nucleotide sequence of any one of claims 1 to 6, wherein the nucleotide sequence encodes a VH region having at least 99% sequence identity to SEQ ID NO:18; or (ii) both (i) and a VL region having at least 99% sequence identity to SEQ ID NO:19 or SEQ ID NO:20.

11. The nucleotide sequence of any one of claims 1 to 6, wherein the antibody comprises one or more of SEQ ID NO:1-17.

12. An anti-FOXC1 antibody comprising
(i) a variable heavy chain (VH) region comprising a first VH complementarity region (CDR) (VH CDR1) having an amino acid sequence comprising GFSITRDYA; a second VH CDR (VH CDR2) comprising INYSGTT; and a third VH CDR (VH CDR3) comprising VGWAVNYGLDY; or
(ii) both (i) and a variable light chain region comprising a first VL complementarity region (CDR) (VL CDR1) having an amino acid sequence comprising QSLLYSNGKTY or KSVSTSGYSY; a second VL CDR (VL CDR2) comprising LVS; and a third VL CDR (VL CDR3) comprising VQGTHFPHT or QHIRELTRSEGG.

13. The antibody of claim 12, wherein the antibody is an antibody, an antibody fragment, an antibody conjugate, or an antibody fusion.

14. The antibody of claim 11 or 12, wherein the antibody is a monoclonal antibody.

15. The antibody of any one of claims 12 to 14, wherein the antibody is a humanized antibody, a chimeric antibody, or a human antibody.

16. The antibody of any one of claims 12 to 15, wherein the antibody is an scFv

17. The antibody of any one of claims 12 to 16, wherein the antibody comprises one or more of SEQ ID NO:1-17.

18. A method for identifying an effective cancer therapy for a subject having cancer comprising:
(a) determining a level of FOXC1 protein or nucleic acid in a sample obtained from the subject;
(b) determining whether the level of FOXC1 protein or nucleic acid is present in the sample at a level above a predetermined cutoff value;
(c) predicting whether a cancer therapy will be clinically effective to reduce or treat the cancer in the subject based on the determining of step (b); and
(d) developing a treatment plan comprising (1) providing or continuing to provide the cancer therapy if the cancer therapy is determined to be the effective cancer therapy in (c), or (2) altering or stopping the cancer therapy if the cancer therapy is not determined to be the effective cancer therapy in (c).

19. A method for identifying an effective cancer therapy for a subject having cancer wherein the cancer therapy is an antihormonal therapy comprising:
(a) determining a level of FOXC1 protein or nucleic acid in a sample obtained from the subject;
(b) determining whether the level of FOXC1 protein or nucleic acid is present in the sample at a level above a predetermined cutoff value;
(c) predicting whether an antihormonal therapy will be clinically effective to reduce or treat the cancer in the subject based on the determining of step (b); and
(d) developing a treatment plan comprising (1) providing or continuing to provide the antihormonal therapy if the antihormonal therapy is determined to be the effective antihormonal therapy in (c), or (2) altering or stopping the antihormonal therapy if the antihormonal therapy is not determined to be the effective antihormonal therapy in (c).

20. A method for identifying an effective cancer therapy for a subject having cancer wherein the cancer therapy is a chemotherapy comprising:
(a) determining a level of FOXC1 protein or nucleic acid in a sample obtained from the subject;
(b) determining whether the level of FOXC1 protein or nucleic acid is present in the sample at a level above a predetermined cutoff value;
(c) predicting whether a chemotherapy will be clinically effective to reduce or treat the cancer in the subject based on the determining of step (b); and
(d) developing a treatment plan comprising (1) providing or continuing to provide the chemotherapy if the chemotherapy is determined to be the effective chemotherapy in (c), or (2) altering or stopping the chemotherapy if the chemotherapy is not determined to be the effective chemotherapy in (c).

21. A method for identifying an effective cancer therapy for a subject having cancer wherein the cancer therapy is an immunotherapy comprising:
(a) determining a level of FOXC1 protein or nucleic acid in a sample obtained from the subject;
(b) determining whether the level of FOXC1 protein or nucleic acid is present in the sample at a level above a predetermined cutoff value;
(c) predicting whether a immunotherapy will be clinically effective to reduce or treat the cancer in the subject based on the determining of step (b); and
(d) developing a treatment plan comprising (1) providing or continuing to provide the immunotherapy if the immunotherapy is determined to be the effective immunotherapy in (c), or (2) altering or stopping the immunotherapy if the immunotherapy is not determined to be the effective immunotherapy in (c).

22. A method for predicting a prognosis of a cancer in a subject treated with a cancer therapy, the method comprising:
(a) determining a level of FOXC1 protein by contacting in a sample obtained from the subject;
(b) determining whether the level of FOXC1 protein is present in the sample at a level above a predetermined cutoff value; and
(c) predicting whether the subject receiving the cancer therapy has a good prognosis based on the determining step (b), wherein the subject has good prognosis if the expression level of FOXC1 protein is higher than the predetermined cutoff value.

23. The method of any of claims 18 to 22, wherein determining the level of FOXC1 protein comprises providing the antibody of any of claims 12-17 to the tissue sample to detect FOXC1 protein.

24. The method of any one of claims 18 to 21, wherein determining the level of FOXC1 nucleic acid in the tissue sample comprises using a quantitative reverse transcriptase polymerase chain reaction to detect the level of FOXC1 nucleic acid in the tissue sample.

25. The method of any one of claims 18 to 21, wherein the therapy is clinically effective if treating the subject with the therapy results in a clinicopathologic outcome compared to before the cancer therapy was administered, wherein the clinicopathologic outcomes are selected from the group consisting of: (i) a decrease in tumor size or tumor number of the cancer in the subject, (ii) a decrease in cancer cell proliferation in the subject (iii) a decrease in cancer cell invasion or migration in the subject (iv) a decrease in incidence of recurrence of the cancer in the subject, or (v) a decrease in incidence of metastatic spread of the cancer in the subject.

26. The method of any of claims 18 to 21, wherein clinical efficacy is measured as one or more clinicopathologic outcomes in the subject compared to the clinicopathologic outcome in a control subject, wherein the clinicopathologic outcomes are selected from the group consisting of: (i) a decrease in tumor size or tumor number of the cancer in the subject, (ii) a decrease in cancer cell proliferation in the subject (iii) a decrease in cancer cell invasion, migration in the subject (iv) a decrease in incidence of recurrence of the cancer in the subject, (v) a decrease in incidence of metastatic spread of the cancer in the subject (vi) an increase or prolongation of disease-free survival, (vii) an increase or prolongation of recurrence-free survival, (viii) an increase/prolongation of distant metastasis-free survival, (ix) an increase or prolongation of event-free survival, (x) an increase or prolongation of progression-free survival, (xi) an increase or prolongation of disease-specific survival, (xii) an increase or prolongation of overall survival.

27. The method claim 22, wherein good prognosis is measured as one or more clinicopathologic outcomes in the subject compared to the clinicopathologic outcome in a control subject, wherein the clinicopathologic outcomes are selected from the group consisting of: (i) a decrease in tumor size or tumor number of the cancer in the subject, (ii) a decrease in cancer cell proliferation in the subject (iii) a decrease in cancer cell invasion, migration in the subject (iv) a decrease in incidence of recurrence of the cancer in the subject, (v) a decrease in incidence of metastatic spread of the cancer in the subject (vi) an increase or prolongation of disease-free survival, (vii) an increase or prolongation of recurrence-free survival, (viii) an increase/prolongation of distant metastasis-free survival, (ix) an increase or prolongation of event-free survival, (x) an increase or prolongation of progression-free survival, (xi) an increase or prolongation of disease-specific survival, (xii) an increase or prolongation of overall survival.

28. The method of claim 26 or 27, wherein FOXC1 is not detected in the control subject or is detected at a level below the preselected cutoff value.

29. The method of any one of claims 18 to 22, wherein continuing to provide the therapy comprises continuing to administer the same dose of the therapy at the same dose.

30. The method of any one of claims 18 to 22 or 29, wherein continuing to provide the therapy comprises continuing to administer the therapy with the same dosing regimen.

31. The method of any one of claims 18 to 22, wherein altering the therapy comprises reducing or increasing the dose of the therapy.

32. The method of any one of claims 18 to 22, wherein altering the therapy comprises changing the therapy to a different agent.

33. The method of any one of claims 18 to 22, wherein altering the therapy comprises adding an additional therapeutic agent.

31. The methods of any one of claims 18 to 21 further comprising administering the therapy to the subject according to the treatment plan in step (e).

32. The method of any one of claims 18 to 31, wherein the predetermined cutoff value is determined by:
(i) identifying the level of FOXC1 protein or nucleic acid in a plurality of retrospective subjects that were diagnosed with the cancer and were given the cancer therapy to treat the cancer;
(ii) identifying the rate of observed pathologic complete response (pCR) for the plurality of retrospective subjects;
(iii) correlating the level of FOXC1 protein or nucleic acid with the rate of observed pCR; and
(iv) determining the predetermined cutoff value by calculating a Negative Predictive Value (NPV) and sensitivity for pCR prediction based on the predetermined cutoff value, wherein the predetermined cutoff value is selected to maximize the NPV and sensitivity for pCR.

33. The method of any one of claims 18 to 33, wherein the cancer is breast cancer, lung cancer, or colon cancer.

34. The method of any one of claims 18 to 33, wherein the cancer therapy is an agent that inhibits, blocks, or attenuates TGF-β signaling.

35. The method of claim 34, wherein the cancer therapy is galunisertib or pirfenidone.

36. The method of any one of claims 18 to 33, wherein the cancer therapy is an agent that inhibits, blocks, or attenuates the nuclear factor kappa B (NFκB) signaling pathway.

37. The method of claim 36, wherein the cancer therapy is bortezomib, carfilzomib, or Ixazomib.

38. The method of any one of claims 18 to 33, wherein the cancer therapy is an agent that inhibits, blocks, or attenuates the P13K, PTEN, AKT, or mTOR signaling pathways.

39. The method of claim 38, wherein the cancer therapy is an mTOR inhibitor.

40. The method of claim 38, wherein the cancer therapy is ipatasertib, capivasertib, everolimus, or temsirolimus.

41. The method of any one of claims 18 to 33, wherein the cancer therapy is an agent that inhibits, blocks, or attenuates the Wnt signaling pathway.

42. The method of any one of claims 18 to 33, wherein the cancer therapy is an agent that inhibits, blocks, or attenuates the non-canonical hedgehog signaling pathway.

43. The method of claim 42, wherein the cancer therapy is a GLI2 inhibitor.

44. The method of claim 42, wherein the cancer therapy is glasdegib or pirfenidone.

45. The method of any one of claims 18 to 33, wherein the cancer therapy is an agent that inhibits, blocks, or attenuates the non-canonical Notch signaling pathway.

46. The method of any one of claims 18 to 33, wherein the cancer therapy is an agent that inhibits or blocks the PD1/PDL1 immune checkpoint.

47. The method of claim 46, wherein the cancer therapy is a PD1 inhibitor.

48. The method of claim 46, wherein the cancer therapy is a PDL1 inhibitor.

49. The method of claim 46, wherein the cancer therapy is Nivolumab, Pembrolizumab, Cemiplimab, Atezolizumab, Avelumab, or Durvalumab.

50. The method of any one of claims 18 to 33, wherein the cancer therapy is an agent that inhibits or blocks the CTLA4 immune checkpoint.

51. The method of claim 50, wherein the cancer therapy is ipilimumab.

52. The method of any one of claims 18 to 33, wherein the cancer therapy is an agent that inhibits or blocks the CDK4/6 class of cyclin-dependent kinase enzymes.

53. The method of claim 52, wherein the cancer therapy is Palbociclib, ribociclib, or abemaciclib.

54. The method of any one of claims 18 to 33, wherein the cancer therapy is an agent that inhibits or blocks the epidermal growth factor receptor (EGFR).

55. The method of claim 54, wherein the cancer therapy is gefitinib.

56. The method of any one of claims 18 to 33, wherein the cancer therapy is an agent that inhibits or blocks the RAS, RAF, MEK, or ERK kinase enzymes.

57. The method of claim 56, wherein the cancer therapy is a RAS, RAF, MEK, or ERK inhibitor.

58. The method of claim 56, wherein the cancer therapy is a sotorasib, sorafenib, vemurafenib, trametinib, binimetinib, or ulixertinib.

59. The method of any one of claims 18 to 33, wherein the cancer therapy is an agent that inhibits, blocks, or attenuates the IL8 or CXCR1 pathway.

60. The method of claim 59, wherein the cancer therapy is an IL8 or CXCR1 inhibitor.

61. The method of claim 60, wherein the cancer therapy is Humax-IL8 (BMS-986253) or Repertaxi.

62. The method of any one of claims 18 to 33, wherein the cancer therapy is an agent that inhibits, blocks, or attenuates the CCL12/CXCR4 pathway.

63. The method of claim 62, wherein the cancer therapy is a CXCR4 inhibitor.

64. The method of claim 62, wherein the cancer therapy is plerixafor.

65. The method of any one of claims 18 to 33, wherein the cancer therapy is an agent that inhibits, blocks, or attenuates the FGF/FGFR1 pathway.

66. The method of claim 65, wherein the cancer therapy is a FGFR1 inhibitor.

67. The method of claim 65, wherein the cancer therapy is pemigatinib or erdafitinib.

68. The method of any one of claims 18 to 33, wherein the cancer therapy is an agent that inhibits the FGF19 or FGFR4 pathway.

69. The method of claim 68, wherein the cancer therapy is a FGFR4 inhibitor.

70. The method of claim 68, wherein the cancer therapy is BLU9931, BLU554/Fisogatinib, FG401/Roblitinib.

71. The method of claim 18 or 19, wherein the cancer therapy is tamoxifen, toremifene, fulvestrant, an aromatase inhibitor, ovarian suppression, or a luteinizing hormone-releasing hormone (LHRH) agonist.

72. The method of claim 18 or 20, wherein the cancer therapy is paclitaxel, Olaparib, or cisplatin.

73. The method of claim 18 or 21, wherein the cancer therapy is durvalumab, ipilimumab, pembrolizumab, nivolumab, atezolizumab, Interleukin-2, or Interferon-alpha.

74. The method of any one of claims 18 to 22, wherein step (b) further comprises determining the level of K167 protein or nucleic acid in the tissue sample and step (c) further comprises determining whether the level of K167 is present at a level above a predetermined cutoff value, wherein the cancer therapy has elevated clinical efficacy if the level of FOXC1 is present at a level above a predetermined cutoff value and the level of K167 is present above a second predetermined cutoff value.

75. The method of claim 74, wherein the second predetermined cutoff value is determined by:
(i) identifying the level of K167 protein or nucleic acid in a plurality of retrospective subjects that were diagnosed with the cancer and were given the cancer therapy to treat the cancer;
(ii) identifying the rate of observed pathologic complete response (pCR) for the plurality of retrospective subjects;
(iii) correlating the level of K167 protein or nucleic acid with the rate of observed pCR; and
(iv) determining the predetermined cutoff value by calculating a Negative Predictive Value (NPV) and sensitivity for pCR prediction based on the predetermined cutoff value, wherein the predetermined cutoff value is selected to maximize the NPV and sensitivity for pCR.

76. The method of claim 74 or 75, wherein the cancer therapy is a neoadjuvant regimen comprising a PARP inhibitor, a Taxane chemotherapy, or an PDL1 immune checkpoint inhibitor.

77. The method of any one of claims 74 to 76, wherein the cancer is a triple-negative breast cancer or a HER2 negative ER+ cancer.

78. The method of any one of claims 74 to 76, wherein the cancer is a bladder cancer.

79. The method of any one of claims 74 to 76, wherein the cancer therapy is neoadjuvant taxane and platinum.

80. The method of claim 79, wherein the cancer is a triple-negative breast cancer.

81. The method of claim 74 or 75, wherein step (b) further comprises determining the level of PDL1 protein or nucleic acid in the tissue sample and step (c) further comprises determining whether the level of PDL1 is present at a level above a predetermined cutoff value, wherein the cancer therapy has elevated clinical efficacy if the level of FOXC1 is present at a level above a predetermined cutoff value, the level of K167 is present above a second predetermined cutoff value, and the level of PDL1 is present above a third predetermined cutoff value.

82. The method of claim 81, wherein the third predetermined cutoff value is determined by:
(i) identifying the level of PDL1 protein or nucleic acid in a plurality of retrospective subjects that were diagnosed with the cancer and were given the cancer therapy to treat the cancer;
(ii) identifying the rate of observed pathologic complete response (pCR) for the plurality of retrospective subjects;
(iii) correlating the level of PDL1 protein or nucleic acid with the rate of observed pCR; and
(iv) determining the predetermined cutoff value by calculating a Negative Predictive Value (NPV) and sensitivity for pCR prediction based on the predetermined cutoff value, wherein the predetermined cutoff value is selected to maximize the NPV and sensitivity for pCR.

83. The method of claim 81 or 82, wherein the cancer therapy is a neoadjuvant immune checkpoint inhibitor.

84. The method of any one of claims 18 to 23 or 25 to 83, wherein determining the level of FOXC1 protein in the tissue sample comprises performing immunohistochemistry (IHC) on the tissue sample using the antibody of any one of claims 12-17.

85. The method of claim 84, wherein determining whether the level of FOXC1 protein or nucleic acid is present at a level above a predetermined cutoff value comprises identifying the presence of FOXC1 in the tissue sample.

86. The method of claim 84, wherein determining the level of FOXC1 protein in the tissue sample further comprises assigning an IHC score based on the level of FOXC1 protein in the tissue sample.

87. The method of claim 86, wherein the predetermined cutoff value is an IHC score of 1 or more.

88. The method of claim 24, wherein the FOXC1 nucleic acid comprises a nucleic acid having a sequence of SEQ ID NO: 44.

89. The method of any of claims 18 to 88, wherein the sample comprises a cancer cell or a tumor.

90. The method of claim 18 or 20, wherein the cancer therapy is adjuvant chemotherapy and the cancer is ER+ breast cancer.

91. The method of claim 18 or 21, wherein the cancer therapy is neoadjuvant immunotherapy and the cancer is triple negative breast cancer of ER+ breast cancer.

92. The method of claim 18 or 21, wherein the cancer therapy is adjuvant immunotherapy or salvage immunotherapy and the cancer is Non-small cell lung cancer (NSCLC), melanoma, Head and Neck Squamous cell carcinoma (HNSCC), or Renal Cell Carcinoma.

93. The method of claim 18 or 21, wherein the cancer therapy is adjuvant or salvage nivolumab plus pembrolizumab and the cancer is advanced or metastatic melanoma.

94. The method of claim 18 or 21, wherein the cancer therapy is an immune checkpoint inhibitor the cancer is advanced or metastatic melanoma.

95. The method of claim 18 or 21, wherein the cancer therapy is neoadjuvant chemoradiation therapy plus atezolizumab and the cancer is esophageal carcinoma.
